SEQUENCE LISTING (260 sequences)

SEQ ID NO: 1 - DNA, 1011 bp, synthetic construct
  misc_feature 1..1011: note = KRED-NADH-102 variant
  source 1..1011: mol_type = other DNA
atgtctatcccgtcttctcagtatggtttcgtatttaacaaacagtctggtctgaaactgcgcaacgatctgccagtacacaagccgaaagctggtcaactgctgctgaaagtagatgcagtaggtctgtgccactctgatctgcacgttatctacgaaggcctggactgcggtgacaactacgttatgggtcacgaaatcgctggtaccgttgctgctgttggtgatgacgttatcaactacaaagtaggtgaccgtgtagcttgtgttggtccgaacggttgcggtggttgcaaatactgccgtggtgctatcgacaacgtttgcaaaaacgctttcggtgactggttcggtctgggttatgatggtggctatcaacaatacctgctggtaactcgcccgcgtaacctgtctcgtatcccggataacgtatctgctgatgttgctgctgcttctaccgacgctgttctgactccgtaccacgctatcaaaatggctcaggtttctccgacctctaacatcctgctgatcggtgctggtggtctgggtggtaacgctatccaggttgctaaagcattcggtgctaaagttaccgttctggacaaaaaaaaagaagctcgtgaccaggctaaaaaactgggtgctgacgctgtttacgaaaccctgccggaatctatctctccgggttctttctctgcttgcttcgacttcgtttctgttcaggctaccttcgacgtttgccagaaatacgttgaaccgaaaggtgttatcatgccggttggtctgggtgctccgaacctgtcttttaacctgggtgacctggctctgcgtgaaatccgtatcctgggttctttctggggtaccaccaacgacctggacgacgttctgaaactggtttctgaaggtaaagttaaaccggttgtacgttctgctaaactgaaagaactgccggaatacatcgaaaaactgcgtaacaacgcttacgaaggtcgtgttgttttcaacccgtaa

SEQ ID NO: 2 - protein, 336 aa, synthetic construct
  REGION 1..336: note = KRED-NADH-102 variant
MSIPSSQYGFVFNKQSGLKLRNDLPVHKPKAGQLLLKVDAVGLCHSDLHVIYEGLDCGDNYVMGHEIAGTVAAVGDDVINYKVGDRVACVGPNGCGGCKYCRGAIDNVCKNAFGDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQAKKLGADAVYETLPESISPGSFSACFDFVSVQATFDVCQKYVEPKGVIMPVGLGAPNLSFNLGDLALREIRILGSFWGTTNDLDDVLKLVSEGKVKPVVRSAKLKELPEYIEKLRNNAYEGRVVFNP

SEQ ID NO: 3 - DNA, 1011 bp, synthetic construct
  misc_feature 1..1011: note = KRED-NADH-102 variant
  source 1..1011: mol_type = other DNA
atgtctatcccgtcttctcagtatggtttcgtatttaacaaacagtctggtctgaaactgcgcaacgatctgccagtacacaagccgaaagctggtcaactgctgctgaaagtagatgcagtaggtctgtgccactctgatctgcacgttatctacgaaggcctggactgcggtgacaactacgttatgggtcacgaaatcgctggtaccgttgctgctgttggtgatgacgttatcaactacaaagtaggtgaccgtgtagcttgtgttggtccgaacggttgcggtggttgcaaatactgccgtggtgctatcgacaacgtttgcaaaaacgctttcggtgactggttcggtctgggttatgatggtggctatcaacaatacctgctggtaactcgcccgcgtaacctgtctcgtatcccggataacgtatctgctgatgttgctgctgcttctaccgacgctgttctgactccgtaccacgctatcaaaatggctcaggtttctccgacctctaacatcctgctgatcggtgctggtggtctgggtggtaacgctatccaggttgctaaagcattcggtgctaaagttaccgttctggacaaaaaaaaagaagctcgtgaccaggctaaaaaactgggtgctgacgctgtttacgaaaccctgccggaatctatctctccgggttctttctctgcttgcttcgacttcgtttctgttcaggctaccttcgacgtttgccagaaatacgttgaaccgaaaggtgttatcatgccggttggtctgggtgctccgaacctgtcttttaacctgggtgacctggctctgcgtgaaatccgtatcctgggttctttctggggtaccaccaacgacctggacgacgttctgaaactggtttctgaaggtaaagttaaaccggttgtattttctgctaaactgaaagaactgccggaatacatcgaaaaactgcgtaacaacgcttacgaaggtcgtgttgttttcaacccgtaa

SEQ ID NO: 4 - protein, 336 aa, synthetic construct
  REGION 1..336: note = KRED-NADH-102 variant
MSIPSSQYGFVFNKQSGLKLRNDLPVHKPKAGQLLLKVDAVGLCHSDLHVIYEGLDCGDNYVMGHEIAGTVAAVGDDVINYKVGDRVACVGPNGCGGCKYCRGAIDNVCKNAFGDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQAKKLGADAVYETLPESISPGSFSACFDFVSVQATFDVCQKYVEPKGVIMPVGLGAPNLSFNLGDLALREIRILGSFWGTTNDLDDVLKLVSEGKVKPVVFSAKLKELPEYIEKLRNNAYEGRVVFNP

SEQ ID NO: 5 - DNA, 1011 bp, synthetic construct
  misc_feature 1..1011: note = KRED-NADH-102 variant
  source 1..1011: mol_type = other DNA
atgtctatcccgtcttctcagtatggtttcgtatttaacaaacagtctggtctgaaactgcgcaacgatctgccagtacacaagccgaaagctggtcaactgctgctgaaagtagatgcagtaggtctgtgccactctgatctgcacgttatctacgaaggcctggacttgggtgacaactacgttatgggtcacgaaatcgctggtaccgttgctgctgttggtgatgacgttatcaactacaaagtaggtgaccgtgtagcttgtgttggtccgaacggttgcggtggttgcaaatactgccgtggtgctatcgacaacgtttgcaaaaacgctttcggtgactggttcggtctgggttatgatggtggctatcaacaatacctgctggtaactcgcccgcgtaacctgtctcgtatcccggataacgtatctgctgatgttgctgctgcttctaccgacgctgttctgactccgtaccacgctatcaaaatggctcaggtttctccgacctctaacatcctgctgatcggtgctggtggtctgggtggtaacgctatccaggttgctaaagcattcggtgctaaagttaccgttctggacaaaaaaaaagaagctcgtgaccaggctaaaaaactgggtgctgacgctgtttacgaaaccctgccggaatctatctctccgggttctttctctgcttgcttcgacttcgtttctgttcaggctaccttcgacgtttgccagaaatacgttgaaccgaaaggtgttatcatgccggttggtctgggtgctccgaacctgtcttttaacctgggtgacctggctctgcgtgaaatccgtatcctgggttctttctggggtaccaccaacgacctggacgacgttctgaaactggtttctgaaggtaaagttaaaccggttgtacgttctgctaaactgaaagaactgccggaatacatcgaaaaactgcgtaacaacgcttacgaaggtcgtgttgttttcaacccgtaa

SEQ ID NO: 6 - protein, 336 aa, synthetic construct
  REGION 1..336: note = KRED-NADH-102 variant
MSIPSSQYGFVFNKQSGLKLRNDLPVHKPKAGQLLLKVDAVGLCHSDLHVIYEGLDLGDNYVMGHEIAGTVAAVGDDVINYKVGDRVACVGPNGCGGCKYCRGAIDNVCKNAFGDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQAKKLGADAVYETLPESISPGSFSACFDFVSVQATFDVCQKYVEPKGVIMPVGLGAPNLSFNLGDLALREIRILGSFWGTTNDLDDVLKLVSEGKVKPVVRSAKLKELPEYIEKLRNNAYEGRVVFNP

SEQ ID NO: 7 - DNA, 1011 bp, synthetic construct
  misc_feature 1..1011: note = KRED-NADH-102 variant
  source 1..1011: mol_type = other DNA
atgtctatcccgtcttctcagtatggtttcgtatttaacaaacagtctggtctgaaactgcgcaacgatctgccagtacacaagccgaaagctggtcaactgctgctgaaagtagatgcagtaggtctgtgccactctgatctgcacgttatctacgaaggcctggactgcggtgacaactacgttatgggtcacgaaatcgctggtaccgttgctgctgttggtgatgacgttatcaactacaaagtaggtgaccgtgtagcttgtgttggtccgaacggttgcggtggttgcaaatactgccgtggtgctatcgacaacgtttgcaaaaacgctttcaaagactggttcggtctgggttatgatggtggctatcaacaatacctgctggtaactcgcccgcgtaacctgtctcgtatcccggataacgtatctgctgatgttgctgctgcttctaccgacgctgttctgactccgtaccacgctatcaaaatggctcaggtttctccgacctctaacatcctgctgatcggtgctggtggtctgggtggtaacgctatccaggttgctaaagcattcggtgctaaagttaccgttctggacaaaaaaaaagaagctcgtgaccaggctaaaaaactgggtgctgacgctgtttacgaaaccctgccggaatctatctctccgggttctttctctgcttgcttcgacttcgtttctgttcaggctaccttcgacgtttgccagaaatacgttgaaccgaaaggtgttatcatgccggttggtctgggtgctccgaacctgtcttttaacctgggtgacctggctctgcgtgaaatccgtatcctgggttctttctggggtaccaccaacgacctggacgacgttctgaaactggtttctgaaggtaaagttaaaccggttgtacgttctgctaaactgaaagaactgccggaatacatcgaaaaactgcgtaacaacgcttacgaaggtcgtgttgttttcaacccgtaa

SEQ ID NO: 8 - protein, 336 aa, synthetic construct
  REGION 1..336: note = KRED-NADH-102 variant
MSIPSSQYGFVFNKQSGLKLRNDLPVHKPKAGQLLLKVDAVGLCHSDLHVIYEGLDCGDNYVMGHEIAGTVAAVGDDVINYKVGDRVACVGPNGCGGCKYCRGAIDNVCKNAFKDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQAKKLGADAVYETLPESISPGSFSACFDFVSVQATFDVCQKYVEPKGVIMPVGLGAPNLSFNLGDLALREIRILGSFWGTTNDLDDVLKLVSEGKVKPVVRSAKLKELPEYIEKLRNNAYEGRVVFNP

SEQ ID NO: 9 - DNA, 1011 bp, synthetic construct
  misc_feature 1..1011: note = KRED-NADH-102 variant
  source 1..1011: mol_type = other DNA
atgtctatcccgtcttctcagtatggtttcgtatttaacaaacagtctggtctgaaactgcgcaacgatctgccagtacacaagccgaaagctggtcaactgctgctgaaagtagatgcagtaggtctgtgccactctgatctgcacgttatctacgaaggcctggactgcggtgacaactacgttatgggtcacgaaatcgctggtaccgttgctgctgttggtgatgacgttatcaactacaaagtaggtgaccgtgtagcttgtgttggtccgaacggttgcggtggttgcaaatactgccgtggtgctatcgacaacgtttgcaaaaacgctttcggtgactggttcggtctgggttatgatggtggctatcaacaatacctgctggtaactcgcccgcgtaacctgtctcgtatcccggataacgtatctgctgatgttgctgctgcttctaccgacgctgttctgactccgtaccacgctatcaaaatggctcaggtttctccgacctctaacatcctgctgatcggtgctggtggtctgggtggtaacgctatccaggttgctaaagcattcggtgctaaagttaccgttctggacaaaaaaaaagaagctcgtgaccaggctaaaaaactgggtgctgacgctgtttacgaaaccctgccggaatctatctctccgggttctttctctgcttgcttcgacttcgtttctgttcaggctaccttcgacgtttgccagaaatacgttgaaccgaaaggtgttatcatgccggttggtctgggtgctccgaacctgtcttttaacctggttgacctggctctgcgtgaaatccgtatcctgggttctttctggggtaccaccaacgacctggacgacgttctgaaactggtttctgaaggtaaagttaaaccggttgtacgttctgctaaactgaaagaactgccggaatacatcgaaaaactgcgtaacaacgcttacgaaggtcgtgttgttttcaacccgtaa

SEQ ID NO: 10 - protein, 336 aa, synthetic construct
  REGION 1..336: note = KRED-NADH-102 variant
MSIPSSQYGFVFNKQSGLKLRNDLPVHKPKAGQLLLKVDAVGLCHSDLHVIYEGLDCGDNYVMGHEIAGTVAAVGDDVINYKVGDRVACVGPNGCGGCKYCRGAIDNVCKNAFGDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQAKKLGADAVYETLPESISPGSFSACFDFVSVQATFDVCQKYVEPKGVIMPVGLGAPNLSFNLVDLALREIRILGSFWGTTNDLDDVLKLVSEGKVKPVVRSAKLKELPEYIEKLRNNAYEGRVVFNP

SEQ ID NO: 11 - DNA, 1011 bp, synthetic construct
  misc_feature 1..1011: note = KRED-NADH-102 variant
  source 1..1011: mol_type = other DNA
atgtctatcccgtcttctcagtatggtttcgtatttaacaaacagtctggtctgaaactgcgcaacgatctgccagtacacaagccgaaagctggtcaactgctgctgaaagtagatgcagtaggtctgtgccactctgatctgcacgttatctacgaaggcctggactgcggtgacaactacgttatgggtcacgaaatcgctggtaccgttgctgctgttggtgatgacgttatcaactacaaagtaggtgaccgtgtagcttgtgttggtccgaacggttgcggtggttgcaaatactgccgtggtgctatcgacaacgtttgcaaaaacgctttcggtgactggttcggtctgggttatgatggtggctatcaacaatacctgctggtaactcgcccgcgtaacctgtctcgtatcccggataacgtatctgctgatgttgctgctgcttctaccgacgctgttctgactccgtaccacgctatcaaaatggctcaggtttctccgacctctaacatcctgctgatcggtgctggtggtctgggtggtaacgctatccaggttgctaaagcattcggtgctaaagttaccgttctggacaaaaaaaaagaagctcgtgaccaggctaaaaaactgggtgctgacgctgtttacgaaaccctgccggaatctatctctccgggttctttctctgcttgcttcgacttcgtttctgttcaggctaccttcgacgtttgccagaaatacgttgaaccgaaaggtgttatcatgccggttggtctgtatgctccgaacctgtcttttaacctgggtgacctggctctgcgtgaaatccgtatcctgggttctttctggggtaccaccaacgacctggacgacgttctgaaactggtttctgaaggtaaagttaaaccggttgtacgttctgctaaactgaaagaactgccggaatacatcgaaaaactgcgtaacaacgcttacgaaggtcgtgttgttttcaacccgtaa

SEQ ID NO: 12 - protein, 336 aa, synthetic construct
  REGION 1..336: note = KRED-NADH-102 variant
MSIPSSQYGFVFNKQSGLKLRNDLPVHKPKAGQLLLKVDAVGLCHSDLHVIYEGLDCGDNYVMGHEIAGTVAAVGDDVINYKVGDRVACVGPNGCGGCKYCRGAIDNVCKNAFGDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQAKKLGADAVYETLPESISPGSFSACFDFVSVQATFDVCQKYVEPKGVIMPVGLYAPNLSFNLGDLALREIRILGSFWGTTNDLDDVLKLVSEGKVKPVVRSAKLKELPEYIEKLRNNAYEGRVVFNP

SEQ ID NO: 13 - DNA, 1011 bp, synthetic construct
  misc_feature 1..1011: note = KRED-NADH-102 variant
  source 1..1011: mol_type = other DNA
atgtctatcccgtcttctcagtatggtttcgtatttaacaaacagtctggtctgaaactgcgcaacgatctgccagtacacaagccgaaagctggtcaactgctgctgaaagtagatgcagtaggtctgtgccactctgatctgcacgttatctacgaaggcctggactgcggtgacaactacgttatgggtcacgaaatcgctggtaccgttgctgctgttggtgatgacgttatcaactacaaagtaggtgaccgtgtagcttgtgttggtccgaacggttgcggtggttgcaaatactgccgtggtgctatcgacaacgtttgcaaaaacgctttcggtgactggttcggtctgggttatgatggtggctatcaacaatacctgctggtaactcgcccgcgtaacctgtctcgtatcccggataacgtatctgctgatgttgctgctgcttctaccgacgctgttctgactccgtaccacgctatcaaaatggctcaggtttctccgacctctaacatcctgctgatcggtgctggtggtctgggtggtaacgctatccaggttgctaaagcattcggtgctaaagttaccgttctggacaaaaaaaaagaagctcgtgaccaggctaaaaaactgggtgctgacgctgtttacgaaaccctgccggaatctatctctccgggttctttctctgcttgcttcgacttcgtttctgttcaggctaccttcgacgtttgccagaaatacgttgaaccgaaaggtgttatcatgccggttggtctgggtgctccgaacctgtcttttaacctgggtgacctggcttttcgtgaaatccgtatcctgggttctttctggggtaccaccaacgacctggacgacgttctgaaactggtttctgaaggtaaagttaaaccggttgtacgttctgctaaactgaaagaactgccggaatacatcgaaaaactgcgtaacaacgcttacgaaggtcgtgttgttttcaacccgtaa

SEQ ID NO: 14 - protein, 336 aa, synthetic construct
  REGION 1..336: note = KRED-NADH-102 variant
MSIPSSQYGFVFNKQSGLKLRNDLPVHKPKAGQLLLKVDAVGLCHSDLHVIYEGLDCGDNYVMGHEIAGTVAAVGDDVINYKVGDRVACVGPNGCGGCKYCRGAIDNVCKNAFGDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQAKKLGADAVYETLPESISPGSFSACFDFVSVQATFDVCQKYVEPKGVIMPVGLGAPNLSFNLGDLAFREIRILGSFWGTTNDLDDVLKLVSEGKVKPVVRSAKLKELPEYIEKLRNNAYEGRVVFNP

SEQ ID NO: 15 - DNA, 1011 bp, synthetic construct
  misc_feature 1..1011: note = KRED-NADH-102 variant
  source 1..1011: mol_type = other DNA
atgtctatcccgtcttctcagtatggtttcgtatttaacaaacagtctggtctgaaactgcgcaacgatctgccagtacacaagccgaaagctggtcaactgctgctgaaagtagatgcagtaggtctgtgccactctgatctgcacgttatctacgaaggcctggacattggtgacaactacgttatgggtcacgaaatcgctggtaccgttgctgctgttggtgatgacgttatcaactacaaagtaggtgaccgtgtagcttgtgttggtccgaacggttgcggtggttgcaaatactgccgtggtgctatcgacaacgtttgcaaaaacgctttcggtgactggttcggtctgggttatgatggtggctatcaacaatacctgctggtaactcgcccgcgtaacctgtctcgtatcccggataacgtatctgctgatgttgctgctgcttctaccgacgctgttctgactccgtaccacgctatcaaaatggctcaggtttctccgacctctaacatcctgctgatcggtgctggtggtctgggtggtaacgctatccaggttgctaaagcattcggtgctaaagttaccgttctggacaaaaaaaaagaagctcgtgaccaggctaaaaaactgggtgctgacgctgtttacgaaaccctgccggaatctatctctccgggttctttctctgcttgcttcgacttcgtttctgttcaggctaccttcgacgtttgccagaaatacgttgaaccgaaaggtgttatcatgccggttggtctgggtgctccgaacctgtcttttaacctgggtgacctggctctgcgtgaaatccgtatcctgggttctttctggggtaccaccaacgacctggacgacgttctgaaactggtttctgaaggtaaagttaaaccggttgtacgttctgctaaactgaaagaactgccggaatacatcgaaaaactgcgtaacaacgcttacgaaggtcgtgttgttttcaacccgtaa

SEQ ID NO: 16 - protein, 336 aa, synthetic construct
  REGION 1..336: note = KRED-NADH-102 variant
MSIPSSQYGFVFNKQSGLKLRNDLPVHKPKAGQLLLKVDAVGLCHSDLHVIYEGLDIGDNYVMGHEIAGTVAAVGDDVINYKVGDRVACVGPNGCGGCKYCRGAIDNVCKNAFGDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQAKKLGADAVYETLPESISPGSFSACFDFVSVQATFDVCQKYVEPKGVIMPVGLGAPNLSFNLGDLALREIRILGSFWGTTNDLDDVLKLVSEGKVKPVVRSAKLKELPEYIEKLRNNAYEGRVVFNP

SEQ ID NO: 17 - DNA, 1011 bp, synthetic construct
  misc_feature 1..1011: note = KRED-NADH-102 variant
  source 1..1011: mol_type = other DNA
atgtctatcccgtcttctcagtatggtttcgtatttaacaaacagtctggtctgaaactgcgcaacgatctgccagtacacaagccgaaagctggtcaactgctgctgaaagtagatgcagtaggtctgtgccactctgatctgcacgttatctacgaaggcctggactgcggtgacaactacgttatgggtcacgaaatcgctggtaccgttgctgctgttggtgatgacgttatcaactacaaagtaggtgaccgtgtagcttgtgttggtccgaacggttgcggtggttgcaaatactgccgtggtgctatcgacaacgtttgcaaaaacgctttcggtgactggttcggtctgggttatgatggtggctatcaacaatacctgctggtaactcgcccgcgtaacctgtctcgtatcccggataacgtatctgctgatgttgctgctgcttctaccgacgctgttctgactccgtaccacgctatcaaaatggctcaggtttctccgacctctaacatcctgctgatcggtgctggtggtctgggtggtaacgctatccaggttgctaaagcattcggtgctaaagttaccgttctggacaaaaaaaaagaagctcgtgaccaggctaaaaaactgggtgctgacgctgtttacgaaaccctgccggaatctatctctccgggttctttctctgcttgcttcgacttcgtttctgttcaggctaccttcgacgtttgccagaaatacgttgaaccgaaaggtgttatcatgccggttggtctgggtgctccgaacctgtcttttaacctgccagacctggctctgcgtgaaatccgtatcctgggttctttctggggtaccaccaacgacctggacgacgttctgaaactggtttctgaaggtaaagttaaaccggttgtacgttctgctaaactgaaagaactgccggaatacatcgaaaaactgcgtaacaacgcttacgaaggtcgtgttgttttcaacccgtaa

SEQ ID NO: 18 - protein, 336 aa, synthetic construct
  REGION 1..336: note = KRED-NADH-102 variant
MSIPSSQYGFVFNKQSGLKLRNDLPVHKPKAGQLLLKVDAVGLCHSDLHVIYEGLDCGDNYVMGHEIAGTVAAVGDDVINYKVGDRVACVGPNGCGGCKYCRGAIDNVCKNAFGDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQAKKLGADAVYETLPESISPGSFSACFDFVSVQATFDVCQKYVEPKGVIMPVGLGAPNLSFNLPDLALREIRILGSFWGTTNDLDDVLKLVSEGKVKPVVRSAKLKELPEYIEKLRNNAYEGRVVFNP

SEQ ID NO: 19 - DNA, 1011 bp, synthetic construct
  misc_feature 1..1011: note = KRED-NADH-102 variant
  source 1..1011: mol_type = other DNA
atgtctatcccgtcttctcagtatggtttcgtatttaacaaacagtctggtctgaaactgcgcaacgatctgccagtacacaagccgaaagctggtcaactgctgctgaaagtagatgcagtaggtctgtgccactctgatctgcacgttatctacgaaggcctggactgcggtgacaactacgttatgggtcacgaaatcgctggtaccgttgctgctgttggtgatgacgttatcaactacaaagtaggtgaccgtgtagcttgtgttggtccgaacggttgcggtggttgcaaatactgccgtggtgctatcgacaacgtttgcaaaaacgctttcggtgactggttcggtctgggttatgatggtggctatcaacaatacctgctggtaactcgcccgcgtaacctgtctcgtatcccggataacgtatctgctgatgttgctgctgcttctaccgacgctgttctgactccgtaccacgctatcaaaatggctcaggtttctccgacctctaacatcctgctgatcggtgctggtggtctgggtggtaacgctatccaggttgctaaagcattcggtgctaaagttaccgttctggacaaaaaaaaagaagctcgtgaccaggctaaaaaactgggtgctgacgctgtttacgaaaccctgccggaatctatctctccgggttctttctctgcttgcttcgacttcgtttctgttcaggctaccttcgacgtttgccagaaatacgttgaaccgaaaggtgttatcatgccggttggtctgggtgctccgaacctgtcttttaacctgttggacctggctctgcgtgaaatccgtatcctgggttctttctggggtaccaccaacgacctggacgacgttctgaaactggtttctgaaggtaaagttaaaccggttgtacgttctgctaaactgaaagaactgccggaatacatcgaaaaactgcgtaacaacgcttacgaaggtcgtgttgttttcaacccgtaa

SEQ ID NO: 20 - protein, 336 aa, synthetic construct
  REGION 1..336: note = KRED-NADH-102 variant
MSIPSSQYGFVFNKQSGLKLRNDLPVHKPKAGQLLLKVDAVGLCHSDLHVIYEGLDCGDNYVMGHEIAGTVAAVGDDVINYKVGDRVACVGPNGCGGCKYCRGAIDNVCKNAFGDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQAKKLGADAVYETLPESISPGSFSACFDFVSVQATFDVCQKYVEPKGVIMPVGLGAPNLSFNLLDLALREIRILGSFWGTTNDLDDVLKLVSEGKVKPVVRSAKLKELPEYIEKLRNNAYEGRVVFNP

SEQ ID NO: 21 - DNA, 1011 bp, synthetic construct
  misc_feature 1..1011: note = KRED-NADH-102 variant
  source 1..1011: mol_type = other DNA
atgtctatcccgtcttctcagtatggtttcgtatttaacaaacagtctggtctgaaactgcgcaacgatctgccagtacacaagccgaaagctggtcaactgctgctgaaagtagatgcagtaggtctgtgccactctgatctgcacgttatctacgaaggcctggactgcggtgacaactacgttatgggtcacgaaatcgctggtaccgttgctgctgttggtgatgacgttatcaactacaaagtaggtgaccgtgtagcttgtgttggtccgaacggttgcggtggttgcaaatactgccgtggtgctatcgacaacgtttgcaaaaacgctttcatggactggttcggtctgggttatgatggtggctatcaacaatacctgctggtaactcgcccgcgtaacctgtctcgtatcccggataacgtatctgctgatgttgctgctgcttctaccgacgctgttctgactccgtaccacgctatcaaaatggctcaggtttctccgacctctaacatcctgctgatcggtgctggtggtctgggtggtaacgctatccaggttgctaaagcattcggtgctaaagttaccgttctggacaaaaaaaaagaagctcgtgaccaggctaaaaaactgggtgctgacgctgtttacgaaaccctgccggaatctatctctccgggttctttctctgcttgcttcgacttcgtttctgttcaggctaccttcgacgtttgccagaaatacgttgaaccgaaaggtgttatcatgccggttggtctgggtgctccgaacctgtcttttaacctgggtgacctggctctgcgtgaaatccgtatcctgggttctttctggggtaccaccaacgacctggacgacgttctgaaactggtttctgaaggtaaagttaaaccggttgtacgttctgctaaactgaaagaactgccggaatacatcgaaaaactgcgtaacaacgcttacgaaggtcgtgttgttttcaacccgtaa

SEQ ID NO: 22 - protein, 336 aa, synthetic construct
  REGION 1..336: note = KRED-NADH-102 variant
MSIPSSQYGFVFNKQSGLKLRNDLPVHKPKAGQLLLKVDAVGLCHSDLHVIYEGLDCGDNYVMGHEIAGTVAAVGDDVINYKVGDRVACVGPNGCGGCKYCRGAIDNVCKNAFMDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQAKKLGADAVYETLPESISPGSFSACFDFVSVQATFDVCQKYVEPKGVIMPVGLGAPNLSFNLGDLALREIRILGSFWGTTNDLDDVLKLVSEGKVKPVVRSAKLKELPEYIEKLRNNAYEGRVVFNP

SEQ ID NO: 23 - DNA, 1011 bp, synthetic construct
  misc_feature 1..1011: note = KRED-NADH-102 variant
  source 1..1011: mol_type = other DNA
atgtctatcccgtcttctcagtatggtttcgtatttaacaaacagtctggtctgaaactgcgcaacgatctgccagtacacaagccgaaagctggtcaactgctgctgaaagtagatgcagtaggtctgtgccactctgatctgcacgttatctacgaaggcctggactgcggtgacaactacgttatgggtcacgaaatcgctggtaccgttgctgctgttggtgatgacgttatcaactacaaagtaggtgaccgtgtagcttgtgttggtccgaacggttgcggtggttgcaaatactgccgtggtgctatcgacaacgtttgcaaaaacgctttcggtgactggttcggtctgggttatgatggtggctatcaacaatacctgctggtaactcgcccgcgtaacctgtctcgtatcccggataacgtatctgctgatgttgctgctgcttctaccgacgctgttctgactccgtaccacgctatcaaaatggctcaggtttctccgacctctaacatcctgctgatcggtgctggtggtctgggtggtaacgctatccaggttgctaaagcattcggtgctaaagttaccgttctggacaaaaaaaaagaagctcgtgaccaggctaaaaaactgggtgctgacgctgtttacgaaaccctgccggaatctatctctccgggttctttctctgcttgcttcgacttcgtttctgttcaggctaccttcgacgtttgccagaaatacgttgaaccgaaaggtgttatcatgccggttggtctgggtgctccgaacctgtcttttaacctgagtgacctggctctgcgtgaaatccgtatcctgggttctttctggggtaccaccaacgacctggacgacgttctgaaactggtttctgaaggtaaagttaaaccggttgtacgttctgctaaactgaaagaactgccggaatacatcgaaaaactgcgtaacaacgcttacgaaggtcgtgttgttttcaacccgtaa

SEQ ID NO: 24 - protein, 336 aa, synthetic construct
  REGION 1..336: note = KRED-NADH-102 variant
MSIPSSQYGFVFNKQSGLKLRNDLPVHKPKAGQLLLKVDAVGLCHSDLHVIYEGLDCGDNYVMGHEIAGTVAAVGDDVINYKVGDRVACVGPNGCGGCKYCRGAIDNVCKNAFGDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQAKKLGADAVYETLPESISPGSFSACFDFVSVQATFDVCQKYVEPKGVIMPVGLGAPNLSFNLSDLALREIRILGSFWGTTNDLDDVLKLVSEGKVKPVVRSAKLKELPEYIEKLRNNAYEGRVVFNP

SEQ ID NO: 25 - DNA, 1011 bp, synthetic construct
  misc_feature 1..1011: note = KRED-NADH-102 variant
  source 1..1011: mol_type = other DNA
atgtctatcccgtcttctcagtatggtttcgtatttaacaaacagtctggtctgaaactgcgcaacgatctgccagtacacaagccgaaagctggtcaactgctgctgaaagtagatgcagtaggtctgtgccactctgatctgcacgttatctacgaaggcctggactgcggtgacaactacgttatgggtcacgaaatcgctggtaccgttgctgctgttggtgatgacgttatcaactacaaagtaggtgaccgtgtagcttgtgttggtccgaacggttgcggtggttgcaaatactgccgtggtgctatcgacaacgtttgcaaaaacgctttcggtgactggttcggtctgggttatgatggtggctatcaacaatacctgctggtaactcgcccgcgtaacctgtctcgtatcccggataacgtatctgctgatgttgctgctgcttctaccgacgctgttctgactccgtaccacgctatcaaaatggctcaggtttctccgacctctaacatcctgctgatcggtgctggtggtctgggtggtaacgctatccaggttgctaaagcattcggtgctaaagttaccgttctggacaaaaaaaaagaagctcgtgaccaggctaaaaaactgggtgctgacgctgtttacgaaaccctgccggaatctatctctccgggttctttctctgcttgcttcgacttcgtttctgttcaggctaccttcgacgtttgccagaaatacgttgaaccgaaaggtgttatcatgccggttggtctgggtgctccgaacctgtcttttaacctgcaagacctggctctgcgtgaaatccgtatcctgggttctttctggggtaccaccaacgacctggacgacgttctgaaactggtttctgaaggtaaagttaaaccggttgtacgttctgctaaactgaaagaactgccggaatacatcgaaaaactgcgtaacaacgcttacgaaggtcgtgttgttttcaacccgtaa

SEQ ID NO: 26 - protein, 336 aa, synthetic construct
  REGION 1..336: note = KRED-NADH-102 variant
MSIPSSQYGFVFNKQSGLKLRNDLPVHKPKAGQLLLKVDAVGLCHSDLHVIYEGLDCGDNYVMGHEIAGTVAAVGDDVINYKVGDRVACVGPNGCGGCKYCRGAIDNVCKNAFGDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQAKKLGADAVYETLPESISPGSFSACFDFVSVQATFDVCQKYVEPKGVIMPVGLGAPNLSFNLQDLALREIRILGSFWGTTNDLDDVLKLVSEGKVKPVVRSAKLKELPEYIEKLRNNAYEGRVVFNP

SEQ ID NO: 27 - DNA, 1011 bp, synthetic construct
  misc_feature 1..1011: note = KRED-NADH-102 variant
  source 1..1011: mol_type = other DNA
atgtctatcccgtcttctcagtatggtttcgtatttaacaaacagtctggtctgaaactgcgcaacgatctgccagtacacaagccgaaagctggtcaactgctgctgaaagtagatgcagtaggtctgtgccactctgatctgcacgttatctacgaaggcctggactgcggtgacaactacgttatgggtcacgaaatcgctggtaccgttgctgctgttggtgatgacgttatcaactacaaagtaggtgaccgtgtagcttgtgttggtccgaacggttgcggtggttgcaaatactgccgtggtgctatcgacaacgtttgcaaaaacgctttcggtgactggttcggtctgggttatgatggtggctatcaacaatacctgctggtaactcgcccgcgtaacctgtctcgtatcccggataacgtatctgctgatgttgctgctgcttctaccgacgctgttctgactccgtaccacgctatcaaaatggctcaggtttctccgacctctaacatcctgctgatcggtgctggtggtctgggtggtaacgctatccaggttgctaaagcattcggtgctaaagttaccgttctggacaaaaaaaaagaagctcgtgaccaggctaaaaaactgggtgctgacgctgtttacgaaaccctgccggaatctatctctccgggttctttctctgcttgcttcgacttcgtttctgttcaggctaccttcgacgtttgccagaaatacgttgaaccgaaaggtgttatcatgccggttggtctgggtgctccgaacctgtcttttaacctgcatgacctggctctgcgtgaaatccgtatcctgggttctttctggggtaccaccaacgacctggacgacgttctgaaactggtttctgaaggtaaagttaaaccggttgtacgttctgctaaactgaaagaactgccggaatacatcgaaaaactgcgtaacaacgcttacgaaggtcgtgttgttttcaacccgtaa

SEQ ID NO: 28 - protein, 336 aa, synthetic construct
  REGION 1..336: note = KRED-NADH-102 variant
MSIPSSQYGFVFNKQSGLKLRNDLPVHKPKAGQLLLKVDAVGLCHSDLHVIYEGLDCGDNYVMGHEIAGTVAAVGDDVINYKVGDRVACVGPNGCGGCKYCRGAIDNVCKNAFGDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQAKKLGADAVYETLPESISPGSFSACFDFVSVQATFDVCQKYVEPKGVIMPVGLGAPNLSFNLHDLALREIRILGSFWGTTNDLDDVLKLVSEGKVKPVVRSAKLKELPEYIEKLRNNAYEGRVVFNP

SEQ ID NO: 29 - DNA, 1011 bp, synthetic construct
  misc_feature 1..1011: note = KRED-NADH-102 variant
  source 1..1011: mol_type = other DNA
atgtctatcccgtcttctcagtatggtttcgtatttaacaaacagtctggtctgaaactgcgcaacgatctgccagtacacaagccgaaagctggtcaactgctgctgaaagtagatgcagtaggtctgtgccactctgatctgcacgttatctacgaaggcctggactgcggtgacaactacgttatgggtcacgaaatcgctggtaccgttgctgctgttggtgatgacgttatcaactacaaagtaggtgaccgtgtagcttgtgttggtccgaacggttgcggtggttgcaaatactgccgtggtgctatcgacaacgtttgcaaaaacgctttcggtgactggttcggtctgggttatgatggtggctatcaacaatacctgctggtaactcgcccgcgtaacctgtctcgtatcccggataacgtatctgctgatgttgctgctgcttctaccgacgctgttctgactccgtaccacgctatcaaaatggctcaggtttctccgacctctaacatcctgctgatcggtgctggtggtctgggtggtaacgctatccaggttgctaaagcattcggtgctaaagttaccgttctggacaaaaaaaaagaagctcgtgaccaggctaaaaaactgggtgctgacgctgtttacgaaaccctgccggaatctatctctccgggttctttctctgcttgcttcgacttcgtttctgttcaggctaccttcgacgtttgccagaaatacgttgaaccgaaaggtgttatcatgccggttggtctgggtgctccgaacctgtcttttaacctgacagacctggctctgcgtgaaatccgtatcctgggttctttctggggtaccaccaacgacctggacgacgttctgaaactggtttctgaaggtaaagttaaaccggttgtacgttctgctaaactgaaagaactgccggaatacatcgaaaaactgcgtaacaacgcttacgaaggtcgtgttgttttcaacccgtaa

SEQ ID NO: 30 - protein, 336 aa, synthetic construct
  REGION 1..336: note = KRED-NADH-102 variant
MSIPSSQYGFVFNKQSGLKLRNDLPVHKPKAGQLLLKVDAVGLCHSDLHVIYEGLDCGDNYVMGHEIAGTVAAVGDDVINYKVGDRVACVGPNGCGGCKYCRGAIDNVCKNAFGDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQAKKLGADAVYETLPESISPGSFSACFDFVSVQATFDVCQKYVEPKGVIMPVGLGAPNLSFNLTDLALREIRILGSFWGTTNDLDDVLKLVSEGKVKPVVRSAKLKELPEYIEKLRNNAYEGRVVFNP

SEQ ID NO: 31 - DNA, 1011 bp, synthetic construct
  misc_feature 1..1011: note = KRED-NADH-102 variant
  source 1..1011: mol_type = other DNA
atgtctatcccgtcttctcagtatggtttcgtatttaacaaacagtctggtctgaaactgcgcaacgatctgccagtacacaagccgaaagctggtcaactgctgctgaaagtagatgcagtaggtctgtgccactctgatctgcacgttatctacgaaggcctggactgcggtgacaactacgttatgggtcacgaaatcgctggtaccgttgctgctgttggtgatgacgttatcaactacaaagtaggtgaccgtgtagcttgtgttggtccgaacggttgcggtggttgcaaatactgccgtggtgctatcgacaacgtttgcaaaaacgctttccatgactggttcggtctgggttatgatggtggctatcaacaatacctgctggtaactcgcccgcgtaacctgtctcgtatcccggataacgtatctgctgatgttgctgctgcttctaccgacgctgttctgactccgtaccacgctatcaaaatggctcaggtttctccgacctctaacatcctgctgatcggtgctggtggtctgggtggtaacgctatccaggttgctaaagcattcggtgctaaagttaccgttctggacaaaaaaaaagaagctcgtgaccaggctaaaaaactgggtgctgacgctgtttacgaaaccctgccggaatctatctctccgggttctttctctgcttgcttcgacttcgtttctgttcaggctaccttcgacgtttgccagaaatacgttgaaccgaaaggtgttatcatgccggttggtctgggtgctccgaacctgtcttttaacctgggtgacctggctctgcgtgaaatccgtatcctgggttctttctggggtaccaccaacgacctggacgacgttctgaaactggtttctgaaggtaaagttaaaccggttgtacgttctgctaaactgaaagaactgccggaatacatcgaaaaactgcgtaacaacgcttacgaaggtcgtgttgttttcaacccgtaa

SEQ ID NO: 32 - protein, 336 aa, synthetic construct
  REGION 1..336: note = KRED-NADH-102 variant
MSIPSSQYGFVFNKQSGLKLRNDLPVHKPKAGQLLLKVDAVGLCHSDLHVIYEGLDCGDNYVMGHEIAGTVAAVGDDVINYKVGDRVACVGPNGCGGCKYCRGAIDNVCKNAFHDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQAKKLGADAVYETLPESISPGSFSACFDFVSVQATFDVCQKYVEPKGVIMPVGLGAPNLSFNLGDLALREIRILGSFWGTTNDLDDVLKLVSEGKVKPVVRSAKLKELPEYIEKLRNNAYEGRVVFNP

SEQ ID NO: 33 - DNA, 1011 bp, synthetic construct
  misc_feature 1..1011: note = KRED-NADH-102 variant
  source 1..1011: mol_type = other DNA
atgtctatcccgtcttctcagtatggtttcgtatttaacaaacagtctggtctgaaactgcgcaacgatctgccagtacacaagccgaaagctggtcaactgctgctgaaagtagatgcagtaggtctgtgccactctgatctgcacgttatctacgaaggcctggactgcggtgacaactacgttatgggtcacgaaatcgctggtaccgttgctgctgttggtgatgacgttatcaactacaaagtaggtgaccgtgtagcttgtgttggtccgaacggttgcggtggttgcaaatactgccgtggtgctatcgacaacgtttgcaaaaacgctttcggtgactggttcggtctgggttatgatggtggctatcaacaatacctgctggtaactcgcccgcgtaacctgtctcgtatcccggataacgtatctgctgatgttgctgctgcttctaccgacgctgttctgactccgtaccacgctatcaaaatggctcaggtttctccgacctctaacatcctgctgatcggtgctggtggtctgggtggtaacgctatccaggttgctaaagcattcggtgctaaagttaccgttctggacaaaaaaaaagaagctcgtgaccaggctaaaaaactgggtgctgacgctgtttacgaaaccctgccggaatctatctctccgggttctttctctgcttgcttcgacttcgtttctgttcaggctaccttcgacgtttgccagaaatacgttgaaccgaaaggtgttatcatgccggttggtctgggtgctccgaacctgtcttttaacctgattgacctggctctgcgtgaaatccgtatcctgggttctttctggggtaccaccaacgacctggacgacgttctgaaactggtttctgaaggtaaagttaaaccggttgtacgttctgctaaactgaaagaactgccggaatacatcgaaaaactgcgtaacaacgcttacgaaggtcgtgttgttttcaacccgtaa

SEQ ID NO: 34 - protein, 336 aa, synthetic construct
  REGION 1..336: note = KRED-NADH-102 variant
MSIPSSQYGFVFNKQSGLKLRNDLPVHKPKAGQLLLKVDAVGLCHSDLHVIYEGLDCGDNYVMGHEIAGTVAAVGDDVINYKVGDRVACVGPNGCGGCKYCRGAIDNVCKNAFGDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQAKKLGADAVYETLPESISPGSFSACFDFVSVQATFDVCQKYVEPKGVIMPVGLGAPNLSFNLIDLALREIRILGSFWGTTNDLDDVLKLVSEGKVKPVVRSAKLKELPEYIEKLRNNAYEGRVVFNP

SEQ ID NO: 35 - DNA, 1011 bp, synthetic construct
  misc_feature 1..1011: note = KRED-NADH-102 variant
  source 1..1011: mol_type = other DNA
atgtctatcccgtcttctcagtatggtttcgtatttaacaaacagtctggtctgaaactgcgcaacgatctgccagtacacaagccgaaagctggtcaactgctgctgaaagtagatgcagtaggtctgtgccactctgatctgcacgttatctacgaaggcctggackkyggtgacaactacgttatgggtcacgaaatcgctggtaccgttgctgctgttggtgatgacgttatcaactacaaagtaggtgaccgtgtagcttgtgttggtccgaacggttgcggtggttgcaaatactgccgtggtgctatcgacaacgtttgcaaaaacgctttcggtgactggttcggtctgggttatgatggtggctatcaacaatacctgctggtaactcgcccgcgtaacctgtctcgtatcccggataacgtatctgctgatgttgctgctgcttctaccgacgctgttctgactccgtaccacgctatcaaaatggctcaggtttctccgacctctaacatcctgctgatcggtgctggtggtctgggtggtaacgctatccaggttgctaaagcattcggtgctaaagttaccgttctggacaaaaaaaaagaagctcgtgaccaggctaaaaaactgggtgctgacgctgtttacgaaaccctgccggaatctatctctccgggttctttctctgcttgcttcgacttcgtttctgttcaggctaccttcgacgtttgccagaaatacgttgaaccgaaaggtgttatcatgccggttggtctgggtgctccgaacctgtcttttaacctgggtgacctggctctgcgtgaaatccgtatcctgggttctttcyrrggtaccaccaacgacctggacgacgttctgaaactggtttctgaaggtaaagttaaaccggttgtacgttctgctaaactgaaagaactgccggaatacatcgaaaaactgcgtaacaacgcttacgaaggtcgtgttgttttcaacccgtaa

SEQ ID NO: 36 - protein, 336 aa, synthetic construct
  SITE 286: note = unsure - KRED-NADH-102 variant, Xaa = Phe, Cys, Gly, Val, Xaa = Gln, Ter, Trp, Arg
MSIPSSQYGFVFNKQSGLKLRNDLPVHKPKAGQLLLKVDAVGLCHSDLHVIYEGLDXGDNYVMGHEIAGTVAAVGDDVINYKVGDRVACVGPNGCGGCKYCRGAIDNVCKNAFGDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQAKKLGADAVYETLPESISPGSFSACFDFVSVQATFDVCQKYVEPKGVIMPVGLGAPNLSFNLGDLALREIRILGSFXGTTNDLDDVLKLVSEGKVKPVVRSAKLKELPEYIEKLRNNAYEGRVVFNP

SEQ ID NO: 37 - DNA, 1011 bp, synthetic construct
  misc_feature 1..1011: note = KRED-NADH-102 variant
  source 1..1011: mol_type = other DNA
atgtctatcccgtcttctcagtatggtttcgtatttaacaaacagtctggtctgaaactgcgcaacgatctgccagtacacaagccgaaagctggtcaactgctgctgaaagtagatgcagtaggtctgtgccactctgatctgcacgttatctacgaaggcctggactgcggtgacaactacgttatgggtcacgaaatcgctggtaccgttgctgctgttggtgatgacgttatcaactacaaagtaggtgaccgtgtagcttgtgttggtccgaacggttgcggtggttgcaaatactgccgtggtgctatcgacaacgtttgcaaaaacgctttcggtgactggttcggtctgggttatgatggtggctatcaacaatacctgctggtaactcgcccgcgtaacctgtctcgtatcccggataacgtatctgctgatgttgctgctgcttctaccgacgctgttctgactccgtaccacgctatcaaaatggctcaggtttctccgacctctaacatcctgctgatcggtgctggtggtctgggtggtaacgctatccaggttgctaaagcattcggtgctaaagttaccgttctggacaaaaaaaaagaagctcgtgaccaggctaaaaaactgggtgctgacgctgtttacgaaaccctgccggaatctatctctccgggttctttctctgcttgcttcgacttcgtttctgttcaggctaccttcgacgtttgccagaaatacgttgaaccgaaaggtgttatcatgccggttggtctgggtgctccgaacctgtcttttaacctgtgggacctggctctgcgtgaaatccgtatcctgggttctttctggggtaccaccaacgacctggacgacgttctgaaactggtttctgaaggtaaagttaaaccggttgtacgttctgctaaactgaaagaactgccggaatacatcgaaaaactgcgtaacaacgcttacgaaggtcgtgttgttttcaacccgtaa

SEQ ID NO: 38 - protein, 336 aa, synthetic construct
  REGION 1..336: note = KRED-NADH-102 variant
MSIPSSQYGFVFNKQSGLKLRNDLPVHKPKAGQLLLKVDAVGLCHSDLHVIYEGLDCGDNYVMGHEIAGTVAAVGDDVINYKVGDRVACVGPNGCGGCKYCRGAIDNVCKNAFGDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQAKKLGADAVYETLPESISPGSFSACFDFVSVQATFDVCQKYVEPKGVIMPVGLGAPNLSFNLWDLALREIRILGSFWGTTNDLDDVLKLVSEGKVKPVVRSAKLKELPEYIEKLRNNAYEGRVVFNP

SEQ ID NO: 39 - DNA, 1011 bp, synthetic construct
  misc_feature 1..1011: note = KRED-NADH-102 variant
  source 1..1011: mol_type = other DNA
atgtctatcccgtcttctcagtatggtttcgtatttaacaaacagtctggtctgaaactgcgcaacgatctgccagtacacaagccgaaagctggtcaactgctgctgaaagtagatgcagtaggtctgtgccactctgatctgcacgttatctacgaaggcctggactgcggtgacaactacgttatgggtcacgaaatcgctggtaccgttgctgctgttggtgatgacgttatcaactacaaagtaggtgaccgtgtagcttgtgttggtccgaacggttgcggtggttgcaaatactgccgtggtgctatcgacaacgtttgcaaaaacgctttcggtgactggttcggtctgggttatgatggtggctatcaacaatacctgctggtaactcgcccgcgtaacctgtctcgtatcccggataacgtatctgctgatgttgctgctgcttctaccgacgctgttctgactccgtaccacgctatcaaaatggctcaggtttctccgacctctaacatcctgctgatcggtgctggtggtctgggtggtaacgctatccaggttgctaaagcattcggtgctaaagttaccgttctggacaaaaaaaaagaagctcgtgaccaggctaaaaaactgggtgctgacgctgtttacgaaaccctgccggaatctatctctccgggttctttctctgcttgcttcgacttcgtttctgttcaggctaccttcgacgtttgccagaaatacgttgaaccgaaaggtgttatcatgccggttggtctgggtgctccgaacctgtcttttaacctgggtgacctggctctgcgtgaacatcgtatcctgggttctttctggggtaccaccaacgacctggacgacgttctgaaactggtttctgaaggtaaagttaaaccggttgtacgttctgctaaactgaaagaactgccggaatacatcgaaaaactgcgtaacaacgcttacgaaggtcgtgttgttttcaacccgtaa

SEQ ID NO: 40 - protein, 336 aa, synthetic construct
  REGION 1..336: note = KRED-NADH-102 variant
MSIPSSQYGFVFNKQSGLKLRNDLPVHKPKAGQLLLKVDAVGLCHSDLHVIYEGLDCGDNYVMGHEIAGTVAAVGDDVINYKVGDRVACVGPNGCGGCKYCRGAIDNVCKNAFGDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQAKKLGADAVYETLPESISPGSFSACFDFVSVQATFDVCQKYVEPKGVIMPVGLGAPNLSFNLGDLALREHRILGSFWGTTNDLDDVLKLVSEGKVKPVVRSAKLKELPEYIEKLRNNAYEGRVVFNP

SEQ ID NO: 41 - DNA, 1011 bp, synthetic construct
  misc_feature 1..1011: note = KRED-NADH-102 variant
  source 1..1011: mol_type = other DNA
atgtctatcccgtcttctcagtatggtttcgtatttaacaaacagtctggtctgaaactgcgcaacgatctgccagtacacaagccgaaagctggtcaactgctgctgaaagtagatgcagtaggtctgtgccactctgatctgcacgttatctacgaaggcctggactgcggtgacaactacgttatgggtcacgaaatcgctggtaccgttgctgctgttggtgatgacgttatcaactacaaagtaggtgaccgtgtagcttgtgttggtccgaacggttgcggtggttgcaaatactgccgtggtgctatcgacaacgtttgcaaaaacgctttcggtgactggttcggtctgggttatgatggtggctatcaacaatacctgctggtaactcgcccgcgtaacctgtctcgtatcccggataacgtatctgctgatgttgctgctgcttctaccgacgctgttctgactccgtaccacgctatcaaaatggctcaggtttctccgacctctaacatcctgctgatcggtgctggtggtctgggtggtaacgctatccaggttgctaaagcattcggtgctaaagttaccgttctggacaaaaaaaaagaagctcgtgaccaggctaaaaaactgggtgctgacgctgtttacgaaaccctgccggaatctatctctccgggttctttctctgcttgcttcgacttcgtttctgttcaggctaccttcgacgtttgccagaaatacgttgaaccgaaaggtgttatcatgccggttggtctgcatgctccgaacctgtcttttaacctgggtgacctggctctgcgtgaaatccgtatcctgggttctttctggggtaccaccaacgacctggacgacgttctgaaactggtttctgaaggtaaagttaaaccggttgtacgttctgctaaactgaaagaactgccggaatacatcgaaaaactgcgtaacaacgcttacgaaggtcgtgttgttttcaacccgtaa

SEQ ID NO: 42 - protein, 336 aa, synthetic construct
  REGION 1..336: note = KRED-NADH-102 variant
MSIPSSQYGFVFNKQSGLKLRNDLPVHKPKAGQLLLKVDAVGLCHSDLHVIYEGLDCGDNYVMGHEIAGTVAAVGDDVINYKVGDRVACVGPNGCGGCKYCRGAIDNVCKNAFGDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQAKKLGADAVYETLPESISPGSFSACFDFVSVQATFDVCQKYVEPKGVIMPVGLHAPNLSFNLGDLALREIRILGSFWGTTNDLDDVLKLVSEGKVKPVVRSAKLKELPEYIEKLRNNAYEGRVVFNP

SEQ ID NO: 43 - DNA, 1011 bp, synthetic construct
  misc_feature 1..1011: note = KRED-NADH-102 variant
  source 1..1011: mol_type = other DNA
atgtctatcccgtcttctcagtatggtttcgtatttaacaaacagtctggtctgaaactgcgcaacgatctgccagtacacaagccgaaagctggtcaactgctgctgaaagtagatgcagtaggtctgtgccgttctgatctgcacgttatctacgaaggcctggactgcggtgacaactacgttatgggtcacgaaatcgctggtaccgttgctgctgttggtgatgacgttatcaactacaaagtaggtgaccgtgtagcttgtgttggtccgaacggttgcggtggttgcaaatactgccgtggtgctatcgacaacgtttgcaaaaacgctttcggtgactggttcggtctgggttatgatggtggctatcaacaatacctgctggtaactcgcccgcgtaacctgtctcgtatcccggataacgtatctgctgatgttgctgctgcttctaccgacgctgttctgactccgtaccacgctatcaaaatggctcaggtttctccgacctctaacatcctgctgatcggtgctggtggtctgggtggtaacgctatccaggttgctaaagcattcggtgctaaagttaccgttctggacaaaaaaaaagaagctcgtgaccaggctaaaaaactgggtgctgacgctgtttacgaaaccctgccggaatctatctctccgggttctttctctgcttgcttcgacttcgtttctgttcaggctaccttcgacgtttgccagaaatacgttgaaccgaaaggtgttatcatgccggttggtctgggtgctccgaacctgtcttttaacctgggtgacctggctctgcgtgaaatccgtatcctgggttctttctggggtaccaccaacgacctggacgacgttctgaaactggtttctgaaggtaaagttaaaccggttgtacgttctgctaaactgaaagaactgccggaatacatcgaaaaactgcgtaacaacgcttacgaaggtcgtgttgttttcaacccgtaa

SEQ ID NO: 44 - protein, 336 aa, synthetic construct
  REGION 1..336: note = KRED-NADH-102 variant
MSIPSSQYGFVFNKQSGLKLRNDLPVHKPKAGQLLLKVDAVGLCRSDLHVIYEGLDCGDNYVMGHEIAGTVAAVGDDVINYKVGDRVACVGPNGCGGCKYCRGAIDNVCKNAFGDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQAKKLGADAVYETLPESISPGSFSACFDFVSVQATFDVCQKYVEPKGVIMPVGLGAPNLSFNLGDLALREIRILGSFWGTTNDLDDVLKLVSEGKVKPVVRSAKLKELPEYIEKLRNNAYEGRVVFNP

SEQ ID NO: 45 - DNA, 1011 bp, synthetic construct
  misc_feature 1..1011: note = KRED-NADH-102 variant
  source 1..1011: mol_type = other DNA
atgtctatcccgtcttctcagtatggtttcgtatttaacaaacagtctggtctgaaactgcgcaacgatctgccagtacacaagccgaaagctggtcaactgctgctgaaagtagatgcagtaggtctgtgccactctgatctgcacgttatctacgaaggcctggactgcggtgacaactacgttatgggtcacgaaatcgctggtaccgttgctgctgttggtgatgacgttatcaactacaaagtaggtgaccgtgtagcttgtgttggtccgaacggttgcggtggttgcaaatactgccgtggtgctatcgacaacgtttgcaaaaacgctttcggtgactggttcggtctgggttatgatggtggctatcaacaatacctgctggtaactcgcccgcgtaacctgtctcgtatcccggataacgtatctgctgatgttgctgctgcttctaccgacgctgttctgactccgtaccacgctatcaaaatggctcaggtttctccgacctctaacatcctgctgatcggtgctggtggtctgggtggtaacgctatccaggttgctaaagcattcggtgctaaagttaccgttctggacaaaaaaaaagaagctcgtgaccaggctaaaaaactgggtgctgacgctgtttacgaaaccctgccggaatctatctctccgggttctttctctgcttgcttcgacttcgtttctgttcaggctaccttcgacgtttgccagaaatacgttgaaccgaaaggtgttatcatgccggttggtctgggtgctccgaacctgatgtttaacctgggtgacctggctctgcgtgaaatccgtatcctgggttctttctggggtaccaccaacgacctggacgacgttctgaaactggtttctgaaggtaaagttaaaccggttgtacgttctgctaaactgaaagaactgccggaatacatcgaaaaactgcgtaacaacgcttacgaaggtcgtgttgttttcaacccgtaa

SEQ ID NO: 46 - protein, 336 aa, synthetic construct
  REGION 1..336: note = KRED-NADH-102 variant
MSIPSSQYGFVFNKQSGLKLRNDLPVHKPKAGQLLLKVDAVGLCHSDLHVIYEGLDCGDNYVMGHEIAGTVAAVGDDVINYKVGDRVACVGPNGCGGCKYCRGAIDNVCKNAFGDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQAKKLGADAVYETLPESISPGSFSACFDFVSVQATFDVCQKYVEPKGVIMPVGLGAPNLMFNLGDLALREIRILGSFWGTTNDLDDVLKLVSEGKVKPVVRSAKLKELPEYIEKLRNNAYEGRVVFNP

SEQ ID NO: 47 - DNA, 1011 bp, synthetic construct
  misc_feature 1..1011: note = KRED-NADH-102 variant
  source 1..1011: mol_type = other DNA
atgtctatcccgtcttctcagtatggtttcgtatttaacaaacagtctggtctgaaactgcgcaacgatctgccagtacacaagccgaaagctggtcaactgctgctgaaagtagatgcagtaggtctgtgccactctgatctgcacgttatctacgaaggcctggactgcggtgacaactacgttatgggtcacgaaatcgctggtaccgttgctgctgttggtgatgacgttatcaactacaaagtaggtgaccgtgtagcttgtgttggtccgaacggttgcggtggttgcaaatactgccgtggtgctatcgacaacgtttgcaaaaacgctttcggtgactggttcggtctgggttatgatggtggctatcaacaatacctgctggtaactcgcccgcgtaacctgtctcgtatcccggataacgtatctgctgatgttgctgctgcttctaccgacgctgttctgactccgtaccacgctatcaaaatggctcaggtttctccgacctctaacatcctgctgatcggtgctggtggtctgggtggtaacgctatccaggttgctaaagcattcggtgctaaagttaccgttctggacaaaaaaaaagaagctcgtgaccaggctaaaaaactgggtgctgacgctgtttacgaaaccctgccggaatctatctctccgggttctttctctgcttgcttcgacttcgtttctgttcaggctaccttcgacgtttgccagaaatacgttgaaccgaaaggtgttatcatgccggttggtctgggtgctccgaacctgtggtttaacctgggtgacctggctctgcgtgaaatccgtatcctgggttctttctggggtaccaccaacgacctggacgacgttctgaaactggtttctgaaggtaaagttaaaccggttgtacgttctgctaaactgaaagaactgccggaatacatcgaaaaactgcgtaacaacgcttacgaaggtcgtgttgttttcaacccgtaa

SEQ ID NO: 48 - protein, 336 aa, synthetic construct
  REGION 1..336: note = KRED-NADH-102 variant
MSIPSSQYGFVFNKQSGLKLRNDLPVHKPKAGQLLLKVDAVGLCHSDLHVIYEGLDCGDNYVMGHEIAGTVAAVGDDVINYKVGDRVACVGPNGCGGCKYCRGAIDNVCKNAFGDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQAKKLGADAVYETLPESISPGSFSACFDFVSVQATFDVCQKYVEPKGVIMPVGLGAPNLWFNLGDLALREIRILGSFWGTTNDLDDVLKLVSEGKVKPVVRSAKLKELPEYIEKLRNNAYEGRVVFNP

SEQ ID NO: 49 - DNA, 1011 bp, synthetic construct
  misc_feature 1..1011: note = KRED-NADH-102 variant
  source 1..1011: mol_type = other DNA
atgtctatcccgtcttctcagtatggtttcgtatttaacaaacagtctggtctgaaactgcgcaacgatctgccagtacacaagccgaaagctggtcaactgctgctgaaagtagatgcagtaggtctgtgccactctgatctgcacgttatctacgaaggcctggactgcggtgacaactacgttatgggtcacgaaatcgctggtaccgttgctgctgttggtgatgacgttatcaactacaaagtaggtgaccgtgtagcttgtgttggtccgaacggttgcggtggttgcaaatactgccgtggtgctatcgacaacgtttgcaaaaacgctttcggtgactggttcggtctgggttatgatggtggctatcaacaatacctgctggtaactcgcccgcgtaacctgtctcgtatcccggataacgtatctgctgatgttgctgctgcttctaccgacgctgttctgactccgtaccacgctatcaaaatggctcaggtttctccgacctctaacatcctgctgatcggtgctggtggtctgggtggtaacgctatccaggttgctaaagcattcggtgctaaagttaccgttctggacaaaaaaaaagaagctcgtgaccaggctaaaaaactgggtgctgacgctgtttacgaaaccctgccggaatctatctctccgggttctttctctgcttgcttcgacttcgtttctgttcaggctaccttcgacgtttgccagaaatacgttgaaccgaaaggtgttatcatgccggttggtctgggtgctccgaacctgtcttttaacctgggtgacgttgctctgcgtgaaatccgtatcctgggttctttctggggtaccaccaacgacctggacgacgttctgaaactggtttctgaaggtaaagttaaaccggttgtacgttctgctaaactgaaagaactgccggaatacatcgaaaaactgcgtaacaacgcttacgaaggtcgtgttgttttcaacccgtaa

SEQ ID NO: 50 - protein, 336 aa, synthetic construct
  REGION 1..336: note = KRED-NADH-102 variant
MSIPSSQYGFVFNKQSGLKLRNDLPVHKPKAGQLLLKVDAVGLCHSDLHVIYEGLDCGDNYVMGHEIAGTVAAVGDDVINYKVGDRVACVGPNGCGGCKYCRGAIDNVCKNAFGDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQAKKLGADAVYETLPESISPGSFSACFDFVSVQATFDVCQKYVEPKGVIMPVGLGAPNLSFNLGDVALREIRILGSFWGTTNDLDDVLKLVSEGKVKPVVRSAKLKELPEYIEKLRNNAYEGRVVFNP

SEQ ID NO: 51 - DNA, 1011 bp, synthetic construct
  misc_feature 1..1011: note = KRED-NADH-102 variant
  source 1..1011: mol_type = other DNA
atgtctatcccgtcttctcagtatggtttcgtatttaacaaacagtctggtctgaaactgcgcaacgatctgccagtacacaagccgaaagctggtcaactgctgctgaaagtagatgcagtaggtctgtgccactctgatctgcacgttatctacgaaggcctggactgcggtgacaactacgttatgggtcacgaaatcgctggtaccgttgctgctgttggtgatgacgttatcaactacaaaattggtgaccgtgtagcttgtgttggtccgaacggttgcggtggttgcaaatactgccgtggtgctatcgacaacgtttgcaaaaacgctttcggtgactggttcggtctgggttatgatggtggctatcaacaatacctgctggtaactcgcccgcgtaacctgtctcgtatcccggataacgtatctgctgatgttgctgctgcttctaccgacgctgttctgactccgtaccacgctatcaaaatggctcaggtttctccgacctctaacatcctgctgatcggtgctggtggtctgggtggtaacgctatccaggttgctaaagcattcggtgctaaagttaccgttctggacaaaaaaaaagaagctcgtgaccaggctaaaaaactgggtgctgacgctgtttacgaaaccctgccggaatctatctctccgggttctttctctgcttgcttcgacttcgtttctgttcaggctaccttcgacgtttgccagaaatacgttgaaccgaaaggtgttatcatgccggttggtctgggtgctccgaacctgtcttttaacctgggtgacctggctctgcgtgaaatccgtatcctgggttctttctggggtaccaccaacgacctggacgacgttctgaaactggtttctgaaggtaaagttaaaccggttgtacgttctgctaaactgaaagaactgccggaatacatcgaaaaactgcgtaacaacgcttacgaaggtcgtgttgttttcaacccgtaa

SEQ ID NO: 52 - protein, 336 aa, synthetic construct
  REGION 1..336: note = KRED-NADH-102 variant
MSIPSSQYGFVFNKQSGLKLRNDLPVHKPKAGQLLLKVDAVGLCHSDLHVIYEGLDCGDNYVMGHEIAGTVAAVGDDVINYKIGDRVACVGPNGCGGCKYCRGAIDNVCKNAFGDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQAKKLGADAVYETLPESISPGSFSACFDFVSVQATFDVCQKYVEPKGVIMPVGLGAPNLSFNLGDLALREIRILGSFWGTTNDLDDVLKLVSEGKVKPVVRSAKLKELPEYIEKLRNNAYEGRVVFNP

SEQ ID NO: 53 - DNA, 1011 bp, synthetic construct
  misc_feature 1..1011: note = KRED-NADH-102 variant
  source 1..1011: mol_type = other DNA
atgtctatcccgtcttctcagtatggtttcgtatttaacaaacagtctggtctgaaactgcgcaacgatctgccagtacacaagccgaaagctggtcaactgctgctgaaagtagatgcagtaggtctgtgccactctgatctgcacgttatcgatgaaggcctggactgcggtgacaactacgttatgggtcacgaaatcgctggtaccgttgctgctgttggtgatgacgttatcaactacaaagtaggtgaccgtgtagcttgtgttggtccgaacggttgcggtggttgcaaatactgccgtggtgctatcgacaacgtttgcaaaaacgctttcggtgactggttcggtctgggttatgatggtggctatcaacaatacctgctggtaactcgcccgcgtaacctgtctcgtatcccggataacgtatctgctgatgttgctgctgcttctaccgacgctgttctgactccgtaccacgctatcaaaatggctcaggtttctccgacctctaacatcctgctgatcggtgctggtggtctgggtggtaacgctatccaggttgctaaagcattcggtgctaaagttaccgttctggacaaaaaaaaagaagctcgtgaccaggctaaaaaactgggtgctgacgctgtttacgaaaccctgccggaatctatctctccgggttctttctctgcttgcttcgacttcgtttctgttcaggctaccttcgacgtttgccagaaatacgttgaaccgaaaggtgttatcatgccggttggtctgggtgctccgaacctgtcttttaacctgggtgacctggctctgcgtgaaatccgtatcctgggttctttctggggtaccaccaacgacctggacgacgttctgaaactggtttctgaaggtaaagttaaaccggttgtacgttctgctaaactgaaagaactgccggaatacatcgaaaaactgcgtaacaacgcttacgaaggtcgtgttgttttcaacccgtaa

SEQ ID NO: 54 - protein, 336 aa, synthetic construct
  REGION 1..336: note = KRED-NADH-102 variant
MSIPSSQYGFVFNKQSGLKLRNDLPVHKPKAGQLLLKVDAVGLCHSDLHVIDEGLDCGDNYVMGHEIAGTVAAVGDDVINYKVGDRVACVGPNGCGGCKYCRGAIDNVCKNAFGDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQAKKLGADAVYETLPESISPGSFSACFDFVSVQATFDVCQKYVEPKGVIMPVGLGAPNLSFNLGDLALREIRILGSFWGTTNDLDDVLKLVSEGKVKPVVRSAKLKELPEYIEKLRNNAYEGRVVFNP

SEQ ID NO: 55 - DNA, 1011 bp, synthetic construct
  misc_feature 1..1011: note = KRED-NADH-102 variant
  source 1..1011: mol_type = other DNA
atgtctatcccgtcttctcagtatggtttcgtatttaacaaacagtctggtctgaaactgcgcaacgatctgccagtacacaagccgaaagctggtcaactgctgctgaaagtagatgcagtaggtctgtgccactctgatctgcacgttatctacgaaggcctggactgcggtgacaactacgttatgggtcacgaaatcgctggtaccgttgctgctgttggtgatgacgttatcaactacaaagtaggtgaccgtgtagcttgtgttggtccgaacggttgcggtggttgcaaatactgccgtggtgctatcgacaacgtttgcaaaaacgctttcggtgactggttcggtctgggttatgatggtggctatcaacaatacctgctggtaactcgcccgcgtaacctgtctcgtatcccggataacgtatctgctgatgttgctgctgcttctaccgacgctgttctgactccgtaccacgctatcaaaatggctcaggtttctccgacctctaacatcctgctgatcggtgctggtggtctgggtggtaacgctatccaggttgctaaagcattcggtgctaaagttaccgttctggacaaaaaaaaagaagctcgtgaccaggctaaaaaactgggtgctgacgctgtttacgaaaccctgccggaatctatctctccgggttctttctctgcttgcttcgacttcgtttctgttcaggctaccttcgacgtttgccagaaatacgttgaaccgaaaggtgttatcatgccggttggtctgggtgctccgaacctgtcttttaacctgggtgacctggctctgcgtgaaaggcgtatcctgggttctttctggggtaccaccaacgacctggacgacgttctgaaactggtttctgaaggtaaagttaaaccggttgtacgttctgctaaactgaaagaactgccggaatacatcgaaaaactgcgtaacaacgcttacgaaggtcgtgttgttttcaacccgtaa

SEQ ID NO: 56 - protein, 336 aa, synthetic construct
  REGION 1..336: note = KRED-NADH-102 variant
MSIPSSQYGFVFNKQSGLKLRNDLPVHKPKAGQLLLKVDAVGLCHSDLHVIYEGLDCGDNYVMGHEIAGTVAAVGDDVINYKVGDRVACVGPNGCGGCKYCRGAIDNVCKNAFGDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQAKKLGADAVYETLPESISPGSFSACFDFVSVQATFDVCQKYVEPKGVIMPVGLGAPNLSFNLGDLALRERRILGSFWGTTNDLDDVLKLVSEGKVKPVVRSAKLKELPEYIEKLRNNAYEGRVVFNP

SEQ ID NO: 57 - DNA, 1011 bp, synthetic construct
  misc_feature 1..1011: note = KRED-NADH-102 variant
  source 1..1011: mol_type = other DNA
atgtctatcccgtcttctcagtatggtttcgtatttaacaaacagtctggtctgaaactgcgcaacgatctgccagtacacaagccgaaagctggtcaactgctgctgaaagtagatgcagtaggtctgtgccactctgatctgcacgttatcagtgaaggcctggactgcggtgacaactacgttatgggtcacgaaatcgctggtaccgttgctgctgttggtgatgacgttatcaactacaaagtaggtgaccgtgtagcttgtgttggtccgaacggttgcggtggttgcaaatactgccgtggtgctatcgacaacgtttgcaaaaacgctttcggtgactggttcggtctgggttatgatggtggctatcaacaatacctgctggtaactcgcccgcgtaacctgtctcgtatcccggataacgtatctgctgatgttgctgctgcttctaccgacgctgttctgactccgtaccacgctatcaaaatggctcaggtttctccgacctctaacatcctgctgatcggtgctggtggtctgggtggtaacgctatccaggttgctaaagcattcggtgctaaagttaccgttctggacaaaaaaaaagaagctcgtgaccaggctaaaaaactgggtgctgacgctgtttacgaaaccctgccggaatctatctctccgggttctttctctgcttgcttcgacttcgtttctgttcaggctaccttcgacgtttgccagaaatacgttgaaccgaaaggtgttatcatgccggttggtctgggtgctccgaacctgtcttttaacctgggtgacctggctctgcgtgaaatccgtatcctgggttctttctggggtaccaccaacgacctggacgacgttctgaaactggtttctgaaggtaaagttaaaccggttgtacgttctgctaaactgaaagaactgccggaatacatcgaaaaactgcgtaacaacgcttacgaaggtcgtgttgttttcaacccgtaa

SEQ ID NO: 58 - protein, 336 aa, synthetic construct
  REGION 1..336: note = KRED-NADH-102 variant
MSIPSSQYGFVFNKQSGLKLRNDLPVHKPKAGQLLLKVDAVGLCHSDLHVISEGLDCGDNYVMGHEIAGTVAAVGDDVINYKVGDRVACVGPNGCGGCKYCRGAIDNVCKNAFGDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQAKKLGADAVYETLPESISPGSFSACFDFVSVQATFDVCQKYVEPKGVIMPVGLGAPNLSFNLGDLALREIRILGSFWGTTNDLDDVLKLVSEGKVKPVVRSAKLKELPEYIEKLRNNAYEGRVVFNP

SEQ ID NO: 59 - DNA, 1011 bp, synthetic construct
  misc_feature 1..1011: note = KRED-NADH-102 variant
  source 1..1011: mol_type = other DNA
atgtctatcccgtcttctcagtatggtttcgtatttaacaaacagtctggtctgaaactgcgcaacgatctgccagtacacaagccgaaagctggtcaactgctgctgaaagtagatgcagtaggtctgtgccactctgatctgcacgttatctacgaaggcctggactgcggtgacaactacgttatgggtcacgaaatcgctggtaccgttgctgctgttggtgatgacgttatcaactacaaagtaggtgaccgtgtagcttgtgttggtccgaacggttgcggtggttgcaaatactgccgtggtgctatcgacaacgtttgcaaaaacgctttcggtgactggttcggtctgggttatgatggtggctatcaacaatacctgctggtaactcgcccgcgtaacctgtctcgtatcccggataacgtatctgctgatgttgctgctgcttctaccgacgctgttctgactccgtaccacgctatcaaaatggctcaggtttctccgacctctaacatcctgctgatcggtgctggtggtctgggtggtaacgctatccaggttgctaaagcattcggtgctaaagttaccgttctggacaaaaaaaaagaagctcgtgaccaggctaaaaaactgggtgctgacgctgtttacgaaaccctgccggaatctatctctccgggttctttctctgcttgcttcgacttcgtttctgttcaggctaccttcgacgtttgccagaaatacgttgaaccgaaaggtgttatcatgccggttggtctgggtgctccgaacctgtcttttaacctgggtgacctggctctgcgtgaacaacgtatcctgggttctttctggggtaccaccaacgacctggacgacgttctgaaactggtttctgaaggtaaagttaaaccggttgtacgttctgctaaactgaaagaactgccggaatacatcgaaaaactgcgtaacaacgcttacgaaggtcgtgttgttttcaacccgtaa

SEQ ID NO: 60 - protein, 336 aa, synthetic construct
  REGION 1..336: note = KRED-NADH-102 variant
MSIPSSQYGFVFNKQSGLKLRNDLPVHKPKAGQLLLKVDAVGLCHSDLHVIYEGLDCGDNYVMGHEIAGTVAAVGDDVINYKVGDRVACVGPNGCGGCKYCRGAIDNVCKNAFGDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQAKKLGADAVYETLPESISPGSFSACFDFVSVQATFDVCQKYVEPKGVIMPVGLGAPNLSFNLGDLALREQRILGSFWGTTNDLDDVLKLVSEGKVKPVVRSAKLKELPEYIEKLRNNAYEGRVVFNP

SEQ ID NO: 61 - DNA, 1011 bp, synthetic construct
  misc_feature 1..1011: note = KRED-NADH-102 variant
  source 1..1011: mol_type = other DNA
atgtctatcccgtcttctcagtatggtttcgtatttaacaaacagtctggtctgaaactgcgcaacgatctgccagtacacaagccgaaagctggtcaactgctgctgaaagtagatgcagtaggtctgtgccactctgatctgcacgttatctacgaaggcctggactgcggtgacaactacgttatgggtcacgaaatcgctggtaccgttgctgctgttggtgatgacgttatcaactacaaagtaggtgaccgtgtagcttgtgttggtccgaacggttgcggtggttgcaaatactgccgtggtgctatcgacaacgtttgcaaaaacgctttcggtgactggttcggtctgggttatgatggtggctatcaacaatacctgctggtaactcgcccgcgtaacctgtctcgtatcccggataacgtatctgctgatgttgctgctgcttctaccgacgctgttctgactccgtaccacgctatcaaaatggctcaggtttctccgacctctaacatcctgctgatcggtgctggtggtctgggtggtaacgctatccaggttgctaaagcattcggtgctaaagttaccgttctggacaaaaaaaaagaagctcgtgaccaggctaaaaaactgggtgctgacgctgtttacgaaaccctgccggaatctatctctccgggttctttctctgcttgcttcgacttcgtttctgttcaggctaccttcgacgtttgccagaaatacgttgaaccgaaaggtgttatcatgccggttggtctgggtgctccgaacctgtcttttaacctgggtgacattgctctgcgtgaaatccgtatcctgggttctttctggggtaccaccaacgacctggacgacgttctgaaactggtttctgaaggtaaagttaaaccggttgtacgttctgctaaactgaaagaactgccggaatacatcgaaaaactgcgtaacaacgcttacgaaggtcgtgttgttttcaacccgtaa

SEQ ID NO: 62 - protein, 336 aa, synthetic construct
  REGION 1..336: note = KRED-NADH-102 variant
MSIPSSQYGFVFNKQSGLKLRNDLPVHKPKAGQLLLKVDAVGLCHSDLHVIYEGLDCGDNYVMGHEIAGTVAAVGDDVINYKVGDRVACVGPNGCGGCKYCRGAIDNVCKNAFGDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQAKKLGADAVYETLPESISPGSFSACFDFVSVQATFDVCQKYVEPKGVIMPVGLGAPNLSFNLGDIALREIRILGSFWGTTNDLDDVLKLVSEGKVKPVVRSAKLKELPEYIEKLRNNAYEGRVVFNP

SEQ ID NO: 63 - DNA, 1011 bp, synthetic construct
  misc_feature 1..1011: note = KRED-NADH-102 variant
  source 1..1011: mol_type = other DNA
atgtctatcccgtcttctcagtatggtttcgtatttaacaaacagtctggtctgaaactgcgcaacgatctgccagtacacaagccgaaagctggtcaactgctgctgaaagtagatgcagtaggtctgtgccactctgatctgcacgttatctacgaaggcctgctttgcggtgacaactacgttatgggtcacgaaatcgctggtaccgttgctgctgttggtgatgacgttatcaactacaaagtaggtgaccgtgtagcttgtgttggtccgaacggttgcggtggttgcaaatactgccgtggtgctatcgacaacgtttgcaaaaacgctttcggtgactggttcggtctgggttatgatggtggctatcaacaatacctgctggtaactcgcccgcgtaacctgtctcgtatcccggataacgtatctgctgatgttgctgctgcttctaccgacgctgttctgactccgtaccacgctatcaaaatggctcaggtttctccgacctctaacatcctgctgatcggtgctggtggtctgggtggtaacgctatccaggttgctaaagcattcggtgctaaagttaccgttctggacaaaaaaaaagaagctcgtgaccaggctaaaaaactgggtgctgacgctgtttacgaaaccctgccggaatctatctctccgggttctttctctgcttgcttcgacttcgtttctgttcaggctaccttcgacgtttgccagaaatacgttgaaccgaaaggtgttatcatgccggttggtctgggtgctccgaacctgtcttttaacctgggtgacctggctctgcgtgaaatccgtatcctgggttctttctggggtaccaccaacgacctggacgacgttctgaaactggtttctgaaggtaaagttaaaccggttgtacgttctgctaaactgaaagaactgccggaatacatcgaaaaactgcgtaacaacgcttacgaaggtcgtgttgttttcaacccgtaa

SEQ ID NO: 64 - protein, 336 aa, synthetic construct
  REGION 1..336: note = KRED-NADH-102 variant
MSIPSSQYGFVFNKQSGLKLRNDLPVHKPKAGQLLLKVDAVGLCHSDLHVIYEGLLCGDNYVMGHEIAGTVAAVGDDVINYKVGDRVACVGPNGCGGCKYCRGAIDNVCKNAFGDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQAKKLGADAVYETLPESISPGSFSACFDFVSVQATFDVCQKYVEPKGVIMPVGLGAPNLSFNLGDLALREIRILGSFWGTTNDLDDVLKLVSEGKVKPVVRSAKLKELPEYIEKLRNNAYEGRVVFNP

SEQ ID NO: 65 - DNA, 1011 bp, synthetic construct
  misc_feature 1..1011: note = KRED-NADH-102 variant
  source 1..1011: mol_type = other DNA
atgtctatcccgtcttctcagtatggtttcgtatttaacaaacagtctggtctgaaactgcgcaacgatctgccagtacacaagccgaaagctggtcaactgctgctgaaagtagatgcagtaggtctgtgccactctgatctgcacgttatctacgaaggcctggactgcggtgacaactacgttatgggtcacgaaatcgctggtaccgttgctgctgttggtgatgacgttatcaactacaaagtaggtgaccgtgtagcttgtgttggtccgaacggttgcggtggttgcaaatactgccgtggtgctatcgacaacgtttgcacaaacgctttcggtgactggttcggtctgggttatgatggtggctatcaacaatacctgctggtaactcgcccgcgtaacctgtctcgtatcccggataacgtatctgctgatgttgctgctgcttctaccgacgctgttctgactccgtaccacgctatcaaaatggctcaggtttctccgacctctaacatcctgctgatcggtgctggtggtctgggtggtaacgctatccaggttgctaaagcattcggtgctaaagttaccgttctggacaaaaaaaaagaagctcgtgaccaggctaaaaaactgggtgctgacgctgtttacgaaaccctgccggaatctatctctccgggttctttctctgcttgcttcgacttcgtttctgttcaggctaccttcgacgtttgccagaaatacgttgaaccgaaaggtgttatcatgccggttggtctgggtgctccgaacctgtcttttaacctgggtgacctggctctgcgtgaaatccgtatcctgggttctttctggggtaccaccaacgacctggacgacgttctgaaactggtttctgaaggtaaagttaaaccggttgtacgttctgctaaactgaaagaactgccggaatacatcgaaaaactgcgtaacaacgcttacgaaggtcgtgttgttttcaacccgtaa

SEQ ID NO: 66 - protein, 336 aa, synthetic construct
  REGION 1..336: note = KRED-NADH-102 variant
MSIPSSQYGFVFNKQSGLKLRNDLPVHKPKAGQLLLKVDAVGLCHSDLHVIYEGLDCGDNYVMGHEIAGTVAAVGDDVINYKVGDRVACVGPNGCGGCKYCRGAIDNVCTNAFGDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQAKKLGADAVYETLPESISPGSFSACFDFVSVQATFDVCQKYVEPKGVIMPVGLGAPNLSFNLGDLALREIRILGSFWGTTNDLDDVLKLVSEGKVKPVVRSAKLKELPEYIEKLRNNAYEGRVVFNP

SEQ ID NO: 67 - DNA, 1011 bp, synthetic construct
  misc_feature 1..1011: note = KRED-NADH-102 variant
  source 1..1011: mol_type = other DNA
atgtctatcccgtcttctcagtatggtttcgtatttaacaaacagtctggtctgaaactgcgcaacgatctgccagtacacaagccgaaagctggtcaactgctgctgaaagtagatgcagtaggtctgtgccactctgatctgcacgttatctacgaaggcctggactgcggtgacaactacgttatgggtcacgaaatcgctggtaccgttgctgctgttggtgatgacgttatcaactacaaagtaggtgaccgtgtagcttgtgttggtccgaacggttgcggtggttgcaaatactgccgtggtgctatcgacaacgtttgcaaaaacgctttcggtgactggttcggtctgggttatgatggtggctatcaacaatacctgctggtaactcgcccgcgtaacctgtctcgtatcccggataacgtatctgctgatgttgctgctgcttctaccgacgctgttctgactccgtaccacgctatcaaaatggctcaggtttctccgacctctaacatcctgctgatcggtgctggtggtctgggtggtaacgctatccaggttgctaaagcattcggtgctaaagttaccgttctggacaaaaaaaaagaagctcgtgaccaggctaaaaaactgggtgctgacgctgtttacgaaaccctgccggaatctatctctagtggttctttctctgcttgcttcgacttcgtttctgttcaggctaccttcgacgtttgccagaaatacgttgaaccgaaaggtgttatcatgccggttggtctgggtgctccgaacctgtcttttaacctgggtgacctggctctgcgtgaaatccgtatcctgggttctttctggggtaccaccaacgacctggacgacgttctgaaactggtttctgaaggtaaagttaaaccggttgtacgttctgctaaactgaaagaactgccggaatacatcgaaaaactgcgtaacaacgcttacgaaggtcgtgttgttttcaacccgtaa

SEQ ID NO: 68 - protein, 336 aa, synthetic construct
  REGION 1..336: note = KRED-NADH-102 variant
MSIPSSQYGFVFNKQSGLKLRNDLPVHKPKAGQLLLKVDAVGLCHSDLHVIYEGLDCGDNYVMGHEIAGTVAAVGDDVINYKVGDRVACVGPNGCGGCKYCRGAIDNVCKNAFGDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQAKKLGADAVYETLPESISSGSFSACFDFVSVQATFDVCQKYVEPKGVIMPVGLGAPNLSFNLGDLALREIRILGSFWGTTNDLDDVLKLVSEGKVKPVVRSAKLKELPEYIEKLRNNAYEGRVVFNP

SEQ ID NO: 69 - DNA, 1011 bp, synthetic construct
  misc_feature 1..1011: note = KRED-NADH-102 variant
  source 1..1011: mol_type = other DNA
atgtctatcccgtcttctcagtatggtttcgtatttaacaaacagtctggtctgaaactgcgcaacgatctgccagtacacaagccgaaagctggtcaactgctgctgaaagtagatgcagtaggtctgtgccactctgatctgcacgttatctacgaaggcctggactgcggtgacaactacgttatgggtcacgaaatcgctggtaccgttgctgctgttggtgatgacgttatcaactacaaagtaggtgaccgtgtagcttgtgttggtccgaacggttgcggtggttgcaaatactgccgtggtgccatcgacaacgtttgcaaaaacgctttcggtgactggttcggtctgggttatgatggtggctatcaacaatacctgctggtaactcgcccgcgtaacctggtccgtatcccggataacgtatcttcggatgttgctgctgcttctaccgacgctgttctgactccgtaccacgctatcaaaatggctcaggtttctccgacctctaacatcctgctgatcggtgctggtggtctgggtggtaacgctatccaggttgctaaagcattcggtgctaaagttaccgttctggacaaaaaaaaagaagctcgtgaccaggctaaaaaactgggtgctgacgctgtttacgaaaccctgccggaatctatctctccgggttctttctctgcttgcttcgacttcgtttctgttcaggctaccttcgacgtttgccagaaatacgttgaaccgaaaggtgttatcgtgccggttggtctgggtgctccgaacctgtcttttaacctgggtgacctggctctgcgtgaaatccgtatcctgggttctttctggggtaccagcaacgacctggacgacgttctgaaactggtttctgaaggtaaagttaaaccggttgtacgttctgctaaactgaaagaactgccggaatacatcgaaaaactgcgtaacaacgcttacgaaggtcgtgttgttttcaacccgtaa

SEQ ID NO: 70 - protein, 336 aa, synthetic construct
  REGION 1..336: note = KRED-NADH-102 variant
MSIPSSQYGFVFNKQSGLKLRNDLPVHKPKAGQLLLKVDAVGLCHSDLHVIYEGLDCGDNYVMGHEIAGTVAAVGDDVINYKVGDRVACVGPNGCGGCKYCRGAIDNVCKNAFGDWFGLGYDGGYQQYLLVTRPRNLVRIPDNVSSDVAAASTDAVLTPYHAIKMAQVSPTSNILLIGAGGLGGNAIQVAKAFGAKVTVLDKKKEARDQAKKLGADAVYETLPESISPGSFSACFDFVSVQATFDVCQKYVEPKGVIVPVGLGAPNLSFNLGDLALREIRILGSFWGTSNDLDDVLKLVSEGKVKPVVRSAKLKELPEYIEKLRNNAYEGRVVFNP

SEQ ID NO: 71 - DNA, 1011 bp, synthetic construct
  misc_feature 1..1011: note = KRED-NADH-102 variant
  source 1..1011: mol_type = other DNA
atgtcgatcccgtcttctcagtatggtttcgtatttaacaaacagtctggtctgaaactgcgcaacgatctgccagtacacaagccgaaagctggtcaactgctgctgaaagtagatgcagtaggtctgtgccactctgatctgcacgttatctacgaaggcctggactgcggtgacaactacgttatgggtcacgaaatcgctggtaccgttgctgctgttggtgatgacgttatcaactacaaagtaggtgaccgtgtagcttgtgttggtccgaacggttgcggtggttgcaaatactgccgtggtgctatcgacaacgtttgcaaaaacgctttcggtgactggttcggtctgggttatgatggtggctatcaacaatacctgctggtaactcgcccgcgtaacctgtctcgtatcccggataacgtatctgctgatgttgctgctgcttctaccgacgctgttctgactccgtaccacgctatcaaaatggctcaggtttctccgacctctaacatcctgctgatcggtgctggtggtctgggtggtaacgctatccaggttgctaaagcattcggtgctaaagttaccgttctggacaaaaaaaaagaagctcgtgaccaggctagaaaactgggtgctgacgctgtttacgaaaccctgccggaatctatctctccgggttctttctctgcttgcttcgacttcgtttctgttcaggctaccttcgacgtttgccagaaatacgttgaaccgaaaggtgttatcatgccggttggtctgggtgctccgaacctgtcttttaacctgggtgacctggctctgcgtgaaatccgtatcctgggttctttctggggtaccaccaacgacctggacgacgttctgaaactggtttctgaaggtaaagttaaaccggttgtacgttctgctaaactgaaagaactgccggaatacatcgaaaaactgcgtaacaacgcttacgaaggtcgtgttgttttcaacccgtaa

SEQ ID NO: 72 - protein, 336 aa, synthetic construct
  REGION 1..336: note = KRED-NADH-102 variant
MSIPSSQYGFVFNKQSGLKLRNDLPVHKPKAGQLLLKVDAVGLCHSDLHVIYEGLDCGDNYVMGHEIAGTVAAVGDDVINYKVGDRVACVGPNGCGGCKYCRGAIDNVCKNAFGDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQARKLGADAVYETLPESISPGSFSACFDFVSVQATFDVCQKYVEPKGVIMPVGLGAPNLSFNLGDLALREIRILGSFWGTTNDLDDVLKLVSEGKVKPVVRSAKLKELPEYIEKLRNNAYEGRVVFNP

SEQ ID NO: 73 - DNA, 1011 bp, synthetic construct
  misc_feature 1..1011: note = KRED-NADH-102 variant
  source 1..1011: mol_type = other DNA
atgtcgatcccgtcttctcagtatggtttcgtatttaacaaacagtctggtctgaaactgcgcaacgatctgccagtacacaagccgaaagctggtcaactgctgctgagagtagatgcagtaggtctgtgccactctgatctgcacgttatctacgaaggcctggactgcggtgacaactacgttatgggtcacgaaatcgctggtaccgttgctgctgttggtgatgacgttatcaactacaaagtaggtgaccgtgtagcttgtgttggtccgaacggttgcggtggttgcaaatactgccgtggtgctatcgacaacgtttgcaaaaacgctttcggtgactggttcggtctgggttatgatggtggctatcaacaatacctgctggtaactcgcccgcgtaacctgtctcgtatcccggataacgtatctgctgatgttgctgctgcttctaccgacgctgttctgactccgtaccacgctatcaaaatggctcaggtttctccgacctctaacatcctgctgatcggtgctggtggtctgggtggtaacgctatccaggttgctaaagcattcggtgctaaagttaccgttctggacaaaaaaaaagaagctcgtgaccaggctaaaaaactgggtgctgacgctgtttacgaaaccctgccggaatctatctctccgggttctttctctgcttgcttcgacttcgtttctgttcaggctaccttcgacgtttgccagaaatacgttgaaccgaaaggtgttatcatgccggttggtctgggtgctccgaacctgtcttttaacctgggtgacctggctctgcgtgaaatccgtatcctgggttctttctggggtaccaccaacgacctggacgacgttctgaaactggtttctgaaggtaaagttaaaccggttgtacgttctgctaaactgaaagaactgccggaatacatcgaaaaactgcgtaacaacgcttacgaaggtcgtgttgttttcaacccgtaa

SEQ ID NO: 74 - protein, 336 aa, synthetic construct
  REGION 1..336: note = KRED-NADH-102 variant
MSIPSSQYGFVFNKQSGLKLRNDLPVHKPKAGQLLLRVDAVGLCHSDLHVIYEGLDCGDNYVMGHEIAGTVAAVGDDVINYKVGDRVACVGPNGCGGCKYCRGAIDNVCKNAFGDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQAKKLGADAVYETLPESISPGSFSACFDFVSVQATFDVCQKYVEPKGVIMPVGLGAPNLSFNLGDLALREIRILGSFWGTTNDLDDVLKLVSEGKVKPVVRSAKLKELPEYIEKLRNNAYEGRVVFNP

SEQ ID NO: 75 - DNA, 1011 bp, synthetic construct
  misc_feature 1..1011: note = KRED-NADH-102 variant
  source 1..1011: mol_type = other DNA
atgtctatcccgtcttctcagtatggtttcgtatttaacaaacagtctggtctgaaactgcgcaacgatctgccagtacacaagccgaaagctggtcaactgctgctgagagtagatgcagtaggtctgtgccactctgatctgcacgttatctacgaaggcctggactgcggtgacaactacgttatgggtcacgaaatcgctggtaccgttgctgctgttggtgatgacgttatcaactacaaagtaggtgaccgtgtagcttgtgttggtccgaacggttgcggtggttgcaaatactgccgtggtgctatcgacaacgtttgcaaaaacgctttcggtgactggttcggtctgggttatgacggtggctatcaacaatacctgctggtaactcgcccgcgtaacctgtctcgtatcccggataacgtatctgctgatgttgctgctgcttctaccgacgctgttctgactccgtaccacgctatcaaaatggctcaggtttctccgacctctaacatcctgctgatcggtgctggtggtctgggtggtaacgctatccaggttgctaaagcattcggtgctaaagttaccgttctggacaaaaaaaaagaagctcgtgaccaggctagaaaactgggtgctgacgctgtttacgaaaccctgccggaatctatctctccgcgttctttctctgcttgcttcgacttcgtttctgttcaggctaccttcgacgtttgccagaaatacgttgaaccgaaaggtgttatcatgccggttggtctgggtgctccgaacctgtcttttaacctgggtgacctggctctgcgtgaaatccgtatcctgggttctttctggggtaccaccaacgacctggacgacgttctgaaactggtttctgaaggtaaagttaaaccggttgtacgttctgctaaactgaaagaactgccggaatacatcgaaaaactgcgtaacaacgcttacgaaggtcgtgttgttttcaacccgtaa

SEQ ID NO: 76 - protein, 336 aa, synthetic construct
  REGION 1..336: note = KRED-NADH-102 variant
MSIPSSQYGFVFNKQSGLKLRNDLPVHKPKAGQLLLRVDAVGLCHSDLHVIYEGLDCGDNYVMGHEIAGTVAAVGDDVINYKVGDRVACVGPNGCGGCKYCRGAIDNVCKNAFGDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQARKLGADAVYETLPESISPRSFSACFDFVSVQATFDVCQKYVEPKGVIMPVGLGAPNLSFNLGDLALREIRILGSFWGTTNDLDDVLKLVSEGKVKPVVRSAKLKELPEYIEKLRNNAYEGRVVFNP

SEQ ID NO: 77 - DNA, 1011 bp, synthetic construct
  misc_feature 1..1011: note = KRED-NADH-102 variant
  source 1..1011: mol_type = other DNA
atgtcgatcccgtcttctcagtatggtttcgtatttaacaaacagtctggtctgaaactgcgcaacgatctgccagtacacaagccgaaagctggtcaactgctgctgaaagtagatgcagtaggtctgtgccactctgatctgcacgttatctacgaaggcctggactgcggtgacaactacgttatgggtcacgaaatcgctggtaccgttgctgctgttggtgatgacgttatcaactacaaagtaggtgaccgtgtagcttgtgttggtccgaacggttgcggtggttgcaaatactgccgtggtgctatcgacaacgtttgcaaaaacgctttcggtgactggttcggtctgggttatgacggtggctatcaacaatacctgctggtaactcgcccgcgtaacctgtctcgtatcccggataacgtatctgctgatgttgctgctgcttctaccgacgctgttctgactccgtaccacgctatcaaaatggctcaggtttctccgacctctaacatcctgctgatcggtgctggtggtctgggtggtaacgctatccaggttgctaaagcattcggtgctaaagttaccgttctggacaaaaaaaaagaagctcgtgaccaggctagaaaactgggtgctgacgctgtttacgaaaccctgccggaatctatctctccgcgttctttctctgcttgcttcgacttcgtttctgttcaggctaccttcgacgtttgccagaaatacgttgaaccgaaaggtgttatcatgccggttggtctgggtgctccgaacctgtcttttaacctgggtgacctggctctgcgtgaaatccgtatcctgggttctttctggggtaccaccaacgacctggacgacgttctgaaactggtttctgaaggtaaagttaaaccggttgtacgttctgctaaactgaaagaactgccggaatacatcgaaaaactgcgtaacaacgcttacgaaggtcgtgttgttttcaacccgtaa

SEQ ID NO: 78 - protein, 336 aa, synthetic construct
  REGION 1..336: note = KRED-NADH-102 variant
MSIPSSQYGFVFNKQSGLKLRNDLPVHKPKAGQLLLKVDAVGLCHSDLHVIYEGLDCGDNYVMGHEIAGTVAAVGDDVINYKVGDRVACVGPNGCGGCKYCRGAIDNVCKNAFGDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQARKLGADAVYETLPESISPRSFSACFDFVSVQATFDVCQKYVEPKGVIMPVGLGAPNLSFNLGDLALREIRILGSFWGTTNDLDDVLKLVSEGKVKPVVRSAKLKELPEYIEKLRNNAYEGRVVFNP

SEQ ID NO: 79 - DNA, 1011 bp, synthetic construct
  misc_feature 1..1011: note = KRED-NADH-102 variant
  source 1..1011: mol_type = other DNA
atgtcgatcccgtcttctcagtatggtttcgtatttaacaaacagtctggtctgaaactgcgcaacgatctgccagtacacaagccgaaagctggtcaactgctgctgaaagtagatgcagtaggtctgtgccactctgatctgcacgttatctacgaaggcctggactgcggtgacaactacgttatgggtcacgaaatcgctggtaccgttgctgctgttggtgatgacgttatcaactacaaagtaggtgaccgtgtagcttgtgttggtccgaacggttgcggtggttgcaaatactgccgtggtgctatcgacaacgtttgcaaaaacgctttcggtgactggttcggtctgggttatgatggtggctatcaacaatacctgctggtaactcgcccgcgtaacctgtctcgtatcccggataacgtatctgctgatgttgctgctgcttctaccgacgctgttctgactccgtaccacgctatcaaaatggctcaggtttctccgacctctaacatcctgctgatcggtgctggtggtctgggtggtaacgctatccaggttgctaaagcattcggtgctaaagttaccgttctggacaaaaaaaaagaagctcgtgaccaggctaaaaaactgggtgctgacgctgtttacgaaaccctgccggaatctatctctccgcgttctttctctgcttgcttcgacttcgtttctgttcaggctaccttcgacgtttgccagaaatacgttgaaccgaaaggtgttatcatgccggttggtctgggtgctccgaacctgtcttttaacctgggtgacctggctctgcgtgaaatccgtatcctgggttctttctggggtaccaccaacgacctggacgacgttctgaaactggtttctgaaggtaaagttaaaccggttgtacgttctgctaaactgaaagaactgccggaatacatcgaaaaactgcgtaacaacgcttacgaaggtcgtgttgttttcaacccgtaa

SEQ ID NO: 80 - protein, 336 aa, synthetic construct
  REGION 1..336: note = KRED-NADH-102 variant
MSIPSSQYGFVFNKQSGLKLRNDLPVHKPKAGQLLLKVDAVGLCHSDLHVIYEGLDCGDNYVMGHEIAGTVAAVGDDVINYKVGDRVACVGPNGCGGCKYCRGAIDNVCKNAFGDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQAKKLGADAVYETLPESISPRSFSACFDFVSVQATFDVCQKYVEPKGVIMPVGLGAPNLSFNLGDLALREIRILGSFWGTTNDLDDVLKLVSEGKVKPVVRSAKLKELPEYIEKLRNNAYEGRVVFNP

SEQ ID NO: 81 - DNA, 1011 bp, synthetic construct
  misc_feature 1..1011: note = KRED-NADH-102 variant
  source 1..1011: mol_type = other DNA
atgtctatcccgtcttctcagtatggtttcgtatttaacaaacagtctggtctgaaactgcgcaacgatctgccagtacacaagccgaaagctggtcaactgctgctgagagtagatgcagtaggtctgtgccactctgatctgcacgttatctacgaaggcctggactgcggtgacaactacgttatgggtcacgaaatcgctggtaccgttgctgctgttggtgatgacgttatcaactacaaagtaggtgaccgtgtagcttgtgttggtccgaacggttgcggtggttgcaaatactgccgtggtgctatcgacaacgtttgcaaaaacgctttcggtgactggttcggtctgggttatgacggtggctatcaacaatacctgctggtaactcgcccgcgtaacctgtctcgtatcccggataacgtatctgctgatgttgctgctgcttctaccgacgctgttctgactccgtaccacgctatcaaaatggctcaggtttctccgacctctaacatcctgctgatcggtgctggtggtctgggtggtaacgctatccaggttgctaaagcattcggtgctaaagttaccgttctggacaaaaaaaaagaagctcgtgaccaggctaaaaaactgggtgctgacgctgtttacgaaaccctgccggaatctatctctccgcgttctttctctgcttgcttcgacttcgtttctgttcaggctaccttcgacgtttgccagaaatacgttgaaccgaaaggtgttatcatgccggttggtctgggtgctccgaacctgtcttttaacctgggtgacctggctctgcgtgaaatccgtatcctgggttctttctggggtaccaccaacgacctggacgacgttctgaaactggtttctgaaggtaaagttaaaccggttgtacgttctgctaaactgaaagaactgccggaatacatcgaaaaactgcgtaacaacgcttacgaaggtcgtgttgttttcaacccgtaa

SEQ ID NO: 82 - protein, 336 aa, synthetic construct
  REGION 1..336: note = KRED-NADH-102 variant
MSIPSSQYGFVFNKQSGLKLRNDLPVHKPKAGQLLLRVDAVGLCHSDLHVIYEGLDCGDNYVMGHEIAGTVAAVGDDVINYKVGDRVACVGPNGCGGCKYCRGAIDNVCKNAFGDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQAKKLGADAVYETLPESISPRSFSACFDFVSVQATFDVCQKYVEPKGVIMPVGLGAPNLSFNLGDLALREIRILGSFWGTTNDLDDVLKLVSEGKVKPVVRSAKLKELPEYIEKLRNNAYEGRVVFNP

SEQ ID NO: 83 - DNA, 1011 bp, synthetic construct
  misc_feature 1..1011: note = KRED-NADH-102 variant
  source 1..1011: mol_type = other DNA
atgtcgatcccgtcttctcagtatggtttcgtatttaacaaacagtctggtctgaaactgcgcaacgatctgccagtacacaagccgaaagctggtcaactgctgctgagagtagatgcagtaggtctgtgccactctgatctgcacgttatctacgaaggcctggactgcggtgacaactacgttatgggtcacgaaatcgctggtaccgttgctgctgttggtgatgacgttatcaactacaaagtaggtgaccgtgtagcttgtgttggtccgaacggttgcggtggttgcaaatactgccgtggtgctatcgacaacgtttgcaaaaacgctttcggtgactggttcggtctgggttatgatggtggctatcaacaatacctgctggtaactcgcccgcgtaacctgtctcgtatcccggataacgtatctgctgatgttgctgctgcttctaccgacgctgttctgactccgtaccacgctatcaaaatggctcaggtttctccgacctctaacatcctgctgatcggtgctggtggtctgggtggtaacgctatccaggttgctaaagcattcggtgctaaagttaccgttctggacaaaaaaaaagaagctcgtgaccaggctagaaaactgggtgctgacgctgtttacgaaaccctgccggaatctatctctccgggttctttctctgcttgcttcgacttcgtttctgttcaggctaccttcgacgtttgccagaaatacgttgaaccgaaaggtgttatcatgccggttggtctgggtgctccgaacctgtcttttaacctgggtgacctggctctgcgtgaaatccgtatcctgggttctttctggggtaccaccaacgacctggacgacgttctgaaactggtttctgaaggtaaagttaaaccggttgtacgttctgctaaactgaaagaactgccggaatacatcgaaaaactgcgtaacaacgcttacgaaggtcgtgttgttttcaacccgtaa

SEQ ID NO: 84 - protein, 336 aa, synthetic construct
  REGION 1..336: note = KRED-NADH-102 variant
MSIPSSQYGFVFNKQSGLKLRNDLPVHKPKAGQLLLRVDAVGLCHSDLHVIYEGLDCGDNYVMGHEIAGTVAAVGDDVINYKVGDRVACVGPNGCGGCKYCRGAIDNVCKNAFGDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQARKLGADAVYETLPESISPGSFSACFDFVSVQATFDVCQKYVEPKGVIMPVGLGAPNLSFNLGDLALREIRILGSFWGTTNDLDDVLKLVSEGKVKPVVRSAKLKELPEYIEKLRNNAYEGRVVFNP

SEQ ID NO: 85 - DNA, 1011 bp, synthetic construct
  misc_feature 1..1011: note = KRED-NADH-102 variant
  source 1..1011: mol_type = other DNA
atgtctatcccgtcttctcagtatggtttcgtatttaacaaacagtctggtctgaaactgcgcaacgatctgccagtacacaagccgaaagctggtcaactgctgctgaaagtagatgcagtaggtctgtgccactctgatctgcacgttatctacgaaggcctggactgcggtgacaactacgttatgggtcacgaaatcgctggtaccgttgctgctgttggtgatgacgttatcaactacaaagtaggtgaccgtgtagcttgtgttggtccgaacggttgcggtggttgcaaatactgccgtggtgctatcgacaacgtttgcaaaaacgctttcggtgactggttcggtctgggttatgatggtggctatcaacaatacctgctggtaactcgcccgcgtaacctgtctcgtatcccggataacgtatctgctgatgttgctgctgcttctaccgacgctgttctgactccgtaccacgctatcaaaatggctcaggtttctccgacctctaacatcctgctgatcggtgctggtggtctgggtggtaacgctatccaggttgctaaagcattcggtgctaaagttaccgttctggacaaaaaaaaagaagctcgtgaccaggctaaaaaactgggtgctgacgctgtttacgaaaccctgccggaatctatctctccgggttctttctctgcttgcttcgacttcgtttctgttcaggctaccttcgacgtttgccagaaatacgttgaaccgaaaggtgttatcatgccggttggtctgggtgctccgaacctgtcttttaacctgggtgacctggcgatgcgtgaaatccgtatcctgggttctttctggggtaccaccaacgacctggacgacgttctgaaactggtttctgaaggtaaagttaaaccggttgtacgttctgctaaactgaaagaactgccggaatacatcgaaaaactgcgtaacaacgcttacgaaggtcgtgttgttttcaacccgtaa

SEQ ID NO: 86 - protein, 336 aa, synthetic construct
  REGION 1..336: note = KRED-NADH-102 variant
MSIPSSQYGFVFNKQSGLKLRNDLPVHKPKAGQLLLKVDAVGLCHSDLHVIYEGLDCGDNYVMGHEIAGTVAAVGDDVINYKVGDRVACVGPNGCGGCKYCRGAIDNVCKNAFGDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQAKKLGADAVYETLPESISPGSFSACFDFVSVQATFDVCQKYVEPKGVIMPVGLGAPNLSFNLGDLAMREIRILGSFWGTTNDLDDVLKLVSEGKVKPVVRSAKLKELPEYIEKLRNNAYEGRVVFNP

SEQ ID NO: 87 - DNA, 1011 bp, synthetic construct
  misc_feature 1..1011: note = KRED-NADH-102 variant
  source 1..1011: mol_type = other DNA
atgtctatcccgtcttctcagtatggtttcgtatttaacaaacagtctggtctgaaactgcgcaacgatctgccagtacacaagccgaaagctggtcaactgctgctgaaagtagatgcagtaggtctgtgccactctgatctgcacgttatctacgaaggcctggactgtggtgacaactacgttatgggtcacgaaatcgctggtaccgttgctgctgttggtgatgacgttaccaactacaaatccggtgaccgtgtagcttgtgttggtccgaacggttgcggtggttgcaaatactgccgtggtgctatcgacaacgtttgcaaaaacgctttcggtgactggttcggtctgggttatgatggtggctatcaacaatacctgctggtaactcgcccgcgtaacctgtctcgtatcccggataacgtatctgctgatgttgctgctgcttctaccgacgctgttctgactccgtaccacgctatcaaaatggctcaggtttctccgacctctaacatcctgctgatcggtgctggtggtctgggtggtaacgctatccaggttgctaaagcattcggtgctaaagttaccgttctggacaaaaaaaaagaagctcgtgaccaggctaaaaaactgggtgctgacgctgtttacgaaaccctgccggaatctatctctccgggttctttctctgcttgcttcgacttcgtttctgttcaggctaccttcgacgtttgccagaaatacgttgaaccgaaaggtgttatcatgccggttggtctgggtgctccgaacctgtcttttaacctgggtgacctgaacatgcgtgaaatccgtatcctgggttctttctggggtaccaccaacgacctggacgacgttctgaaactggtttctgaaggtaaagttaaaccggttgtacgttctgctaaactgaaagaactgccggaatacatcgaaaaactgcgtaacaacgcttacgaaggtcgtgttgttttcaacccgtaa

SEQ ID NO: 88 - protein, 336 aa, synthetic construct
  REGION 1..336: note = KRED-NADH-102 variant
MSIPSSQYGFVFNKQSGLKLRNDLPVHKPKAGQLLLKVDAVGLCHSDLHVIYEGLDCGDNYVMGHEIAGTVAAVGDDVTNYKSGDRVACVGPNGCGGCKYCRGAIDNVCKNAFGDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQAKKLGADAVYETLPESISPGSFSACFDFVSVQATFDVCQKYVEPKGVIMPVGLGAPNLSFNLGDLNMREIRILGSFWGTTNDLDDVLKLVSEGKVKPVVRSAKLKELPEYIEKLRNNAYEGRVVFNP

SEQ ID NO: 89 - DNA, 1011 bp, synthetic construct
  misc_feature 1..1011: note = KRED-NADH-102 variant
  source 1..1011: mol_type = other DNA
atgtctatcccgtcttctcagtatggtttcgtatttaacaaacagtctggtctgaaactgcgcaacgatctgccagtacacaagccgaaagctggtcaactgctgctgaaagtagatgcagtaggtctgtgccactctgatctgcacgttatctacgaaggccttgactgtggtgacaactacgttatgggtcacgaaatcgctggtaccgttgctgctgttggtgatgacgttatcaactacaaatccggtgaccgtgtagcttgtgttggtccgaacggttgcggtggttgcaaatactgccgtggtgctatcgacaacgtttgcaaaaacgctttcggtgactggttcggtctgggttatgatggtggctatcaacaatacctgctggtaactcgcccgcgtaacctgtctcgtatcccggataacgtatctgctgatgttgctgctgcttctaccgacgctgttctgactccgtaccacgctatcaaaatggctcaggtttctccgacctctaacatcctgctgatcggtgctggtggtctgggtggtaacgctatccaggttgctaaagcattcggtgctaaagttaccgttctggacaaaaaaaaagaagctcgtgaccaggctaaaaaactgggtgctgacgctgtttacgaaaccctgccggaatctatctctccgggttctttctctgcttgcttcgacttcgtttctgttcaggctaccttcgacgtttgccagaaatacgttgaaccgaaaggtgttatcatgccggttggtctgggtgctccgaacctgtcttttaacctgggtgacctgaacatgcgtgaaatccgtatcctgggttctttctggggtaccaccaacgacctggacgacgttctgaaactggtttctgaaggtaaagttaaaccggttgtacgttctgctaaactgaaagaactgccggaatacatcgaaaaactgcgtaacaacgcttacgaaggtcgtgttgttttcaacccgtaa

SEQ ID NO: 90 - protein, 336 aa, synthetic construct
  REGION 1..336: note = KRED-NADH-102 variant
MSIPSSQYGFVFNKQSGLKLRNDLPVHKPKAGQLLLKVDAVGLCHSDLHVIYEGLDCGDNYVMGHEIAGTVAAVGDDVINYKSGDRVACVGPNGCGGCKYCRGAIDNVCKNAFGDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQAKKLGADAVYETLPESISPGSFSACFDFVSVQATFDVCQKYVEPKGVIMPVGLGAPNLSFNLGDLNMREIRILGSFWGTTNDLDDVLKLVSEGKVKPVVRSAKLKELPEYIEKLRNNAYEGRVVFNP

SEQ ID NO: 91 - DNA, 1011 bp, synthetic construct
  misc_feature 1..1011: note = KRED-NADH-102 variant
  source 1..1011: mol_type = other DNA
atgtctatcccgtcttctcagtatggtttcgtatttaacaaacagtctggtctgaaactgcgcaacgatctgccagtacacaagccgaaagctggtcaactgctgctgaaagtagatgcagtaggtctgtgccactctgatctgcacgttatctacgaaggcctggactgcggtgacaactacgttatgggtcacgaaatcgctggtaccgttgctgctgttggtgatgacgttatcaactacaaatccggtgaccgtgtagcttgtgttggtccgaacggttgcggtggttgcaaatactgccgtggtgctatcgacaacgtttgcaaaaacgctttcggtgactggttcggtctgggttatgatggtggctatcaacaatacctgctggtaactcgcccgcgtaacctgtctcgtatcccggataacgtatctgctgatgttgctgctgcttctaccgacgctgttctgactccgtaccacgctatcaaaatggctcaggtttctccgacctctaacatcctgctgatcggtgctggtggtctgggtggtaacgctatccaggttgctaaagcattcggtgctaaagttaccgttctggacaaaaaaaaagaagctcgtgaccaggctaaaaaactgggtgctgacgctgtttacgaaaccctgccggaatctatctctccgggttctttctctgcttgcttcgacttcgtttctgttcaggctaccttcgacgtttgccagaaatacgttgaaccgaaaggtgttatcatgccggttggtctgggtgctccgaacctgtcttttaacctgggtgacctggcgatgcgtgaaatccgtatcctgggttctttctggggtaccaccaacgacctggacgacgttctgaaactggtttctgaaggtaaagttaaaccggttgtacgttctgctaaactgaaagaactgccggaatacatcgaaaaactgcgtaacaacgcttacgaaggtcgtgttgttttcaacccgtaa

SEQ ID NO: 92 - protein, 336 aa, synthetic construct
  REGION 1..336: note = KRED-NADH-102 variant
MSIPSSQYGFVFNKQSGLKLRNDLPVHKPKAGQLLLKVDAVGLCHSDLHVIYEGLDCGDNYVMGHEIAGTVAAVGDDVINYKSGDRVACVGPNGCGGCKYCRGAIDNVCKNAFGDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQAKKLGADAVYETLPESISPGSFSACFDFVSVQATFDVCQKYVEPKGVIMPVGLGAPNLSFNLGDLAMREIRILGSFWGTTNDLDDVLKLVSEGKVKPVVRSAKLKELPEYIEKLRNNAYEGRVVFNP

SEQ ID NO: 93 - DNA, 1011 bp, synthetic construct
  misc_feature 1..1011: note = KRED-NADH-102 variant
  source 1..1011: mol_type = other DNA
atgtctatcccgtcttctcagtatggtttcgtatttaacaaacagtctggtctgaaactgcgcaacgatctgccagtacacaagccgaaagctggtcaactgctgctgaaagtagatgcagtaggtctgtgccattctgatctgcacgttatctacgaaggcctggactgtggtgacaactacgttatgggtcacgaaatcgctggtaccgttgctgctgttggtgatgacgttatcaactacaaagtaggtgaccgtgtagcttgtgttggtccgaacggttgcggtggttgcaaatactgccgtggtgctatcgacaacgtttgcaaaaacgctttcggtgactggttcggtctgggttatgatggtggctatcaacaatacctgctggtaactcgcccgcgtaacctgtctcgtatcccggataacgtatctgctgatgttgctgctgcttctaccgacgctgttctgactccgtaccacgctatcaaaatggctcaggtttctccgacctctaacatcctgctgatcggtgctggtggtctgggtggtaacgctatccaggttgctaaagcattcggtgctaaagttaccgttctggacaaaaaaaaagaagctcgtgaccaggctaaaaaactgggtgctgacgctgtttacgaaaccctgccggaatctatctctccgggttctttctctgcttgcttcgacttcgtttctgttcaggctaccttcgacgtttgccagaaatacgttgaaccgaaaggtgttatcatgccggttggtctgggtgctccgaacctgtcttttaacctgggtgacctgaatatgcgtgaaatccgtatcctgggttctttctggggtaccaccaacgacctggacgacgttctgaaactggtttctgaaggtaaagttaaaccggttgtacgttctgctaaactgaaagaactgccggaatacatcgaaaaactgcgtaacaacgcttacgaaggtcgtgttgttttcaacccgtaa

SEQ ID NO: 94 - protein, 336 aa, synthetic construct
  REGION 1..336: note = KRED-NADH-102 variant
MSIPSSQYGFVFNKQSGLKLRNDLPVHKPKAGQLLLKVDAVGLCHSDLHVIYEGLDCGDNYVMGHEIAGTVAAVGDDVINYKVGDRVACVGPNGCGGCKYCRGAIDNVCKNAFGDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQAKKLGADAVYETLPESISPGSFSACFDFVSVQATFDVCQKYVEPKGVIMPVGLGAPNLSFNLGDLNMREIRILGSFWGTTNDLDDVLKLVSEGKVKPVVRSAKLKELPEYIEKLRNNAYEGRVVFNP

SEQ ID NO: 95 - DNA, 1011 bp, synthetic construct
  misc_feature 1..1011: note = KRED-NADH-102 variant
  source 1..1011: mol_type = other DNA
atgtctatcccgtcttctcagtatggtttcgtatttaacaaacagtctggtctgaaactgcgcaacgatctgccagtacacaagccgaaagctggtcaactgctgctgaaagtagatgcagtaggtctgtgccactctgatctgcacgttatctacgaaggctttgacgcgggtgacaactacgttatgggtcacgaaatcgctggtaccgttgctgctgttggtgatgacgttatcaactacaaagttggtgaccgtgtagcttgtgttggtccgaacggttgcggtggttgcaaatactgccgtggtgctatcgacaacgtttgcaaaaacgctttcggtgactggttcggtctgggttatgatggtggctatcaacaatacctgctggtaactcgcccgcgtaacctgtctcgtatcccggataacgtatctgctgatgttgctgctgcttctaccgacgctgttctgactccgtaccacgctatcaaaatggctcaggtttctccgacctctaacatcctgctgatcggtgctggtggtctgggtggtaacgctatccaggttgctaaagcattcggtgctaaagttaccgttctggacaaaaaaaaagaagctcgtgaccaggctaaaaaactgggtgctgacgctgtttacgaaaccctgccggaatctatctctccgggttctttctctgcttgcttcgacttcgtttctgttcaggctaccttcgacgtttgccagaaatacgttgaaccgaaaggtgttatcatgccggttggtctgggtgctccgaacctgtcttttaacctgggtgacctggcgatgcgtgaaatccgtatcctgggttctttctggggtaccaccaacgacctggacgacgttctgaaactggtttctgaaggtaaagttaaaccggttgtacgttctgctaaactgaaagaactgccggaatacatcgaaaaactgcgtaacaacgcttacgaaggtcgtgttgttttcaacccgtaa

SEQ ID NO: 96 - protein, 336 aa, synthetic construct
  REGION 1..336: note = KRED-NADH-102 variant
MSIPSSQYGFVFNKQSGLKLRNDLPVHKPKAGQLLLKVDAVGLCHSDLHVIYEGFDAGDNYVMGHEIAGTVAAVGDDVINYKVGDRVACVGPNGCGGCKYCRGAIDNVCKNAFGDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQAKKLGADAVYETLPESISPGSFSACFDFVSVQATFDVCQKYVEPKGVIMPVGLGAPNLSFNLGDLAMREIRILGSFWGTTNDLDDVLKLVSEGKVKPVVRSAKLKELPEYIEKLRNNAYEGRVVFNP

SEQ ID NO: 97 - DNA, 1011 bp, synthetic construct
  misc_feature 1..1011: note = KRED-NADH-102 variant
  source 1..1011: mol_type = other DNA
atgtctatcccgtcttctcagtatggtttcgtatttaacaaacagtctggtctgaaactgcgcaacgatctgccagtacacaagccgaaagctggtcaactgctgctgaaagtagatgcagtaggtctgtgccactctgatctgcacgttatctacgaaggcctggactgcggtgacaactacgttatgggtcacgaaatcgctggtaccgttgctgctgttggtgatgacgttatcaactacaaagtaggtgaccgtgtagcttgtgttggtccgaacggttgcggtggttgcaaatactgccgtggtggcatcgacaacgtttgcaaaaacgctttcggtgactggttcggtctgggttatgatggtggctatcaacaatacctgctggtaactcgcccgcgtaacctgtctcgtatcccggataacgtatctgctgatgttgctgctgcttctaccgacgctgttctgactccgtaccacgctatcaaaatggctcaggtttctccgacctctaacatcctgctgatcggtgctggtggtctgggtggtaacgctatccaggttgctaaagcattcggtgctaaagttaccgttctggacaaaaaaaaagaagctcgtgaccaggctaaaaaactgggtgctgacgctgtttacgaaaccctgccggaatctatctctccgggttctttctctgcttgcttcgacttcgtttctgttcaggctaccttcgacgtttgccagaaatacgttgaaccgaaaggtgttatcatgccggttggtctgggtgctccgaacctgtcttttaacctgggtgacctggctctgcgtgaaatccgtatcctgggttctttctggggtaccaccaacgacctggacgacgttctgaaactggtttctgaaggtaaagttaaaccggttgtacgttctgctaaactgaaagaactgccggaatacatcgaaaaactgcgtaacaacgcttacgaaggtcgtgttgttttcaacccgtaa

SEQ ID NO: 98 - protein, 336 aa, synthetic construct
  REGION 1..336: note = KRED-NADH-102 variant
MSIPSSQYGFVFNKQSGLKLRNDLPVHKPKAGQLLLKVDAVGLCHSDLHVIYEGLDCGDNYVMGHEIAGTVAAVGDDVINYKVGDRVACVGPNGCGGCKYCRGGIDNVCKNAFGDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQAKKLGADAVYETLPESISPGSFSACFDFVSVQATFDVCQKYVEPKGVIMPVGLGAPNLSFNLGDLALREIRILGSFWGTTNDLDDVLKLVSEGKVKPVVRSAKLKELPEYIEKLRNNAYEGRVVFNP

SEQ ID NO: 99 - DNA, 1011 bp, synthetic construct
  misc_feature 1..1011: note = KRED-NADH-102 variant
  source 1..1011: mol_type = other DNA
atgtctatcccgtcttctcagtatggtttcgtatttaacaaacagtctggtctgaaactgcgcaacgatctgccagtacacaagccgaaagctggtcaactgctgctgaaagtagatgcagtaggtctgtgccattctgatctgcacgttatctacgaaggcctggacataggtgacaactacgttatgggtcacgaaatcgctggtaccgttgctgctgttggtgatgacgttatcaactacaaagtaggtgaccgtgtagcttgtgttggtccgaacggttgcggtggttgcaaatactgccgtggtggcatcgacaacgtttgcaaaaacgctttccatgactggttcggtctgggttatgatggtggctatcaacaatacctgctggtaactcgcccgcgtaacctgtctcgtatcccggataacgtatctgctgatgttgctgctgcttctaccgacgctgttctgactccgtaccacgctatcaaaatggctcaggtttctccgacctctaacatcctgctgatcggtgctggtggtctgggtggtaacgctatccaggttgctaaagcattcggtgctaaagttaccgttctggacaaaaaaaaagaagctcgtgaccaggctaaaaaactgggtgctgacgctgtttacgaaaccctgccggaatctatctctccgggttctttctctgcttgcttcgacttcgtttctgttcaggctaccttcgacgtttgccagaaatacgttgaaccgaaaggtgttatcatgccggttggtctgggcgctccgaacctgtcttttaacctgggtgacctggctctgcgtgaaatccgtatcctgggttctttctggggtaccaccaacgacctggacgacgttctgaaactggtttctgaaggtaaagttaaaccggttgtacgttctgctaaactgaaagaactgccggaatacatcgaaaaactgcgtaacaacgcttacgaaggtcgtgttgttttcaacccgtaa

SEQ ID NO: 100 - protein, 336 aa, synthetic construct
  REGION 1..336: note = KRED-NADH-102 variant
MSIPSSQYGFVFNKQSGLKLRNDLPVHKPKAGQLLLKVDAVGLCHSDLHVIYEGLDIGDNYVMGHEIAGTVAAVGDDVINYKVGDRVACVGPNGCGGCKYCRGGIDNVCKNAFHDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQAKKLGADAVYETLPESISPGSFSACFDFVSVQATFDVCQKYVEPKGVIMPVGLGAPNLSFNLGDLALREIRILGSFWGTTNDLDDVLKLVSEGKVKPVVRSAKLKELPEYIEKLRNNAYEGRVVFNP

SEQ ID NO: 101 - DNA, 1011 bp, synthetic construct
  misc_feature 1..1011: note = KRED-NADH-102 variant
  source 1..1011: mol_type = other DNA
atgtctatcccgtcttctcagtatggtttcgtatttaacaaacagtctggtctgaaactgcgcaacgatctgccagtacacaagccgaaagctggtcaactgctgctgaaagtagatgcagtaggtctgtgccattctgatctgcacgttatctacgaaggcctggacttaggtgacaactacgttatgggtcacgaaatcgctggtaccgttgctgctgttggtgatgacgttatcaactacaaagtaggtgaccgtgtagcttgtgttggtccgaacggttgcggtggttgcaaatactgccgtggtggcatcgacaacgtttgcaaaaacgctttccatgactggttcggtctgggttatgatggtggctatcaacaatacctgctggtaactcgcccgcgtaacctgtctcgtatcccggataacgtatctgctgatgttgctgctgcttctaccgacgctgttctgactccgtaccacgctatcaaaatggctcaggtttctccgacctctaacatcctgctgatcggtgctggtggtctgggtggtaacgctatccaggttgctaaagcattcggtgctaaagttaccgttctggacaaaaaaaaagaagctcgtgaccaggctaaaaaactgggtgctgacgctgtttacgaaaccctgccggaatctatctctccgcgttctttctctgcttgcttcgacttcgtttctgttcaggctaccttcgacgtttgccagaaatacgttgaaccgaaaggtgttatcatgccggttggtctgggcgctccgaacctgtcttttaacctgggtgacctggcgttgcgtgaaatccgtatcctgggttctttctggggtaccaccaacgacctggacgacgttctgaaactggtttctgaaggtaaagttaaaccggttgtacgttctgctaaactgaaagaactgccggaatacatcgaaaaactgcgtaacaacgcttacgaaggtcgtgttgttttcaacccgtaa

SEQ ID NO: 102 - protein, 336 aa, synthetic construct
  REGION 1..336: note = KRED-NADH-102 variant
MSIPSSQYGFVFNKQSGLKLRNDLPVHKPKAGQLLLKVDAVGLCHSDLHVIYEGLDLGDNYVMGHEIAGTVAAVGDDVINYKVGDRVACVGPNGCGGCKYCRGGIDNVCKNAFHDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQAKKLGADAVYETLPESISPRSFSACFDFVSVQATFDVCQKYVEPKGVIMPVGLGAPNLSFNLGDLALREIRILGSFWGTTNDLDDVLKLVSEGKVKPVVRSAKLKELPEYIEKLRNNAYEGRVVFNP

SEQ ID NO: 103 - DNA, 1011 bp, synthetic construct
  misc_feature 1..1011: note = KRED-NADH-102 variant
  source 1..1011: mol_type = other DNA
atgtctatcccgtcttctcagtatggtttcgtatttaacaaacagtctggtctgaaactgcgcaacgatctgccagtacacaagccgaaagctggtcaactgctgctgaaagtagatgcagtaggtctgtgccactctgatctgcacgttatctcagaaggcctggatttgggtgacaactacgttatgggtcacgaaatcgctggtaccgttgctgctgttggtgatgacgttatcaactacaaagtaggtgaccgtgtagcttgtgttggtccgaacggttgcggtggttgcaaatactgccgtggtgctatcgacaacgtttgcaaaaacgctttcggtgactggttcggtctgggttatgatggtggctatcaacaatacctgctggtaactcgcccgcgtaacctgtctcgtatcccggataacgtatctgctgatgttgctgctgcttctaccgacgctgttctgactccgtaccacgctatcaaaatggctcaggtttctccgacctctaacatcctgctgatcggtgctggtggtctgggtggtaacgctatccaggttgctaaagcattcggtgctaaagttaccgttctggacaaaaaaaaagaagctcgtgaccaggctaaaaaactgggtgctgacgctgtttacgaaaccctgccggaatctatctctccgggttctttctctgcttgcttcgacttcgtttctgttcaggctaccttcgacgtttgccagaaatacgttgaaccgaaaggtgttatcatgccggttggtctgggtgctccgaacctgtcttttaacctgcatgaccttgctctgcgtgaacaccgtatcctgggttctttctggggtaccaccaacgacctggacgacgtttttaaactggtttctgaaggtaaagttaaaccggttgtacgatctgctaaactgaaagaactgccggaatacatcgaaaaactgcgtaacaacgcttacgaaggtcgtgttgttttcaacccgtaa

SEQ ID NO: 104 - protein, 336 aa, synthetic construct
  REGION 1..336: note = KRED-NADH-102 variant
MSIPSSQYGFVFNKQSGLKLRNDLPVHKPKAGQLLLKVDAVGLCHSDLHVISEGLDLGDNYVMGHEIAGTVAAVGDDVINYKVGDRVACVGPNGCGGCKYCRGAIDNVCKNAFGDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQAKKLGADAVYETLPESISPGSFSACFDFVSVQATFDVCQKYVEPKGVIMPVGLGAPNLSFNLHDLALREHRILGSFWGTTNDLDDVFKLVSEGKVKPVVRSAKLKELPEYIEKLRNNAYEGRVVFNP

SEQ ID NO: 105 - DNA, 1011 bp, synthetic construct
  misc_feature 1..1011: note = KRED-NADH-102 variant
  source 1..1011: mol_type = other DNA
atgtctatcccgtcttctcagtatggtttcgtatttaacaaacagtctggtctgaaactgcgcaacgatctgccagtacacaagccgaaagctggtcaactgctgctgaaagtagatgcagtaggtctgtgccactctgatctgcacgttatcgatgaaggcctggatttgggtgacaactacgttatgggtcacgaaatcgctggtaccgttgctgctgttggtgatgacgttatcaactacaaagtaggtgaccgtgtagcttgtgttggtccgaacggttgcggtggttgcaaatactgccgtggtgctatcgacaacgtttgcaaaaacgctttcggtgactggttcggtctgggttatgatggtggctatcaacaatacctgctggtaactcgcccgcgtaacctgtctcgtatcccggataacgtatctgctgatgttgctgctgcttctaccgacgctgttctgactccgtaccacgctatcaaaatggctcaggtttctccgacctctaacatcctgctgatcggtgctggtggtctgggtggtaacgctatccaggttgctaaagcattcggtgctaaagttaccgttctggacaaaaaaaaagaagctcgtgaccaggctaaaaaactgggtgctgacgctgtttacgaaaccctgccggaatctatctctccgggttctttctctgcttgcttcgacttcgtttctgttcaggctaccttcgacgtttgccagaaatacgttgaaccgaaaggtgttatcatgccggttggtctgggtgctccgaacctgtcttttaacctgcatgaccttgctctgcgtgaaatacgtatcctgggttctttctggggtaccaccaacgacctggacgacgttttgaaactggtttctgaaggtaaagttaaaccggttgtacgttctgctaaactgaaagaactgccggaatacatcgaaaaactgcgtaacaacgcttacgaaggtcgtgttgttttcaacccgtaa

SEQ ID NO: 106 - protein, 336 aa, synthetic construct
  REGION 1..336: note = KRED-NADH-102 variant
MSIPSSQYGFVFNKQSGLKLRNDLPVHKPKAGQLLLKVDAVGLCHSDLHVIDEGLDLGDNYVMGHEIAGTVAAVGDDVINYKVGDRVACVGPNGCGGCKYCRGAIDNVCKNAFGDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQAKKLGADAVYETLPESISPGSFSACFDFVSVQATFDVCQKYVEPKGVIMPVGLGAPNLSFNLHDLALREIRILGSFWGTTNDLDDVLKLVSEGKVKPVVRSAKLKELPEYIEKLRNNAYEGRVVFNP

SEQ ID NO: 107 - DNA, 1011 bp, synthetic construct
  misc_feature 1..1011: note = KRED-NADH-102 variant
  source 1..1011: mol_type = other DNA
atgtctatcccgtcttctcagtatggtttcgtatttaacaaacagtctggtctgaaactgcgcaacgatctgccagtacacaagccgaaagctggtcaactgctgctgaaagtagatgcagtaggtctgtgccactctgatctgcacgttatctcagaaggcctggatttgggtgacaactacgttatgggtcacgaaatcgctggtaccgttgctgctgttggtgatgacgttatcaactacaaagtaggtgaccgtgtagcttgtgttggtccgaacggttgcggtggttgcaaatactgccgtggtgctatcgacaacgtttgcaaaaacgctttcggtgactggttcggtctgggttatgatggtggctatcaacaatacctgctggtaactcgcccgcgtaacctgtctcgtatcccggataacgtatctgctgatgttgctgctgcttctaccgacgctgttctgactccgtaccacgctatcaaaatggctcaggtttctccgacctctaacatcctgctgatcggtgctggtggtctgggtggtaacgctatccaggttgctaaagcattcggtgctaaagttaccgttctggacaaaaaaaaagaagctcgtgaccaggctaaaaaactgggtgctgacgctgtttacgaaaccctgccggaatctatctctccgggttctttctctgcttgcttcgacttcgtttctgttcaggctaccttcgacgtttgccagaaatacgttgaaccgaaaggtgttatcatgccggttggtctgggtgctccgaacctgtcttttaacctgcatgacgttgctctgcgtgaacaccgtatcctgggttctttctggggtaccaccaacgacctggacgacgttttcaaactggtttctgaaggtaaagttaaaccggttgtacgatctgctaaactgaaagaactgccggaatacatcgaaaaactgcgtaacaacgcttacgaaggtcgtgttgttttcaacccgtaa

SEQ ID NO: 108 - protein, 336 aa, synthetic construct
  REGION 1..336: note = KRED-NADH-102 variant
MSIPSSQYGFVFNKQSGLKLRNDLPVHKPKAGQLLLKVDAVGLCHSDLHVISEGLDLGDNYVMGHEIAGTVAAVGDDVINYKVGDRVACVGPNGCGGCKYCRGAIDNVCKNAFGDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQAKKLGADAVYETLPESISPGSFSACFDFVSVQATFDVCQKYVEPKGVIMPVGLGAPNLSFNLHDVALREHRILGSFWGTTNDLDDVFKLVSEGKVKPVVRSAKLKELPEYIEKLRNNAYEGRVVFNP

SEQ ID NO: 109 - DNA, 1011 bp, synthetic construct
  misc_feature 1..1011: note = KRED-NADH-102 variant
  source 1..1011: mol_type = other DNA
atgtctatcccgtcttctcagtatggtttcgtatttaacaaacagtctggtctgaaactgcgcaacgatctgccagtacacaagccgaaagctggtcaactgctgctgaaagtagatgcagtaggtctgtgccactctgatctgcacgttatctcagaaggcctggatttgggtgacaactacgttatgggtcacgaaatcgctggtaccgttgctgctgttggtgatgacgttatcaactacaaagtaggtgaccgtgtagcttgtgttggtccgaacggttgcggtggttgcaaatactgccgtggtgctatcgacaacgtttgcacaaacgctttcggtgactggttcggtctgggttatgatggtggctatcaacaatacctgctggtaactcgcccgcgtaacctgtctcgtatcccggataacgtatctgctgatgttgctgctgcttctaccgacgctgttctgactccgtaccacgctatcaaaatggctcaggtttctccgacctctaacatcctgctgatcggtgctggtggtctgggtggtaacgctatccaggttgctaaagcattcggtgctaaagttaccgttctggacaaaaaaaaagaagctcgtgaccaggctaaaaaactgggtgctgacgctgtttacgaaaccctgccggaatctatctctccgggttctttctctgcttgcttcgacttcgtttctgttcaggctaccttcgacgtttgccagaaatacgttgaaccgaaaggtgttatcatgccggttggtctgggtgctccgaacctgtcttttaacctgcatgaccttgctctgcgtgaaatccgtatcctgggttctttctggggtaccaccaacgacctggacgacgttttcaaactggtttctgaaggtaaagttaaaccggttgtacgttctgctaaactgaaagaactgccggaatacatcgaaaaactgcgtaacaacgcttacgaaggtcgtgttgttttcaacccgtaa

SEQ ID NO: 110 - protein, 336 aa, synthetic construct
  REGION 1..336: note = KRED-NADH-102 variant
MSIPSSQYGFVFNKQSGLKLRNDLPVHKPKAGQLLLKVDAVGLCHSDLHVISEGLDLGDNYVMGHEIAGTVAAVGDDVINYKVGDRVACVGPNGCGGCKYCRGAIDNVCTNAFGDWFGLGYDGGYQQYLLVTRPRNLSRIPDNVSADVAAASTDAVLTPYHAIKMAQVSPTSNILLIGAGGLGGNAIQVAKAFGAKVTVLDKKKEARDQAKKLGADAVYETLPESISPGSFSACFDFVSVQATFDVCQKYVEPKGVIMPVGLGAPNLSFNLHDLALREIRILGSFWGTTNDLDDVFKLVSEGKVKPVVRSAKLKELPEYIEKLRNNAYEGRVVFNP

SEQ ID NO: 111 - DNA, 1032 bp, synthetic construct
  misc_feature 1..1032: note = KRED-101 variant
  source 1..1032: mol_type = other DNA
atggctaaaatcgataacgcagttctgccggaaggttccctggttctggttaccggtgctaacggtttcgttggttcccacgttgttgaacagctgctggaacacggttacaaagttcgtggtaccgctcgttccgcttccaaactggctaacctgcagaaacgttgggacgctaaatacccgggtcgtttcgaaaccgctgttgttgaagacatgctgaaacagggtgcttacgacgaagttatcaaaggtgctgctggtgttgctcacatcgcttccgttgtttccttctccaacaaatacgacgaagttgttaccccggctatcggtggtaccttgaacgctctgcgtgctgctgctgctaccccgtccgttaaacgtttcgttctgacctcctccaccgtttccgctctgattccgaaaccgaacgttgaaggtatctacctggacgaaaaatcctggaacctggaatccatcgacaaagctaaaaccctgccggaatccgacccgcagaaatccctgtgggtatacgctgcatccaagaccgaagctgaactggctgcatggaaatttatggatgagaacaagccacacttcactctgaacgctgtactgccaaactacactattggcactattttcgatccggaaactcagtccggttccacctccggttggatgatgtccctgtttaacggcgaggtttccccggctctggctctgatgccaccgcagtactacgtttccgctgttgatattggcctgctgcacctgggttgcctggttctgccacaaatcgaacgtcgtcgtgtttacggtacggctggtactttcgattggaacaccgttctggctaccttccgtaaactgtacccgtccaaaaccttcccggctgacttcccagatcaaggtcaggacctgtctaaattcgacaccgctccgtccctggaaattctgaaatctctgggtcgcccaggttggcgttccatcgaagaatccatcaaagacctggttggttccgaaaccgcttaa

SEQ ID NO: 112 - protein, 343 aa, synthetic construct
  REGION 1..343: note = KRED-101 variant
MAKIDNAVLPEGSLVLVTGANGFVGSHVVEQLLEHGYKVRGTARSASKLANLQKRWDAKYPGRFETAVVEDMLKQGAYDEVIKGAAGVAHIASVVSFSNKYDEVVTPAIGGTLNALRAAAATPSVKRFVLTSSTVSALIPKPNVEGIYLDEKSWNLESIDKAKTLPESDPQKSLWVYAASKTEAELAAWKFMDENKPHFTLNAVLPNYTIGTIFDPETQSGSTSGWMMSLFNGEVSPALALMPPQYYVSAVDIGLLHLGCLVLPQIERRRVYGTAGTFDWNTVLATFRKLYPSKTFPADFPDQGQDLSKFDTAPSLEILKSLGRPGWRSIEESIKDLVGSETA

SEQ ID NO: 113 - DNA, 1032 bp, synthetic construct
  misc_feature 1..1032: note = KRED-101 variant
  source 1..1032: mol_type = other DNA
atggctaaaatcgataacgcagttctgccggaaggttccctggttctggttaccggtgctaacggtttcattggttcccacgttgttgaacagctgctggaacacggttacaaagttcgtggtaccgctcgttccgcttccaaactggctaacctgcagaaacgttgggacgctaaatacccgggtcgtttcgaaaccgctgttgttgaagacatgctgaaacagggtgcttacgacgaagttatcaaaggtgctgctggtgttgctcacatcgcttccgttgtttccttctccaacaaatacgacgaagttgttaccccggctatcggtggtaccttgaacgctctgcgtgctgctgctgctaccccgtccgttaaacgtttcgttctgacctcctccaccgtttcggctctgattccgaaaccgaacgttgaaggtatctacctggacgaaaaatcctggaacctggaatccatcgacaaagctaaaaccctgccggaatccgacccgcagaaatccctgtgggtatacgctgcatccaagaccgaagctgaactggctgcatggaaatttatggatgagaacaagccacacttcactctgaacgctgtactgccaaactacactattggcactattttcgatccggaaactcagggcggttccacctccggttggatgatgtccctgtttaacggcgaggtttccccggctctggctctgatgccaccgcagtactacgtttccgctgttgatattggcctgctgcacctgggttgcctggttctgccacaaatcgaacgtcgtcgtgtttacggtacggctggtactttcgattggaacaccgttctggctaccttccgtaaactgtacccgtccaaaaccttcccggctgacttcccagatcaaggtcaggacctgtctaaattcgacaccgctcgggcgctggaaattctgaaatctctgggtcgcccaggttggcgttccatcgaagaatccatcaaagacctggttggttccgaaaccgcttaa

SEQ ID NO: 114 - protein, 343 aa, synthetic construct
  REGION 1..343: note = KRED-101 variant
MAKIDNAVLPEGSLVLVTGANGFIGSHVVEQLLEHGYKVRGTARSASKLANLQKRWDAKYPGRFETAVVEDMLKQGAYDEVIKGAAGVAHIASVVSFSNKYDEVVTPAIGGTLNALRAAAATPSVKRFVLTSSTVSALIPKPNVEGIYLDEKSWNLESIDKAKTLPESDPQKSLWVYAASKTEAELAAWKFMDENKPHFTLNAVLPNYTIGTIFDPETQGGSTSGWMMSLFNGEVSPALALMPPQYYVSAVDIGLLHLGCLVLPQIERRRVYGTAGTFDWNTVLATFRKLYPSKTFPADFPDQGQDLSKFDTARALEILKSLGRPGWRSIEESIKDLVGSETA

SEQ ID NO: 115 - DNA, 1032 bp, synthetic construct
  misc_feature 1..1032: note = KRED-101 variant
  source 1..1032: mol_type = other DNA
atggctaaaatcgataacgcagttctgccggaaggttccctggttctggttaccggtgctaacggtttcattggttcccacgttgttgaacagctgctggaacacggttacaaagttcgtggtaccgctcgttccgcttccaaactggctaacctgcagaaacgttgggacgctaaatacccgggtcgtttcgaaaccgctgttgttgaagacatgctgaaacagggtgcttatgacgaagttatcaaaggtgctgctggtgttgctcacatcgcttccgttgtttccttctccaacaaatacgacgaagttgttccgccggctatcggtggtaccttgaacgctctgcgtgctgctgctgctaccccgtccgttaaacgtttcgttctgacctcctccaccgttgcggctctgattccgaaaccgaacgttgaaggtatctacctggacgaaaaatcctggaacctggaatccatcgacaaagctaaaaccctgccggaatccgacccgcagaaatccctgtgggtatacgctgcatccaagaccgaagctgaactggctgcatggaaatttatggatgagaacaagccacacttcactctgaacgctgtactgccaaactacactattggcactattttcgatccggaaactcagggcggttccacctccggttggatgatgtccctgtttaacggcgaggtttccccggctctggctctgatgccaccgcagtactacgtttccgctgttgatattggcctgctgcacgtgggtgcactggttctgccacaaatcgaacgtcgtcgtgtttacggtacggctggtactttcgattggaacaccgttctggctaccttccgtaaactgtacccgtccaaaaccttcccggctgacttcccagatcaaggtcaggacctgtctaaattcgacaccgctcgggcgctggaaattctgaaatctctgggtcgcccaggttggcgttccatcgaagaatccatcaaagacctggttggttccgaaaccgcttaa

SEQ ID NO: 116 - protein, 343 aa, synthetic construct
  REGION 1..343: note = KRED-101 variant
MAKIDNAVLPEGSLVLVTGANGFIGSHVVEQLLEHGYKVRGTARSASKLANLQKRWDAKYPGRFETAVVEDMLKQGAYDEVIKGAAGVAHIASVVSFSNKYDEVVPPAIGGTLNALRAAAATPSVKRFVLTSSTVAALIPKPNVEGIYLDEKSWNLESIDKAKTLPESDPQKSLWVYAASKTEAELAAWKFMDENKPHFTLNAVLPNYTIGTIFDPETQGGSTSGWMMSLFNGEVSPALALMPPQYYVSAVDIGLLHVGALVLPQIERRRVYGTAGTFDWNTVLATFRKLYPSKTFPADFPDQGQDLSKFDTARALEILKSLGRPGWRSIEESIKDLVGSETA

SEQ ID NO: 117 - DNA, 1032 bp, synthetic construct
  misc_feature 1..1032: note = KRED-101 variant
  source 1..1032: mol_type = other DNA
atggctaaaatcgataacgcagttctgccggaaggttccctggttctggttaccggtgctaacggtttcattggttcccacgttgttgaacagctgctggaacacggttacaaagttcgtggtaccgctcgttccgcttccaaactggctaacctgcagaaacgttgggacgctaaatacccgggtcgtttcgaaaccgctgttgttgaagacatgctgaaacagggtgcttatgacgaagttatcaaaggtgctgctggtgttgctcacatcgcttccgttgtttccttctccaacaaatacgacgaagttgttccgccggctatcggtggtaccttgaacgctctgcgtgctgctgctgctaccccgtccgttaaacgtttcgttctgacctcctccaccgtttccgctctgattccgaaaccgaacgttgaaggtatctacctggacgaaaaatcctggaacctggaatccatcgacaaagctaaaaccctgccggaatccgacccgcagaaatccctgtgggtatacgctgcatccaagaccgaagctgaactggctgcatggaaatttatggatgagaacaagccacacttcactctgaacgctgtactgccaaactacactattggcactattttagatccggaaactcagtccggttccacctccggttggatgatgtccctgtttaacggcgaggtttccccggctctggctctgatgccaccgcagtactacgtttccgtggttgatattggcctgctgcacgtgggtgcactggttctgccacaaatcgaacgtcgtcgtgtttacggtacggctggtactttcgattggaacaccgttctggctaccttccgtaaactgtacccgtccaaaaccttcccggctgacttcccagatcaaggtcaggacctgtctaaattcgacaccgctcgggcgctggaaattctgaaatctctgggtcgcccaggttggcgttccatcgaagaatccatcaaagacctggttggttccgaaaccgcttaa

SEQ ID NO: 118 - protein, 343 aa, synthetic construct
  REGION 1..343: note = KRED-101 variant
MAKIDNAVLPEGSLVLVTGANGFIGSHVVEQLLEHGYKVRGTARSASKLANLQKRWDAKYPGRFETAVVEDMLKQGAYDEVIKGAAGVAHIASVVSFSNKYDEVVPPAIGGTLNALRAAAATPSVKRFVLTSSTVSALIPKPNVEGIYLDEKSWNLESIDKAKTLPESDPQKSLWVYAASKTEAELAAWKFMDENKPHFTLNAVLPNYTIGTILDPETQSGSTSGWMMSLFNGEVSPALALMPPQYYVSVVDIGLLHVGALVLPQIERRRVYGTAGTFDWNTVLATFRKLYPSKTFPADFPDQGQDLSKFDTARALEILKSLGRPGWRSIEESIKDLVGSETA

SEQ ID NO: 119 - DNA, 1032 bp, synthetic construct
  misc_feature 1..1032: note = KRED-101 variant
  source 1..1032: mol_type = other DNA
atggctaaaatcgataacgcagttcttccggaaggttccctggttctggttaccggtgctaacggtttcgttggttcccacgttgttgaacagctgctggaacacggttacaaagttcgtggtaccgctcgttccgcttccaaactggctaatctgcagaaacgttgggacgctaaatacccgggtcgtttcgaaaccgctgttgttgaagacatgctgaaacagggtgcttacgacgaagttatcaaaggtgctgctggtgttgctcacatcgcttccgttgtttccttctccaacaaatacgacgaagttgttaccccggctatcggtggtaccttgaacgctctgcgtgctgctgctgctgagccgtccgttaaacgtttcgttctgacctcctccaccgtttccgctctgattccgaaaccgaacgttgaaggtatctacctggacgaaaaatcctggaacctggaatccgttgacaaagctaaaaccctgccggaatccgacccgcagaaatccctgtgggtatacgctgcatccaagaccgaagctgaactggctgcatggaaatttatggatgagaacaagccacacttcactctgaacgctgtactgccaaactacactattggcactattttcgatccggaaactcagtccggttccacctccggttggatgatgtccctgtttaacggcgaggtttccccggctctggctctgatgccaccgcagtactacgtttccgctgtagatattggcctgctgcacctgggttgcctggttctgccacaaatcgaacgtcgtcgtgtttacggtacggctggtactttcgattggaacaccgttctggctaccttccgtaaactgtacccgtccaaaaccttcccggctgacttcccagatcaaggtcaggacctgtctaaattcgacaccgctccgtccgaagaacttctgaaatctctgggtcgcccaggttggcgttccatcgaagaatccatcaaagacctggttggttccgaaaccgcttaa

SEQ ID NO: 120 - protein, 343 aa, synthetic construct
  REGION 1..343: note = KRED-101 variant
MAKIDNAVLPEGSLVLVTGANGFVGSHVVEQLLEHGYKVRGTARSASKLANLQKRWDAKYPGRFETAVVEDMLKQGAYDEVIKGAAGVAHIASVVSFSNKYDEVVTPAIGGTLNALRAAAAEPSVKRFVLTSSTVSALIPKPNVEGIYLDEKSWNLESVDKAKTLPESDPQKSLWVYAASKTEAELAAWKFMDENKPHFTLNAVLPNYTIGTIFDPETQSGSTSGWMMSLFNGEVSPALALMPPQYYVSAVDIGLLHLGCLVLPQIERRRVYGTAGTFDWNTVLATFRKLYPSKTFPADFPDQGQDLSKFDTAPSEELLKSLGRPGWRSIEESIKDLVGSETA

SEQ ID NO: 121 - DNA, 1032 bp, synthetic construct
  misc_feature 1..1032: note = KRED-101 variant
  source 1..1032: mol_type = other DNA
atggctaaaatcgataacgcagttcttccggaaggttccctggttctggttaccggtgctaacggtttcgttggttcccacgttgttgaacagctgctggaacacggttacaaagttcgtggtaccgctcgttccgcttccaaactggctaacctgcagaaacgttgggacgctaaatacccgggtcgtttcgaaaccgctgttgttgaagacatgctgaaacagggtgcttacgacgaagttatcaaaggtgctgctggtgttgctcacatcgcttccgttgtttccttctccaacaaatacgacgaagttgttaccccggctatcggtggtaccttgaacgctctgcgtgctgctgctgctaccccgtccgttaaacgtttcgttctgacctcctccaccgtttccgctctgattccgaaaccgaacgttgaaggtatctacctggacgaaaaatcctggaacctggaatccgttgacaaagctaaaaccctgccggaatccgacccgcagaaatccctgtgggtatacgctgcatccaagaccgaagctgaactggctgcatggaaatttatggatgagaacaagccacacttcactctgaacgctgtactgccaaactacaccattggcactattttcgatccggaaactcagtccggttccacctccggttggatgatgtccctgtttaacggcgaggtttccccggctctggctctgatgccaccgcagtactacgtttccgctcaggatattggcctgctgcacctgggttgcctggttctgccacaaatcgaacgtcgtcgtgtttttggtacggctggtccgttcgattggaacaccgttctggctaccttccgtaaactgtacccgtccaaaaccttcccggctgacttcccagatcaaggtcaggacctgtctaaattcgacaccgctccgtccgaagaacttctgaaatctctgggtcgcccaggttggcgttcccttgaagaatccatcaaagacctggttggttccgaaaccgcttaa

SEQ ID NO: 122 - protein, 343 aa, synthetic construct
  REGION 1..343: note = KRED-101 variant
MAKIDNAVLPEGSLVLVTGANGFVGSHVVEQLLEHGYKVRGTARSASKLANLQKRWDAKYPGRFETAVVEDMLKQGAYDEVIKGAAGVAHIASVVSFSNKYDEVVTPAIGGTLNALRAAAATPSVKRFVLTSSTVSALIPKPNVEGIYLDEKSWNLESVDKAKTLPESDPQKSLWVYAASKTEAELAAWKFMDENKPHFTLNAVLPNYTIGTIFDPETQSGSTSGWMMSLFNGEVSPALALMPPQYYVSAQDIGLLHLGCLVLPQIERRRVFGTAGPFDWNTVLATFRKLYPSKTFPADFPDQGQDLSKFDTAPSEELLKSLGRPGWRSLEESIKDLVGSETA

SEQ ID NO: 123 - DNA, 1032 bp, synthetic construct
  misc_feature 1..1032: note = KRED-101 variant
  source 1..1032: mol_type = other DNA
atggctaaaatcgataacgcagttctgccggaaggttccctggttctggttaccggtgctaacggtttcgttggttcccacgttgttgaacagctgctggaacacggttacaaagttcgtggtaccgctcgttccgcttccaaactggctaacctgcagaaacgttgggacgctaaatacccgggtcgtttcgaaaccgctgttgttgaagacatgctgaaacagggtgcttacgacgaagttatcaaaggtgctgctggtgttgctcacatcgcttccgttgtttccttctccaacaaatacgacgaagttgttaccccggctatcggtggtaccttgaacgctctgcgtgctgctgctgctaccccgtccgttaaacgtttcgttctgacctcctccaccgtttccgctctgattccgaaaccgaacgttgaaggtatctacctggacgaaaaatcctggaacctggaatccatcgacaaagctaaaaccctgccggaatccgacccgcagaaatccctgtgggtatacgctgcatccaagaccgaagctgaactggctgcatggaaatttatggatgagaacaagccacacttcactctgaacgctgtactgccagggtacactattggcactattttcgatccggaaactcagtccggttccacctccggttggatgatgtccctgtttaacggcgaggtttccccggctctggctctgatgccaccgcagtactacgtttccgctgttgatattggcctgctgcacctgggttgcctggttctgccacaaatcgaacgtcgtcgtgtttacggtacggctggtactttcgattggaacaccgttctggctaccttccgtaaactgtacccgtccaaaaccttcccggctgacttcccagatcaaggtcaggacctgtctaaattcgacaccgctccgtccctggaaattctgaaatctctgggtcgcccaggttggcgttccatcgaagaatccatcaaagacctggttggttccgaaaccgcttaa

SEQ ID NO: 124 - protein, 343 aa, synthetic construct
  REGION 1..343: note = KRED-101 variant
MAKIDNAVLPEGSLVLVTGANGFVGSHVVEQLLEHGYKVRGTARSASKLANLQKRWDAKYPGRFETAVVEDMLKQGAYDEVIKGAAGVAHIASVVSFSNKYDEVVTPAIGGTLNALRAAAATPSVKRFVLTSSTVSALIPKPNVEGIYLDEKSWNLESIDKAKTLPESDPQKSLWVYAASKTEAELAAWKFMDENKPHFTLNAVLPGYTIGTIFDPETQSGSTSGWMMSLFNGEVSPALALMPPQYYVSAVDIGLLHLGCLVLPQIERRRVYGTAGTFDWNTVLATFRKLYPSKTFPADFPDQGQDLSKFDTAPSLEILKSLGRPGWRSIEESIKDLVGSETA

SEQ ID NO: 125 - DNA, 1032 bp, synthetic construct; identical to SEQ ID NO: 123
  misc_feature 1..1032: note = KRED-101 variant
  source 1..1032: mol_type = other DNA

SEQ ID NO: 126 - protein, 343 aa, synthetic construct; identical to SEQ ID NO: 124
  REGION 1..343: note = KRED-101 variant

SEQ ID NO: 127 - DNA, 1032 bp, synthetic construct
  misc_feature 1..1032: note = KRED-101 variant
  source 1..1032: mol_type = other DNA
atggctaaaatcgataacgcagttctgccggaaggttccctggttctggttaccggtgctaacggtttcgttggttcccacgttgttgaacagctgctggaacacggttacaaagttcgtggtaccgctcgttccgcttccaaactggctaacctgcagaaacgttgggacgctaaatacccgggtcgtttcgaaaccgctgttgttgaagacatgctgaaacagggtgcttacgacgaagttatcaaaggtgctgctggtgttgctcacatcgcttccgttgtttccttctccaacaaatacgacgaagttgttaccccggctatcggtggtaccttgaacgctctgcgtgctgctgctgctaccccgtccgttaaacgtttcgttctgacctcctccaccttttccgctctgattccgaaaccgaacgttgaaggtatctacctggacgaaaaatcctggaacctggaatccatcgacaaagctaaaaccctgccggaatccgacccgcagaaatccctgtgggtatacgctgcatccaagaccgaagctgaactggctgcatggaaatttatggatgagaacaagccacacttcactctgaacgctgtactgccaaactacactattggcactattttcgatccggaaactcagtccggttccacctccggttggatgatgtccctgtttaacggcgaggtttccccggctctggctctgatgccaccgcagtactacgtttccgctgttgatattggcctgctgcacctgggttgcctggttctgccacaaatcgaacgtcgtcgtgtttacggtacggctggtactttcgattggaacaccgttctggctaccttccgtaaactgtacccgtccaaaaccttcccggctgacttcccagatcaaggtcaggacctgtctaaattcgacaccgctccgtccctggaaattttgaaatctctgggtcgcccaggttggcgttccatcgaagaatccatcaaagacctggttggttccgaaaccgcttaa

SEQ ID NO: 128 - protein, 343 aa, synthetic construct
  REGION 1..343: note = KRED-101 variant
MAKIDNAVLPEGSLVLVTGANGFVGSHVVEQLLEHGYKVRGTARSASKLANLQKRWDAKYPGRFETAVVEDMLKQGAYDEVIKGAAGVAHIASVVSFSNKYDEVVTPAIGGTLNALRAAAATPSVKRFVLTSSTFSALIPKPNVEGIYLDEKSWNLESIDKAKTLPESDPQKSLWVYAASKTEAELAAWKFMDENKPHFTLNAVLPNYTIGTIFDPETQSGSTSGWMMSLFNGEVSPALALMPPQYYVSAVDIGLLHLGCLVLPQIERRRVYGTAGTFDWNTVLATFRKLYPSKTFPADFPDQGQDLSKFDTAPSLEILKSLGRPGWRSIEESIKDLVGSETA

SEQ ID NO: 129 - DNA, 1032 bp, synthetic construct
  misc_feature 1..1032: note = KRED-101 variant
  source 1..1032: mol_type = other DNA
atggctaaaatcgataacgcagttctgccggaaggttccctggttctggttaccggtgctaacggtttcgttggttcccacgttgttgaacagctgctggaacacggttacaaagttcgtggtaccgctcgttccgcttccaaactggctaacctgcagaaacgttgggacgctaaatacccgggtcgtttcgaaaccgctgttgttgaagacatgctgaaacagggtgcttacgacgaagttatcaaaggtgctgctggtgttgctcacatcgcttccgttgtttccttctccaacaaatacgacgaagttgttaccccggctatcggtggtaccttgaacgctctgcgtgctgctgctgctaccccgtccgttaaacgtttcgttctgacctcctccaccttttccgctctgattccgaaaccgaacgttgaaggtatctacctggacgaaaaatcctggaacctggaatccatcgacaaagctaaaaccctgccggaatccgacccgcagaaatccctgtgggtatacgctgcatccaagaccgaagctgaactggctgcatggaaatttatggatgagaacaagccacacttcactctgaacgctgtactgccaaactacactattggcactattttcgatccggaaactcagtccggttccacctccggttggatgatgtccctgtttaacggcgaggtttccccggctctggctctgatgccaccgcagtactacgtttccgctgttgatattggcctgctgcacctgggttgcctggttctgccacaaatcgaacgtcgtcgtgtttacggtacggctggtactttcgattggaacaccgttctggctaccttccgtaaactgtacccgtccaaaaccttcccggctgacttcccagatcaaggtcaggacctgtctaaattcgacaccgctccgtccctggaaattctgaaatctctgggtcgcccaggttggcgttccatcgaagaatccatcaaagacctggttggttccgaaaccgcttaa

SEQ ID NO: 130 - protein, 343 aa, synthetic construct; identical to SEQ ID NO: 128
  REGION 1..343: note = KRED-101 variant

SEQ ID NO: 131 - DNA, 1032 bp, synthetic construct
  misc_feature 1..1032: note = KRED-101 variant
  source 1..1032: mol_type = other DNA
atggctaaaatcgataacgcagttctgccggaaggttccctggttctggttaccggtgctaacggtttcgttggttcccacgttgttgaacagctgctggaacacggttacaaagttcgtggtaccgctcgttccgcttccaaactggctaacctgcagaaacgttgggacgctaaatacccgggtcgtttcgaaaccgctgttgttgaagacatgctgaaacagggtgcttacgacgaagttatcaaaggtgctgctggtgttgctcacatcgcttccgttgtttccttctccaacaaatacgacgaagttgttaccccggctatcggtggtaccttgaacgctctgcgtgctgctgctgctaccccgtccgttaaacgtttcgttctgacctcctccaccgtttccgctctggttccgaaaccgaacgttgaaggtatctacctggacgaaaaatcctggaacctggaatccatcgacaaagctaaaaccctgccggaatccgacccgcagaaatccctgtgggtatacgctgcatccaagaccgaagctgaactggctgcatggaaatttatggatgagaacaagccacacttcactctgaacgctgtactgccatcgtacactattggcactattttcgatccggaaactcagtccggttccacctccggttggatgatgtccctgtttaacggcgaggtttccccggctctggctctgatgccaccgcagtactacgtttccgctgttgatattggcctgctgcacctgggttgcctggttctgccacaaatcgaacgtcgtcgtgtttacggtacggctggtactttcgattggaacaccgttctggctaccttccgtaaactgtacccgtccaaaaccttcccggctgacttcccagatcaaggtcaggacctgtctaaattcgacaccgctccgtccctggaaattttgaaatctttgggtcgcccaggttggcgttccatcgaagaatccatcaaagacctggttggttccgaaaccgcttaa

SEQ ID NO: 132 - protein, 343 aa, synthetic construct
  REGION 1..343: note = KRED-101 variant
MAKIDNAVLPEGSLVLVTGANGFVGSHVVEQLLEHGYKVRGTARSASKLANLQKRWDAKYPGRFETAVVEDMLKQGAYDEVIKGAAGVAHIASVVSFSNKYDEVVTPAIGGTLNALRAAAATPSVKRFVLTSSTVSALVPKPNVEGIYLDEKSWNLESIDKAKTLPESDPQKSLWVYAASKTEAELAAWKFMDENKPHFTLNAVLPSYTIGTIFDPETQSGSTSGWMMSLFNGEVSPALALMPPQYYVSAVDIGLLHLGCLVLPQIERRRVYGTAGTFDWNTVLATFRKLYPSKTFPADFPDQGQDLSKFDTAPSLEILKSLGRPGWRSIEESIKDLVGSETA

SEQ ID NO: 133 - DNA, 1032 bp, synthetic construct
  misc_feature 1..1032: note = KRED-101 variant
  source 1..1032: mol_type = other DNA
atggcaaagatcgataacgcagttctgccggaaggttccctggttctggttaccggtgctaacggtttcgttggttcccacgttgttgaacagctgctggaacacggttacaaagttcgtggtaccgctcgttccgcttccaaactggctaacctgcagaaacgttgggacgctaaatacccgggtcgtttcgaaaccgctgttgttgaagacatgctgaaacagggtgcttacgacgaagttatcaaaggtgctgctggtgttgctcacatcgcttccgttacatccttctccaacaaatacgacgaagttgttaccccggctatcggtggtaccttgaacgctctgcgtgctgctgctgctaccccgtccgttaaacgtttcgttctgacctcctccaccgtttccgctctgattccgaaaccgaacgttgaaggtatctacctggacgaaaaatcctggaacctggaatccatcgacaaagctaaaaccctgccggaatccgacccgcagaaatccctgtgggtatacgctgcatccaagaccgaagctgaactggctgcatggaaatttatggatgagaacaagccacacttcactctgaacgctgtactgccagggtacactattggcactattttcgatccggaaactcagtccggttccacctccggttggatgatgtccctgtttaacggcgaggtttccccggccctggctctgatgccaccgcagtactacgtttccgctgttgatattggcctgctgcacctgggttgcctggttctgccacaaatcgaacgtcgtcgtgtttacggtacggctggtactttcgattggaacaccgttctggctaccttccgtaaactgtacccgtccaaaaccttcccggctgacttcccagatcaaggtcaggacctgtctaaattcgacaccgctccgtccctggaaattctgaaatctctgggtcgcccaggttggcgttccatcgaagaatccatcaaagacctggttggttccgaaaccgcttaa

SEQ ID NO: 134 - protein, 343 aa, synthetic construct
  REGION 1..343: note = KRED-101 variant
MAKIDNAVLPEGSLVLVTGANGFVGSHVVEQLLEHGYKVRGTARSASKLANLQKRWDAKYPGRFETAVVEDMLKQGAYDEVIKGAAGVAHIASVTSFSNKYDEVVTPAIGGTLNALRAAAATPSVKRFVLTSSTVSALIPKPNVEGIYLDEKSWNLESIDKAKTLPESDPQKSLWVYAASKTEAELAAWKFMDENKPHFTLNAVLPGYTIGTIFDPETQSGSTSGWMMSLFNGEVSPALALMPPQYYVSAVDIGLLHLGCLVLPQIERRRVYGTAGTFDWNTVLATFRKLYPSKTFPADFPDQGQDLSKFDTAPSLEILKSLGRPGWRSIEESIKDLVGSETA

SEQ ID NO: 135 - DNA, 1032 bp, synthetic construct
  misc_feature 1..1032: note = KRED-101 variant
  source 1..1032: mol_type = other DNA
atggcaaagatcgataacgcagttctgccggaaggttccctggttctggttaccggtgctaacggtttcattggttcccacgttgttgaacagctgctggaacacggttacaaagttcgtggtaccgctcgttccgcttccaaactggctaacctgcagaaacgttgggacgctaaatacccgggtcgtttcgaaaccgctgttgttgaagacatgctgaaacagggtgcttacgacgaagttatcaaaggtgctgctggtgttgctcacatcgcttccgttacatccttctccaacaaatacgacgaagttgttaccccggctatcggtggtaccttgaacgctctgcgtgctgctgctgctaccccgtccgttaaacgtttcgttctgacctcctccaccgtttccgctctgattccgaaaccgaacgttgaaggtatctacctggacgaaaaatcctggaacctggaatccatcgacaaagctaaaaccctgccggaatccgacccgcagaaatccctgtgggtatacgctgcatccaagaccgaagctgaactggctgcatggaaatttatggatgagaacaagccacacttcactctgaacgctgtactgccaggatacactattggcactattttcgatccggaaactcagtccggttccacctccggttggatgacgtccctgtttaacggcgaggtttccccggccctggctctgatgccaccgcagtactacgtttccgctgttgatattggcctgctgcacctgggttgcctggttctgccacaaatcgaacgtcgtcgtgtttacggtacggctggtactttcgattggaacaccgttctggctaccttccgtaaactgtacccgtccaaaaccttcccggctgacttcccagatcaaggtcaggacctgtctaaattcgacaccgctccgtccctggaaattctgaaatctctgggtcgcccaggttggcgttccatcgaagaatccatcaaagacctggttggttccgaaaccgcttaa

SEQ ID NO: 136 - protein, 343 aa, synthetic construct
  REGION 1..343: note = KRED-101 variant
MAKIDNAVLPEGSLVLVTGANGFIGSHVVEQLLEHGYKVRGTARSASKLANLQKRWDAKYPGRFETAVVEDMLKQGAYDEVIKGAAGVAHIASVTSFSNKYDEVVTPAIGGTLNALRAAAATPSVKRFVLTSSTVSALIPKPNVEGIYLDEKSWNLESIDKAKTLPESDPQKSLWVYAASKTEAELAAWKFMDENKPHFTLNAVLPGYTIGTIFDPETQSGSTSGWMTSLFNGEVSPALALMPPQYYVSAVDIGLLHLGCLVLPQIERRRVYGTAGTFDWNTVLATFRKLYPSKTFPADFPDQGQDLSKFDTAPSLEILKSLGRPGWRSIEESIKDLVGSETA

SEQ ID NO: 137 - DNA, 1032 bp, synthetic construct
  misc_feature 1..1032: note = KRED-101 variant
  source 1..1032: mol_type = other DNA
atggcaaagatcgataacgcagttctgccggaaggttccctggttctggttaccggtgctaacggtttcgttggttcccacgttgttgaacagctgctggaacacggttacaaagttcgtggtaccgctcgttccgcttccaaactggctaacctgcagaaacgttgggacgctaaatacccgggtcgtttcgaaaccgctgttgttgaagacatgctgaaacagggtgcttacgacgaagttatcaaaggtgctgctggtgttgctcacatcgcttccgttacatccttctccaacaaatacgacgaagttgttaccccggctatcggtggtaccttgaacgctctgcgtgctgctgctgctaccccgtccgttaaacgtttcgttctgacctcctccaccttttccgctctggttccgaaaccgaacgttgaaggtatctacctggacgaaaaatcctggaacctggaatccatcgacaaagctaaaaccctgccggaatccgacccgcagaaatccctgtgggtatacgctgcgtccaagaccgaagctgaactggctgcatggaaatttatggatgagaacaagccacacttcactctgaacgctgtactgccaaactacactattggcactattttcgatccggaaactcagtccggttccacctccggttggatgatgtccctgtttaacggcgaggtttccccggctctggctctgatgccaccgcagtactacgtttccgctgttgatattggcctgctgcacctgggttgcctggttctgccacaaatcgaacgtcgtcgtgtttacggtacggctggtactttcgattggaacaccgttctggctaccttccgtaaactgtacccgtccaaaaccttcccggctgacttcccagatcaaggtcaggacctgtctaaattcgacaccgctccgtccctggaaattctgaaatctctgggtcgcccaggttggcgttccatcgaagaatccatcaaagacctggttggttccgaaaccgcttaa

SEQ ID NO: 138 - protein, 343 aa, synthetic construct
  REGION 1..343: note = KRED-101 variant
MAKIDNAVLPEGSLVLVTGANGFVGSHVVEQLLEHGYKVRGTARSASKLANLQKRWDAKYPGRFETAVVEDMLKQGAYDEVIKGAAGVAHIASVTSFSNKYDEVVTPAIGGTLNALRAAAATPSVKRFVLTSSTFSALVPKPNVEGIYLDEKSWNLESIDKAKTLPESDPQKSLWVYAASKTEAELAAWKFMDENKPHFTLNAVLPNYTIGTIFDPETQSGSTSGWMMSLFNGEVSPALALMPPQYYVSAVDIGLLHLGCLVLPQIERRRVYGTAGTFDWNTVLATFRKLYPSKTFPADFPDQGQDLSKFDTAPSLEILKSLGRPGWRSIEESIKDLVGSETA

SEQ ID NO: 139 - DNA, 1032 bp, synthetic construct
  misc_feature 1..1032: note = KRED-101 variant
  source 1..1032: mol_type = other DNA
atggcatacatcgataacgcagttctgccggaaggttccctggttctggttaccggtgctaacggtttcgttggttcccacgttgttgaacagctgctggaacacggttacaaagttcgtggtaccgctcgttccgcttccaaactggctaacctgcagaaacgttgggacgctaaatacccgggtcgtttcgaaaccgctgttgttgaagacatgctgaaacagggtgcttacgacgaagttatcaaaggtgctgctggtgttgctcacatcgcttccgttacatccttctccaacaaatacgacgaagttgttaccccggctatcggtggtaccttgaacgctctgcgtgctgctgctgctaccccgtccgttaaacgtttcgttctgacctcctccaccgtttccgctctgattccgaaaccgaacgttgaaggtatctacctggacgaaaaatcctggaacctggaatccatcgacaaagctaaaaccctgccggaatccgacccgcagaaatccctgtgggtatacgctgcatccaagaccgaagctgaactggctgcatggaaatttatggatgagaacaagccacacttcactctgaacgctgtactgccagggtacactattggcactattttcgatccggaaactcagtccggttccacctccggttggatgatgtccctgtttaacggcgaggtttccccggccctggctctgatgccaccgcagtactacgtttccgctgttgatattggcctgctgcacctgggttgcctggttctgccacaaatcgaacgtcgtcgtgtttacggtacggctggtactttcgattggaacaccgttctggctaccttccgtaaactgtacccgtccaaaaccttcccggctgacttcccagatcaaggtcaggacctgtctaaattcgacaccgctccgtccctggaaattctgaaatctctgggtcgcccaggttggcgttccatcgaagaatccatcaaagacctggttggttccgaaaccgcttaa

SEQ ID NO: 140 - protein, 343 aa, synthetic construct
  REGION 1..343: note = KRED-101 variant
MAYIDNAVLPEGSLVLVTGANGFVGSHVVEQLLEHGYKVRGTARSASKLANLQKRWDAKYPGRFETAVVEDMLKQGAYDEVIKGAAGVAHIASVTSFSNKYDEVVTPAIGGTLNALRAAAATPSVKRFVLTSSTVSALIPKPNVEGIYLDEKSWNLESIDKAKTLPESDPQKSLWVYAASKTEAELAAWKFMDENKPHFTLNAVLPGYTIGTIFDPETQSGSTSGWMMSLFNGEVSPALALMPPQYYVSAVDIGLLHLGCLVLPQIERRRVYGTAGTFDWNTVLATFRKLYPSKTFPADFPDQGQDLSKFDTAPSLEILKSLGRPGWRSIEESIKDLVGSETA

SEQ ID NO: 141 - DNA, 1032 bp, synthetic construct
  misc_feature 1..1032: note = KRED-101 variant
  source 1..1032: mol_type = other DNA
atggcatacatcgataacgcagttctgccggaaggttccctggttctggttaccggtgctaacggtttcgttggttcccacgttgttgaacagctgctggaacacggttacaaagttcgtggtaccgctcgttccgcttccaaactggctaacctgcagaaacgttgggacgctaaatacccgggtcgtttcgaaaccgctgttgttgaagacatgctgaaacagggtgcttacgacgaagttatcaaaggtgctgctggtgttgctcacatcgcttccgttacatccttctccaacaaatacgacgaagttgttaccccggctatcggtggtaccttgaacgctctgcgtgctgctgctgctaccccgtccgttaaacgtttcgttctgacctcctccaccgtttccgctctgattccgaaaccgaacgttgaaggtatctacctggacgaaaaatcctggaacctggaatccatcgacaaagctaaaaccctgccggaatccgacccgcagaaatccctgtgggtatacgctgcatccaagaccgaagctgaactggctgcatggaaatttatggatgagaacaagccacacttcactctgaacgctgtactgccaggatacactattggcactattttcgatccggaaactcagtccggttccacctccggttggatgacgtccctgtttaacggcgaggtttccccggccctggctctgatgccaccgcagtactacgtttccgctgttgatattggcctgctgcacctgggttgcctggttctgccacaaatcgaacgtcgtcgtgtttacggtacggctggtactttcgattggaacaccgttctggctaccttccgtaaactgtacccgtccaaaaccttcccggctgacttcccagatcaaggtcaggacctgtctaaattcgacaccgctcggtccctggaaattctgaaatctctgggtcgcccaggttggcgttccatcgaagaatccatcaaagacctggttggttccgaaaccgcttaa

SEQ ID NO: 142 - protein, 343 aa, synthetic construct
  REGION 1..343: note = KRED-101 variant
MAYIDNAVLPEGSLVLVTGANGFVGSHVVEQLLEHGYKVRGTARSASKLANLQKRWDAKYPGRFETAVVEDMLKQGAYDEVIKGAAGVAHIASVTSFSNKYDEVVTPAIGGTLNALRAAAATPSVKRFVLTSSTVSALIPKPNVEGIYLDEKSWNLESIDKAKTLPESDPQKSLWVYAASKTEAELAAWKFMDENKPHFTLNAVLPGYTIGTIFDPETQSGSTSGWMTSLFNGEVSPALALMPPQYYVSAVDIGLLHLGCLVLPQIERRRVYGTAGTFDWNTVLATFRKLYPSKTFPADFPDQGQDLSKFDTARSLEILKSLGRPGWRSIEESIKDLVGSETA

SEQ ID NO: 143 - DNA, 1032 bp, synthetic construct
  misc_feature 1..1032: note = KRED-101 variant
  source 1..1032: mol_type = other DNA
atgactaagatcgataacgcagttctgccggaaggttccctggttctggttaccggtgctaacggtttcgttggttcccacgttgttgaacagctgctggaacacggttacaaagttcgtggtaccgctcgttccgcttccaaactggctaacctgcagaaacgttgggacgctaaatacccgggtcgtttcgaaaccgctgttgttgaagacatgctgaaacagggtgcttacgacgaagttatcaaaggtgctgctggtgttgctcacatcgcttccgttgtttccttctccaacaaacctgacgaagttgttaccccggctatcggtggtaccttgaacgctctgcgtgctgctgctgctaccccgtccgttaaacgtttcgttctgacctcctccaccgtttccgctctgattccgaaaccgaacgttgaaggtatctacctggacgaaaaatcctggaacctggaatccatcgacaaagctaaaaccctgccggaatccgacccgcagaaatccctgtgggtatacgctctgtccaagatggaagctgaactggctgcatggaaatttatggatgagaacaagccacacttcactctgaacgctgtactgccagggtacactattggcactattttcgatccggaaactcagtccggttccacctccggttggatgaggtccctgtttaacggcgaggtttccccgttgctggctctgatgccaccgcagtactacgtttccgctgttgatattggcctgctgcacctgggttgcctggttctgccacaaatcgaacgtcgtcgtgtttacggtacggctggtactttcgattggaacgaagttctggctaccttccgtaaactgtacccgtccaaaaccttcccggctgacttcccagatcaaggtcaggacctgtctaaattcgacaccgctccgtccctggaaattctgaaatctctgggtcgcccaggttggcgttccatcgaagaatccatcaaagacctggttggttccgaaaccgcttaa

SEQ ID NO: 144 - protein, 343 aa, synthetic construct
  REGION 1..343: note = KRED-101 variant
MTKIDNAVLPEGSLVLVTGANGFVGSHVVEQLLEHGYKVRGTARSASKLANLQKRWDAKYPGRFETAVVEDMLKQGAYDEVIKGAAGVAHIASVVSFSNKPDEVVTPAIGGTLNALRAAAATPSVKRFVLTSSTVSALIPKPNVEGIYLDEKSWNLESIDKAKTLPESDPQKSLWVYALSKMEAELAAWKFMDENKPHFTLNAVLPGYTIGTIFDPETQSGSTSGWMRSLFNGEVSPLLALMPPQYYVSAVDIGLLHLGCLVLPQIERRRVYGTAGTFDWNEVLATFRKLYPSKTFPADFPDQGQDLSKFDTAPSLEILKSLGRPGWRSIEESIKDLVGSETA

SEQ ID NO: 145 - DNA, 1032 bp, synthetic construct
  misc_feature 1..1032: note = KRED-101 variant
  source 1..1032: mol_type = other DNA
atggctaaaatcgataacgcagttctgccggaaggttccctggttctggttaccggtgctaacggtttcgttggttcccacgttgttgaacagctgctggaacacggttacaaagttcgtggtaccgctcgttccgcttccaaactggctaacctgcagaaacgttgggacgctaaatacccgggtcgtttcgaaaccgctgttgttgaagacatgctgaaacagggtgcttacgacgaagttatcaaaggtgctgctggtgttgctcacatcgcttccgttgtttccttctccaacaaatacgacgaagttgttaccccggctatcggtggtaccttgaacgctctgcgtgctgctgctgctaccccgtccgttaaacgtttcgttctgacctcctccaccgtttccgctctgattccgaagccgaacgttgaaggtatctacctggacgaaaagtcctggaacctggaatccgttgacaaagctaaaaccctgccggaatccgacccgcagaaatccctgtgggtatacgctgcatccaagaccgaagctgaactggctgcatggaaatttatggatgagaacaagccacacttcactctgaacgctgtactgccagggtacactattggcactattttcgatccggaaactcagtccggttccacctccggttggatgctgtccctgtttaacggcgaggtttcgccggctctggctctgatgccaccgcagtactacgtttccgctgttgatattggcctgctgcacctgggttgcctggttctgccacaaatcgaacgtcgtcgtgtttacggtacggctggtactttcgattggaacaccgttctggctaccttccgtaaactgtacccgtccaaaaccttcccggctgacttcccagatcaaggtcaggacctgtctcaattcgacaccgctccgtccctggaaattctgaaatctctgggtcgcccaggttggcgttcccttgaagaatccatcaaagacctggttggttccgaaaccgcttaa

SEQ ID NO: 146 - protein, 343 aa, synthetic construct
  REGION 1..343: note = KRED-101 variant
MAKIDNAVLPEGSLVLVTGANGFVGSHVVEQLLEHGYKVRGTARSASKLANLQKRWDAKYPGRFETAVVEDMLKQGAYDEVIKGAAGVAHIASVVSFSNKYDEVVTPAIGGTLNALRAAAATPSVKRFVLTSSTVSALIPKPNVEGIYLDEKSWNLESVDKAKTLPESDPQKSLWVYAASKTEAELAAWKFMDENKPHFTLNAVLPGYTIGTIFDPETQSGSTSGWMLSLFNGEVSPALALMPPQYYVSAVDIGLLHLGCLVLPQIERRRVYGTAGTFDWNTVLATFRKLYPSKTFPADFPDQGQDLSQFDTAPSLEILKSLGRPGWRSLEESIKDLVGSETA

SEQ ID NO: 147 - DNA, 1029 bp, synthetic construct
  misc_feature 1..1029: note = KRED-101 variant
  source 1..1029: mol_type = other DNA
atggcaaagatcgataacgcagttctgccggaaggttccctggttctggttaccggtgctaacggtttcattggttcccacgttgttgaacagctgctggaacacggttacaaagttcgtggtaccgtgcgttccgctgaaaaaaatgctaacctgcagaaacgttgggacgctaaatacccgggtcgtttcgaaaccgtggaagttccagacatgctgaaacagggtgcttacgacgaagttatcaaaggtgctgctggtgttgctcacgttgcttccgtaacatccttctccaacaaatacgacgaagttgttaccccggctatcggtggtaccttgaacgctctgcgtgctgctgctgctaccccgtccgttaaacgtttcgttctgacctcctccaccttttccgctctggttccgaaaccgaacgttgaaggtatctacctggacgaaaaatcctggaacctggaatccatcgacaaagctaaaaccctgccggaatccgacccgcagaaatccctgtgggtatacgctgcgtccaagaccgaagctgaactggctgcatggaaatttatggatgagaacaagccacacttcactctgaacgctgtactgccaaactacactattggcactattttcgatccggaaacccagggcgggtccacctccggttggatgatgtccctgtttaacggcgaggtttccccggctctggctctgatgccaccgcagtactacgtttccgctgttgatattggcctgctgcacctgggttgcctggttctgccacaaatcgaacgtcgtcgtgtttacggtacggctggtactttcgattggaacaccgttctggctaccttccgtaaactgtacccgtccaaaaccttcccggctgacttcccagatcaaggtcaggacctgtctaaattcgacaccgctccgtccctggaaattctgaaatctctgggtcgcccaggttggcgttccatcgaagaatccatcaaagacctggttggttccgaaaccgct

SEQ ID NO: 148 - protein, 343 aa, synthetic construct
  REGION 1..343: note = KRED-101 variant
MAKIDNAVLPEGSLVLVTGANGFIGSHVVEQLLEHGYKVRGTVRSAEKNANLQKRWDAKYPGRFETVEVPDMLKQGAYDEVIKGAAGVAHVASVTSFSNKYDEVVTPAIGGTLNALRAAAATPSVKRFVLTSSTFSALVPKPNVEGIYLDEKSWNLESIDKAKTLPESDPQKSLWVYAASKTEAELAAWKFMDENKPHFTLNAVLPNYTIGTIFDPETQGGSTSGWMMSLFNGEVSPALALMPPQYYVSAVDIGLLHLGCLVLPQIERRRVYGTAGTFDWNTVLATFRKLYPSKTFPADFPDQGQDLSKFDTAPSLEILKSLGRPGWRSIEESIKDLVGSETA

SEQ ID NO: 149 - DNA, 1029 bp, synthetic construct
  misc_feature 1..1029: note = KRED-101 variant
  source 1..1029: mol_type = other DNA
atggcaaagatcgataacgcagttctgccggaaggttccctggttctggttaccggtgctaacggtttcattggttcccacgttgttgaacagctgctggaacacggttacaaagttcgtggtaccgcgcgttccgcttcaaaacttgctaacctgcagaaacgttgggacgctaaatacccgggtcgtttcgaaaccgcggaagttgaggacatgctgaaacagggtgcttacgacgaagttatcaaaggtgctgctggtgttgctcacgttgcttccgtaacatccttctccaacaaatacgacgaagttgttaccccggctatcggtggtaccttgaacgctctgcgtgctgctgctgctaccccgtccgttaaacgtttcgttctgacctcctccaccttttccgctctggttccgaaaccgaacgttgaaggtatctacctggacgaaaaatcctggaacctggaatccatcgacaaagctaaaaccctgccggaatccgacccgcagaaatccctgtgggtatacgctgcgtccaagaccgaagctgaactggctgcatggaaatttatggatgagaacaagccacacttcactctgaacgctgtactgccaaactacactattggcactattttcgatccggaaaaccagggcgggtccacctccggttggatgatgtccctgtttaacggcgaggtttccccggctctggctctgatgccaccgcagtactacgtttccgctgttgatattggcctgctgcacctgggttgcctggttctgccacaaatcgaacgtcgtcgtgtttacggtacggctggtactttcgattggaacaccgttctggctaccttccgtaaactgtacccgtccaaaaccttcccggctgacttcccagatcaaggtcaggacctgtctaaattcgacaccgctccgtccctggaaattctgaaatctctgggtcgcccaggttggcgttccatcgaagaatccatcaaagacctggttggttccgaaaccgct

SEQ ID NO: 150 - protein, 343 aa, synthetic construct
  REGION 1..343: note = KRED-101 variant
MAKIDNAVLPEGSLVLVTGANGFIGSHVVEQLLEHGYKVRGTARSASKLANLQKRWDAKYPGRFETAEVEDMLKQGAYDEVIKGAAGVAHVASVTSFSNKYDEVVTPAIGGTLNALRAAAATPSVKRFVLTSSTFSALVPKPNVEGIYLDEKSWNLESIDKAKTLPESDPQKSLWVYAASKTEAELAAWKFMDENKPHFTLNAVLPNYTIGTIFDPENQGGSTSGWMMSLFNGEVSPALALMPPQYYVSAVDIGLLHLGCLVLPQIERRRVYGTAGTFDWNTVLATFRKLYPSKTFPADFPDQGQDLSKFDTAPSLEILKSLGRPGWRSIEESIKDLVGSETA

SEQ ID NO: 151 - DNA, 1029 bp, synthetic construct
  misc_feature 1..1029: note = KRED-101 variant
  source 1..1029: mol_type = other DNA
atggcaaagatcgataacgcagttctgccggaaggttccctggttctggttaccggtgctaacggtttcgttggttcccacgttgttgaacagctgctggaacacggttacaaagttcgtggtaccgctcgttccgcttccaaactggctaacctgcagaaacgttgggacgctaaatacccgggtcgtttcgaaaccgctgttgttgaagacatgcttaagcaaggtgcttttgacgaagttatcaaaggtgctgctggtgttgctcacatcgcttccgttacatccttttccaacaaatacgacgaggttgttaccggtgcgatcggtggtaccttgaacgctctgcgtgctgctgctgctaccccgtccgttaaacgtttcgttctgacctcctccaccttttccgctctggttccgaaaccgaacgttgaaggtatctacctggacgaaaaatcctggaacctggaatccatcgacaaagctaaaaccctgccggaatccgacccgcagaaatccctgtgggtatacgctgcgtccaagaccgaagctgaactggctgcatggaaatttatggatgagaacaagccacacttcactctgaacgctgtactgccaaactacactattggcactatctttgatccggaaactcagtccggttccacctccggttggatgatgtccctgtttaacggcgaggtttccccggctctggctctgatgccaccgcagtactacgtttccgctgttgatattggcctgctgcacctgggttgcctggttctgccacaaatcgaacgtcgtcgtgtttacggtacggctggtactttcgattggaacaccgttctggctaccttccgtaaactgtacccgtccaaaaccttcccggctgacttcccagatcaaggtcaggacctgtctaaattcgacaccgctccgtccctggaaattctgaaatctctgggtcgcccaggttggcgttccatcgaagaatccatcaaagacctggttggttccgaaaccgct

SEQ ID NO: 152 - protein, 343 aa, synthetic construct
  REGION 1..343: note = KRED-101 variant
MAKIDNAVLPEGSLVLVTGANGFVGSHVVEQLLEHGYKVRGTARSASKLANLQKRWDAKYPGRFETAVVEDMLKQGAFDEVIKGAAGVAHIASVTSFSNKYDEVVTGAIGGTLNALRAAAATPSVKRFVLTSSTFSALVPKPNVEGIYLDEKSWNLESIDKAKTLPESDPQKSLWVYAASKTEAELAAWKFMDENKPHFTLNAVLPNYTIGTIFDPETQSGSTSGWMMSLFNGEVSPALALMPPQYYVSAVDIGLLHLGCLVLPQIERRRVYGTAGTFDWNTVLATFRKLYPSKTFPADFPDQGQDLSKFDTAPSLEILKSLGRPGWRSIEESIKDLVGSETA

SEQ ID NO: 153 - DNA, 1029 bp, synthetic construct
  misc_feature 1..1029: note = KRED-101 variant
  source 1..1029: mol_type = other DNA
atggcaaagatcgataacgcagttctgccggaaggttccctggttctggttaccggtgctaacggtttcgttggttcccacgttgttgaacagctgctggaacacggttacaaagttcgtggtaccgctcgttccgcttccaaactggctaacctgcagaaacgttgggacgctaaatacccgggtcgtttcgaaaccgctgttgttgaagacatgcttgcagaaggtgcttttgacgaagttatcaaaggtgctgctggtgttgctcacatcgcttccgttacatccttctccaacaaatacgacgaggttgttacccccgtgatcggtggtaccttgaacgctctgcgtgctgctgctgctaccccgtccgttaaacgtttcgttctgacctcctccaccttttccgctctggttccgaaaccgaacgttgaaggtatctacctggacgaaaaatcctggaacctggaatccatcgacaaagctaaaaccctgccggaatccgacccgcagaaatccctgtgggtatacgctgcgtccaagaccgaagctgaactggctgcatggaaatttatggatgagaacaagccacacttcactctgaacgctgtactgccaaactacactattggcactattttcgatccggaaactcagtccggttccacctcgggttggatgatgtccctgtttaacggcgaggtttccccggctctggctctgatgccaccgcaatactacgtttccgctgttgatattggcctgctgcacctgggttgcctggttctgccacaaatcgaacgtcgtcgtgtttacggtacggctggtactttcgattggaacaccgttctggctaccttccgtaaactgtacccgtccaaaaccttcccggctgacttcccagatcaaggtcaggacctgtctaaattcgacaccgctccgtccctggaaattctgaaatctctgggtcgcccaggttggcgttccatcgaagaatccatcaaagacctggttggttccgaaaccgct

SEQ ID NO: 154 - protein, 343 aa, synthetic construct
  REGION 1..343: note = KRED-101 variant
MAKIDNAVLPEGSLVLVTGANGFVGSHVVEQLLEHGYKVRGTARSASKLANLQKRWDAKYPGRFETAVVEDMLAEGAFDEVIKGAAGVAHIASVTSFSNKYDEVVTPVIGGTLNALRAAAATPSVKRFVLTSSTFSALVPKPNVEGIYLDEKSWNLESIDKAKTLPESDPQKSLWVYAASKTEAELAAWKFMDENKPHFTLNAVLPNYTIGTIFDPETQSGSTSGWMMSLFNGEVSPALALMPPQYYVSAVDIGLLHLGCLVLPQIERRRVYGTAGTFDWNTVLATFRKLYPSKTFPADFPDQGQDLSKFDTAPSLEILKSLGRPGWRSIEESIKDLVGSETA

SEQ ID NO: 155 - DNA, 1029 bp, synthetic construct
  misc_feature 1..1029: note = KRED-101 variant
  source 1..1029: mol_type = other DNA
atggcaaagatcgataacgcagttctgccggaaggttccctggttctggttaccggtgctaacggtttcgttggttcccacgttgttgaacagctgctggaacacggttacaaagttcgtggtaccgctcgttccgcttccaaactggctaacctgcagaaacgttgggacgctaaatacccgggtcgtttcgaaaccgctgttgttgaagacatgcttaaggaaggtgcttttgacgaagttatcaaaggtgctgctggtgttgctcacatcgcttccgttacatccttctcccccaaatacgacgaggttgttacccccgtgatcggtggtaccttgaacgctctgcgtgctgctgctgctaccccgtccgttaaacgtttcgttctgacctcctccaccttttccgctctggttccgaaaccgaacgttgaaggtatctacctggacgaaaaatcctggaacctggaatccatcgacaaagctaaaaccctgccggaatccgacccgcagaaatccctgtgggtatacgctgcgtccaagaccgaagctgaactggctgcatggaaatttatggatgagaacaagccacacttcactctgaacgctgtactgccaaactacactattggcactatcttttctccggaaactcagtccggttccaccgcgggttggatgatgtccctgtttaacggcgaggtttccccggctctggctctgatgccaccgcagtactacgtttccgctgttgatattggcctgctgcacctgggttgcctggttctgccacaaatcgaacgtcgtcgtgtttacggtacggctggtactttcgattggaacaccgttctggctaccttccgtaaactgtacccgtccaaaaccttcccggctgacttcccagatcaaggtcaggacctgtctaaattcgacaccgctccgtccctggaaattctgaaatctctgggtcgcccaggttggcgttccatcgaagaatccatcaaagacctggttggttccgaaaccgct

SEQ ID NO: 156 - protein, 343 aa, synthetic construct
  REGION 1..343: note = KRED-101 variant
MAKIDNAVLPEGSLVLVTGANGFVGSHVVEQLLEHGYKVRGTARSASKLANLQKRWDAKYPGRFETAVVEDMLKEGAFDEVIKGAAGVAHIASVTSFSPKYDEVVTPVIGGTLNALRAAAATPSVKRFVLTSSTFSALVPKPNVEGIYLDEKSWNLESIDKAKTLPESDPQKSLWVYAASKTEAELAAWKFMDENKPHFTLNAVLPNYTIGTIFSPETQSGSTAGWMMSLFNGEVSPALALMPPQYYVSAVDIGLLHLGCLVLPQIERRRVYGTAGTFDWNTVLATFRKLYPSKTFPADFPDQGQDLSKFDTAPSLEILKSLGRPGWRSIEESIKDLVGSETA

SEQ ID NO: 157 - DNA, 1029 bp, synthetic construct
  misc_feature 1..1029: note = KRED-101 variant
  source 1..1029: mol_type = other DNA
atggcaaagatcgataacgcagttctgccggaaggttccctggttctggttacctcggctaacggtttcgttggttcccacgttgttgaacagctgctggaacacggttacaaagttcgtggtaccgctcgttccgcttccaaactggctaacctgcagaaacgttgggacgctaaatacccgggtcgtttcgaaaccgctgttgttgaagacatgctgaaacagggtgcttacgacgaagttatcaaaggtgctgctggtgttgctcacatcgcttccgttacatccttctccaacaaatacgacgaagttgttaccccggctatcggtggtaccttgaacgctctgcgtgctgctgctgctaccccgtccgttaaacgtttcgttctgacctcctccaccttttccgctctggttccgaaaccgaacgttgaaggtatctacctggacgaaaaatcctggaacctggaatccatcgacaaagctaaaaccctgccggaatccgacccgcagaaatccctgtgggtatacgctgcgtccaagaccgaagctgaactggctgcatggaaatttatggatgagaacaagccacacttcactctgaacgctgtactgccaaactacactattggcactattttcgatccggaaactcagtccggttccacctccggttggatgatgtccctgtttaacggcgaggtttccccggctctggctctgatgccaccgcagtactacgtttccgctgttgatattggcctgctgcacctgggttgcctggttctgccacaaatcgaacgtcgtcgtgtttacggtacggctggtactttcgattggaacaccgttctggctaccttccgtaaactgtacccgtccaaaaccttcccggctgacttcccagatcaaggtcaggacctgtctaaattcgacaccgctccgtccctggaaattctgaaatctctgggtcgcccaggttggcgttccatcgaagaatccatcaaagacctggttggttccgaaaccgct

SEQ ID NO: 158 - protein, 343 aa, synthetic construct
  REGION 1..343: note = KRED-101 variant
MAKIDNAVLPEGSLVLVTSANGFVGSHVVEQLLEHGYKVRGTARSASKLANLQKRWDAKYPGRFETAVVEDMLKQGAYDEVIKGAAGVAHIASVTSFSNKYDEVVTPAIGGTLNALRAAAATPSVKRFVLTSSTFSALVPKPNVEGIYLDEKSWNLESIDKAKTLPESDPQKSLWVYAASKTEAELAAWKFMDENKPHFTLNAVLPNYTIGTIFDPETQSGSTSGWMMSLFNGEVSPALALMPPQYYVSAVDIGLLHLGCLVLPQIERRRVYGTAGTFDWNTVLATFRKLYPSKTFPADFPDQGQDLSKFDTAPSLEILKSLGRPGWRSIEESIKDLVGSETA

SEQ ID NO: 159 - DNA, 1029 bp, synthetic construct
  misc_feature 1..1029: note = KRED-101 variant
  source 1..1029: mol_type = other DNA
atggcaaagatcgataacgcagttctgccggaaggttccctggttctggttaccggtgctaacggtttcgttggttcccacgttgttgaacagctgctggaacacggttacaaagttcgtggtaccgctcgttccgcttccaaactggctaacctgcagaaacgttgggacgctaaatacccgggtcgtttcgaaaccgctgttgttgaagacatgctgaaacagggtgcttacgacgaagttatcaaaggtgctgctggtgttgctcacatcgcttccgtttgttccttctccaacaaatacgacgaagttgttaccccggctatcggtggtaccttgaacgctctgcgtgctgctgctgctaccccgtccgttaaacgtttcgttctgacctcctccaccttttccgctctggttccgaaaccgaacgttgaaggtatctacctggacgaaaaatcctggaacctggaatccatcgacaaagctaaaaccctgccggaatccgacccgcagaaatccctgtgggtatacgctgcgtccaagaccgaagctgaactggctgcatggaaatttatggatgagaacaagccacacttcactctgaacgctgtactgccaaactacactattggcactattttcgatccggaaactcagtccggttccacctccggttggatgatgtccctgtttaacggcgaggtttccccggctctggctctgatgccaccgcagtactacgtttccgctgttgatattggcctgctgcacctgggttgcctggttctgccacaaatcgaacgtcgtcgtgtttacggtacggctggtactttcgattggaacaccgttctggctaccttccgtaaactgtacccgtccaaaaccttcccggctgacttcccagatcaaggtcaggacctgtctaaattcgacaccgctccgtccctggaaattctgaaatctctgggtcgcccaggttggcgttccatcgaagaatccatcaaagacctggttggttccgaaaccgct

SEQ ID NO: 160 - protein, 343 aa, synthetic construct
  REGION 1..343: note = KRED-101 variant
MAKIDNAVLPEGSLVLVTGANGFVGSHVVEQLLEHGYKVRGTARSASKLANLQKRWDAKYPGRFETAVVEDMLKQGAYDEVIKGAAGVAHIASVCSFSNKYDEVVTPAIGGTLNALRAAAATPSVKRFVLTSSTFSALVPKPNVEGIYLDEKSWNLESIDKAKTLPESDPQKSLWVYAASKTEAELAAWKFMDENKPHFTLNAVLPNYTIGTIFDPETQSGSTSGWMMSLFNGEVSPALALMPPQYYVSAVDIGLLHLGCLVLPQIERRRVYGTAGTFDWNTVLATFRKLYPSKTFPADFPDQGQDLSKFDTAPSLEILKSLGRPGWRSIEESIKDLVGSETA

SEQ ID NO: 161 - DNA, 1029 bp, synthetic construct
  misc_feature 1..1029: note = KRED-101 variant
  source 1..1029: mol_type = other DNA
atggcaaagatcgataacgcagttctgccggaaggttccctggttctggttaccggtgctaacggtttcgttggttcccacgttgttgaacagctgctggaacacggttacaaagttcgtggtaccgctcgttccgcttccaaactggctaacctgcagaaacgttgggacgctaaatacccgggtcgtttcgaaaccgctgttgttgaagacatgctgaaacagggtgcttacgacgaagttatcaaaggtgctgctggtgttgctcacatcgcttccgttacagggttctccaacaaatacgacgaagttgttaccccggctatcggtggtaccttgaacgctctgcgtgctgctgctgctaccccgtccgttaaacgtttcgttctgacctcctccaccttttccgctctggttccgaaaccgaacgttgaaggtatctacctggacgaaaaatcctggaacctggaatccatcgacaaagctaaaaccctgccggaatccgacccgcagaaatccctgtgggtatacgctgcgtccaagaccgaagctgaactggctgcatggaaatttatggatgagaacaagccacacttcactctgaacgctgtactgccaaactacactattggcactattttcgatccggaaactcagtccggttccacctccggttggatgatgtccctgtttaacggcgaggtttccccggctctggctctgatgccaccgcagtactacgtttccgctgttgatattggcctgctgcacctgggttgcctggttctgccacaaatcgaacgtcgtcgtgtttacggtacggctggtactttcgattggaacaccgttctggctaccttccgtaaactgtacccgtccaaaaccttcccggctgacttcccagatcaaggtcaggacctgtctaaattcgacaccgctccgtccctggaaattctgaaatctctgggtcgcccaggttggcgttccatcgaagaatccatcaaagacctggttggttccgaaaccgct

SEQ ID NO: 162 - protein, 343 aa, synthetic construct
  REGION 1..343: note = KRED-101 variant
MAKIDNAVLPEGSLVLVTGANGFVGSHVVEQLLEHGYKVRGTARSASKLANLQKRWDAKYPGRFETAVVEDMLKQGAYDEVIKGAAGVAHIASVTGFSNKYDEVVTPAIGGTLNALRAAAATPSVKRFVLTSSTFSALVPKPNVEGIYLDEKSWNLESIDKAKTLPESDPQKSLWVYAASKTEAELAAWKFMDENKPHFTLNAVLPNYTIGTIFDPETQSGSTSGWMMSLFNGEVSPALALMPPQYYVSAVDIGLLHLGCLVLPQIERRRVYGTAGTFDWNTVLATFRKLYPSKTFPADFPDQGQDLSKFDTAPSLEILKSLGRPGWRSIEESIKDLVGSETA

SEQ ID NO: 163 - DNA, 1029 bp, synthetic construct; identical to SEQ ID NO: 157
  misc_feature 1..1029: note = KRED-101 variant
  source 1..1029: mol_type = other DNA

SEQ ID NO: 164 - protein, 343 aa, synthetic construct; identical to SEQ ID NO: 158
  REGION 1..343: note = KRED-101 variant

SEQ ID NO: 165 - DNA, 1029 bp, synthetic construct
  misc_feature 1..1029: note = KRED-101 variant
  source 1..1029: mol_type = other DNA
atggcaaagatcgataacgcagttctgccggaaggttccctggttctggttaccggtgctaacggtttcgttggttcccacgttgttgaacagctgctggaacacggttacaaagttcgtggtaccgctcgttccgcttccaaactggctaacctgcagaaacgttgggacgctaaatacccgggtcgtttcgaaaccgctgttgttgaagaccagctgaaacagggtgcttacgacgaagttatcaaaggtgctgctggtgttgctcacatcgcttccgttacatccttctccaacaaatacgacgaagttgttaccccggctatcggtggtaccttgaacgctctgcgtgctgctgctgctaccccgtccgttaaacgtttcgttctgacctcctccaccttttccgctctggttccgaaaccgaacgttgaaggtatctacctggacgaaaaatcctggaacctggaatccatcgacaaagctaaaaccctgccggaatccgacccgcagaaatccctgtgggtatacgctgcgtccaagaccgaagctgaactggctgcatggaaatttatggatgagaacaagccacacttcactctgaacgctgtactgccaaactacactattggcactattttcgatccggaaactcagtccggttccacctccggttggatgatgtccctgtttaacggcgaggtttccccggctctggctctgatgccaccgcagtactacgtttccgctgttgatattggcctgctgcacctgggttgcctggttctgccacaaatcgaacgtcgtcgtgtttacggtacggctggtactttcgattggaacaccgttctggctaccttccgtaaactgtacccgtccaaaaccttcccggctgacttcccagatcaaggtcaggacctgtctaaattcgacaccgctccgtccctggaaattctgaaatctctgggtcgcccaggttggcgttccatcgaagaatccatcaaagacctggttggttccgaaaccgct

SEQ ID NO: 166 - protein, 343 aa, synthetic construct
  REGION 1..343: note = KRED-101 variant
MAKIDNAVLPEGSLVLVTGANGFVGSHVVEQLLEHGYKVRGTARSASKLANLQKRWDAKYPGRFETAVVEDQLKQGAYDEVIKGAAGVAHIASVTSFSNKYDEVVTPAIGGTLNALRAAAATPSVKRFVLTSSTFSALVPKPNVEGIYLDEKSWNLESIDKAKTLPESDPQKSLWVYAASKTEAELAAWKFMDENKPHFTLNAVLPNYTIGTIFDPETQSGSTSGWMMSLFNGEVSPALALMPPQYYVSAVDIGLLHLGCLVLPQIERRRVYGTAGTFDWNTVLATFRKLYPSKTFPADFPDQGQDLSKFDTAPSLEILKSLGRPGWRSIEESIKDLVGSETA

SEQ ID NO: 167 - DNA, 1029 bp, synthetic construct
  misc_feature 1..1029: note = KRED-101 variant
  source 1..1029: mol_type = other DNA
atggcaaagatcgataacgcagttctgccggaaggttccctggttctggttaccggtgctaacggtttcgttggttcccacgttgttgaacagctgctggaacacggttacaaagttcgtggtaccgctcgttccgcttccaaactggctaacctgcagaaacgttgggacgctaaatacccgggtcgtttcgaaacctgggttgttgaagacatgctgaaacagggtgcttacgacgaagttatcaaaggtgctgctggtgttgctcacatcgcttccgttacatccttctccaacaaatacgacgaagttgttaccccggctatcggtggtaccttgaacgctctgcgtgctgctgctgctaccccgtccgttaaacgtttcgttctgacctcctccaccttttccgctctggttccgaaaccgaacgttgaaggtatctacctggacgaaaaatcctggaacctggaatccatcgacaaagctaaaaccctgccggaatccgacccgcagaaatccctgtgggtatacgctgcgtccaagaccgaagctgaactggctgcatggaaatttatggatgagaacaagccacacttcactctgaacgctgtactgccaaactacactattggcactattttcgatccggaaactcagtccggttccacctccggttggatgatgtccctgtttaacggcgaggtttccccggctctggctctgatgccaccgcagtactacgtttccgctgttgatattggcctgctgcacctgggttgcctggttctgccacaaatcgaacgtcgtcgtgtttacggtacggctggtactttcgattggaacaccgttctggctaccttccgtaaactgtacccgtccaaaaccttcccggctgacttcccagatcaaggtcaggacctgtctaaattcgacaccgctccgtccctggaaattctgaaatctctgggtcgcccaggttggcgttccatcgaagaatccatcaaagacctggttggttccgaaaccgct

SEQ ID NO: 168 - protein, 343 aa, synthetic construct
  REGION 1..343: note = KRED-101 variant
MAKIDNAVLPEGSLVLVTGANGFVGSHVVEQLLEHGYKVRGTARSASKLANLQKRWDAKYPGRFETWVVEDMLKQGAYDEVIKGAAGVAHIASVTSFSNKYDEVVTPAIGGTLNALRAAAATPSVKRFVLTSSTFSALVPKPNVEGIYLDEKSWNLESIDKAKTLPESDPQKSLWVYAASKTEAELAAWKFMDENKPHFTLNAVLPNYTIGTIFDPETQSGSTSGWMMSLFNGEVSPALALMPPQYYVSAVDIGLLHLGCLVLPQIERRRVYGTAGTFDWNTVLATFRKLYPSKTFPADFPDQGQDLSKFDTAPSLEILKSLGRPGWRSIEESIKDLVGSETA

SEQ ID NO: 169 - DNA, 1029 bp, synthetic construct
  misc_feature 1..1029: note = KRED-101 variant
  source 1..1029: mol_type = other DNA
atggcaaagatcgataacgcagttctgccggaaggttccctggttctggttaccggtgctaacggtttcgttggttcccacgttgttgaacagctgctggaacacggttacaaagttcgtggtaccgctcgttccgcttccaaactggctaacctgcagaaacgttgggacgctaaatacccgggtcgtttcgaaaccgctgttgttgaagacatgctgaaacagggtgcttacgacgaagttatcaaaggtgctgctggtgttgctcacatcgcttccgttacatccttctccaacaaatacgacgaagttgttaccccggctatcggtggtaccttggttgctctgcgtgctgctgctgctaccccgtccgttaaacgtttcgttctgacctcctccaccttttccgctctggttccgaaaccgaacgttgaaggtatctacctggacgaaaaatcctggaacctggaatccatcgacaaagctaaaaccctgccggaatccgacccgcagaaatccctgtgggtatacgctgcgtccaagaccgaagctgaactggctgcatggaaatttatggatgagaacaagccacacttcactctgaacgctgtactgccaaactacactattggcactattttcgatccggaaactcagtccggttccacctccggttggatgatgtccctgtttaacggcgaggtttccccggctctggctctgatgccaccgcagtactacgtttccgctgttgatattggcctgctgcacctgggttgcctggttctgccacaaatcgaacgtcgtcgtgtttacggtacggctggtactttcgattggaacaccgttctggctaccttccgtaaactgtacccgtccaaaaccttcccggctgacttcccagatcaaggtcaggacctgtctaaattcgacaccgctccgtccctggaaattctgaaatctctgggtcgcccaggttggcgttccatcgaagaatccatcaaagacctggttggttccgaaaccgct

SEQ ID NO: 170 - protein, 343 aa, synthetic construct
  REGION 1..343: note = KRED-101 variant
MAKIDNAVLPEGSLVLVTGANGFVGSHVVEQLLEHGYKVRGTARSASKLANLQKRWDAKYPGRFETAVVEDMLKQGAYDEVIKGAAGVAHIASVTSFSNKYDEVVTPAIGGTLVALRAAAATPSVKRFVLTSSTFSALVPKPNVEGIYLDEKSWNLESIDKAKTLPESDPQKSLWVYAASKTEAELAAWKFMDENKPHFTLNAVLPNYTIGTIFDPETQSGSTSGWMMSLFNGEVSPALALMPPQYYVSAVDIGLLHLGCLVLPQIERRRVYGTAGTFDWNTVLATFRKLYPSKTFPADFPDQGQDLSKFDTAPSLEILKSLGRPGWRSIEESIKDLVGSETA

SEQ ID NO: 171 - DNA, 1011 bp, synthetic construct
  misc_feature 1..1011: note = PDH-101 variant
  source 1..1011: mol_type = other DNA
atgctgccgaaactcgttataactcaccgagtacacgaagagatcctgcaactgctggcgccacattgcgagctgataaccaaccagaccgacagcacgctgacgcgcgaggaaattctgcgccgctgtcgcgatgctcaggcgatgatggcgttcatgcccgatcgggtcgatgcagactttcttcaagcctgccctgagctgcgtgtaatcggctgcgcgctcaagggcttcgacaatttcgatgtggacgcctgtactgcccgcggggtctggctgaccttcgtgcctgatctgttgacggtcccgactgccgagctggcgatcggactggcggtggggctggggcggcatctgcgggcagcagatgcgttcgtccgctctggcaagttccggggctggcaaccacggttctacggcacggggctggataacgctacggtcggcttccttggcatgggcgccatcggactggccatggctgatcgcttgcagggatggggcgcgaccctgcagtaccacgcgcggaaggctctggatacacaaaccgagcaacggctcggcctgcgccaggtggcgtgcagcgaactcttcgccagctcggacttcatcctgctggcgcttcccttgaatgccgataccctgcatctggtcaacgccgagctgcttgccctcgtacggccgggcgctctgcttgtaaacccctgtcgtggctcggtagtggatgaagccgccgtgctcgcggcgcttgagcgaggccagctcggcgggtatgcggcggatgtattcgaaatggaagactgggctcgcgcggaccggccgcagcagatcgatcctgcgctgctcgcgcatccgaatacgctgttcactccgcacatagggtcggcagtgcgcgcggtgcgcctggagattgaacgttgtgcagcgcagaacatcctccaggcattggcaggtgagcgcccaatcaacgctgtgaaccgtctgcccaaggccgagcctgccgcatgttga

SEQ ID NO: 172 - protein, 336 aa, synthetic construct
  REGION 1..336: note = PDH-101 variant
MLPKLVITHRVHEEILQLLAPHCELITNQTDSTLTREEILRRCRDAQAMMAFMPDRVDADFLQACPELRVIGCALKGFDNFDVDACTARGVWLTFVPDLLTVPTAELAIGLAVGLGRHLRAADAFVRSGKFRGWQPRFYGTGLDNATVGFLGMGAIGLAMADRLQGWGATLQYHARKALDTQTEQRLGLRQVACSELFASSDFILLALPLNADTLHLVNAELLALVRPGALLVNPCRGSVVDEAAVLAALERGQLGGYAADVFEMEDWARADRPQQIDPALLAHPNTLFTPHIGSAVRAVRLEIERCAAQNILQALAGERPINAVNRLPKAEPAAC

SEQ ID NO: 173 - DNA, 1011 bp, synthetic construct
  misc_feature 1..1011: note = PDH-101 variant
  source 1..1011: mol_type = other DNA
atgctgccgaaactcgttataactcacaaagtacacgaagagatcctgcaactgctggcgccacattgcgagctgataaccaaccagaccgacagcacgctgacgcgcgaggaaattctgcgccgctgtcgcgatgctcaggcgatgatggcgttcatgcccgatcgggtcgatgcagactttcttcaagcctgccctgagctgcgtgtaatcggcgctgcgctcaagggctttgacaatttcgatgtggacgcctgtactgcccgcggggtctggctgaccttcgtgcctgatctgttgacggtcccgactgccgagctggcgatcggactggcggtggggctggggcggcatctgcgggcagcagatgcgttcgtccgctctggcaagttccggggctggcaaccacagttctacggcacggggctggataacgctacggtcggcttccttggcatgggcgccatcggactggccatggctgatcgcttgcagggatggggcgcgaccctgcagtaccacgcgcggaaggctctggatacacaaaccgagcaacggctcggcctgcgccaggtggcgtgcagcgaactcttcgccagctcggacttcatcctgctggcgcttcccttgaatgccgataccctgcatctggtcaacgccgagctgcttgccctcgtacggccgggcgctctgcttgttaacccctgtcgtggctcggtagtggatgaagccgccgtgctcgcggcgcttgagcgaggccagctcggcgggtatgcggcggatgtattcgaaatggaagactgggctcgcgcggaccggccgcagcagatcgatcctgcgctgctcgcgcatccgaatacgctgttcactccgcacatagggtcggcagtgcgcgcggtgcgcctggaaattgaacgttgcgcagcgcagaacatcctccaggcattggcaggtgagcgcccaatcaacgctgtgaaccgtctgcccaaggccgagcctgccgcatgttga

SEQ ID NO: 174 - protein, 336 aa, synthetic construct
  REGION 1..336: note = PDH-101 variant
MLPKLVITHKVHEEILQLLAPHCELITNQTDSTLTREEILRRCRDAQAMMAFMPDRVDADFLQACPELRVIGAALKGFDNFDVDACTARGVWLTFVPDLLTVPTAELAIGLAVGLGRHLRAADAFVRSGKFRGWQPQFYGTGLDNATVGFLGMGAIGLAMADRLQGWGATLQYHARKALDTQTEQRLGLRQVACSELFASSDFILLALPLNADTLHLVNAELLALVRPGALLVNPCRGSVVDEAAVLAALERGQLGGYAADVFEMEDWARADRPQQIDPALLAHPNTLFTPHIGSAVRAVRLEIERCAAQNILQALAGERPINAVNRLPKAEPAAC

SEQ ID NO: 175 - DNA, 1011 bp, synthetic construct
  misc_feature 1..1011: note = PDH-101 variant
  source 1..1011: mol_type = other DNA
atgctgccgaaactcgttataactcacaaagtacacgaagagatcctgcaactgctggcgccacattgcgagctgataaccaaccagaccgacagcacgctgacgcgcgaggaaattctgcgccgctgtcgcgatgctcaggcgatgatggcgttcatgcccgatcgggtcgatgcagactttcttcaagcctgccctgagctgcgtgtaatcggcgctgcgctcaagggctatgacaatttcgatgtggacgcctgtactgcccgcggggtctggctgaccttcgtgcctgatctgttgacggtcccgactgccgagctggccatcggactggcggtggggctggggcggcatctgcgggcagcagatgcgttcgtccgctctggcaagttccggggctggcaaccacggttctacggcacggggctggataacgctacggtcggcttccttggcatgggcgccatcggactggccatggctgatcgcttgcagggatggggcgcgaccctgcagtaccacgcgcggaaggctctggatacacaaaccgagcaacggctcggcctgcgccaggtggcgtgcagcgaactcttcgccagctcggacttcatcctgctggcgcttcccttgaatgccgataccctgcatctggtcaacgccgagctgcttgccctcgtacggccgggcgctctgcttattaacccctgtcgtggctcggtagtggatgaagccgccgtgctcgcggcgcttgagcgaggccagctcggcgggtatgcggcggatgtattcgaaatggaagactgggctcgcgcggaccggccgcagcagatcgatcctgcgctgctcgcgcatccgaatacgctgttcactccgcacatagggtcggcagtgcgcgcggtgcgcctggagattgaacgttgcgcagcgcagaacatcctccaggcattggcaggtgagcgcccaatcgacgctgtgaaccgtctgcccaaggccgagcctgccgcatgttga

SEQ ID NO: 176 - protein, 336 aa, synthetic construct
  REGION 1..336: note = PDH-101 variant
MLPKLVITHKVHEEILQLLAPHCELITNQTDSTLTREEILRRCRDAQAMMAFMPDRVDADFLQACPELRVIGAALKGYDNFDVDACTARGVWLTFVPDLLTVPTAELAIGLAVGLGRHLRAADAFVRSGKFRGWQPRFYGTGLDNATVGFLGMGAIGLAMADRLQGWGATLQYHARKALDTQTEQRLGLRQVACSELFASSDFILLALPLNADTLHLVNAELLALVRPGALLINPCRGSVVDEAAVLAALERGQLGGYAADVFEMEDWARADRPQQIDPALLAHPNTLFTPHIGSAVRAVRLEIERCAAQNILQALAGERPIDAVNRLPKAEPAAC

SEQ ID NO: 177 - DNA, 1011 bp, synthetic construct
  misc_feature 1..1011: note = PDH-101 variant
  source 1..1011: mol_type = other DNA
atgctgccgaaactcgttataactcacagagtacacgaagagatcctgcaactgctggcgccacattgcgagctgataaccaaccagaccgacagcacgctgacgcgcgaggaaattctgcgccgctgtcgcgatgctcaggcgatgatggcgttcatgcccgatcgggtcgatgcagactttcttcaagcctgccctgagctgcgtgtaatcggctgtgcgctcaagggctttgacaatttcgatgtggacgcctgtactgcccgcggggtctggctgaccttcgtgcctgatctgttgacggtcccgactgccgagctggccatcggactggcggtggggctggggcggcatctgcgggcagcagatgcgttcgtccgctctggcaagttccggggctggcaaccacagttctacggcacggggctggataacgctacggtcggcttccttggcatgggcgccatcggactggccatggctgatcgcttgcagggatggggcgcgaccctgcagtaccacgcgcggaaggctctggatacacaaaccgagcaacggctcggcctgcgccaggtggcgtgcagcgaactcttcgccagctcggacttcatcctgctggcgcttcccttgaatgccgataccctgcatctggtcaacgccgagctgcttgccctcgtacggccgggcgctctgcttattaacccctgtcgtggctcggtagtggatgaagccgccgtgctcgcggcgcttgagcgaggccagctcggcgggtatgcggcggatgtattcgaaatggaagactgggctcgcgcggaccggccgcagcagatcgatcctgcgctgctcgcgcatccgaatacgctgttcactccgcacatagggtcggcagtgcgcgcggtgcgcctggcaattgaacgttgcgcagcgcagaacatcctccaggcattggcaggtgagcgcccaatcgacgctgtgaaccgtctgcccaaggccgagcctgccgcatgttga

SEQ ID NO: 178 - protein, 336 aa, synthetic construct
  REGION 1..336: note = PDH-101 variant
MLPKLVITHRVHEEILQLLAPHCELITNQTDSTLTREEILRRCRDAQAMMAFMPDRVDADFLQACPELRVIGCALKGFDNFDVDACTARGVWLTFVPDLLTVPTAELAIGLAVGLGRHLRAADAFVRSGKFRGWQPQFYGTGLDNATVGFLGMGAIGLAMADRLQGWGATLQYHARKALDTQTEQRLGLRQVACSELFASSDFILLALPLNADTLHLVNAELLALVRPGALLINPCRGSVVDEAAVLAALERGQLGGYAADVFEMEDWARADRPQQIDPALLAHPNTLFTPHIGSAVRAVRLAIERCAAQNILQALAGERPIDAVNRLPKAEPAAC

SEQ ID NO: 179 - DNA, 1011 bp, synthetic construct
  misc_feature 1..1011: note = PDH-101 variant
  source 1..1011: mol_type = other DNA
atgctgccgaaactcgttataactcacaaagtacacgaagagatcctgcaactgctggcgccacattgcgagctgataaccaaccagaccgacagcacgctgacgcgcgaggaaattctgcgccgctgtcgcgatgctcaggcgatgatggcgttcatgcccgatcgggtcgatgcagactttcttcaagcctgccctgagctgcgtgtaatcggcgctgcgctcaagggctatgacaatttcgatgtggacgcctgtactgcccgcggggtctggctgaccttcgtgcctgatctgttgacggtcccgactgccgagctggcgatcggactggcggtggggctggggcggcatctgcgggcagcagatgcgttcgtccgctctggcaagttccggggctggcaaccacagttctacggcacggggctggataacgctacggtcggcttccttggcatgggcgccatcggactggccatggctgatcgcttgcagggatggggcgcgaccctgcagtaccacgcgcggaaggctctggatacacaaaccgagcaacggctcggcctgcgccaggtggcgtgcagcgaactcttcgccagctcggacttcatcctgctggcgcttcccttgaatgccgataccctgcatctggtcaacgccgagctgcttgccctcgtacggccgggcgctctgcttgtaaacccctgtcgtggctcggtagtggatgaagccgccgtgctcgcggcgcttgagcgaggccagctcggcgggtatgcggcggatgtattcgaaatggaagactgggctcgcgcggaccggccgcagcagatcgatcctgcgctgctcgcgcatccgaatacgctgttcactccgcacatagggtcggcagtgcgcgcggtgcgcctggagattgaacgttgtgcagcgcagaacatcctccaggcattggcaggtgagcgcccaatcgacgctgcgaaccgtctgcccaaggccgagcctgccgcatgttga

SEQ ID NO: 180 - protein, 336 aa, synthetic construct
  REGION 1..336: note = PDH-101 variant
MLPKLVITHKVHEEILQLLAPHCELITNQTDSTLTREEILRRCRDAQAMMAFMPDRVDADFLQACPELRVIGAALKGYDNFDVDACTARGVWLTFVPDLLTVPTAELAIGLAVGLGRHLRAADAFVRSGKFRGWQPQFYGTGLDNATVGFLGMGAIGLAMADRLQGWGATLQYHARKALDTQTEQRLGLRQVACSELFASSDFILLALPLNADTLHLVNAELLALVRPGALLVNPCRGSVVDEAAVLAALERGQLGGYAADVFEMEDWARADRPQQIDPALLAHPNTLFTPHIGSAVRAVRLEIERCAAQNILQALAGERPIDAANRLPKAEPAAC

SEQ ID NO: 181 - DNA, 1011 bp, synthetic construct
  misc_feature 1..1011: note = PDH-101 variant
  source 1..1011: mol_type = other DNA
atgctgccgaaactcgttataactcaccgagtacacgaagagatcctgcaactgctggcgccacattgcgagctgataaccaaccagaccgacagcacgctgacgcgcgaggaaattctgcggcgctgtgcagatgctcaggcgatgatggcgttcatgcccgatcgggtcgatgcagactttcttcaagcctgccctgagctgcgtgtaatcggctgcgcgctcaagggcttcgacaatttcgatgtggacgcctgtactgcacgcggggtctggctgaccttcgtgcctgatctgttgacggtcccgactgccgagctggcgatcggactggcggtggggctggggcggcatctgcgggcagcagatgcgttcgtccgctctggcaagttccagggctggcaaccacggttctacggcacggggctggatggagctacggtcggcttccttggcatgggcgccatcggactggccatggctgatcgcttgcagggatggggcgcgaccctgcagtaccacgcgcggaaggctctggatacacaaaccgagcaacggctcggcctgcgccaggtggcgtgcagcgaactcttcgccagctcggacttcatcctgctggcgcttcccttgaatgccgataccctgcatctggtcaacgccgagctgcttgccctcgtacggccgggcgctctgcttgtaaacccctgtcgtggctcggtagtggatgaagccgccgtgctcgcggcgcttgagcgaggccagctcggcgggtatgcggcggatgtattcgaaatggaagactgggctcgcgcggaccggccgcagcagatcgatcctgcgctgctcgcgcatccgaatacgctgttcactccgcacatagggtcggcagtgcgcgcggtgcgcctggagattgaacgttgtgcagcgcagaacatcctccaggcattggcaggtgagcgcccaattaacgctgtgaaccgtctgcccaaggccgagcctgccgcatgttga

SEQ ID NO: 182 - protein, 336 aa, synthetic construct
  REGION 1..336: note = PDH-101 variant
MLPKLVITHRVHEEILQLLAPHCELITNQTDSTLTREEILRRCADAQAMMAFMPDRVDADFLQACPELRVIGCALKGFDNFDVDACTARGVWLTFVPDLLTVPTAELAIGLAVGLGRHLRAADAFVRSGKFQGWQPRFYGTGLDGATVGFLGMGAIGLAMADRLQGWGATLQYHARKALDTQTEQRLGLRQVACSELFASSDFILLALPLNADTLHLVNAELLALVRPGALLVNPCRGSVVDEAAVLAALERGQLGGYAADVFEMEDWARADRPQQIDPALLAHPNTLFTPHIGSAVRAVRLEIERCAAQNILQALAGERPINAVNRLPKAEPAAC

SEQ ID NO: 183 - DNA, 1011 bp, synthetic construct
  misc_feature 1..1011: note = PDH-101 variant
  source 1..1011: mol_type = other DNA
atgctgccgaaactcgttataactcaccgagtacacgatgagatcctgcaactgctggcgccacattgcgagctgataaccaaccagaccgacagcacgctgacgcgcgaggaaattctggcacgctgtcgggatgctcaggcgatgatggcgttcatgcccgatcgggtcgatgcagattttcttgctgcctgccctgagctgcgtgtaatcggctgcgcgctcaagggcttcgacaatttcgatgtggacgcctgtactgcacgcggggtctggctgaccttcgtgcctgatctgttgacggtcccgactgccgagctggcgatcggactggcggtggggctggggcggcatctgcgggcagcagatgcgttcgtccgctctggcaagttccagggctggcaaccacggttctacggcacggggctggataacgctacggtcggcttccttggcatgggcgccatcggactggccatggctgatcgcttgcagggatggggcgcgaccctgcagtaccacgcgcggaaggctctggatacacaaaccgagcaacggctcggcctgcgccaggtgtcgtgcgaagaactcttcgccagctcggacttcatcctgctggcgcttcccttgaatgccgataccctgcatctggtcaacgccgagctgcttgccctcgtacggccgggcgctctgcttgtaaacccctgtcgtggctcggtagtggatgaagccgccgtgctcgcggcgcttgagcgaggccagctcggcgggtatgcggcggatgtattcgaaatggaagactgggctcgcgcggaccggccgcagcagatcgatcctgcgctgctcgcgcatccgaatacgctgttcactccgcacatagggtcggcagtgcgcgcggtgcgcctggagattgaacgttgtgcagcgcagaacatcctccaggcattggcaggtgagcgcccaatcaacgctgtgaaccgtctgcccaaggccgagcctgccgcatgttga

SEQ ID NO: 184 - protein, 336 aa, synthetic construct
  REGION 1..336: note = PDH-101 variant
MLPKLVITHRVHDEILQLLAPHCELITNQTDSTLTREEILARCRDAQAMMAFMPDRVDADFLAACPELRVIGCALKGFDNFDVDACTARGVWLTFVPDLLTVPTAELAIGLAVGLGRHLRAADAFVRSGKFQGWQPRFYGTGLDNATVGFLGMGAIGLAMADRLQGWGATLQYHARKALDTQTEQRLGLRQVSCEELFASSDFILLALPLNADTLHLVNAELLALVRPGALLVNPCRGSVVDEAAVLAALERGQLGGYAADVFEMEDWARADRPQQIDPALLAHPNTLFTPHIGSAVRAVRLEIERCAAQNILQALAGERPINAVNRLPKAEPAAC

SEQ ID NO: 185 - DNA, 1011 bp, synthetic construct
  misc_feature 1..1011: note = PDH-101 variant
  source 1..1011: mol_type = other DNA
atgctgccgaaactcgttataactcaccgagtacacgaagagatcctgcaactgctggcgccacattgcgagctgataaccaaccagaccgacagcacgctgacgcgcgaggaaattctggcacgctgtgcagatgctcaggcgatgatggcgttcatgcccgatcgggtcgatgcagattttcttcaggcctgccctgagctgcgtgtaatcggctgcgcgctcaagggcttcgacaatttcgatgtggacgcctgtactcggcgcggggtctggctgaccttcgtgcctgatctgttgacggtcccgactgccgagctggcgatcggactggcggtggggctggggcggcatctgcgggcagcagatgcgttcgtccgctctggcaagttccggggctggcaaccacggttctacggcacggggctggataacgctacggtcggcttccttggcatgggcgccatcggactggccatggctgatcgcttgcagggatggggcgcgaccctgcagtaccacgcgcggaaggctctggatacacaaaccgagcaacggctcggcctgcgccaggtgtcgtgcgaagaactcttcgccagctcggacttcatcctgctggcgcttcccttgaatgccgataccctgcatctggtcaacgccgagctgcttgccctcgtacggccgggcgctctgcttgtaaacccctgtcgtggctcggtagtggatgaagccgccgtgctcgcggcgcttgagcgaggccagctcggcgggtatgcggcggatgtattcgaaatggaagactgggctcgcgcggaccggccgcagcagatcgatcctgcgctgctcgcgcatccgaatacgctgttcactccgcacatagggtcggcagtgcgcgcggtgcgcctggagattgaacgttgtgcagcgcagaacatcctccaggcattggcaggtgagcgcccaatcaacgctgtgaaccgtctgcccaaggccgagcctgccgcatgttga

SEQ ID NO: 186 - protein, 336 aa, synthetic construct
  REGION 1..336: note = PDH-101 variant
MLPKLVITHRVHEEILQLLAPHCELITNQTDSTLTREEILARCADAQAMMAFMPDRVDADFLQACPELRVIGCALKGFDNFDVDACTRRGVWLTFVPDLLTVPTAELAIGLAVGLGRHLRAADAFVRSGKFRGWQPRFYGTGLDNATVGFLGMGAIGLAMADRLQGWGATLQYHARKALDTQTEQRLGLRQVSCEELFASSDFILLALPLNADTLHLVNAELLALVRPGALLVNPCRGSVVDEAAVLAALERGQLGGYAADVFEMEDWARADRPQQIDPALLAHPNTLFTPHIGSAVRAVRLEIERCAAQNILQALAGERPINAVNRLPKAEPAAC

SEQ ID NO: 187 - DNA, 1011 bp, synthetic construct
  misc_feature 1..1011: note = PDH-101 variant
  source 1..1011: mol_type = other DNA
atgctgccgaaactcgttataactcaccgagtacacgaagagatcctgcaactgctggcgccacattgcgagctgataaccaaccagaccgacagcacgctgacgcgcgaggaaattctgcgccgctgtcgcgatgctcaggcgatgatggcgttcatgcccgatcgggtcgatgcagactttcttcaagcctgccctgagctgcgtgtaatcggctgcgcgctcaagggcttcgacaatttcgatgtggacgcctgtactgcccgcggggtctggctgaccttcgtgcctgatctgttgacggtcccgactgccgagctggcgatcggactggcggtggggctggggcggcatctgcgggcagcagatgcgttcgtccgctctggcaagttccggggctggcaaccacggttctacggcacggggctggataacgctacggtcggcttccttggcatgggcgccatcggactggccatggctgatcgcttgcagggatggggcgcgaccctgcagtaccacgcgcggaaggctctggatacacaaaccgagcaacggctcggcctgcgccaggtggcgtgcagcgaactcttcgccagctcggacttcatcctgctggcgcttcccttgaatgccgataccctgcatctggtcaacgccgagctgcttgccctcgtacggccgggcgctctgcttgtaaacccctgtcgtggctcggtagtggatgaagccgccgtgctcgcggcgcttgagcgaggccagctcggcgggtatgcggcggatgtattcgaaatggttgactgggctcgcgcggaccggccgcagcagatcgatcctgcgctgctcgcgcatccgaatacgctgttcactccgcacatagggtcggcagtgcgcgcggtgcgcctggagattgaacgttgtgcagcgcagaacatcctccaggcattggcaggtgagcgcccaatcaacgctgtgaaccgtctgcccaaggccgagcctgccgcatgttga

SEQ ID NO: 188 - protein, 336 aa, synthetic construct
  REGION 1..336: note = PDH-101 variant
MLPKLVITHRVHEEILQLLAPHCELITNQTDSTLTREEILRRCRDAQAMMAFMPDRVDADFLQACPELRVIGCALKGFDNFDVDACTARGVWLTFVPDLLTVPTAELAIGLAVGLGRHLRAADAFVRSGKFRGWQPRFYGTGLDNATVGFLGMGAIGLAMADRLQGWGATLQYHARKALDTQTEQRLGLRQVACSELFASSDFILLALPLNADTLHLVNAELLALVRPGALLVNPCRGSVVDEAAVLAALERGQLGGYAADVFEMVDWARADRPQQIDPALLAHPNTLFTPHIGSAVRAVRLEIERCAAQNILQALAGERPINAVNRLPKAEPAAC

SEQ ID NO: 189 - DNA, 1011 bp, synthetic construct
  misc_feature 1..1011: note = PDH-101 variant
  source 1..1011: mol_type = other DNA
atgctgccgaaactcgttataactcaccgagtacacgaagagatcctgcaactgctggcgccacattgcgagctgataaccaaccagaccgacagcacgctgacgcgcgaggaaattctgcgccgctgtcgcgatgctcaggcgatgatggcgttcatgcccgatcgggtcgatgcagactttcttcaagcctgccctgagctgcgtgtaatcggctgcgcgctcaagggcttcgacaatttcgatgtggacgcctgtactgcccgcggggtctggctgaccttcgtgcctgatctgttgacggtcccgactgccgagctggcgatcggactggcggtggggctggggcggcatctgcgggcagcagatgcgttcgtccgctctggcaagttccggggctggcaaccacggttctacggcacggggctggataacgctacggtcggcttccttggcatgggcgccatcggactggccatggctgatcgcttgcagggatggggcgcgaccctgcagtaccacgcgcggaaggctctggatacacaaaccgagcaacggctcggcctgcgccaggtggcgtgcagcgaactcttcgccagctcggacttcatcctgctggcgcttcccttgaatgccgataccctgcatctggtcaacgccgagctgcttgccctcgtacggccgggcgctctgcttgtaaacccctgtcgtggctcggtagtggatgaagccgccgtgctcgcggcgcttgagcgaggccagctcggcgggtatgcggcggatgtattcgaaatgtgggactgggctcgcgcggaccggccgcagcagatcgatcctgcgctgctcgcgcatccgaatacgctgttcactccgcacatagggtcggcagtgcgcgcggtgcgcctggagattgaacgttgtgcagcgcagaacatcctccaggcattggcaggtgagcgcccaatcaacgctgtgaaccgtctgcccaaggccgagcctgccgcatgttga

SEQ ID NO: 190 - protein, 336 aa, synthetic construct
  REGION 1..336: note = PDH-101 variant
MLPKLVITHRVHEEILQLLAPHCELITNQTDSTLTREEILRRCRDAQAMMAFMPDRVDADFLQACPELRVIGCALKGFDNFDVDACTARGVWLTFVPDLLTVPTAELAIGLAVGLGRHLRAADAFVRSGKFRGWQPRFYGTGLDNATVGFLGMGAIGLAMADRLQGWGATLQYHARKALDTQTEQRLGLRQVACSELFASSDFILLALPLNADTLHLVNAELLALVRPGALLVNPCRGSVVDEAAVLAALERGQLGGYAADVFEMWDWARADRPQQIDPALLAHPNTLFTPHIGSAVRAVRLEIERCAAQNILQALAGERPINAVNRLPKAEPAAC

SEQ ID NO: 191 - DNA, 1011 bp, synthetic construct
  misc_feature 1..1011: note = PDH-101 variant
  source 1..1011: mol_type = other DNA
atgctgccgaaactcgttataactcaccgagtacacgaagagatcctgcaactgctggcgccacattgcgagctgataaccaaccagaccgacagcacgctgacgcgcgaggaaattctgcgccgctgtcgcgatgctcaggcgatgatggcgttcatgcccgatcgggtcgatgcagactttcttcaagcctgccctgagctgcgtgtaatcggctgcgcgctcaagggcttcgacaatttcgatgtggacgcctgtactgcccgcggggtctggctgaccttcgtgcctgatctgttgacggtcccgactgccgagctggcgatcggactggcggtggggctggggcggcatctgcgggcagcagatgcgttcgtccgctctggcaagttccggggctggcaaccacggttctacggcacggggctggataacgctacggtcggcttccttggcatgggcgccatcggactggccatggctgatcgcttgcagggatggggcgcgaccctgcagtaccacgcgcggaaggctctggatacacaaaccgagcaacggctcggcctgcgccaggtggcgtgcagcgaactcttcgccagctcggacttcatcctgctggcgcttcccttgaatgccgataccctgcatctggtcaacgccgagctgcttgccctcgtacggccgggcgctctgcttgtaaacccctgtcgtggctcggtagtggatgaagccgccgtgctcgcggcgcttgagcgaggccagctcggcgggtatgcggcggatgtattcgaaatgtctgactgggctcgcgcggaccggccgcagcagatcgatcctgcgctgctcgcgcatccgaatacgctgttcactccgcacatagggtcggcagtgcgcgcggtgcgcctggagattgaacgttgtgcagcgcagaacatcctccaggcattggcaggtgagcgcccaatcaacgctgtgaaccgtctgcccaaggccgagcctgccgcatgttga

SEQ ID NO: 192 - protein, 336 aa, synthetic construct
  REGION 1..336: note = PDH-101 variant
MLPKLVITHRVHEEILQLLAPHCELITNQTDSTLTREEILRRCRDAQAMMAFMPDRVDADFLQACPELRVIGCALKGFDNFDVDACTARGVWLTFVPDLLTVPTAELAIGLAVGLGRHLRAADAFVRSGKFRGWQPRFYGTGLDNATVGFLGMGAIGLAMADRLQGWGATLQYHARKALDTQTEQRLGLRQVACSELFASSDFILLALPLNADTLHLVNAELLALVRPGALLVNPCRGSVVDEAAVLAALERGQLGGYAADVFEMSDWARADRPQQIDPALLAHPNTLFTPHIGSAVRAVRLEIERCAAQNILQALAGERPINAVNRLPKAEPAAC

SEQ ID NO: 193 - DNA, 1011 bp, synthetic construct
  misc_feature 1..1011: note = PDH-101 variant
  source 1..1011: mol_type = other DNA
atgctgccgaaactcgttataactcaccgagtacacgaagagatcctgcaactgctggcgccacattgcgagctgataaccaaccagaccgacagcacgctgacgcgcgaggaaattctgcggcgctgtgcagatgctcaggcgatgatggcgttcatgcccgatcgggtcgatgcagactttcttcaagcctgccctgagctgcgtgtaatcggctgcgcgctcaagggcttcgacaatttcgatgtggacgcctgtactgcacgcggggtctggctgaccttcgtgcctgatctgttgacggtcccgactgccgagctggcgatcggactggcggtggggctggggcggcatctgcgggcagcagatgcgttcgtccgctctggcaagttccagggctggcaggaccagttctacggcacggggctggatggagctacggtcggcttccttggcatgggcgccatcggactggccatggctgatcgcttgcagggatggggcgcgaccctgcagtaccacgcgcggaaggctctggatacacaaaccgagcaacggctcggcctgcgccaggtggcgtgcagcgaactcttcgccagctcggacttcatcctgctggcgcttcccttgaatgccgataccctgcatctggtcaacgccgagctgcttgccctcgtacggccgggcgctctgcttgttaacccctgtcgtggctcggtagtggatgaagccgccgtgctcgcggcgcttgagcgaggccagctcggcgggtatgcggcggatgtattcgaaatggaagactgggctcgcgcggaccggccgcagcagatcgatcctgcgctgctcgcgcatccgaatacgctgttcactccgcaccttgggtcggcagtgcgcgcggtgcgcctggagattgaacgttgtgcggcgcagaacatcctccaggcattggcaggtgagcgcccaattaacgctgtgaaccgtctgcccaaggccgagcctgccgcatgttga

SEQ ID NO: 194 - protein, 336 aa, synthetic construct
  REGION 1..336: note = PDH-101 variant
MLPKLVITHRVHEEILQLLAPHCELITNQTDSTLTREEILRRCADAQAMMAFMPDRVDADFLQACPELRVIGCALKGFDNFDVDACTARGVWLTFVPDLLTVPTAELAIGLAVGLGRHLRAADAFVRSGKFQGWQDQFYGTGLDGATVGFLGMGAIGLAMADRLQGWGATLQYHARKALDTQTEQRLGLRQVACSELFASSDFILLALPLNADTLHLVNAELLALVRPGALLVNPCRGSVVDEAAVLAALERGQLGGYAADVFEMEDWARADRPQQIDPALLAHPNTLFTPHLGSAVRAVRLEIERCAAQNILQALAGERPINAVNRLPKAEPAAC

SEQ ID NO: 195 - DNA, 1011 bp, synthetic construct
  misc_feature 1..1011: note = PDH-101 variant
  source 1..1011: mol_type = other DNA
atgctgccgaaactcgttataactcaccgagtacacgaagagatcctgcaactgctggcgccacattgcgagctgataaccaaccagaccgacagcacgctgacgcgcgaggaaattctgcggcgctgtgcagatgctcaggcgatgatggcgttcatgcccgatcgggtcgatgcagactttcttcaagcctgccctgagctgcgtgtaatcggctgcgcgctcaagggcttcgacaatttcgatgtggacgcctgtactgcacgcggggtctggctgaccttcgtgcctgatctgttgacggtcccgactgccgagctggcgatcggactggcggtggggctggggcggcatctgcgggcagcagatgcgttcgtccgctctggcaagttccagggctggcagccaattttctacggcacggggctggatggagctacggtcggcttccttggcatgggcgccatcggactggccatggctgatcgcttgcagggatggggcgcgaccctgcagtaccacgcgcggaaggctctggatacacaaaccgagcaacggctcggcctgcgccaggtggcgtgcagcgaactcttcgccagctcggacttcatcctgctggcgcttcccttgaatgccgataccctgcatctggtcaacgccgagctgcttgccctcgtacggccgggcgctctgcttattaacccctgtcgtggctcggtagtggatgaagccgccgtgctcgcggcgcttgagcgaggccagctcggcgggtatgcggcggatgtattcgaaatggaagactgggctcgcgcggaccggccgcagcagatcgatcctgcgctgctcgcgcatccgaatacgctgttcactccgcacatagggtcggcagtgcgcgcggtgcgcctggagattgaacgttgtgtggcgcagaacatcctccaggcattggcaggtgagcgcccaattgacgctgtgaaccgtctgcccaaggccgagcctgccgcatgttga

SEQ ID NO: 196 - protein, 336 aa, synthetic construct
  REGION 1..336: note = PDH-101 variant
MLPKLVITHRVHEEILQLLAPHCELITNQTDSTLTREEILRRCADAQAMMAFMPDRVDADFLQACPELRVIGCALKGFDNFDVDACTARGVWLTFVPDLLTVPTAELAIGLAVGLGRHLRAADAFVRSGKFQGWQPIFYGTGLDGATVGFLGMGAIGLAMADRLQGWGATLQYHARKALDTQTEQRLGLRQVACSELFASSDFILLALPLNADTLHLVNAELLALVRPGALLINPCRGSVVDEAAVLAALERGQLGGYAADVFEMEDWARADRPQQIDPALLAHPNTLFTPHIGSAVRAVRLEIERCVAQNILQALAGERPIDAVNRLPKAEPAAC

SEQ ID NO: 197 - DNA, 1011 bp, synthetic construct
  misc_feature 1..1011: note = PDH-101 variant
  source 1..1011: mol_type = other DNA
atgctgccgaaactcgttataactcaccgagtacacgaagagatcctgcaactgctggcgccacattgcgagctgataaccaaccagaccgacagcacgctgacgcgcgaggaaattctgcggcgctgtgcagatgctcaggcgatgatggcgttcatgcccgatcgggtcgatgcagactttcttcaagcctgccctgagctgcgtgtaatcggctgcgcgctcaagggcttcgacaatttcgatgtggacgcctgtactgcacgcggggtctggctgaccttcgtgcctgatctgttgacggtcccgactgccgagctggcgatcggactggcggtggggctggggcggcatctgcgggcagcagatgcgttcgtccgctctggcaagttccagggctgggctgacattttctacggcacggggctggatggagctacggtcggcttccttggcatgggcgccatcggactggccatggctgatcgcttgcagggatggggcgcgaccctgcagtaccacgcgcggaaggctctggatacacaaaccgagcaacggctcggcctgcgccaggtggcgtgcagcgaactcttcgccagctcggacttcatcctgctggcgcttcccttgaatgccgataccctgcatctggtcaacgccgagctgcttgccctcgtacggccgggcgctctgcttgttaacccctgtcgtggctcggtagtggatgaagccgccgtgctcgcggcgcttgagcgaggccagctcggcgggtatgcggcggatgtattcgaaatggaagactgggctcgcgcggaccggccgcagcagatcgatcctgcgctgctcgcgcatccgaatacgctgttcactccgcaccttgggtcggcagtgcgcgcggtgcgcctggagattgaacgttgtgcagcgcagaacatcctccaggcattggcaggtgagcgcccaattaacgctgtgaaccgtctgcccaaggccgagcctgccgcatgttga

SEQ ID NO: 198 - protein, 336 aa, synthetic construct
  REGION 1..336: note = PDH-101 variant
MLPKLVITHRVHEEILQLLAPHCELITNQTDSTLTREEILRRCADAQAMMAFMPDRVDADFLQACPELRVIGCALKGFDNFDVDACTARGVWLTFVPDLLTVPTAELAIGLAVGLGRHLRAADAFVRSGKFQGWADIFYGTGLDGATVGFLGMGAIGLAMADRLQGWGATLQYHARKALDTQTEQRLGLRQVACSELFASSDFILLALPLNADTLHLVNAELLALVRPGALLVNPCRGSVVDEAAVLAALERGQLGGYAADVFEMEDWARADRPQQIDPALLAHPNTLFTPHLGSAVRAVRLEIERCAAQNILQALAGERPINAVNRLPKAEPAAC

SEQ ID NO: 199 - DNA, 1011 bp, synthetic construct
  misc_feature 1..1011: note = PDH-101 variant
  source 1..1011: mol_type = other DNA
atgctgccgaaactcgttataactcacagagtacacgaagagatcctgcaactgctggcgccacattgcgagctgataaccaaccagaccgacagcacgctgacgcgcgaggaaattctgcggcgctgtgcagatgctcaggcgatgatggcgttcatgcccgatcgggtcgatgcagactttcttcaagcctgccctgagctgcgtgtaatcggctgtgcgctcaagggcttcgacaatttcgatgtggacgcctgtactgcacgcggggtctggctgaccttcgtgcctgatctgttgacggtcccgactgccgagctggcgatcggactggcggtggggctggggcggcatctgcgggcagcagatgcgttcgtccgctctggcaagttccagggctggcagccaattttctacggcacggggctggatggagctacggtcggcttccttggcatgggcgccatcggactggccatggctgatcgcttgcagggatggggcgcgaccctgcagtaccacgcgcggaaggctctggatacacaaaccgagcaacggctcggcctgcgccaggtggcgtgcagcgaactcttcgccagctcggacttcatcctgctggcgcttcccttgaatgccgataccctgcatctggtcaacgccgagctgcttgccctcgtacggccgggcgctctgcttgtaaacccctgtcgtggctctgtagtggatgaagccgccgtgctcgcggcgcttgagcgaggccagctcggcgggtatgcggcggatgtattcgaaatggaagactgggctcgcgcggaccggccgcagcagatcgatcctgcgctgctcgcgcatccgaatacgctgttcactccgcaccttgggtcggcagtgcgcgcggtgcgcctggagattgaacgttgtgcagcgcagaacatcctccaggcattggcaggtgagcgcccaattgacgctgtgaaccgtctgcccaaggccgagcctgccgcatgttga

SEQ ID NO: 200 - protein, 336 aa, synthetic construct
  REGION 1..336: note = PDH-101 variant
MLPKLVITHRVHEEILQLLAPHCELITNQTDSTLTREEILRRCADAQAMMAFMPDRVDADFLQACPELRVIGCALKGFDNFDVDACTARGVWLTFVPDLLTVPTAELAIGLAVGLGRHLRAADAFVRSGKFQGWQPIFYGTGLDGATVGFLGMGAIGLAMADRLQGWGATLQYHARKALDTQTEQRLGLRQVACSELFASSDFILLALPLNADTLHLVNAELLALVRPGALLVNPCRGSVVDEAAVLAALERGQLGGYAADVFEMEDWARADRPQQIDPALLAHPNTLFTPHLGSAVRAVRLEIERCAAQNILQALAGERPIDAVNRLPKAEPAAC

SEQ ID NO: 201 - DNA, 1011 bp, synthetic construct
  misc_feature 1..1011: note = PDH-101 variant
  source 1..1011: mol_type = other DNA
atgctgccgaaactcgttataactcaccgagtacacgaagagatcctgcaactgctggcgccacattgcgagctgataaccaaccagaccgacagcacgctgacgcgcgaggaaattctgcggcgctgtgcagatgctcaggcgatgatggcgttcatgcccgatcgggtcgatgcagactttcttcaagcctgccctgagctgcgtgtaatcggctgcgcgctcaagggcttcgacaatttcgatgtggacgcctgtactgcacgcggggtctggctgaccttcgtgcctgatctgttgacggtcccgactgccgagctggcgatcggactggcggtggggctggggcggcatctgcgggcagcagatgcgttcgtccgctctggcaagttccagggctggcaaccacggttctacggcacggggctggatggagctacggtcggcttccttggcatgggcgccatcggactggccatggctgatcgcttgcagggatggggcgcgaccctgcagtaccacgcgcggaaggctctggatacacaaaccgagcaacggctcggcctgcgccaggtggcgtgcgaggaactcttcgccagctcggacttcatcctgctggcgcttcccttgaatgccgataccctgcatctggtcaacgccgagctgcttgccctcgtacggccgggcgctctgcttgttaacccctgtcgtggctcggtagtggatgaagccgccgtgctcgcggcgcttgagcgaggccagctcggcgggtatgcggcggatgtattcgaaatggaagactgggctcgcgcggaccggccgcagcagatcgatcctgcgctgctcgcgcatccgaatacgctgttcactccgcaccttgggtcggcagtgcgcgcggtgcgcctggagattgaacgttgtgcagcgcagaacatcctccaggcattggcaggtgagcgcccaattgacgctgtgaaccgtctgcccaaggccgagcctgccgcatgttga

SEQ ID NO: 202 - protein, 336 aa, synthetic construct
  REGION 1..336: note = PDH-101 variant
MLPKLVITHRVHEEILQLLAPHCELITNQTDSTLTREEILRRCADAQAMMAFMPDRVDADFLQACPELRVIGCALKGFDNFDVDACTARGVWLTFVPDLLTVPTAELAIGLAVGLGRHLRAADAFVRSGKFQGWQPRFYGTGLDGATVGFLGMGAIGLAMADRLQGWGATLQYHARKALDTQTEQRLGLRQVACEELFASSDFILLALPLNADTLHLVNAELLALVRPGALLVNPCRGSVVDEAAVLAALERGQLGGYAADVFEMEDWARADRPQQIDPALLAHPNTLFTPHLGSAVRAVRLEIERCAAQNILQALAGERPIDAVNRLPKAEPAAC

SEQ ID NO: 203 - DNA, 1011 bp, synthetic construct
  misc_feature 1..1011: note = PDH-101 variant
  source 1..1011: mol_type = other DNA
atgctgccgaaactcgttataactcaccgagtacacgaagagatcctgcaactgctggcgccacattgcgagctgataaccaaccagaccgacagcacgctgacgcgcgaggaaattctgcggcgctgtgcagatgctcaggcgatgatggcgttcatgcccgatcgggtcgatgcagactttcttcaagcctgccctgagctgcgtgtaatcggctgcgcgctcaagggcttcgacaatttcgatgtggacgcctgtactgcacgcggggtctggctgaccttcgtgcctgatctgttgacggtcccgactgccgagctggcgatcggactggcgtcggggctggggcggcatctgcgggcagcagatgcgttcgtccgctctggcaagttccagggctggcaaccacggttctacggcacggggctggatggagctacggtcggcttccttggcatgggcgccatcggactggccatggctgatcgcttgcagggatggggcgcgaccctgcagtaccacgcgcggaaggctctggatacacaaaccgagcaacggctcggcctgcgccaggtggcgtgcagcgaactcttcgccagctcggacttcatcctgctggcgcttcccttgaatgccgataccctgcatctggtcaacgccgagctgcttgccctcgtacggccgggcgctctgcttgtaaacccctgtcgtggctcggtagtggatgaagccgccgtgctcgcggcgcttgagcgaggccagctcggcgggtatgcggcggatgtattcgaaatggaagactgggctcgcgcggaccggccgcagcagatcgatcctgcgctgctcgcgcatccgaatacgctgttcactccgcacatagggtcggcagtgcgcgcggtgcgcctggagattgaacgttgtgcagcgcagaacatcctccaggcattggcaggtgagcgcccaattaacgctgtgaaccgtctgcccaaggccgagcctgccgcatgttga

SEQ ID NO: 204 - protein, 336 aa, synthetic construct
  REGION 1..336: note = PDH-101 variant
MLPKLVITHRVHEEILQLLAPHCELITNQTDSTLTREEILRRCADAQAMMAFMPDRVDADFLQACPELRVIGCALKGFDNFDVDACTARGVWLTFVPDLLTVPTAELAIGLASGLGRHLRAADAFVRSGKFQGWQPRFYGTGLDGATVGFLGMGAIGLAMADRLQGWGATLQYHARKALDTQTEQRLGLRQVACSELFASSDFILLALPLNADTLHLVNAELLALVRPGALLVNPCRGSVVDEAAVLAALERGQLGGYAADVFEMEDWARADRPQQIDPALLAHPNTLFTPHIGSAVRAVRLEIERCAAQNILQALAGERPINAVNRLPKAEPAAC

SEQ ID NO: 205 - DNA, 1011 bp, synthetic construct
  misc_feature 1..1011: note = PDH-101 variant
  source 1..1011: mol_type = other DNA
atgctgccgaaactcgttataactcacagagtacacgaagagatcctgcaaatgctggcgccacattgcgagctgataaccaaccagaccgacagcacgctgacgcgcgaggaaattctgcggcgctgtgcagatgctcaggcgatgatggcgttcatgcccgatcgggtcgatgcagactttcttcaagcctgccctgagctgcgtgtaatcggctgtgcgctcaagggcttcgacaatttcgatgtggacgcctgtactgcacgcggggtctggctgaccttcgtgcctgatctgttgacggtcccgactgccgagctggcgatcggactggcggtggggctggggcggcatttccgggcagcagatgaattcgtccgctctggcaagttccagggctggcagccaattttctacggcacggggctggatggagcaacggtcggcttccttggcatgggcgccatcggactcgccatggctgatcgcttgcagggatggggcgcgaccctgcagtaccacgcgcggaaggctctggatacacaaaccgagcaacggctcggcctgcgccaggtggcgtgcagcgaactcttcgccagctcggacttcatcctgctggcgcttcccttgaatgccgataccctgcatctggtcaacgccgagctgcttgccctcgtacggccgggcgctctgcttgtaaacccctgtcgtggctctgtagtggatgaagccgccgtgctcgcggcgcttgagcgaggccagctcggcgggtatgcggcggatgtattcgaaatggaagactgggctcgcgcggaccggccgcagcagatcgatcctgcgctgctcgcgcatccgaatacgctgttcactccgcaccttgggtcggcagtgcgcgcggtgcgcctggagattgaacgttgtgcagcgcagaacatcctccaggcattggcaggtgagcgcccaattgacgctgtgaaccgtctgcccaaggccgagcccaaggcccgctga

SEQ ID NO: 206 - protein, 336 aa, synthetic construct
  REGION 1..336: note = PDH-101 variant
MLPKLVITHRVHEEILQMLAPHCELITNQTDSTLTREEILRRCADAQAMMAFMPDRVDADFLQACPELRVIGCALKGFDNFDVDACTARGVWLTFVPDLLTVPTAELAIGLAVGLGRHFRAADEFVRSGKFQGWQPIFYGTGLDGATVGFLGMGAIGLAMADRLQGWGATLQYHARKALDTQTEQRLGLRQVACSELFASSDFILLALPLNADTLHLVNAELLALVRPGALLVNPCRGSVVDEAAVLAALERGQLGGYAADVFEMEDWARADRPQQIDPALLAHPNTLFTPHLGSAVRAVRLEIERCAAQNILQALAGERPIDAVNRLPKAEPKAR

SEQ ID NO: 207 - DNA, 1011 bp, synthetic construct
  misc_feature 1..1011: note = PDH-101 variant
  source 1..1011: mol_type = other DNA
atgctgccgaaactcgttataactcacagagtacacgaagagatcctgcaactgctggcgccacattgcgagctgataaccaaccagaccgacagcacgctgacgcgcgaggaaattctgcggcgctgtgcagatgctcaggcgatgatggcgttcatgcccgatcgggtcgatgcagactttcttcaagcctgccctgagctgcgtgtaatcggctgtgcgctcaagggcttcgacaatttcgatgtggacgcctgtactgcacgcggggtctggctgaccttcgtgcctgatctgttgacggtcccgactgccgagctggcgatcggactggcggtggggctggggcggcatttccgggcagcagatgcattcgtccgctctggcaagttccagggctggcagccaattttctacggcacggggctggatggagcaacggtcggcttccttggcatgggcgccatcggaaaagccatggctgatcgcttgcagggatggggcgcgaccctgcagtaccactcgcggacagctctggatacacaaaccgagcaacggctcggcctgcgccaggtggcgtgcagcgaactcttcgccagctcggacttcatcctgctggcgcttcccttgaatgccgataccctgcatctggtcaacgccgagctgcttgccctcgtacggccgggcgctctgcttgtaaacccctgtcgtggctctgtagtggatgaagccgccgtgctcgcggcgcttgagcgaggccagctcggcgggtatgcggcggatgtattcgaaatggaagactgggctcgcgcggaccggccgcagcagatcgatcctgcgctgctcgcgcatccgaatacgctgttcactccgcaccttgggtcggcagtgcgcgcggtgcgcctggagattgaacgttgtgcagcgcagaacatcctccaggcattgcgaggtgagcgcccaattgacgctgtgaaccgtctgcccaaggccgagcctgccgcatgttga

SEQ ID NO: 208 - protein, 336 aa, synthetic construct
  REGION 1..336: note = PDH-101 variant
MLPKLVITHRVHEEILQLLAPHCELITNQTDSTLTREEILRRCADAQAMMAFMPDRVDADFLQACPELRVIGCALKGFDNFDVDACTARGVWLTFVPDLLTVPTAELAIGLAVGLGRHFRAADAFVRSGKFQGWQPIFYGTGLDGATVGFLGMGAIGKAMADRLQGWGATLQYHSRTALDTQTEQRLGLRQVACSELFASSDFILLALPLNADTLHLVNAELLALVRPGALLVNPCRGSVVDEAAVLAALERGQLGGYAADVFEMEDWARADRPQQIDPALLAHPNTLFTPHLGSAVRAVRLEIERCAAQNILQALRGERPIDAVNRLPKAEPAAC

SEQ ID NO: 209 - DNA, 1011 bp, synthetic construct
  misc_feature 1..1011: note = PDH-101 variant
  source 1..1011: mol_type = other DNA
atgctgccgaaactcgttataactcacagagtacacgaagagatcctgcaaatgctggcgccacattgcgagctgataaccaaccagaccgacagcacgctgacgcgcgaggaaattctgcggcgctgtgcagatgctcaggcgatgatggcgttcatgcccgatcgggtcgatgcagactttcttcaagcctgccctgagctgcgtgtaatcggctgtgcgctcaagggcttcgacaatttcgatgtggacgcctgtactgcacgcggggtctggctgaccttcgtgcctgatctgttgacggtcccgactgccgagctggcgatcggactggcggtggggctggggcggcatttccgggcagcagatgaattcgtccgctctggcaagttccagggctggcagccaattttctacggcacggggctggatggagcaacggtcggcttccttggcatgggcgccatcggaaaagccatggctgatcgcttgcagggatggggcgcgaccctgcagtaccacgcgcggacagctctggatacacaaaccgagcaacggctcggcctgcgccaggtggcgtgcagcgaactcttcgccagctcggacttcatcctgctggcgcttcccttgaatgccgataccctgcatctggtcaacgccgagctgcttgccctcgtacggccgggcgctctgcttgtaaacccctgtcgtggctctgtagtggatgaagccgccgtgctcgcggcgcttgagcgaggccagctcggcgggtatgcggcggatgtattcgaaatggaagactgggctcgcgcggaccggccgcagcagatcgatcctgcgctgctcgcgcatccgaatacgctgttcactccgcaccttgggtcggcagtgcgcgcggtgcgcctggagattgaacgttgtgcagcgcagaacatcctccaggcattggcaggtgagcgcccaattgacgctgtgaaccgtctgcccaaggccgagcctgccgcatgttga

SEQ ID NO: 210 - protein, 336 aa, synthetic construct
  REGION 1..336: note = PDH-101 variant
MLPKLVITHRVHEEILQMLAPHCELITNQTDSTLTREEILRRCADAQAMMAFMPDRVDADFLQACPELRVIGCALKGFDNFDVDACTARGVWLTFVPDLLTVPTAELAIGLAVGLGRHFRAADEFVRSGKFQGWQPIFYGTGLDGATVGFLGMGAIGKAMADRLQGWGATLQYHARTALDTQTEQRLGLRQVACSELFASSDFILLALPLNADTLHLVNAELLALVRPGALLVNPCRGSVVDEAAVLAALERGQLGGYAADVFEMEDWARADRPQQIDPALLAHPNTLFTPHLGSAVRAVRLEIERCAAQNILQALAGERPIDAVNRLPKAEPAAC

SEQ ID NO: 211 - DNA, 1011 bp, synthetic construct
  misc_feature 1..1011: note = PDH-101 variant
  source 1..1011: mol_type = other DNA
atgctgccgaaactcgttataactcacagagtacacgaagagatcctgcaaatgctggcgccacattgcgagctgataaccaaccagaccgacagcacgctgacgcgcgaggaaattctgcggcgctgtgcagatgctcaggcgatgatggcgttcatgcccgatcgggtcgatgcagactttcttcaagcctgccctgagctgcgtgtaatcggctgtgcgctcaagggcttcgacaatttcgatgtggacgcctgtactgcacgcggggtctggctgaccttcgtgcctgatctgttgacggtcccgactgccgagctggcgatcggactggcggtggggctggggcggcatttccgggcagcagatgaattcgtccgctctggcaagttccagggctggcagccaattttctacggcacggggctggatggagcaacggtcggcttccttggcatgggcgccatcggaaaagccatggctgatcgcttgcagggatggggcgcgaccctgcagtaccactcgcggacagctctggatacacaaaccgagcaacggctcggcctgcgccaggtggcgtgcagtgaactcttcgccagctcggacttcatcctgctggcgcttcccttgaatgccgataccctgcatctggtcaacgccgagctgcttgccctcgtacggccgggcgctctgcttgtaaacccctgtcgtggctctgtagtggatgaagccgccgtgctcgcggcgcttgagcgaggccagctcggcgggtatgcggcggatgtattcgaaatggaagactgggctcgcgcggaccggccgcagcagatcgatcctgcgctgctcgcgcatccgaatacgctgttcactccgcaccttgggtcggcagtgcgcgcggtgcgcctggagattgaacgttgtgcagcgcagaacatcctccaggcattgcgaggtgagcgcccaattgacgctgtgaaccgtctgcccaaggccgagcctgccgcatgttga

SEQ ID NO: 212 - protein, 336 aa, synthetic construct
  REGION 1..336: note = PDH-101 variant
MLPKLVITHRVHEEILQMLAPHCELITNQTDSTLTREEILRRCADAQAMMAFMPDRVDADFLQACPELRVIGCALKGFDNFDVDACTARGVWLTFVPDLLTVPTAELAIGLAVGLGRHFRAADEFVRSGKFQGWQPIFYGTGLDGATVGFLGMGAIGKAMADRLQGWGATLQYHSRTALDTQTEQRLGLRQVACSELFASSDFILLALPLNADTLHLVNAELLALVRPGALLVNPCRGSVVDEAAVLAALERGQLGGYAADVFEMEDWARADRPQQIDPALLAHPNTLFTPHLGSAVRAVRLEIERCAAQNILQALRGERPIDAVNRLPKAEPAAC

SEQ ID NO: 213 - DNA, 1011 bp, synthetic construct
  misc_feature 1..1011: note = PDH-101 variant
  source 1..1011: mol_type = other DNA
atgctgccgaaactcgttataactcacagagtacacgaagagatcctgcaactgctggcgccacattgcgagctgataaccaaccagaccgactcgacgctgacgcgcgaggaaattctgcggcgctgtgcagatgctcaggcgatgatggcgttcatgcccgatcgggtcgatgcagactttcttcaagcctgccctgagctgaaagtaatcggctgtgcgctcaagggcttcgacaatttcgatgtggacgcctgtactgcacgcggggtctggctgaccttcgtgcctgatctgttgacggtcccgactgccgagctggcgatcggactggcggtggggctggggcggcatctggttgcagcagatgcgttcgtccgctctggcaagttccagggctggcagccaattttctacggcacggggctggatggagctacggtcggcttccttggcatgggcgccatcggactggccatggctgatcgcttgcagggatggggcgcgaccctgcagtaccacacccggaaggctctggatacacaaaccgagcaacggctcggcctgcgccaggtggcgtgcgaggaactcttcgccagctcggacttcatcctgctggcgcttcccttgaatgccgataccctgcatctggtcaacgccgagctgcttgccctcgtacggccgggcgctctgcttgttaacccctgtcgtggctctgtagtggatgaagccgccgtgctcgcggcgcttgagcgaggccagctcggcgggtatgcggcggatgtattcgaaatggaagactgggctcgcgcggaccggccgcagcagatcgatcctgcgctgctcgcgcatccgaatacgctgttcactccgcaccttgggtcggcagtgcgcgcggtgcgcctggagattgaacgttgtgcagcgcagaacatcctccaggcattggcaggtgagcgcccaattgacgctgtgaaccgtctgcccaaggccgagcctgccgcatgttga

SEQ ID NO: 214 - protein, 336 aa, synthetic construct
  REGION 1..336: note = PDH-101 variant
MLPKLVITHRVHEEILQLLAPHCELITNQTDSTLTREEILRRCADAQAMMAFMPDRVDADFLQACPELKVIGCALKGFDNFDVDACTARGVWLTFVPDLLTVPTAELAIGLAVGLGRHLVAADAFVRSGKFQGWQPIFYGTGLDGATVGFLGMGAIGLAMADRLQGWGATLQYHTRKALDTQTEQRLGLRQVACEELFASSDFILLALPLNADTLHLVNAELLALVRPGALLVNPCRGSVVDEAAVLAALERGQLGGYAADVFEMEDWARADRPQQIDPALLAHPNTLFTPHLGSAVRAVRLEIERCAAQNILQALAGERPIDAVNRLPKAEPAAC

SEQ ID NO: 215 - DNA, 1011 bp, synthetic construct
  misc_feature 1..1011: note = PDH-101 variant
  source 1..1011: mol_type = other DNA
atgctgccgaaactcgttataactcacagagtacacgaagagatcctgcaactgctggcgccacattgcgagctgataaccaaccagaccgacgtcacgctgacgcgcgaggaaattctgcggcgctgtgcagatgctcaggcgatgatggcgttcatgcccgatcgggtcgatgcagactttcttcaagcctgccctgagctgagagtaatcggctgtgcgctcaagggcttcgacaatttcgatgtggacgcctgtactgcacgcggggtctggctgaccttcgtgcctgatctgttgacggtcccgactgccgagctggcgatcggactggcggtggggctggggcggcatctgcgcgcagcagatgcgttcgtccgctctggcaagttccagggctggcagccaattttctacggcacggggctggatggagctacggtcggcttccttggcatgggcgccatcggactggccatggctgatcgcttgcagggatggggcgcgaccctgcagtaccacgcccggaaggctctggatacacaaactgagcaaaccctcggcctgcgccaggtggcgtgcagcgaactcttcgccagctcggacttcatcctgctggcgcttcccttgaatgccgataccctgcatctggtcaacgccgagctgcttgccctcgtacgtccgggcgctctgcttattaacccctgtcgtggctctgtagtggatgaagccgccgtgctcgcggcgcttgagcgaggccagctcggcgggtatgcggcggatgtattcgaaatggaagactgggctcgcgcggaccggccgcagcagatcgatcctgcgctgctcgcgcatccgaatacgctgttcactccgcaccttgggtcggcagtgcgcgcggtgcgcctggagattgaacgttgtgcagcgcagaacatcctccaggcattggcaggtgagcgcccaattgacgctgtgaaccgtctgcccaaggccgagcctgccgcaagctga

SEQ ID NO: 216 - protein, 336 aa, synthetic construct
  REGION 1..336: note = PDH-101 variant
MLPKLVITHRVHEEILQLLAPHCELITNQTDVTLTREEILRRCADAQAMMAFMPDRVDADFLQACPELRVIGCALKGFDNFDVDACTARGVWLTFVPDLLTVPTAELAIGLAVGLGRHLRAADAFVRSGKFQGWQPIFYGTGLDGATVGFLGMGAIGLAMADRLQGWGATLQYHARKALDTQTEQTLGLRQVACSELFASSDFILLALPLNADTLHLVNAELLALVRPGALLINPCRGSVVDEAAVLAALERGQLGGYAADVFEMEDWARADRPQQIDPALLAHPNTLFTPHLGSAVRAVRLEIERCAAQNILQALAGERPIDAVNRLPKAEPAAS

SEQ ID NO: 217 - DNA, 1011 bp, synthetic construct
  misc_feature 1..1011: note = PDH-101 variant
  source 1..1011: mol_type = other DNA
atgctgccgaaactcgttataactcacagagtacacgaagagatcctgcaactgctggcgccacattgcgagctgataaccaaccagaccgacagcacgctgacgcgcgaggaaattctgcggcgctgtgcagatgctcaggcgatgatggcgttcatgcccgatcgggtcgatgcagactttcttcaagcctgccctgagctgcgtgtaatcggctgtgcgctcaagggctatgacaatttcgatgtggacgcctgtactgcacgcggggtctggctgaccttcgtgcctgatctgttgacggtcccgactgccgagctggcgatcggactggcggtggggctggggcggcatttccgggcagcagatgcattcgtccgctctggcaagttccagggctggcagccaattttctacggcacggggctggatggagcaacggtcggcattctgggcatgggcgccatcggaaaagccatggctgatcgcttgcagggatggggcgcgaccctgcagtaccactcgcggacagctctggatacacaaaccgagcaacggctcggcctgcgccaggtggcgtgcagcgaactcttagccagctcggacttcatcctgctggcgcttcccttgaatgccgataccctgcatctggtcaacgccgagctgcttgccctcgtacggccgggcgctctgcttgtaaacccctgtcgtggctctgtagtggatgaagccgccgtgctcgcggcgcttgagcgaggccagctcggcgggtatgcggcggatgtattcgaaatggaagactgggctcgcgcggaccggccgcagcagatcgatcctgcgctgctcgcgcatccgaatacgctgttcactccgcaccttgggtcggcagtgcgcgcggtgcgcctggagattgaacgttgtgcagcgcagaacatcctccaggcattgcgaggtgagcgcccaattgacgctgtgaacagcccgcccaaggccgagcctgccgcatgttga

SEQ ID NO: 218 - protein, 336 aa, synthetic construct
  REGION 1..336: note = PDH-101 variant
MLPKLVITHRVHEEILQLLAPHCELITNQTDSTLTREEILRRCADAQAMMAFMPDRVDADFLQACPELRVIGCALKGYDNFDVDACTARGVWLTFVPDLLTVPTAELAIGLAVGLGRHFRAADAFVRSGKFQGWQPIFYGTGLDGATVGILGMGAIGKAMADRLQGWGATLQYHSRTALDTQTEQRLGLRQVACSELLASSDFILLALPLNADTLHLVNAELLALVRPGALLVNPCRGSVVDEAAVLAALERGQLGGYAADVFEMEDWARADRPQQIDPALLAHPNTLFTPHLGSAVRAVRLEIERCAAQNILQALRGERPIDAVNSPPKAEPAAC

SEQ ID NO: 219 - DNA, 1011 bp, synthetic construct
  misc_feature 1..1011: note = PDH-101 variant
  source 1..1011: mol_type = other DNA
atgctgccgaaactcgttataactcacagagtacacgaagagatcctgcaactgctggcgccacattgcgagctgataaccaaccagaccgacagcacgctgacgcgcgaggaaattctgcggcgctgtgcagatgctcaggcgatgatggcgttcatgcccgatcgggtcgatgcagactttcttcaagcctgccctgagctgcgtgtaatcggctgtgcgctcaagggcttcgacaatttcgatgtggacgcctgtactgcacgcggggtctggctgacctttgtgcctgatctgttgacggtcccgactgccgagctggcgatcggactggcggtggggctggggcggcatttccgggcagcagatgcattcgtccgctctggcaagttccagggctggcagccaattttctacggcacggggctggatggagcaacggtcggcttccttggcatgggcgccatcggaaaagccatggctgatcgcttgcagggatggggcgcgaccctgcagtaccactcgcggacagctctggatacacaaaccgagcaacggctcggcctgcgccaggtggcgtgcagcgaactcttcgccagctcggacttcatcctgctggcgcttcccttggcggcccagaccctgcatctggtcaacgccgagctgcttgccctcgtacggccgggcgctctgcttgtaaacccctgtcgtggctctgtagtggatgaagccgccgtgctcgcggcgcttgagcgaggccagctcggcgggtatgcggcggatgtattcgaaatggaagactgggctcgcgcggaccggccgcagcagatcgatcctgcgctgctcgcgcatccgaatacgctgttcactccgcaccttgggtcggcagtgcgcgcggtgcgcctggagattgaacgttgtgcagcgcagaacatcctccaggcattgcgaggtgagcgcccacaagacgctgtgaaccgtctgcccaaggccgagcctgccgcatgttga

SEQ ID NO: 220 - protein, 336 aa, synthetic construct
  REGION 1..336: note = PDH-101 variant
MLPKLVITHRVHEEILQLLAPHCELITNQTDSTLTREEILRRCADAQAMMAFMPDRVDADFLQACPELRVIGCALKGFDNFDVDACTARGVWLTFVPDLLTVPTAELAIGLAVGLGRHFRAADAFVRSGKFQGWQPIFYGTGLDGATVGFLGMGAIGKAMADRLQGWGATLQYHSRTALDTQTEQRLGLRQVACSELFASSDFILLALPLAAQTLHLVNAELLALVRPGALLVNPCRGSVVDEAAVLAALERGQLGGYAADVFEMEDWARADRPQQIDPALLAHPNTLFTPHLGSAVRAVRLEIERCAAQNILQALRGERPQDAVNRLPKAEPAAC

SEQ ID NO: 221 - DNA, 1011 bp, synthetic construct
  misc_feature 1..1011: note = PDH-101 variant
  source 1..1011: mol_type = other DNA
atgctgccgaaactcgttataactcacagagtacacgaagagatcctgcaactgctggcgccacattgcgagctgataaccaaccagaccgacagcacgctgacgcgcgaggaaattctgcggcgctgtgcagatgctcaggcgatgatggcgttcatgcccgatcgggtcgatgcagactttcttcaagcctgccctgagctgcgtgtaatcggctgtgcgctcaagggcttcgacaatttcgatgtggacgcctgtactgcacgcggggtctggctgacctttgtgcctgatctgttgacggtcccgactgccgagctggcgatcggactggcggtggggctggggcggcatttccgggcagcagatgcattcgtccgctctggcaagttccagggctggcagccaattttctacggcacggggctggatggagcaacggtcggcttccttggcatgggcgccatcggaaaagccatggctgatcgcttgcagggatggggcgcgaccctgcagtaccactcgcggacccctctggatacacaaacagagcaacggctcggcctgcgccaggtggcgcttagcgaactcttcgccagctcggacttcatcctgctggcgcttcccttggcggcccagaccctgcatctggtcaacgccgagctgcttgccctcgtacggccgggcgctctgcttgtaaacccctgtcgtggctctgtagtggatgaagccgccgtgctcgcggcgcttgagcgaggccagctcggcgggtatgcggcggatgtattcgaaatggaagactgggctcgcgcggaccggccgcagcagatcgatcctgcgctgctcgcgcatccgaatacgctgttcactccgcaccttgggtcggcagtgcgcgcggtgcgcctggagattgaacgttgtgcagcgcagaacatcctccaggcattgcgaggtgagcgcccacaagacgctgtgaaccgtctgcccaaggccgagcccgccgcatgttga

SEQ ID NO: 222 - protein, 336 aa, synthetic construct
  REGION 1..336: note = PDH-101 variant
MLPKLVITHRVHEEILQLLAPHCELITNQTDSTLTREEILRRCADAQAMMAFMPDRVDADFLQACPELRVIGCALKGFDNFDVDACTARGVWLTFVPDLLTVPTAELAIGLAVGLGRHFRAADAFVRSGKFQGWQPIFYGTGLDGATVGFLGMGAIGKAMADRLQGWGATLQYHSRTPLDTQTEQRLGLRQVALSELFASSDFILLALPLAAQTLHLVNAELLALVRPGALLVNPCRGSVVDEAAVLAALERGQLGGYAADVFEMEDWARADRPQQIDPALLAHPNTLFTPHLGSAVRAVRLEIERCAAQNILQALRGERPQDAVNRLPKAEPAAC

SEQ ID NO: 223 - DNA, 1011 bp, synthetic construct
  misc_feature 1..1011: note = PDH-101 variant
  source 1..1011: mol_type = other DNA
atgctgccgaaactcgttataactcacagagtacacgaagagatcctgcaactgctggcgccacattgcgagctgataaccaaccagaccgacagcacgctgacgcgcgaggaaattctgcggcgctgtgcagatgctcaggcgatgatggcgttcatgcccgatcgggtcgatgcagactttcttcaagcctgccctgagctgcgtgtaatcggctgtgcgctcaagggcttcgacaatttcgatgtggacgcctgtactgcacgcggggtctggctgaccattgtgcctgatctgttgacggtcccgactgccgagctggcgatcggactggcggtggggctggggcggcatttccgggcagcagatgcattcgtccgctctggcaagttccagggctggcagccaattttctacggcacggggctggatggagcaacggtcggcttccttggcatgggcgccatcggaaaagccatggctgatcgcttgcagggatggggcgcgaccctgcagtaccactcgcggacagctctggatacacaaaccgagcaacggctcggcctgcgccaggtggcgtgcagcgaactcttcgccagctcggacttcatcctgctggcgcttcccttggcggcccagaccctgcatctggtcaacgccgagctgcttgccctcgtacggccgggcgctctgcttgtaaacccctgtcgtggctctgtagtggatgaagccgccgtgctcgcggcgcttgagcgaggccagctcggcgggtatgcggcggatgtattcgaaatggaagactgggctcgcgcggaccggccgcagcagatcgatcctgcgctgctcgcgcatccgaatacgctgttcactccgcaccttgggtcggcagtgcgcgcggtgcgcctggagattgaacgttgtgcagcgcagaacatcctccaggcattgcgaggtgagcgcccaatggacgctgtgaaccgtctgcccaaggccgagcctgccgcatgttga

SEQ ID NO: 224 - protein, 336 aa, synthetic construct
  REGION 1..336: note = PDH-101 variant
MLPKLVITHRVHEEILQLLAPHCELITNQTDSTLTREEILRRCADAQAMMAFMPDRVDADFLQACPELRVIGCALKGFDNFDVDACTARGVWLTIVPDLLTVPTAELAIGLAVGLGRHFRAADAFVRSGKFQGWQPIFYGTGLDGATVGFLGMGAIGKAMADRLQGWGATLQYHSRTALDTQTEQRLGLRQVACSELFASSDFILLALPLAAQTLHLVNAELLALVRPGALLVNPCRGSVVDEAAVLAALERGQLGGYAADVFEMEDWARADRPQQIDPALLAHPNTLFTPHLGSAVRAVRLEIERCAAQNILQALRGERPMDAVNRLPKAEPAAC

SEQ ID NO: 225 - DNA, 1011 bp, synthetic construct
  misc_feature 1..1011: note = PDH-101 variant
  source 1..1011: mol_type = other DNA
atgctgccgaaactcgttataactcacagagtacacgaagagatcctgcaactgctggcgccacattgcgagctgataaccaaccagaccgacgtcacgctgacgcgcgaggaaattctgcggcgctgtgcagatgctcaggcgatgatggcgttcatgcccgatcgggtcgatatggactttcttcaagcctgccctgagctgagagtaatcggctgtgcgctcaagggcttcgacaatttcgatgtggacgcctgtactgcacgcggggtctggctgaccttcgtgcctgatctgttgacggtcccgactgccgagctggcgatcggactggcggtggggctggggcgacatttccgggcagcagatgaattcgtccgctctggcaagttccagggctggcagccaattttctacggcacggggctggatggagcaacggtcggcttccttggcatgggcgccatcggaaaagccatggctgatcgcttgcagggatggggcgcgaccctgcagtaccactcgcggagcgctctggatacacaaaccgagcaacggctcggcctgcgccatgtggcgtgcagcgaactcttcgccagctcggacttcatcctgctggcgcttcccttgaatgccgataccctgcatctggtcaacgccgagctgcttgccctcgtacggccgggcgctctgcttgtaaacccctgtcgtggctctgtagtggatgaagccgccgtgctcgcggcgcttgagcgaggccagctcggcgggtatgcggcggatgtattcgaaatggaagactgggctcgcgcggaccggccgcagcagatcgatcctgcgctgctcgcgcatccgaatacgctgttcactccgcaccttgggtcggcagtgcgcgcggtgcgcctggagattgaacgttgtgcagcgcagaacatcctccaggcattgcgaggtgagcgcccaattgacgctgtgaacgatctgcccaaggccgagcctgccgcatgttga

SEQ ID NO: 226 - protein, 336 aa, synthetic construct
  REGION 1..336: note = PDH-101 variant
MLPKLVITHRVHEEILQLLAPHCELITNQTDVTLTREEILRRCADAQAMMAFMPDRVDMDFLQACPELRVIGCALKGFDNFDVDACTARGVWLTFVPDLLTVPTAELAIGLAVGLGRHFRAADEFVRSGKFQGWQPIFYGTGLDGATVGFLGMGAIGKAMADRLQGWGATLQYHSRSALDTQTEQRLGLRHVACSELFASSDFILLALPLNADTLHLVNAELLALVRPGALLVNPCRGSVVDEAAVLAALERGQLGGYAADVFEMEDWARADRPQQIDPALLAHPNTLFTPHLGSAVRAVRLEIERCAAQNILQALRGERPIDAVNDLPKAEPAAC

SEQ ID NO: 227 - DNA, 1011 bp, synthetic construct
  misc_feature 1..1011: note = PDH-101 variant
  source 1..1011: mol_type = other DNA
atgctgccgaaactcgttataactcacagagtacacgaagagatcctgcaactgctggcgccacattgcgagctgataaccaaccagaccgacagcacgctgacgcgcgaggaaattctgcggcgctgtgcagatgctcaggcgatgatggcgttcatgcccgatcgggtcgatgcagactttcttcaagcctgccctgagctgcgtgtaatcggctgtgcgctcaagggcttcgacaatttcgatgtggacgcctgtactgcacgcggggtctggctgaccttcgtgcctgatctgttgacggtcccgactgccgagctggcgatcggactggcggtggggctggggcggcatttccgggcagcagatgcattcgtccgctctggcaagttccagggctggcagccaattttctacggcacggggctggatggagcaacggtcggcttccttggcatgggcgccatcggaaaagccatggctgatcgcttgcagggatggggcgcgaccctgcagtaccactcgcggacagctctggatacacaaaccgagcaacggctcggcctgcgccaggtggcgtgcagcgaactcttcgccagctcggacttcatcctgctggcgcttcccttgaatgccgataccccgcatctggtcaacgccgagctgcttgccctcgtacggccgggcgctctgcttgtaaacccctgtcgtggctctgtagtggatgaagccgccgtgctcgcggcgcttgagcgaggccagctcggcgggtatgcggcggatgtattcgaaatggaagactgggctcgcgcggaccggccgcagcagatcgatcctgcgctgctcgcgcatccgaatacgctgttcactccgcaccttgggtcggcagtgcgcgcggtgcgcctggagattgaacgttgtgcagcgcagaacatcctccaggcattgcgaggtgagcgcccaattgacgctgtgaaccgtctgcccaaggccgagcctgccgcatgttga

SEQ ID NO: 228 - protein, 336 aa, synthetic construct
  REGION 1..336: note = PDH-101 variant
MLPKLVITHRVHEEILQLLAPHCELITNQTDSTLTREEILRRCADAQAMMAFMPDRVDADFLQACPELRVIGCALKGFDNFDVDACTARGVWLTFVPDLLTVPTAELAIGLAVGLGRHFRAADAFVRSGKFQGWQPIFYGTGLDGATVGFLGMGAIGKAMADRLQGWGATLQYHSRTALDTQTEQRLGLRQVACSELFASSDFILLALPLNADTPHLVNAELLALVRPGALLVNPCRGSVVDEAAVLAALERGQLGGYAADVFEMEDWARADRPQQIDPALLAHPNTLFTPHLGSAVRAVRLEIERCAAQNILQALRGERPIDAVNRLPKAEPAAC

SEQ ID NO: 229 - DNA, 1011 bp, synthetic construct
  misc_feature 1..1011: note = PDH-101 variant
  source 1..1011: mol_type = other DNA
atgctgccgaaactcgttataactcacagagtacacgaagagatcctgcaactgctggcgccacattgcgagctgataaccaaccagaccgacagcacgctgacgcgcgaggaaattctgcggcgctgtgcagatgctcaggcgatgatggcgttcatgcccgatcgggtcgatgcagactttcttcaagcctgccctgagctgcgtgtaatcggctgtgcgctcaagggcttcgacaatttcgatgtggacgcctgtactgcacgcggggtctggctgaccttcgtgcctgatctgttgacggtcccgactgccgagctggcgatcggactggcggtggggctggggcggcatttccgggcagcagatgcattcgtccgctctggcaagttccagggctggcagccaattttctacggcacggggctggatggagcaacggtcggcttccttggcatgggcgccatcggaaaagccatggctgatcgcttgcagggatggggcgcgaccctgcagtaccactcgcggacagctctggatacacaaaccgagcaacggctcggcctgcgccaggtggcgtgcagcgaactcttcgccagctcggacttcatcctgaatgcgcttcccttgaatgccgataccctgcatctggtcaacgccgagctgcttgccctcgtacggccgggcgctctgcttgtaaacccctgtcgtggctctgtagtggatgaagccgccgtgctcgcggcgcttgagcgaggccagctcggcgggtatgcggcggatgtattcgaaatggaagactgggctcgcgcggaccggccgcagcagatcgatcctgcgctgctcgcgcatccgaatacgctgttcactccgcaccttgggtcggcagtgcgcgcggtgcgcctggagattgaacgttgtgcagcgcagaacatcctccaggcattgcgaggtgagcgcccaattgacgctgtgaaccgtctgcccaaggccgagcctgccgcatgttga

SEQ ID NO: 230 - protein, 336 aa, synthetic construct
  REGION 1..336: note = PDH-101 variant
MLPKLVITHRVHEEILQLLAPHCELITNQTDSTLTREEILRRCADAQAMMAFMPDRVDADFLQACPELRVIGCALKGFDNFDVDACTARGVWLTFVPDLLTVPTAELAIGLAVGLGRHFRAADAFVRSGKFQGWQPIFYGTGLDGATVGFLGMGAIGKAMADRLQGWGATLQYHSRTALDTQTEQRLGLRQVACSELFASSDFILNALPLNADTLHLVNAELLALVRPGALLVNPCRGSVVDEAAVLAALERGQLGGYAADVFEMEDWARADRPQQIDPALLAHPNTLFTPHLGSAVRAVRLEIERCAAQNILQALRGERPIDAVNRLPKAEPAAC

SEQ ID NO: 231 - DNA, 1011 bp, synthetic construct
  misc_feature 1..1011: note = PDH-101 variant
  source 1..1011: mol_type = other DNA
atgctgccgaaactcgttataactcacagagtacacgaagagatcctgcaactgctggcgccacattgcgagctgataaccaaccagaccgacagcacgctgacgcgcgaggaaattctgcggcgctgtgcagatgctcaggcgatgatggcgttcatgcccgatcgggtcgatgcagactttcttcaagcctgccctgagctgcgtgtaatcggctgtgcgctcaagggcttcgacaatttcgatgtggacgcctgtactgcacgcggggtctggctgaccttcgtgcctgatctgttgacggtcccgtttgccgagctggcgatcggactggcggtggggctggggcggcatttccgggcagcagatgcattcgtccgctctggcaagttccagggctggcagccaattttctacggcacggggctggatggagcaacggtcggcttccttggcatgggcgccatcggaaaagccatggctgatcgcttgcagggatggggcgcgaccctgcagtaccactcgcggacagctctggatacacaaaccgagcaacggctcggcctgcgccaggtggcgtgcagcgaactcttcgccagctcggacttcatcctgctggcgcttcccttgaatgccgataccctgcatctggtcaacgccgagctgcttgccctcgtacggccgggcgctctgcttgtaaacccctgtcgtggctctgtagtggatgaagccgccgtgctcgcggcgcttgagcgaggccagctcggcgggtatgcggcggatgtattcgaaatggaagactgggctcgcgcggaccggccgcagcagatcgatcctgcgctgctcgcgcatccgaatacgctgttcactccgcaccttgggtcggcagtgcgcgcggtgcgcctggagattgaacgttgtgcagcgcagaacatcctccaggcattgcgaggtgagcgcccaattgacgctgtgaaccgtctgcccaaggccgagcctgccgcatgttga

SEQ ID NO: 232 - protein, 336 aa, synthetic construct
  REGION 1..336: note = PDH-101 variant
MLPKLVITHRVHEEILQLLAPHCELITNQTDSTLTREEILRRCADAQAMMAFMPDRVDADFLQACPELRVIGCALKGFDNFDVDACTARGVWLTFVPDLLTVPFAELAIGLAVGLGRHFRAADAFVRSGKFQGWQPIFYGTGLDGATVGFLGMGAIGKAMADRLQGWGATLQYHSRTALDTQTEQRLGLRQVACSELFASSDFILLALPLNADTLHLVNAELLALVRPGALLVNPCRGSVVDEAAVLAALERGQLGGYAADVFEMEDWARADRPQQIDPALLAHPNTLFTPHLGSAVRAVRLEIERCAAQNILQALRGERPIDAVNRLPKAEPAAC

SEQ ID NO: 233 - DNA, 1011 bp, synthetic construct
  misc_feature 1..1011: note = PDH-101 variant
  source 1..1011: mol_type = other DNA
atgctgccgaaactcgttataactcacagagtacacgaagagatcctgcaactgctggcgccacattgcgagctgataaccaaccagaccgacagcacgctgacgcgcgaggaaattctgcggcgctgtgcagatgctcaggcgatgatggcgttcatgcccgatcgggtcgatgcagactttcttcaagcctgccctgagctgcgtgtaatcggctgtgcgctcaagggcttcgacaatttcgatgtggacgcctgtactgcacgcggggtctggctgaccttcgtgcctgatctgttgacggtcccgcttgccgagctggcgatcggactggcggtggggctggggcggcatttccgggcagcagatgcattcgtccgctctggcaagttccagggctggcagccaattttctacggcacggggctggatggagcaacggtcggcttccttggcatgggcgccatcggaaaagccatggctgatcgcttgcagggatggggcgcgaccctgcagtaccactcgcggacagctctggatacacaaaccgagcaacggctcggcctgcgccaggtggcgtgcagcgaactcttcgccagctcggacttcatcctgctggcgcttcccttgaatgccgataccctgcatctggtcaacgccgagctgcttgccctcgtacggccgggcgctctgcttgtaaacccctgtcgtggctctgtagtggatgaagccgccgtgctcgcggcgcttgagcgaggccagctcggcgggtatgcggcggatgtattcgaaatggaagactgggctcgcgcggaccggccgcagcagatcgatcctgcgctgctcgcgcatccgaatacgctgttcactccgcaccttgggtcggcagtgcgcgcggtgcgcctggagattgaacgttgtgcagcgcagaacatcctccaggcattgcgaggtgagcgcccaattgacgctgtgaaccgtctgcccaaggccgagcctgccgcatgttga

SEQ ID NO: 234 - protein, 336 aa, synthetic construct
  REGION 1..336: note = PDH-101 variant
MLPKLVITHRVHEEILQLLAPHCELITNQTDSTLTREEILRRCADAQAMMAFMPDRVDADFLQACPELRVIGCALKGFDNFDVDACTARGVWLTFVPDLLTVPLAELAIGLAVGLGRHFRAADAFVRSGKFQGWQPIFYGTGLDGATVGFLGMGAIGKAMADRLQGWGATLQYHSRTALDTQTEQRLGLRQVACSELFASSDFILLALPLNADTLHLVNAELLALVRPGALLVNPCRGSVVDEAAVLAALERGQLGGYAADVFEMEDWARADRPQQIDPALLAHPNTLFTPHLGSAVRAVRLEIERCAAQNILQALRGERPIDAVNRLPKAEPAAC

SEQ ID NO: 235 - DNA, 1011 bp, synthetic construct
  misc_feature 1..1011: note = PDH-101 variant
  source 1..1011: mol_type = other DNA
atgctgccgaaactcgttataactcacagagtacacgaagagatcctgcaactgctggcgccacattgcgagctgataaccaaccagaccgacagcacgctgacgcgcgaggaaattctgcggcgctgtgcagatgctcaggcgatgatggcgttcatgcccgatcgggtcgatgcagactttcttcaagcctgccctgagctgcgtgtaatcggctgtgcgctcaagggcttcgacaatttcgatgtggacgcctgtactgcacgcggggtctggctgaccttcgtgcctgatctgttgacggtcccgactgccgagctggcgatcggactggcggtggggctggggcggcatttccgggcagcagatgcattcgtccgctctggcaagttccagggctggcagccaattttctacggcacggggctggatggagcaacggtcggcttccttggcatgggcgccatcggaaaagccatggctgatcgcttgcagggatggggcgcgaccctgcagtaccactcgcggacagctctggatacacaaaccgagcaacggctcggcctgcgccaggtggcgtgcagcgaactcttcgccagctcggacttcatcctgctggcgcttcccttgaatgccgataccctgcatctggtcaacgccgagctgcttgccctcgtacggccgggcgctctgcttgtaaacccctgtcgtggctctgtagtggatgaagccgccgtgctcgcggcgcttgagcgaggccagctcggcgggtatgcggcggatgtattcgaaatgtctgactgggctcgcgcggaccggccgcagcagatcgatcctgcgctgctcgcgcatccgaatacgctgttcactccgcaccttgggtcggcagtgcgcgcggtgcgcctggagattgaacgttgtgcagcgcagaacatcctccaggcattgcgaggtgagcgcccaattgacgctgtgaaccgtctgcccaaggccgagcctgccgcatgttga

SEQ ID NO: 236 - protein, 336 aa, synthetic construct
  REGION 1..336: note = PDH-101 variant
MLPKLVITHRVHEEILQLLAPHCELITNQTDSTLTREEILRRCADAQAMMAFMPDRVDADFLQACPELRVIGCALKGFDNFDVDACTARGVWLTFVPDLLTVPTAELAIGLAVGLGRHFRAADAFVRSGKFQGWQPIFYGTGLDGATVGFLGMGAIGKAMADRLQGWGATLQYHSRTALDTQTEQRLGLRQVACSELFASSDFILLALPLNADTLHLVNAELLALVRPGALLVNPCRGSVVDEAAVLAALERGQLGGYAADVFEMSDWARADRPQQIDPALLAHPNTLFTPHLGSAVRAVRLEIERCAAQNILQALRGERPIDAVNRLPKAEPAAC

SEQ ID NO: 237 - DNA, 1011 bp, synthetic construct
  misc_feature 1..1011: note = PDH-101 variant
  source 1..1011: mol_type = other DNA
atgctgccgaaactcgttataactcacagagtacacgaagagatcctgcaactgctggcgccacattgcgagctgataaccaaccagaccgacagcacgctgacgcgcgaggaaattctgcggcgctgtgcagatgctcaggcgatgatggcgttcatgcccgatcgggtcgatgcagactttcttcaagcctgccctgagctgcgtgtaatcggctgtgcgctcaagggcttcgacaatttcgatgtggacgcctgtactgcacgcggggtctggctgaccttcgtgcctgatctgttgacggtcccgactgccgagctggcgatcggactggcggtggggctggggcggcatttccgggcagcagatgcattcgtccgctctggcaagttccagggctggcagccaattttctacggcacggggctggatggagcaacggtcggcttccttggcatgggcgccatcggaaaagccatggctgatcgcttgcagggatggggcgcgaccctgcagtaccactcgcggacagctctggatacacaaaccgagcaacggctcggcctgcgccaggtggcgtgcagcgaactcttcgccagctcggacttcatcctgctggcgcttcccttgaatgccgataccctgcatctggtcaacgccgagctgcttgccctcgtacggccgggcgctctgcttgtaaacccctgtcgtggctctgtagtggatgaagccgccgtgctcgcggcgcttgagcgaggccagctcggcgggtatgcggcggatcctttcgaaatggaagactgggctcgcgcggaccggccgcagcagatcgatcctgcgctgctcgcgcatccgaatacgctgttcactccgcaccttgggtcggcagtgcgcgcggtgcgcctggagattgaacgttgtgcagcgcagaacatcctccaggcattgcgaggtgagcgcccaattgacgctgtgaaccgtctgcccaaggccgagcctgccgcatgttga

SEQ ID NO: 238 - protein, 336 aa, synthetic construct
  REGION 1..336: note = PDH-101 variant
MLPKLVITHRVHEEILQLLAPHCELITNQTDSTLTREEILRRCADAQAMMAFMPDRVDADFLQACPELRVIGCALKGFDNFDVDACTARGVWLTFVPDLLTVPTAELAIGLAVGLGRHFRAADAFVRSGKFQGWQPIFYGTGLDGATVGFLGMGAIGKAMADRLQGWGATLQYHSRTALDTQTEQRLGLRQVACSELFASSDFILLALPLNADTLHLVNAELLALVRPGALLVNPCRGSVVDEAAVLAALERGQLGGYAADPFEMEDWARADRPQQIDPALLAHPNTLFTPHLGSAVRAVRLEIERCAAQNILQALRGERPIDAVNRLPKAEPAAC

SEQ ID NO: 239 - DNA, 1011 bp, synthetic construct
  misc_feature 1..1011: note = PDH-101 variant
  source 1..1011: mol_type = other DNA
atgctgccgaaactcgttataactcacagagtacacgaagagatcctgcaactgctggcgccacattgcgagctgataaccaaccagaccgacagcacgctgacgcgcgaggaaattctgcggcgctgtgcagatgctcaggcgatgatggcgttcatgcccgatcgggtcgatgcagactttcttcaagcctgccctgagctgcgtgtaatcggctgtgcgctcaagggcttcgacaatttcgatgtggacgcctgtactgcacgcggggtctggctgaccttcgtgcctgatctgttgacggtcccgactgccgagctggcgatcggactggcggtggggctggggcggcatttccgggcagcagatgcattcgtccgctctggcaagttccagggctggcagccaattttctacggcacggggctggatggagcaacggtcggcttccttggcatgggcgccatcggaaaagccatggctgatcgcttgcagggatggggcgcgaccctgcagtaccactcgcggacagctctggatacacaaaccgagcaacggctcggcctgcgccaggtggcgtgcagcgaactcttcgccagctcggacttcatcctgctggcgcttcccttgaatgccgataccctgcatctggtcaacgccgagctgcttgccctcgtacggccgggcgctctgcttgtaaacccctgtcgtggctctgtagtggatgaagccgccgtgctcgcggcgcttgagcgaggccagctcggcgggtatgcggcggatgatttcgaaatggaagactgggctcgcgcggaccggccgcagcagatcgatcctgcgctgctcgcgcatccgaatacgctgttcactccgcaccttgggtcggcagtgcgcgcggtgcgcctggagattgaacgttgtgcagcgcagaacatcctccaggcattgcgaggtgagcgcccaattgacgctgtgaaccgtctgcccaaggccgagcctgccgcatgttga

SEQ ID NO: 240 - protein, 336 aa, synthetic construct
  REGION 1..336: note = PDH-101 variant
MLPKLVITHRVHEEILQLLAPHCELITNQTDSTLTREEILRRCADAQAMMAFMPDRVDADFLQACPELRVIGCALKGFDNFDVDACTARGVWLTFVPDLLTVPTAELAIGLAVGLGRHFRAADAFVRSGKFQGWQPIFYGTGLDGATVGFLGMGAIGKAMADRLQGWGATLQYHSRTALDTQTEQRLGLRQVACSELFASSDFILLALPLNADTLHLVNAELLALVRPGALLVNPCRGSVVDEAAVLAALERGQLGGYAADDFEMEDWARADRPQQIDPALLAHPNTLFTPHLGSAVRAVRLEIERCAAQNILQALRGERPIDAVNRLPKAEPAAC

SEQ ID NO: 241 - DNA, 1011 bp, synthetic construct
  misc_feature 1..1011: note = PDH-101 variant
  source 1..1011: mol_type = other DNA
atgctgccgaaactcgttataactcacagagtacacgaagagatcctgcaactgctggcgccacattgcgagctgataaccaaccagaccgacagcacgctgacgcgcgaggaaattctgcggcgctgtgcagatgctcaggcgatgatggcgttcatgcccgatcgggtcgatgcagactttcttcaagcctgccctgagctgcgtgtaatcggctgtgcgctcaagggcttcgacaatttcgatgcggacgcctgtactgcacgcggggtctggctgaccttcgtgcctgatctgttgacggtcccgactgccgagctggcgatcggactggcggtggggctggggcggcatttccgggcagcagatgcattcgtccgctctggcaagttccagggctggcagccaattttctacggcacggggctggatggagcaacggtcggcttccttggcatgggcgccatcggaaaagccatggctgatcgcttgcagggatggggcgcgaccctgcagtaccactcgcggacagctctggatacacaaaccgagcaacggctcggcctgcgccaggtggcgtgcagcgaactcttcgccagctcggacttcatcctgctggcgcttcccttgaatgccgataccctgcatctggtcaacgccgagctgcttgccctcgtacggccgggcgctctgcttgtaaacccctgtcgtggctctgtagtggatgaagccgccgtgctcgcggcgcttgagcgaggccagctcggcgggtatgcggcggatgtattcgaaatggcggactgggctcgcgcggaccggccgcagcagatcgatcctgcgctgctcgcgcatccgaatacgctgttcactccgcaccttgggtcggcagtgcgcgcggtgcgcctggagattgaacgttgtgcagcgcagaacatcctccaggcattgcgaggtgagcgcccaattgacgctgtgaaccgtctgcccaaggccgagcctgccgcatgttga

SEQ ID NO: 242 - protein, 336 aa, synthetic construct
  REGION 1..336: note = PDH-101 variant
MLPKLVITHRVHEEILQLLAPHCELITNQTDSTLTREEILRRCADAQAMMAFMPDRVDADFLQACPELRVIGCALKGFDNFDADACTARGVWLTFVPDLLTVPTAELAIGLAVGLGRHFRAADAFVRSGKFQGWQPIFYGTGLDGATVGFLGMGAIGKAMADRLQGWGATLQYHSRTALDTQTEQRLGLRQVACSELFASSDFILLALPLNADTLHLVNAELLALVRPGALLVNPCRGSVVDEAAVLAALERGQLGGYAADVFEMADWARADRPQQIDPALLAHPNTLFTPHLGSAVRAVRLEIERCAAQNILQALRGERPIDAVNRLPKAEPAAC

SEQ ID NO: 243 - DNA, 1011 bp, synthetic construct
  misc_feature 1..1011: note = PDH-101 variant
  source 1..1011: mol_type = other DNA
atgctgccgaaactcgttataactcacagagtacacgaagagatcctgcaactgctggcgccacattgcgagctgataaccaaccagaccgacagcacgctgacgcgcgaggaaattctgcggcgctgtgcagatgctcaggcgatgatggcgttcatgcccgatcgggtcgatgcagactttcttcaagcctgccctgagctgcgtgtaatcggctgtgcgctcaagggcttcgacaatttcgatgtggacgcctgtactgcacgcggggtctggctgaccttcgtgcctgatctgttgacggtcccgactgccgagctggcgatcggactggcggtggggctggggcggcatttccgggcagcagatgcattcgtccgctctggcaagttccagggctggcagccaattttctacggcacggggctggatggagcaacggtcggcttccttggcatgggcgccatcggaaaagccatggctgatcgcttgcagggatggggcgcgaccctgcagtaccactcgcggacagctctggatacacaaaccgagcaacggctcggcctgcgccaggtggcgtgcagcgaactcttcgccagctcggacttcatcctgctggcgcttcccttgaatgccgataccctgcatctggtcaacgccgagctgcttgccctcgtacggccgggcgctctgcttgtaaacccctgtcgtggctctgtagtggatgaagccgccgtgctcgcggcgcttgagcgaggccagctcggcgggtatgcggcggatgtattcgaaatggaagactgggctcgcgcggaccggccgcagcagatcgatcctgcgctgctcgcgcatccgaatacgctgttcactccgcaccttgggtcggcagtgcgcgcggtgcgcctggagattgaacgttgtgcagcgcagaacatcctccaggcattgcgaggtgagcgcccaattaatgcggtgaaccgtctgcccaaggccgagcctgccgcatgttga

SEQ ID NO: 244 - protein, 336 aa, synthetic construct
  REGION 1..336: note = PDH-101 variant
MLPKLVITHRVHEEILQLLAPHCELITNQTDSTLTREEILRRCADAQAMMAFMPDRVDADFLQACPELRVIGCALKGFDNFDVDACTARGVWLTFVPDLLTVPTAELAIGLAVGLGRHFRAADAFVRSGKFQGWQPIFYGTGLDGATVGFLGMGAIGKAMADRLQGWGATLQYHSRTALDTQTEQRLGLRQVACSELFASSDFILLALPLNADTLHLVNAELLALVRPGALLVNPCRGSVVDEAAVLAALERGQLGGYAADVFEMEDWARADRPQQIDPALLAHPNTLFTPHLGSAVRAVRLEIERCAAQNILQALRGERPINAVNRLPKAEPAAC

SEQ ID NO: 245 - DNA, 1008 bp, synthetic construct
  misc_feature 1..1008: note = PDH-101 variant
  source 1..1008: mol_type = other DNA
atgctgccgaaactcgttataactcacagagtacacgaagagatcctgcaactgctggcgccacattgcgagctgataaccaaccagaccgacagcacgctgacgcgcgaggaaattctgcggcgctgtgcagatgctcaggcgatgatggcgttcatgcccgatcgggtcgatgcagactttcttcaagcctgccctgagctgcgtgtaatcggctgtgcgctcaagggcttcgacaatttcgatgcggacgcctgtactgcacgcggggtctggctgaccttcgtgcctgatctgttgacggtcccgttggccgagctggcgatcggactggcggtggggctggggcggcatttccgggcagcagatgcattcgtccgctctggcaagttccagggctggcagccaattttctacggcacggggctggatggagcaacggtcggcttccttggcatgggcgccatcggaaaagccatggctgatcgcttgcagggatggggcgcgaccctgcagtaccactcgcggacagctctggatacacaaaccgagcaacggctcggcctgcgccaggtggcgtgcagcgaactcttcgccagctcggacttcatcctgaatgcgcttcccttgaatgccgataccctgcatctggtcaacgccgagctgcttgccctcgtacggccgggcgctctgcttgtaaacccctgtcgtggctctgtagtggatgaagccgccgtgctcgcggcgcttgagcgaggccagctcggcgggtatgcggcggatgtgttcgaaatggaggactgggctcgcgcggaccggccgcagcagatcgatcctgcgctgctcgcgcatccgaatacgctgttcactccgcaccttgggtcggcagtgcgcgcggtgcgcctggagattgaacgttgtgcagcgcagaacatcctccaggcattgcgaggtgagcgcccaattgacgctgtgaaccgtctgcccaaggccgagcctgccgcatgt

SEQ ID NO: 246 - protein, 336 aa, synthetic construct
  REGION 1..336: note = PDH-101 variant
MLPKLVITHRVHEEILQLLAPHCELITNQTDSTLTREEILRRCADAQAMMAFMPDRVDADFLQACPELRVIGCALKGFDNFDADACTARGVWLTFVPDLLTVPLAELAIGLAVGLGRHFRAADAFVRSGKFQGWQPIFYGTGLDGATVGFLGMGAIGKAMADRLQGWGATLQYHSRTALDTQTEQRLGLRQVACSELFASSDFILNALPLNADTLHLVNAELLALVRPGALLVNPCRGSVVDEAAVLAALERGQLGGYAADVFEMEDWARADRPQQIDPALLAHPNTLFTPHLGSAVRAVRLEIERCAAQNILQALRGERPIDAVNRLPKAEPAAC

SEQ ID NO: 247 - DNA, 1008 bp, synthetic construct
  misc_feature 1..1008: note = PDH-101 variant
  source 1..1008: mol_type = other DNA
atgctgccgaaactcgttataactcacagagtacacgaagagatcctgcaactgctggcgccacattgcgagctgataaccaaccagaccgacagcacgctgacgcgcgaggaaattctgcggcgctgtgcagatgctcaggcgatgatggcgttcatgcccgatcgggtcgatgcagactttcttcaagcctgccctgagctgcgtgtaatcggctgtaccctcaagggcttcgacaatttcgatgcggacgcctgtactgcacgcggggtctggctgaccttcgtgcctgatctgttgacggtcccgactgccgagctggcgatcggactggcggtggggctggggcggcatttccgggcagcagatgcattcgtccgctctggcaagttccagggctggcagccaattttctacggcacggggctggatggagcaacggtcggcttccttggcatgggcgccatcggaaaagccatggctgatcgcttgcagggatggggcgcgaccctgcagtaccactcgcggacagctctggatacacaaaccgagcaacggctcggcctgcgccaggtggcgtgcagcgaactcttcgccagctcggacttcatcctgaatgcgcttcccttgaatgccgataccctgcatctggtcaacgccgagctgcttgccctcgtacggccgggcgctctgcttgtaaacccctgtcgtggctctgtagtggatgaagccgccgtgctcgcggcgcttgagcgaggccagctcggcgggtatgcggcggatgtgttcgaaatggaggactgggctcgcgcggaccggccgcagcagatcgatcctgcgctgctcgcgcatccgaatacgctgttcactccgcaccttgggtcggcagtgcgcgcggtgcgcctggagattgaacgttgtgcagcgcagaacatcctccaggcattgcgaggtgagcgcccaattgacgctgtgaaccgtctgcccaaggccgagcctgccgcatgt

SEQ ID NO: 248 - protein, 336 aa, synthetic construct
  REGION 1..336: note = PDH-101 variant
MLPKLVITHRVHEEILQLLAPHCELITNQTDSTLTREEILRRCADAQAMMAFMPDRVDADFLQACPELRVIGCTLKGFDNFDADACTARGVWLTFVPDLLTVPTAELAIGLAVGLGRHFRAADAFVRSGKFQGWQPIFYGTGLDGATVGFLGMGAIGKAMADRLQGWGATLQYHSRTALDTQTEQRLGLRQVACSELFASSDFILNALPLNADTLHLVNAELLALVRPGALLVNPCRGSVVDEAAVLAALERGQLGGYAADVFEMEDWARADRPQQIDPALLAHPNTLFTPHLGSAVRAVRLEIERCAAQNILQALRGERPIDAVNRLPKAEPAAC

SEQ ID NO: 249 - DNA, 1008 bp, synthetic construct
  misc_feature 1..1008: note = PDH-101 variant
  source 1..1008: mol_type = other DNA
atgctgccgaaactcgttataactcacagagtacacgaagagatcctgcaactgctggcgccacattgcgagctgataaccaaccagaccgacagcacgctgacgcgcgaggaaattctgcggcgctgtgcagatgctcaggcgatgatggcgttcatgcccgatcgggtcgatgcagactttcttcaagcctgccctgagctgcgtgtaatcggctgtgcgctcaagggcttcgacaatttcgatgtggacgcctgtactgcacgcggggtctggctgaccttcgtgcctgatctgttgacggtcccgttggccgagctggcgatcggactggcggtggggctggggcggcatttccgggcagcagatgcattcgtccgctctggcaagttccagggctggcagccaattttctacggcacggggctggatggagcaacggtcggcttccttggcatgggcgccatcggaaaagccatggctgatcgcttgcagggatggggcgcgaccctgcagtaccactcgcggacagctctggatacacaaaccgagcaacggctcggcctgcgccaggtggcgtgcagcgaactcttcgccagctcggacttcatcctgctggcgcttcccttgaatgccgataccctgcatctggtcaacgccgagctgcttgccctcgtacggccgggcgctctgcttgtaaacccctgtcgtggctctgtagtggatgaagccgccgtgctcgcggcgcttgagcgaggccagctcggcgggtatgcggcggatctgttcgaaatggaggactgggctcgcgcggaccggccgcagcagatcgatcctgcgctgctcgcgcatccgaatacgctgttcactccgcaccttgggtcggcagtgcgcgcggtgcgcctggagattgaacgttgtgcagcgcagaacatcctccaggcattgcgaggtgagcgcccaattgacgctgtgaaccgtctgcccaaggccgagcctgccgcatgt

SEQ ID NO: 250 - protein, 336 aa, synthetic construct
  REGION 1..336: note = PDH-101 variant
MLPKLVITHRVHEEILQLLAPHCELITNQTDSTLTREEILRRCADAQAMMAFMPDRVDADFLQACPELRVIGCALKGFDNFDVDACTARGVWLTFVPDLLTVPLAELAIGLAVGLGRHFRAADAFVRSGKFQGWQPIFYGTGLDGATVGFLGMGAIGKAMADRLQGWGATLQYHSRTALDTQTEQRLGLRQVACSELFASSDFILLALPLNADTLHLVNAELLALVRPGALLVNPCRGSVVDEAAVLAALERGQLGGYAADLFEMEDWARADRPQQIDPALLAHPNTLFTPHLGSAVRAVRLEIERCAAQNILQALRGERPIDAVNRLPKAEPAAC

SEQ ID NO: 251 - DNA, 1008 bp, synthetic construct
  misc_feature 1..1008: note = PDH-101 variant
  source 1..1008: mol_type = other DNA
atgctgccgaaactcgttataactcacagagtacacgaagagatcctgcaactgctggcgccacattgcgagctgataaccaaccagaccgacagcacgctgacgcgcgaggaaattctgcggcgctgtgcagatgctcaggcgatgatggcgttcatgcccgatcgggtcgatgcagactttcttcaagcctgccctgagctgcgtgtaatcggctgtgccctcaagggcttcgacaatttcgatgtggacgcctgtactgcacgcggggtctggctgaccttcgtgcctgatctgttgacggtcccgttggccgagctggcgatcggactggcggtggggctggggcggcatttccgggcagcagatgcattcgtccgctctggcaagttccagggctggcagccaattttctacggcacggggctggatggagcaacggtcggcttccttggcatgggcgccatcggaaaagccatggctgatcgcttgcagggatggggcgcgaccctgcagtaccactcgcggacagctctggatacacaaaccgagcaacggctcggcctgcgccaggtggcgtgcagcgaactcttcgccagctcggacttcatcctgaatgcgcttcccttgaatgccgataccctgcatctggtcaacgccgagctgcttgccctcgtacggccgggcgctctgcttgtaaacccctgtcgtggctctgtagtggatgaagccgccgtgctcgcggcgcttgagcgaggccagctcggcgggtatgcggcggatgtgttcgaaatggaggactgggctcgcgcggaccggccgcagcagatcgatcctgcgctgctcgcgcatccgaatacgctgttcactccgcaccttgggtcggcagtgcgcgcggtgcgcctggagattgaacgttgtgcagcgcagaacatcctccaggcattgcgaggtgagcgcccaattgacgctgtgaaccgtctgcccaaggccgagcctgccgcatgt

SEQ ID NO: 252 - protein, 336 aa, synthetic construct
  REGION 1..336: note = PDH-101 variant
MLPKLVITHRVHEEILQLLAPHCELITNQTDSTLTREEILRRCADAQAMMAFMPDRVDADFLQACPELRVIGCALKGFDNFDVDACTARGVWLTFVPDLLTVPLAELAIGLAVGLGRHFRAADAFVRSGKFQGWQPIFYGTGLDGATVGFLGMGAIGKAMADRLQGWGATLQYHSRTALDTQTEQRLGLRQVACSELFASSDFILNALPLNADTLHLVNAELLALVRPGALLVNPCRGSVVDEAAVLAALERGQLGGYAADVFEMEDWARADRPQQIDPALLAHPNTLFTPHLGSAVRAVRLEIERCAAQNILQALRGERPIDAVNRLPKAEPAAC

SEQ ID NO: 253 - DNA, 1008 bp, synthetic construct
  misc_feature 1..1008: note = PDH-101 variant
  source 1..1008: mol_type = other DNA
atgctgccgaaactcgttataactcacagagtacacgaagagatcctgcaactgctggcgccacattgcgagctgataaccaaccagaccgacagcacgctgacgcgcgaggaaattctgcggcgctgtgcagatgctcaggcgatgatggcgttcatgcccgatcgggtcgatgcagactttcttcaagcctgccctgagctgcgtgtaatcggctgtgcgctcaagggcttcgacaatttcgatgtggacgcctgtactgcacgcggggtctggctgaccttcgtgcctgatctgttgacggtcccgactgccgagctggcgatcggactggcggtggggctggggcggcatttccgggcagcagatgcattcgtccgctctggcaagttccagggctggcagccaattttctacggcacggggctggatggagcaacggtcggcttccttggcatgggcgccatcggaaaagccatggctgatcgcttgcagggatggggcgcgaccctgcagtaccactcgcggacagctctggatacacaaaccgagcaacggctcggcctgcgccaggtggcgtgcagcgaactcttcgccagctcggacttcatcctgctggcgcttcccttgaatgccgataccctgcatctggtcaacgccgagctgcttgccctcgtacggccgggcgctctgcttgtaaacccctgtcgtggctctgtagtggatgaagccgccgtgctcgcggcgcttgagcgaggccagctcggcgggtatgcggcggatgtattcgaaatggaagactgggctcgcgcggaccggccgcagcagatcgatcctgcgctgctcgcgcatccgaatacgctgttcactccgcaccttgggcgggcagtgcgcgcggtgcgcctggagattgaacgttgtgcagcgcagaacatcctccaggcattgcgaggtgagcgcccaattgacgctgtgaaccgtctgcccaaggccgagcctgccgcatgt

SEQ ID NO: 254 - protein, 336 aa, synthetic construct
  REGION 1..336: note = PDH-101 variant
MLPKLVITHRVHEEILQLLAPHCELITNQTDSTLTREEILRRCADAQAMMAFMPDRVDADFLQACPELRVIGCALKGFDNFDVDACTARGVWLTFVPDLLTVPTAELAIGLAVGLGRHFRAADAFVRSGKFQGWQPIFYGTGLDGATVGFLGMGAIGKAMADRLQGWGATLQYHSRTALDTQTEQRLGLRQVACSELFASSDFILLALPLNADTLHLVNAELLALVRPGALLVNPCRGSVVDEAAVLAALERGQLGGYAADVFEMEDWARADRPQQIDPALLAHPNTLFTPHLGRAVRAVRLEIERCAAQNILQALRGERPIDAVNRLPKAEPAAC

SEQ ID NO: 255 - DNA, 1008 bp, synthetic construct
  misc_feature 1..1008: note = PDH-101 variant
  source 1..1008: mol_type = other DNA
atgctgccgaaactcgttataactcacagagtacacgaagagatcctgcaactgctggcgccacattgcgagctgataaccaaccagaccgacagcacgctgacgcgcgaggaaattctgcggcgctgtgcagatgctcaggcgatgatggcgttcatgcccgatcgggtcgatgcagactttcttcaagcctgccctgagctgcgtgtaatcggctgtgcgctcaagggcttcgacaatttcgatgtggacgcctgtactgcacgcggggtctggctgaccttcgggcctgatctgttgacggtcccgactgccgagctggcgatcggactggcggtggggctggggcggcatttccgggcagcagatgcattcgtccgctctggcaagttccagggctggcagccaattttctacggcacggggctggatggagcaacggtcggcttccttggcatgggcgccatcggaaaagccatggctgatcgcttgcagggatggggcgcgaccctgcagtaccactcgcggacagctctggatacacaaaccgagcaacggctcggcctgcgccaggtggcgtgcagcgaactcttcgccagctcggacttcatcctgctggcgcttcccttgaatgccgataccctgcatctggtcaacgccgagctgcttgccctcgtacggccgggcgctctgcttgtaaacccctgtcgtggctctgtagtggatgaagccgccgtgctcgcggcgcttgagcgaggccagctcggcgggtatgcggcggatgtattcgaaatggaagactgggctcgcgcggaccggccgcagcagatcgatcctgcgctgctcgcgcatccgaatacgctgttcactccgcaccttgggtcggcagtgcgcgcggtgcgcctggagattgaacgttgtgcagcgcagaacatcctccaggcattgcgaggtgagcgcccaattgacgctgtgaaccgtctgcccaaggccgagcctgccgcatgt

SEQ ID NO: 256 - protein, 336 aa, synthetic construct
  REGION 1..336: note = PDH-101 variant
MLPKLVITHRVHEEILQLLAPHCELITNQTDSTLTREEILRRCADAQAMMAFMPDRVDADFLQACPELRVIGCALKGFDNFDVDACTARGVWLTFGPDLLTVPTAELAIGLAVGLGRHFRAADAFVRSGKFQGWQPIFYGTGLDGATVGFLGMGAIGKAMADRLQGWGATLQYHSRTALDTQTEQRLGLRQVACSELFASSDFILLALPLNADTLHLVNAELLALVRPGALLVNPCRGSVVDEAAVLAALERGQLGGYAADVFEMEDWARADRPQQIDPALLAHPNTLFTPHLGSAVRAVRLEIERCAAQNILQALRGERPIDAVNRLPKAEPAAC

SEQ ID NO: 257 - DNA, 1008 bp, synthetic construct
  misc_feature 1..1008: note = PDH-101 variant
  source 1..1008: mol_type = other DNA
atgctgccgaaactcgttataactcacagagtacacgaagagatcctgcaactgctggcgccacattgcgagctgataaccaaccagaccgacagcacgctgacgcgcgaggaaattctgcggcgctgtgcagatgctcaggcgatgatggcgttcatgcccgatcgggtcgatgcagactttcttcaagcctgccctgagctgcgtgtaatcggctgtgcgctcaagggcttcgacaatttcgatgtggacgcctgtactgcacgcggggtctggctgaccttcgtgcctgatctgttgacggtcccgatggccgagctggcgatcggactggcggtggggctggggcggcatttccgggcagcagatgcattcgtccgctctggcaagttccagggctggcagccaattttctacggcacggggctggatggagcaacggtcggcttccttggcatgggcgccatcggaaaagccatggctgatcgcttgcagggatggggcgcgaccctgcagtaccactcgcggacagctctggatacacaaaccgagcaacggctcggcctgcgccaggtggcgtgcagcgaactcttcgccagctcggacttcatcctgctggcgcttcccttgaatgccgataccctgcatctggtcaacgccgagctgcttgccctcgtacggccgggcgctctgcttgtaaacccctgtcgtggctctgtagtggatgaagccgccgtgctcgcggcgcttgagcgaggccagctcggcgggtatgcggcggatgtattcgaaatggaagactgggctcgcgcggaccggccgcagcagatcgatcctgcgctgctcgcgcatccgaatacgctgttcactccgcaccttgggtcggcagtgcgcgcggtgcgcctggagattgaacgttgtgcagcgcagaacatcctccaggcattgcgaggtgagcgcccaattgacgctgtgaaccgtctgcccaaggccgagcctgccgcatgt

SEQ ID NO: 258 - protein, 336 aa, synthetic construct
  REGION 1..336: note = PDH-101 variant
MLPKLVITHRVHEEILQLLAPHCELITNQTDSTLTREEILRRCADAQAMMAFMPDRVDADFLQACPELRVIGCALKGFDNFDVDACTARGVWLTFVPDLLTVPMAELAIGLAVGLGRHFRAADAFVRSGKFQGWQPIFYGTGLDGATVGFLGMGAIGKAMADRLQGWGATLQYHSRTALDTQTEQRLGLRQVACSELFASSDFILLALPLNADTLHLVNAELLALVRPGALLVNPCRGSVVDEAAVLAALERGQLGGYAADVFEMEDWARADRPQQIDPALLAHPNTLFTPHLGSAVRAVRLEIERCAAQNILQALRGERPIDAVNRLPKAEPAAC

SEQ ID NO: 259 - DNA, 1008 bp, synthetic construct
  misc_feature 1..1008: note = PDH-101 variant
  source 1..1008: mol_type = other DNA
atgctgccgaaactcgttataactcacagagtacacgaagagatcctgcaactgctggcgccacattgcgagctgataaccaaccagaccgacagcacgctgacgcgcgaggaaattctgcggcgctgtgcagatgctcaggcgatgatggcgttcatgcccgatcgggtcgatgcagactttcttcaagcctgccctgagctgcgtgtaatcggctgtgcgctcaagggcttcgacaatttcgatgtggacgcctgtactgcacgcggggtctggctgaccttcgtgcctgatctgttgacggtcccgactgccgagctggcgatcggactggcggtggggctggggcggcatttccgggcagcagatgcattcgtccgctctggcaagttccagggctggcagccaattttctacggcacggggctggatggagcaacggtcggcttccttggcatgggcgccatcggaaaagccatggctgatcgcttgcagggatggggcgcgaccctgcagtaccacgaagcgacagctctggatacacaaaccgagcaacggctcggcctgcgccaggtggcgtgcagcgaactcttcgccagctcggacttcatcctgctggcgcttcccttgaatgccgataccctgcatctggtcaacgccgagctgcttgccctcgtacggccgggcgctctgcttgtaaacccctgtcgtggctctgtagtggatgaagccgccgtgctcgcggcgcttgagcgaggccagctcggcgggtatgcggcggatgtattcgaaatggaagactgggctcgcgcggaccggccgcagcagatcgatcctgcgctgctcgcgcatccgaatacgctgttcactccgcaccttgggtcggcagtgcgcgcggtgcgcctggagattgaacgttgtgcagcgcagaacatcctccaggcattgcgaggtgagcgcccaattgacgctgtgaaccgtctgcccaaggccgagcctgccgcatgt

SEQ ID NO: 260 - protein, 336 aa, synthetic construct
  REGION 1..336: note = PDH-101 variant
MLPKLVITHRVHEEILQLLAPHCELITNQTDSTLTREEILRRCADAQAMMAFMPDRVDADFLQACPELRVIGCALKGFDNFDVDACTARGVWLTFVPDLLTVPTAELAIGLAVGLGRHFRAADAFVRSGKFQGWQPIFYGTGLDGATVGFLGMGAIGKAMADRLQGWGATLQYHEATALDTQTEQRLGLRQVACSELFASSDFILLALPLNADTLHLVNAELLALVRPGALLVNPCRGSVVDEAAVLAALERGQLGGYAADVFEMEDWARADRPQQIDPALLAHPNTLFTPHLGSAVRAVRLEIERCAAQNILQALRGERPIDAVNRLPKAEPAAC

(19) United States
(12) Patent Application Publication
(10) Pub. No.: US 2024/0068005 A1
(43) Pub. Date: Feb. 29, 2024

(54) KETOREDUCTASE POLYPEPTIDES AND POLYNUCLEOTIDES

(71) Applicant: Codexis, Inc., Redwood City, CA (US)

(72) Inventors: Jeffrey C. Moore, Westfield, NJ (US); Jack Liang, South San Francisco, CA (US); Jonathan Penfield, Truckee, CA (US); Jovana Nazor, Milpitas, CA (US); Nikki Dellas, San Carlos, CA (US); Vesna Mitchell, Santa Clara, CA (US); Da Duan, Newark, CA (US); Iman Farasat, Rahway, NJ (US); Agustina Rodriguez-Granillo, Rahway, NJ (US); Grant Murphy, Rahway, NJ (US); Nicholas Marshall, Rahway, NJ (US)

(21) Appl. No.: 18/352,899

(22) Filed: Jul. 14, 2023

Related U.S. Application Data
(63) Continuation of application No. 17/239,953, filed on Apr. 26, 2021, now Pat. No. 11,746,369.
Application No. 17/239,953 is a division of application No. 16/605,576, filed on Oct. 16, 2019, now Pat. No. 11,021,729.
(60) Provisional application No. 62/491,161, filed on Apr. 27, 2017.

(51) Int. Cl. C12P 41/00; C12N 9/02; C12N 9/04; C12N 15/70

(52) U.S. Cl. CPC C12P 41/002; C12N 9/0004; C12N 9/0006; C12N 15/70; C12Y 101/01002; C12Y 120/01001

(57) ABSTRACT

The present invention provides engineered ketoreductase and phosphite dehydrogenase enzymes having improved properties as compared to a naturally occurring wild-type ketoreductase and phosphite dehydrogenase enzymes, as well as polynucleotides encoding the engineered ketoreductase and phosphite dehydrogenase enzymes, host cells capable of expressing the engineered ketoreductase and phosphite dehydrogenase enzymes, and methods of using the engineered ketoreductase and phosphite dehydrogenase enzymes to synthesize a chiral catalyst used in the synthesis of antiviral compounds, such as nucleoside inhibitors. The present invention further provides methods of using the engineered enzymes to deracemize a chiral alcohol in a one-pot, multi-enzyme system.

The present application is a Continuation of co-pending of U.S. patent application Ser. No. 17/239,953, filed Apr. 26, 2021, which is a Divisional of U.S. patent application Ser. No. 16/605,576, filed Oct. 16, 2019, now U.S. Pat. No. 11,021,729, a national stage application filed under 35 USC § 371 and claims priority to international application to PCT International Application No. PCT/US2018/027450, filed Apr. 13, 2018, which claims priority to U.S. Prov. Pat. Appln. Ser. No. 62/491,161, filed Apr. 27, 2017, all of which are hereby incorporated by reference, in their entireties and for all purposes.

### REFERENCE TO SEQUENCE LISTING, TABLE OR COMPUTER PROGRAM

The Sequence Listing concurrently submitted herewith under 37 C.F.R. § 1.821 in a computer readable form (CRF) as file name CX2-166WO1UDC1 ST26.xml is herein incorporated by reference. The electronic copy of the Sequence Listing was created on Jul. 14, 2023, with a file size of 500,455 bytes.

### FIELD OF THE INVENTION

The present invention provides engineered ketoreductase and phosphite dehydrogenase enzymes having improved properties as compared to a naturally occurring wild-type ketoreductase and phosphite dehydrogenase enzymes, as well as polynucleotides encoding the engineered ketoreductase and phosphite dehydrogenase enzymes, host cells capable of expressing the engineered ketoreductase and phosphite dehydrogenase enzymes, and methods of using the engineered ketoreductase and phosphite dehydrogenase enzymes to synthesize a chiral catalyst used in the synthesis of antiviral compounds, such as nucleoside inhibitors. The present invention further provides methods of using the engineered enzymes to deracemize a chiral alcohol in a one-pot, multi-enzyme system.

### BACKGROUND

Enzymes belonging to the ketoreductase (KRED) or carbonyl reductase class (EC1.1.1.184) are useful for the synthesis of optically active alcohols from the corresponding prochiral ketone substrate and by stereoselective reduction of corresponding racemic aldehyde substrates. KREDs typically convert ketone and aldehyde substrates to the corresponding alcohol product, but may also catalyze the reverse reaction, oxidation of an alcohol substrate to the corresponding ketone/aldehyde product. The reduction of ketones and aldehydes and the oxidation of alcohols by enzymes such as KRED requires a co-factor, most commonly reduced nicotinamide adenine dinucleotide (NADH) or reduced nicotinamide adenine dinucleotide phosphate (NADPH), and nicotinamide adenine dinucleotide (NAD) or nicotinamide adenine dinucleotide phosphate (NADP) for the oxidation reaction. NADH and NADPH serve as electron donors, while NAD and NADP serve as electron acceptors. It is frequently observed that ketoreductases and alcohol dehydrogenases accept either the phosphorylated or the non-phosphorylated co-factor (in its oxidized and reduced state), but most often not both.

In order to circumvent many chemical synthetic procedures for the production of key compounds, ketoreductases are being increasingly employed for the enzymatic conversion of different keto and aldehyde substrates to chiral alcohol products. These applications can employ whole cells expressing the ketoreductase for biocatalytic ketone and aldehyde reductions or for biocatalytic alcohol oxidation, or by use of purified enzymes in those instances where presence of multiple ketoreductases in whole cells would adversely affect the stereopurity and yield of the desired product. For in vitro applications, a co-factor (NADH or NADPH) regenerating enzyme such as glucose dehydrogenase (GDH), formate dehydrogenase, phosphite dehydrogenase etc. can be used in conjunction with the ketoreductase. It is desirable to identify other ketoreductase enzymes that can be used to carryout conversion of various keto substrates to corresponding chiral alcohol products or conversion of various alcohol substrates to corresponding ketone products.

### SUMMARY OF THE INVENTION

The present invention provides engineered ketoreductase and phosphite dehydrogenase enzymes having improved properties as compared to a naturally occurring wild-type ketoreductase and phosphite dehydrogenase enzymes, as well as polynucleotides encoding the engineered ketoreductase and phosphite dehydrogenase enzymes, host cells capable of expressing the engineered ketoreductase and phosphite dehydrogenase enzymes, and methods of using the engineered ketoreductase and phosphite dehydrogenase enzymes to synthesize a chiral catalyst used in the synthesis of antiviral compounds, such as nucleoside inhibitors. The present invention further provides methods of using the engineered enzymes to deracemize a chiral alcohol in a one-pot, multi-enzyme system.

In addition, the present invention provides engineered phosphite dehydrogenase enzymes having improved properties as compared to a naturally occurring wild-type phosphite dehydrogenase enzyme, as well as polynucleotides encoding the engineered phosphite dehydrogenase enzymes, host cells capable of expressing the engineered phosphite dehydrogenase enzymes, and methods of using the engineered phosphite dehydrogenase enzymes to deracemize a chiral alcohol in a one-pot, multi-enzyme system.

The present invention provides engineered ketoreductase (“KRED”) enzymes that are capable of stereoselectively deracemizing a racemic alcohol substrate to an optically pure alcohol product in a one-pot, multi-enzyme system, and having an improved property when compared with the naturally-occurring, wild-type KRED enzyme obtained from Candida parapsilosis (SEQ ID NO:2), wild-type KRED enzyme obtained from Sporidiobolus salmonicolor (SEQ ID NO: 112), or when compared with other engineered ketoreductase enzymes. In addition, the present invention provides engineered phosphite dehydrogenase (“PDH”) enzymes capable of preferentially recycling NADPH in the same one-pot, multi-enzyme system.

In some further embodiments, the engineered enzymes have one or more improved properties in addition to altered enzymatic activity. For example, in some embodiments, the engineered ketoreductase polypeptides have increased stereoselectivity, as compared to the wild-type ketoreductase enzyme for reducing the substrate to the product and/or preferentially oxidize the (S) enantiomer. Improvements in enzyme properties include, but are not limited to increases in thermostability, solvent stability, and/or reduced product inhibition.

The present invention provides engineered ketoreductase variants having at least 85%, 90%, 91%, 92%, 93%, 94%, 95%, 96%, 97%, 98%, 99% or more sequence identity to SEQ ID NO: 2, 112, 124, and/or 138.

The present invention also provides engineered ketoreductase variants have at least 85%, 90%, 91%, 92%, 93%, 94%, 95%, 96%, 97%, 98%, 99% or more sequence identity to SEQ ID NO:2, and at least one substitution or substitution set at one or more positions selected from positions 37, 37/211, 37/211/229, 37/229, 45, 52, 52/57/110/272/296, 52/57/272, 52/57/272/274/279/296, 52/57/272/279/296, 55/57/276, 56, 57, 57/104/114, 57/104/114/229, 57/286, 79/83/275/276, 83, 83/275/276, 83/276, 104, 110, 114, 138/146/258/289, 211, 211/229, 228, 229, 263, 268, 272, 274, 275/276, 276, 279, and 309, wherein the positions are numbered with reference to SEQ ID NO:2. In some additional embodiments, the engineered ketoreductase variants comprise at least one substitution or substitution set selected from 37R, 37R/21 IR, 37R/211R/229R, 37R/229R, 45R, 52D, 52D/57L/272H, 52S, 52S/57L/110T/272H/296F, 52S/57L/272H/279H/296F, 52S/57L/272H/274V/279H/296F, 55F/57A/276M, 56L, 571, 571/104G/114H, 57L, 57L/104G/114H/229R, 57X/286X, 79T/83S/275N/276M, 831, 83S/275N/276M, 83S/276M, 104G, 110T, 114H/K/M, 138V/146S/258V/289S, 211R, 211R/229R, 228S, 229R, 263H/Y, 268M/W, 272H/I/L/P/Q/S/T/V/W, 2741/V, 275N/276M, 276/M, 279H/Q/R and 309F, wherein the positions are numbered with reference to SEQ ID NO:2. In some further embodiments, the engineered ketoreductase variants comprise at least one substitution or substitution set selected from K37R, K37R/K211R, K37R/K211R/G229R, K37R/G229R, H45R, Y52D, Y52D/C57L/G272H, Y52S, Y52S/C57L/K110T/G272H/L296F, Y52S/C57L/G272H/I279H/L296F, Y52S/C57L/G272H/L274V/1279H/L296F, L55F/C57A/L276M, D56L, C571, C571/A104G/G114H, C57L, C57L/A104G/G114H/G229R, C57X/W286X, 179T/V83S/A275N/L276M, V831, V83S/A275N/L276M, V83S/L276M, A104G, K110T, GI14H/K/M, S138V/A146S/M258V/T289S, K21 IR, K211R/G229R, P228S, G229R, G263H/Y, S268M/W, G272H/I/L/P/Q/S/T/V/W, L2741/V, A275N/L276M, L276F/M, I279H/Q/R, and R309F, wherein the positions are numbered with reference to SEQ ID NO:2.

The present invention also provides engineered ketoreductase variants having at least 85%, 90%, 91%, 92%, 93%, 94%, 95%, 96%, 97%, 98%, 99% or more sequence identity to SEQ ID NO: 112, and at least one substitution or substitution set at one or more positions selected from positions 24/106/136/220/258/260/314/315, 24/106/214/250/258/260/314/315, 24/220/314/315, 122/159/316/318, 135, 139/207, 159/251/272/277/316/318/330, and 207, wherein the positions are numbered with reference to SEQ ID NO: 112. In some embodiments, the engineered ketoreductase variants comprise at least one substitution or substitution set selected from 24I/106P/136A/220G/258V/260A/314R/315A, 24I/106P/214L/250V/258V/260A/314R/315A, 24I/220G/314R/315A, 122E/159V/316E/318L, 135F, 139V/207S, 159V/251Q/272F/277P/316E/318L/330L, and 207G, wherein the positions are numbered with reference to SEQ ID NO: 112. In some additional embodiments, the engineered ketoreductase variants comprise at least one substitution or substitution set selected from V24I/T106P/S136A/S220G/L258V/C260A/P314R/S315A, V24I/T106P/F214L/A250V/L258V/C260A/P314R/S315A, V24I/S220G/P314R/S315A, T122E/I159V/L316E/I318L, V135F, I139V/N207S, I159V/V251Q/Y272F/T277P/L316E/I318L/I330L, and N207G, wherein the positions are numbered with reference to SEQ ID NO: 112.

The present invention also provides engineered ketoreductase variants having at least 85%, 90%, 91%, 92%, 93%, 94%, 95%, 96%, 97%, 98%, 99% or more sequence identity to SEQ ID NO: 124, and at least one substitution set selected from positions 2/101/179/182/228/238/282, 3/95, 3/95/228/314, 24/95/228, 95, 95/135/139/207, and 159/228/309/330, wherein the positions are numbered with reference to SEQ ID NO: 124. In some embodiments, the engineered ketoreductase variants comprise at least one substitution or substitution set selected from 2T/101P/179L/182M/228R/238L/282E, 3Y/95T, 3Y/95T/228T/314R, 24I/95T/228T, 95T, 95T/135F/139V/207N, and 159V/228L/309Q/330L, wherein the positions are numbered with reference to SEQ ID NO: 124. In some further embodiments, the engineered ketoreductase variants comprise at least one substitution or substitution set selected from A2T/Y101P/A179L/T182M/M228R/A238L/T282E, K3Y/V95T, K3Y/V95T/M228T/P314R, V24I/V95T/M228T, V95T, V95T/V135F/I139V/G207N, and I159V/M228L/K309Q/I330L, wherein the positions are numbered with reference to SEQ ID NO: 124.

The present invention also provides engineered ketoreductase variants having at least 85%, 90%, 91%, 92%, 93%, 94%, 95%, 96%, 97%, 98%, 99% or more sequence identity to SEQ ID NO:138, and at least one substitution or substitution set at one or more positions selected from positions 19, 24/43/47/49/67/68/70/91/220, 24/68/91/218/220, 67, 72, 74/75/78/108, 75/78/99/108/215/224, 78/107, 95, 96, and 114, wherein the positions are numbered with reference to SEQ ID NO:138. In some embodiments, the engineered ketoreductase variants comprise at least one substitution or substitution set selected from 19S, 24I/43V/47E/49N/67V/68E/70P/91V/220G, 24I/68E/91V/218N/220G, 67W, 72Q, 74A/75E/78F/108V, 75E/78F/99P/108V/215S/224A, 78F/107G, 95C, 96G, and 114V, wherein the positions are numbered with reference to SEQ ID NO: 138. In some further embodiments, the engineered ketoreductase variants comprise at least one substitution or substitution set selected from G19S, V24I/A43V/S47E/L49N/A67V/V68E/E70P/I91V/S220G, V24I/V68E/I91V/T218N/S220G, A67W, M72Q, K74A/Q75E/Y78F/A108V, Q75E/Y78F/N99P/A108V/D215S/S224A, Y78F/P107G, T95C, S96G, and N114V, wherein the positions are numbered with reference to SEQ ID NO:138.

The present invention also provides engineered ketoreductase variants comprising polypeptide sequences comprising sequences having at least 90% sequence identity to SEQ ID NO:2, 112, 124, and/or 138. In some embodiments, the engineered ketoreductase variants comprise polypeptide sequences comprising sequences having at least 95% sequence identity to SEQ ID NO:2, 112, 124, and/or 138. In some further embodiments, the engineered ketoreductase variants comprise polypeptide sequences set forth in SEQ ID NO:2, 112, 124, or 138. In some additional embodiments, the engineered ketoreductase variants comprise polypeptide sequences encoding variants provided in Table 5.1, 6.1, 7.1, and/or 8.1. In some further embodiments, the engineered ketoreductase variants comprise polypeptide sequences selected from the even-numbered sequences set forth in SEQ ID NOS: 4 to 170.

The present invention also provides engineered polynucleotide sequences encoding the engineered ketoreductase variants provided herein. In some embodiments, the engineered polynucleotide sequence comprises a polynucleotide sequence that is at least 85%, 90%, 91%, 92%, 93%, 94%, 95%, 96%, 97%, 98%, 99% or more identical to a sequence selected from the odd-numbered sequences set forth in SEQ ID NOS: 3 to 169. The present invention also provides vectors comprising the engineered polynucleotide sequences encoding the engineered ketoreductase variants provided herein. In some embodiments, the vectors further comprise at least one control sequence.

The present invention also provides host cells comprising the vectors comprising polynucleotides encoding the engineered ketoreductase variants provided herein.

The present invention also provides methods producing the engineered ketoreductase variants provided herein, comprising culturing the host cells provided herein under conditions that the engineered ketoreductase variant is produced by the host cell. In some embodiments, the methods further comprise the step of recovering the engineered ketoreductase variant produced by the host cell.

The present invention also provides immobilized engineered ketoreductase variants.

The present invention further provides compositions comprising at least one engineered ketoreductase variant provided herein. In some embodiments, the compositions comprise at least one immobilized engineered ketodreductase variant provided herein.

The present invention also provides engineered phosphite dehydrogenase variants having at least 85%, 90%, 91%, 92%, 93%, 94%, 95%, 96%, 97%, 98%, 99% or more sequence identity to SEQ ID NO: 172 and/or 208.

The present invention also provides engineered phosphite dehydrogenase variants having at least 85%, 90%, 91%, 92%, 93%, 94%, 95%, 96%, 97%, 98%, 99% or more sequence identity to SEQ ID NO: 172, and at least one substitution or substitution set at one or more positions selected from positions 10/73/78/137/323/325, 10/73/78/233/323, 10/73/137, 13/41/63/132/193/195, 18/44/119/124/132/137/145/158/175/177/293/317/323, 18/44/119/124/132/137/145/158/177/293/323, 18/44/119/124/132/137/145/293/323/334/336, 32/44/132/137/145/186/233/293/323/336, 41/44/88/193/195, 44/69/120/132/137/145/175/195/293/323, 44/113/132/145, 44/119/132/137/145/158/175/177/293/317/323, 44/132/135/136/137/145/293, 44/132/136/137/145/293, 44/132/137/145/233/308/323, 44/132/137/145/293/323, 44/132/145, 44/132/145/195/293/323, 137/233/303/323, and 266, wherein the positions are numbered with reference to SEQ ID NO: 172. In some embodiments, the engineered phosphite dehydrogenase variants comprise at least one substitution or substitution set selected from 10K/73A/78Y/137Q/323D/325A, 10K/73A/78Y/233I/323D, 10K/73A/137Q, 13D/41A/63A/132Q/193S/195E, 18M/44A/119F/124E/132Q/137I/145G/158K/175S/177T/293L/317R/323D, 18M/44A/119F/124E/132Q/137I/145G/158K/177T/293L/323D, 18M/44A/119F/124E/132Q/137I/145G/293L/323D/334K/336R, 32V/44A/132Q/137I/145G/186T/233I/293L/323D/336S, 41A/44A/88R/193S/195E, 44A/69K/120V/132Q/137I/145G/175T/195E/293L/323D, 44A/113S/132Q/145G, 44A/119F/132Q/137I/145G/158K/175S/177T/293L/317R/323D, 44A/132Q/135A/136D/137I/145G/293L, 44A/132Q/136D/137Q/145G/293L, 44A/132Q/137I/145G/233I/308V/323D, 44A/132Q/137I/145G/293L/323D, 44A/132Q/145G, 44A/132Q/145G/195E/293L/323D, 137Q/233I/303A/323D, and 266S/V/W, wherein the positions are numbered with reference to SEQ ID NO: 172. In some further embodiments, the engineered phosphite dehydrogenase variants comprise at least one substitution or substitution set selected from R10K/C73A/F78Y/R137Q/N323D/V325A, R10K/C73A/F78Y/V233I/N323D, R10K/C73A/R137Q, E13D/R41A/Q63A/R132Q/A193S/S195E, L18M/R44A/L 119F/A124E/R132Q/R137I/N145G/L158K/A175S/K177T/I293L/A317R/N323D, L18M/R44A/L 119F/A124E/R132Q/R137I/N145G/L158K/K177T/I293L/N323D, L18M/R44A/L 119F/A124E/R132Q/R137I/N145G/I293L/N323D/A334K/C336R, S32V/R44A/R132Q/R137I/N145G/R186T/V233I/I293L/N323D/C336S, R41A/R44A/A88R/A193S/S195E, R44A/R69K/R120V/R132Q/R137I/N145G/A175T/S195E/I293L/N323D, R44A/V113S/R132Q/N145G, R44A/L 119F/R132Q/R137I/N145G/L158K/A175S/K177T/I293L/A317R/N323D, R44A/R132Q/Q135A/P136D/R137I/N145G/I293L, R44A/R132Q/P136D/R137Q/N145G/I293L, R44A/R132Q/R137I/N145G/V233I/A308V/N323D, R44A/R132Q/R137I/N145G/I293L/N323D, R44A/R132Q/N145G, R44A/R132Q/N145G/S195E/I293L/N323D, R137Q/V233I/E303A/N323D, and E266S/V/W, wherein the positions are numbered with reference to SEQ ID NO: 172.

The present invention also provides engineered phosphite dehydrogenase variants having at least 85%, 90%, 91%, 92%, 93%, 94%, 95%, 96%, 97%, 98%, 99% or more sequence identity to SEQ ID NO:208, and at least one substitution or substitution set at one or more positions selected from positions 32/59/124/177/191/327, 78/150/198/327/328, 83/266, 95/211/213/322, 104, 178/194/211/213/322, 206, 211/213/322, 215, 262, 266, and 323, wherein the positions are numbered with reference to SEQ ID NO:208. In some embodiments, the engineered phosphite dehydrogenase variants comprise at least one substitution or substitution set selected from 32V/59M/124E/177S/191H/327D, 78Y/150I/198L/327S/328P, 83A/266A, 95I/211A/213Q/322M, 104F/L, 178P/194L/211A/213Q/322Q, 206N, 211A/213Q/322Q, 215P, 262D/P, 266S, and 323N, wherein the positions are numbered with reference to SEQ ID NO:208. In some further embodiments, the engineered phosphite dehydrogenase variants comprise at least one substitution or substitution set selected from S32V/A59M/A124E/T177S/Q191H/R327D, F78Y/F150I/F198L/R327S/L328P, V83A/E266A, F95I/N211A/D213Q/I322M, T104F/L, A178P/C194L/N211A/D213Q/I322Q, L206N, N211A/D213Q/I322Q, L215P, V262D/P, E266S, and D323N, wherein the positions are numbered with reference to SEQ ID NO:208.

The present invention also provides engineered phosphite dehydrogenase variants comprising a polypeptide sequence comprising a sequence having at least 90% sequence identity to SEQ ID NO: 172 and/or 208. In some embodiments, the engineered phosphite dehydrogenase variants comprise polypeptide sequences comprising sequences having at least 95% sequence identity to SEQ ID NO: 172 and/or 208. In some further embodiments, the engineered phosphite dehydrogenase variants comprise polypeptide sequences set forth in SEQ ID NO: 172 or 208. In some additional embodiments, the engineered phosphite dehydrogenase variants comprise polypeptide sequences encoding variants provided in Table 9.1, 10.1, and/or 11.1. In yet some additional embodiments, the engineered phosphite dehydrogenase variants comprise polypeptide sequences selected from the even-numbered sequences set from in SEQ ID NOS: 174 to 260.

The present invention also provides immobilized engineered phosphite dehydrogenase variants. In some embodiments, the present invention provides a mixture of at least one immobilized engineered ketoreductase variant provided herein and at least one engineered phosphite dehydrogenase variant provided herein.

The present invention also provides compositions comprising at least one phosphite dehydrogenase variant provided herein. In some embodiments, the present invention further provides compositions comprising mixtures of at least one engineered ketoreductase variant provided herein and at least one engineered phosphite dehydrogenase provided herein.

The present invention also provides engineered polynucleotide sequences encoding the engineered phosphite dehydrogenase variants provided herein. In some embodiments, the engineered polynucleotide sequences comprise polynucleotide sequences that are at least 85%, 90%, 91%, 92%, 93%, 94%, 95%, 96%, 97%, 98%, 99% or more identical to a sequence selected from the odd-numbered sequences set forth in SEQ ID NOS: 3 to 169.

The present invention also provides vectors comprising the engineered polynucleotide sequence encoding the engineered phosphite dehydrogenase variants provided herein. In some embodiments, the vectors further comprise at least one control sequence. In yet some further embodiments, the vectors comprise at least one engineered polynucleotide sequence encoding an engineered phosphite dehydrogenase variant provided herein and at least one engineered polynucleotide sequence encoding an engineered ketoreductase variant provided herein. The present invention also provides host cells comprising the vectors provided herein.

The present invention also provides methods for producing the engineered phosphite dehydrogenase variants provided herein, comprising culturing the host cell comprising a vector comprising at least one engineered polynucleotide sequence encoding at least one engineered phosphite dehydrogenase of the present invention, under conditions that the engineered phosphite dehydrogenase variant is produced by the host cell. In some embodiments, the host cells comprise vectors comprising polynucleotide sequences comprising at least one engineered ketoreductase and at least one engineered phosphite dehydrogenase provided herein. In some additional embodiments, the host cells comprise at least one ketoreductase not provided herein, but comprise at least one engineered phosphite dehydrogenase variant provided herein. In some further embodiments, the host cells comprise at least on phosphite dehydrogenase not provided herein, but comprise at least one engineered ketoreductase variant provided herein. In some embodiments, the methods further comprise the step of recovering the engineered phosphite dehydrogenase variant produced by the host cell. In embodiments with host cells that produce at least one ketoreductase and at least one phosphite dehydrogenase, some methods further comprise the step of recovering the ketoreductase and/or phosphite dehydrogenase produced by the host cells.

The present invention also provides methods deracemizing chiral alcohols comprising providing at least one engineered ketoreductase variant provided herein, providing at least one engineered phosphite dehydrogenase variant provided herein, at least one chiral alcohol, and at least one co-factor, under conditions such that the chiral alcohol is deracemized. In some embodiments, the methods are conducted in a one pot reaction, while in some alternative embodiments, multiple reaction vessels are used.

### BRIEF DESCRIPTION OF THE FIGURES

FIG. 1 provides the reaction scheme addressed by the present invention.

FIG. 2 provides the structures of substrate and product isomers.

FIG. 3 provides the one-pot, multi-enzyme reaction scheme.

FIGS. 4 and 5 provide the cofactor competition assay schemes.

FIG. 6 provides the HPLC chromatogram of products obtained in one-pot, multi-enzyme reactions.

### DESCRIPTION OF THE INVENTION

The present invention provides engineered ketoreductase enzymes and engineered phosphite dehydrogenase enzymes having improved properties as compared to a naturally occurring wild-type ketoreductase and phosphite dehydrogenase enzyme, as well as polynucleotides encoding the engineered ketoreductase and engineered phosphite dehydrogenase enzymes, host cells capable of expressing the engineered ketoreductase and engineered phosphite dehydrogenase enzymes, and methods of using the engineered ketoreductase and engineered phosphite dehydrogenase enzymes to deracemize a racemic alcohol in a one-pot, multi-enzyme system.

### Definitions

In reference to the present invention, the technical and scientific terms used in the descriptions herein will have the meanings commonly understood by one of ordinary skill in the art, unless specifically defined otherwise. Accordingly, the following terms are intended to have the following meanings. All patents and publications, including all sequences disclosed within such patents and publications, referred to herein are expressly incorporated by reference. Unless otherwise indicated, the practice of the present invention involves conventional techniques commonly used in molecular biology, fermentation, microbiology, and related fields, which are known to those of skill in the art. Unless defined otherwise herein, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described. Indeed, it is intended that the present invention not be limited to the particular methodology, protocols, and reagents described herein, as these may vary, depending upon the context in which they are used. The headings provided herein are not limitations of the various aspects or embodiments of the present invention.

Nonetheless, in order to facilitate understanding of the present invention, a number of terms are defined below. Numeric ranges are inclusive of the numbers defining the range. Thus, every numerical range disclosed herein is intended to encompass every narrower numerical range that falls within such broader numerical range, as if such narrower numerical ranges were all expressly written herein. It is also intended that every maximum (or minimum) numerical limitation disclosed herein includes every lower (or higher) numerical limitation, as if such lower (or higher) numerical limitations were expressly written herein.

As used herein, the term “comprising” and its cognates are used in their inclusive sense (i.e., equivalent to the term “including” and its corresponding cognates).

As used herein and in the appended claims, the singular “a”, “an” and “the” include the plural reference unless the context clearly dictates otherwise. Thus, for example, reference to a “host cell” includes a plurality of such host cells.

Unless otherwise indicated, nucleic acids are written left to right in 5′ to 3′ orientation and amino acid sequences are written left to right in amino to carboxy orientation, respectively.

The headings provided herein are not limitations of the various aspects or embodiments of the invention that can be had by reference to the specification as a whole. Accordingly, the terms defined below are more fully defined by reference to the specification as a whole.

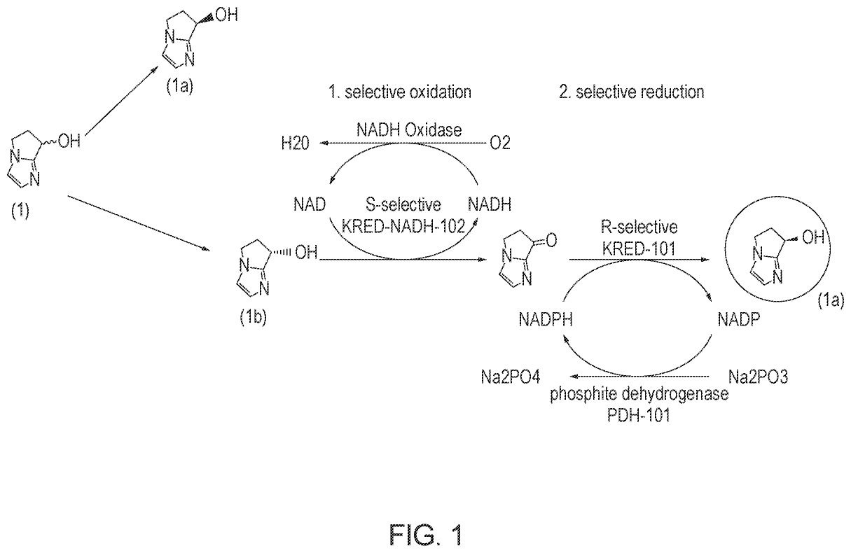

“Ketoreductase” and “KRED” are used interchangeably herein to refer to a polypeptide having an enzymatic capability of reducing a carbonyl group to its corresponding alcohol. More specifically, the ketoreductase polypeptides of the invention are capable of stereoselectively deracemizing an alcohol of formula (I) to the corresponding product of formula (II) in an one-pot, multi-enzyme system, as shown in Scheme 1 (See, FIG. 1).

Phosphite dehydrogenase and “PDH” are used interchangeably herein to refer to a polypeptide having an enzymatic capability of regenerating NADPH co-factor.

As used herein, the term “one-pot reaction” refers to the production of a product from a starting material using multiple enzymes (i.e., KREDs and PDHs) in one reaction vessel.

As used herein, the terms “protein,” “polypeptide,” and “peptide” are used interchangeably herein to denote a polymer of at least two amino acids covalently linked by an amide bond, regardless of length or post-translational modification (e.g., glycosylation, phosphorylation, lipidation, myristilation, ubiquitination, etc.). Included within this definition are D- and L-amino acids, and mixtures of D- and L-amino acids.

As used herein, “polynucleotide” and “nucleic acid’ refer to two or more nucleosides that are covalently linked together. The polynucleotide may be wholly comprised of ribonucleosides (i.e., an RNA), wholly comprised of 2′ deoxyribonucleotides (i.e., a DNA) or mixtures of ribo- and 2′ deoxyribonucleosides. While the nucleosides will typically be linked together via standard phosphodiester linkages, the polynucleotides may include one or more non-standard linkages. The polynucleotide may be single-stranded or double-stranded, or may include both single-stranded regions and double-stranded regions. Moreover, while a polynucleotide will typically be composed of the naturally occurring encoding nucleobases (i.e., adenine, guanine, uracil, thymine, and cytosine), it may include one or more modified and/or synthetic nucleobases (e.g., inosine, xanthine, hypoxanthine, etc.). Preferably, such modified or synthetic nucleobases will be encoding nucleobases.

As used herein, “coding sequence” refers to that portion of a nucleic acid (e.g., a gene) that encodes an amino acid sequence of a protein.

As used herein, “naturally occurring” or “wild-type” refers to the form found in nature. For example, a naturally occurring or wild-type polypeptide or polynucleotide sequence is a sequence present in an organism that can be isolated from a source in nature and which has not been intentionally modified by human manipulation.

As used herein, “non-naturally occurring” or “engineered” or “recombinant” when used in the present invention with reference to (e.g., a cell, nucleic acid, or polypeptide), refers to a material, or a material corresponding to the natural or native form of the material, that has been modified in a manner that would not otherwise exist in nature, or is identical thereto but produced or derived from synthetic materials and/or by manipulation using recombinant techniques. Non-limiting examples include, among others, recombinant cells expressing genes that are not found within the native (non-recombinant) form of the cell or express native genes that are otherwise expressed at a different level.

As used herein, “percentage of sequence identity,” “percent identity,” and “percent identical” refer to comparisons between polynucleotide sequences or polypeptide sequences, and are determined by comparing two optimally aligned sequences over a comparison window, wherein the portion of the polynucleotide or polypeptide sequence in the comparison window may comprise additions or deletions (i.e., gaps) as compared to the reference sequence for optimal alignment of the two sequences. The percentage is calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Determination of optimal alignment and percent sequence identity is performed using the BLAST and BLAST 2.0 algorithms (See e.g., Altschul et al., J. Mol. Biol. 215: 403-410 [1990]; and Altschul et al., Nucleic Acids Res. 3389-3402 [1977]). Software for performing BLAST analyses is publicly available through the National Center for Biotechnology Information website.

Briefly, the BLAST analyses involve first identifying high scoring sequence pairs (HSPs) by identifying short words of length Win the query sequence, which either match or satisfy some positive-valued threshold score T when aligned with a word of the same length in a database sequence. T is referred to as, the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (See e.g., Henikoff and Henikoff, Proc Natl Acad Sci USA 89:10915 [1989]).

Numerous other algorithms are available and known in the art that function similarly to BLAST in providing percent identity for two sequences. Optimal alignment of sequences for comparison can be conducted using any suitable method known in the art (e.g., by the local homology algorithm of Smith and Waterman, Adv. Appl. Math. 2:482 [1981]; by the homology alignment algorithm of Needleman and Wunsch, J. Mol. Biol. 48:443 [1970]; by the search for similarity method of Pearson and Lipman, Proc. Natl. Acad. Sci. USA 85:2444 [1988]; and/or by computerized implementations of these algorithms [GAP, BESTFIT, FASTA, and TFASTA in the GCG Wisconsin Software Package]), or by visual inspection, using methods commonly known in the art. Additionally, determination of sequence alignment and percent sequence identity can employ the BESTFIT or GAP programs in the GCG Wisconsin Software package (Accelrys, Madison WI), using the default parameters provided.

As used herein, “reference sequence” refers to a defined sequence to which another sequence is compared. A reference sequence may be a subset of a larger sequence, for example, a segment of a full-length gene or polypeptide sequence. Generally, a reference sequence is at least 20 nucleotide or amino acid residues in length, at least 25 residues in length, at least 50 residues in length, or the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptide are typically performed by comparing sequences of the two polynucleotides over a comparison window to identify and compare local regions of sequence similarity. The term “reference sequence” is not intended to be limited to wild-type sequences, and can include engineered or altered sequences. For example, in some embodiments, a “reference sequence” can be a previously engineered or altered amino acid sequence.

As used herein, “comparison window” refers to a conceptual segment of at least about 20 contiguous nucleotide positions or amino acids residues wherein a sequence may be compared to a reference sequence of at least 20 contiguous nucleotides or amino acids and wherein the portion of the sequence in the comparison window may comprise additions or deletions (i.e., gaps) of 20 percent or less as compared to the reference sequence (which does not comprise additions or deletions) for optimal alignment of the two sequences. The comparison window can be longer than 20 contiguous residues, and includes, optionally 30, 40, 50, 100, or longer windows.

As used herein, “corresponding to”, “reference to” or “relative to” when used in the context of the numbering of a given amino acid or polynucleotide sequence refers to the numbering of the residues of a specified reference sequence when the given amino acid or polynucleotide sequence is compared to the reference sequence. In other words, the residue number or residue position of a given polymer is designated with respect to the reference sequence rather than by the actual numerical position of the residue within the given amino acid or polynucleotide sequence. For example, a given amino acid sequence, such as that of an engineered ketoreductase, can be aligned to a reference sequence by introducing gaps to optimize residue matches between the two sequences. In these cases, although the gaps are present, the numbering of the residue in the given amino acid or polynucleotide sequence is made with respect to the reference sequence to which it has been aligned. As used herein, a reference to a residue position, such as “Xn” as further described below, is to be construed as referring to “a residue corresponding to”, unless specifically denoted otherwise. Thus, for example, “X94” refers to any amino acid at position 94 in a polypeptide sequence.

As used herein, “stereoselectivity” refers to the preferential formation in a chemical or enzymatic reaction of one stereoisomer over another stereoisomer or another set of stereoisomers. Stereoselectivity can be partial, where the formation of a stereoisomer is favored over another, or it may be complete where only one stereoisomer is formed. When the stereoisomers are enantiomers, the stereoselectivity is referred to as enantioselectivity, the fraction (typically reported as a percentage) of one enantiomer in the sum of both enantiomers. It is commonly alternatively reported in the art (typically as a percentage) as the enantiomeric excess (e.e.) calculated therefrom according to the formula [major enantiomer−minor enantiomer]/[major enantiomer+minor enantiomer]. Where the stereoisomers are diastereoisomers, the stereoselectivity is referred to as diastereoselectivity, the fraction (typically reported as a percentage) of one diastereomer in a mixture of two diastereomers, commonly alternatively reported as the diastereomeric excess (d.e.). Enantiomeric excess and diastereomeric excess are types of stereomeric excess. It is also to be understood that stereoselectivity is not limited to single stereoisomers and can be described for sets of stereoisomers.

As used herein, “highly stereoselective” refers to a chemical or enzymatic reaction that is capable of converting a substrate to its corresponding chiral alcohol product, with at least about 75% stereomeric excess.

As used herein, “increased enzymatic activity” and “increased activity” refer to an improved property of an engineered enzyme, which can be represented by an increase in specific activity (e.g., product produced/time/weight protein) or an increase in percent conversion of the substrate to the product (e.g., percent conversion of starting amount of substrate to product in a specified time period using a specified amount of ketoreductase) as compared to a reference enzyme. Exemplary methods to determine enzyme activity are provided in the Examples. Any property relating to enzyme activity may be affected, including the classical enzyme properties of Km, Vmax or kcat, changes of which can lead to increased enzymatic activity. The ketoreductase activity can be measured by any one of standard assays used for measuring ketoreductases, such as change in substrate or product concentration, or change in concentration of the cofactor (in absence of a cofactor regenerating system). Comparisons of enzyme activities are made using a defined preparation of enzyme, a defined assay under a set condition, and one or more defined substrates, as further described in detail herein. Generally, when enzymes in cell lysates are compared, the numbers of cells and the amount of protein assayed are determined as well as use of identical expression systems and identical host cells to minimize variations in amount of enzyme produced by the host cells and present in the lysates.

As used herein, “conversion” refers to the enzymatic transformation of a substrate to the corresponding product.

As used herein “percent conversion” refers to the percent of the substrate that is converted to the product within a period of time under specified conditions. Thus, for example, the “enzymatic activity” or “activity” of a ketoreductase polypeptide can be expressed as “percent conversion” of the substrate to the product.

As used herein, “thermostable” or “thermal stable” are used interchangeably to refer to a polypeptide that is resistant to inactivation when exposed to a set of temperature conditions (e.g., 40-80° C.) for a period of time (e.g., 0.5-24 hrs) compared to the untreated enzyme, thus retaining a certain level of residual activity (e.g., more than 60% to 80% for example) after exposure to elevated temperatures.

As used herein, “solvent stable” refers to the ability of a polypeptide to maintain similar activity (e.g., more than e.g., 60% to 80%) after exposure to varying concentrations (e.g., 5-99%) of solvent compared to the untreated enzyme.

As used herein, “amino acid difference” or “residue difference” refers to a difference in the amino acid residue at a position of a polypeptide sequence relative to the amino acid residue at a corresponding position in a reference sequence. The positions of amino acid differences generally are referred to herein as “Xn,” where n refers to the corresponding position in the reference sequence upon which the residue difference is based. For example, a “residue difference at position X40 as compared to SEQ ID NO:2” refers to a difference of the amino acid residue at the polypeptide position corresponding to position 40 of SEQ ID NO:2. Thus, if the reference polypeptide of SEQ ID NO:2 has a histidine at position 40, then a “residue difference at position X40 as compared to SEQ ID NO:2” refers to an amino acid substitution of any residue other than histidine at the position of the polypeptide corresponding to position 40 of SEQ ID NO:2. In most instances herein, the specific amino acid residue difference at a position is indicated as “XnY” where “Xn” specified the corresponding position as described above, and “Y” is the single letter identifier of the amino acid found in the engineered polypeptide (i.e., the different residue than in the reference polypeptide). In some instances, the present invention also provides specific amino acid differences denoted by the conventional notation “AnB”, where A is the single letter identifier of the residue in the reference sequence, “n” is the number of the residue position in the reference sequence, and B is the single letter identifier of the residue substitution in the sequence of the engineered polypeptide. In some instances, a polypeptide of the present invention can include one or more amino acid residue differences relative to a reference sequence, which is indicated by a list of the specified positions where residue differences are present relative to the reference sequence. In some embodiments, where more than one amino acid can be used in a specific residue position of a polypeptide, the various amino acid residues that can be used are separated by a “/” (e.g., X192A/G). The present invention includes engineered polypeptide sequences comprising one or more amino acid differences that include either/or both conservative and non-conservative amino acid substitutions. The amino acid sequences of the specific recombinant carbonic anhydrase polypeptides included in the Sequence Listing of the present invention include an initiating methionine (M) residue (i.e., M represents residue position 1). The skilled artisan, however, understands that this initiating methionine residue can be removed by biological processing machinery, such as in a host cell or in vitro translation system, to generate a mature protein lacking the initiating methionine residue, but otherwise retaining the enzyme's properties. Consequently, the term “amino acid residue difference relative to SEQ ID NO:2 at position Xn” as used herein may refer to position “Xn” or to the corresponding position (e.g., position (X−1)n) in a reference sequence that has been processed so as to lack the starting methionine.

As used herein, the phrase “conservative amino acid substitutions” refers to the interchangeability of residues having similar side chains, and thus typically involves substitution of the amino acid in the polypeptide with amino acids within the same or similar defined class of amino acids. By way of example and not limitation, in some embodiments, an amino acid with an aliphatic side chain is substituted with another aliphatic amino acid (e.g., alanine, valine, leucine, and isoleucine); an amino acid with a hydroxyl side chain is substituted with another amino acid with a hydroxyl side chain (e.g., serine and threonine); an amino acids having aromatic side chains is substituted with another amino acid having an aromatic side chain (e.g., phenylalanine, tyrosine, tryptophan, and histidine); an amino acid with a basic side chain is substituted with another amino acid with a basis side chain (e.g., lysine and arginine); an amino acid with an acidic side chain is substituted with another amino acid with an acidic side chain (e.g., aspartic acid or glutamic acid); and/or a hydrophobic or hydrophilic amino acid is replaced with another hydrophobic or hydrophilic amino acid, respectively. Exemplary conservative substitutions are provided in Table 1.

TABLE 1
Exemplary Conservative Amino Acid Substitutions

| Residue | Possible Conservative Substitutions |
|---|---|
| A, L, V, I | Other aliphatic (A, L, V, I) |
| | Other non-polar (A, L, V, I, G, M) |
| G, M | Other non-polar (A, L, V, I, G, M) |
| D, E | Other acidic (D, E) |
| K, R | Other basic (K, R) |
| N, Q, S, T | Other polar |
| H, Y, W, F | Other aromatic (H, Y, W, F) |
| C, P | Non-polar |

As used herein, the phrase “non-conservative substitution” refers to substitution of an amino acid in the polypeptide with an amino acid with significantly differing side chain properties. Non-conservative substitutions may use amino acids between, rather than within, the defined groups and affects (a) the structure of the peptide backbone in the area of the substitution (e.g., proline for glycine) (b) the charge or hydrophobicity, or (c) the bulk of the side chain. By way of example and not limitation, an exemplary non-conservative substitution can be an acidic amino acid substituted with a basic or aliphatic amino acid; an aromatic amino acid substituted with a small amino acid; and a hydrophilic amino acid substituted with a hydrophobic amino acid.

As used herein, “deletion” refers to modification of the polypeptide by removal of one or more amino acids from the reference polypeptide. Deletions can comprise removal of 1 or more amino acids, 2 or more amino acids, 5 or more amino acids, 10 or more amino acids, 15 or more amino acids, or 20 or more amino acids, up to 10% of the total number of amino acids, or up to 20% of the total number of amino acids making up the polypeptide while retaining enzymatic activity and/or retaining the improved properties of an engineered enzyme. Deletions can be directed to the internal portions and/or terminal portions of the polypeptide. In various embodiments, the deletion can comprise a continuous segment or can be discontinuous.

As used herein, “insertion” refers to modification of the polypeptide by addition of one or more amino acids to the reference polypeptide. In some embodiments, the improved engineered ketoreductase enzymes comprise insertions of one or more amino acids to the naturally occurring ketoreductase polypeptide as well as insertions of one or more amino acids to engineered ketoreductase polypeptides. Insertions can be in the internal portions of the polypeptide, or to the carboxy or amino terminus. Insertions as used herein include fusion proteins as is known in the art. The insertion can be a contiguous segment of amino acids or separated by one or more of the amino acids in the naturally occurring polypeptide.

The term “amino acid substitution set” or “substitution set” refers to a group of amino acid substitutions in a polypeptide sequence, as compared to a reference sequence. A substitution set can have 1, 2, 3, 4, 5, 6, 7, 8, 9, 10, 11, 12, 13, 14, 15, or more amino acid substitutions. In some embodiments, a substitution set refers to the set of amino acid substitutions that is present in any of the variant KREDs listed in the Tables provided in the Examples.

As used herein, “fragment” refers to a polypeptide that has an amino-terminal and/or carboxy-terminal deletion, but where the remaining amino acid sequence is identical to the corresponding positions in the sequence. Fragments can typically have about 80%, about 90%, about 95%, about 98%, or about 99% of the full-length ketoreductase polypeptide, for example the polypeptide of SEQ ID NO:4. In some embodiments, the fragment is “biologically active” (i.e., it exhibits the same enzymatic activity as the full-length sequence).

As used herein, “isolated polypeptide” refers to a polypeptide which is substantially separated from other contaminants that naturally accompany it, e.g., protein, lipids, and polynucleotides. The term embraces polypeptides which have been removed or purified from their naturally-occurring environment or expression system (e.g., host cell or in vitro synthesis). The improved ketoreductase enzymes may be present within a cell, present in the cellular medium, or prepared in various forms, such as lysates or isolated preparations. As such, in some embodiments, the engineered ketoreductase polypeptides of the present invention can be an isolated polypeptide.

As used herein, “substantially pure polypeptide” refers to a composition in which the polypeptide species is the predominant species present (i.e., on a molar or weight basis it is more abundant than any other individual macromolecular species in the composition), and is generally a substantially purified composition when the object species comprises at least about 50 percent of the macromolecular species present by mole or % weight. Generally, a substantially pure engineered ketoreductase polypeptide composition will comprise about 60% or more, about 70% or more, about 80% or more, about 90% or more, about 91% or more, about 92% or more, about 93% or more, about 94% or more, about 95% or more, about 96% or more, about 97% or more, about 98% or more, or about 99% of all macromolecular species by mole or % weight present in the composition. Solvent species, small molecules (<500 Daltons), and elemental ion species are not considered macromolecular species. In some embodiments, the isolated improved ketoreductase polypeptide is a substantially pure polypeptide composition.

As used herein, when used with reference to a nucleic acid or polypeptide, the term “heterologous” refers to a sequence that is not normally expressed and secreted by an organism (e.g., a wild-type organism). In some embodiments, the term encompasses a sequence that comprises two or more subsequences which are not found in the same relationship to each other as normally found in nature, or is recombinantly engineered so that its level of expression, or physical relationship to other nucleic acids or other molecules in a cell, or structure, is not normally found in nature. For instance, a heterologous nucleic acid is typically recombinantly produced, having two or more sequences from unrelated genes arranged in a manner not found in nature (e.g., a nucleic acid open reading frame (ORF) of the invention operatively linked to a promoter sequence inserted into an expression cassette, such as a vector). In some embodiments, “heterologous polynucleotide” refers to any polynucleotide that is introduced into a host cell by laboratory techniques, and includes polynucleotides that are removed from a host cell, subjected to laboratory manipulation, and then reintroduced into a host cell.

As used herein, “codon optimized” refers to changes in the codons of the polynucleotide encoding a protein to those preferentially used in a particular organism such that the encoded protein is efficiently expressed in the organism of interest. In some embodiments, the polynucleotides encoding the ketoreductase enzymes may be codon optimized for optimal production from the host organism selected for expression.

As used herein, “control sequence” is defined herein to include all components, which are necessary or advantageous for the expression of a polynucleotide and/or polypeptide of the present invention. Each control sequence may be native or foreign to the polynucleotide of interest. Such control sequences include, but are not limited to, a leader, polyadenylation sequence, propeptide sequence, promoter, signal peptide sequence, and transcription terminator.

As used herein, “operably linked” is defined herein as a configuration in which a control sequence is appropriately placed (i.e., in a functional relationship) at a position relative to a polynucleotide of interest such that the control sequence directs or regulates the expression of the polynucleotide and/or polypeptide of interest.

As used herein, the phrases “cofactor regeneration system” and “cofactor recycling system” refer to a set of reactants that participate in a reaction that reduces the oxidized form of the cofactor (e.g., NADP+ to NADPH). Cofactors oxidized by the ketoreductase-catalyzed reduction of the keto substrate are regenerated in reduced form by the cofactor regeneration system. Cofactor regeneration systems comprise a stoichiometric reductant that is a source of reducing hydrogen equivalents and is capable of reducing the oxidized form of the cofactor. The cofactor regeneration system may further comprise a catalyst, for example an enzyme catalyst that catalyzes the reduction of the oxidized form of the cofactor by the reductant. Cofactor regeneration systems to regenerate NADH or NADPH from NAD+ or NADP+, respectively, are known in the art and may be used in the methods described herein.

As used herein, “suitable reaction conditions” refer to those conditions in the biocatalytic reaction solution (e.g., ranges of enzyme loading, substrate loading, cofactor loading, temperature, pH, buffers, co-solvents, etc.) under which ketoreductase polypeptides of the present invention are capable of stereoselectively deracemizing a substrate compound to a product compound. Exemplary “suitable reaction conditions” are provided in the present invention and illustrated by the Examples.

As used herein, “loading,” such as in “compound loading,” “enzyme loading,” or “cofactor loading” refers to the concentration or amount of a component in a reaction mixture at the start of the reaction.

As used herein, “substrate” in the context of a biocatalyst mediated process refers to the compound or molecule acted on by the biocatalyst. For example, an exemplary substrate for the ketoreductase biocatalyst in the process disclosed herein is compound (1).

As used herein “product” in the context of a biocatalyst mediated process refers to the compound or molecule resulting from the action of the biocatalyst.

As used herein, “equilibration” as used herein refers to the process resulting in a steady state concentration of chemical species in a chemical or enzymatic reaction (e.g., interconversion of two species A and B), including interconversion of stereoisomers, as determined by the forward rate constant and the reverse rate constant of the chemical or enzymatic reaction.

As used herein, “oxo” refers to ═O.

As used herein, “oxy” refers to a divalent group —O—, which may have various substituents to form different oxy groups, including ethers and esters.

As used herein, “carboxy” refers to —COOH.

As used herein, “carbonyl” refers to —C(O)—, which may have a variety of substituents to form different carbonyl groups including acids, acid halides, aldehydes, amides, esters, and ketones.

As used herein, “hydroxy” refers to —OH.

As used herein, “optional” and “optionally” means that the subsequently described event or circumstance may or may not occur, and that the description includes instances where the event or circumstance occurs and instances in which it does not. One of ordinary skill in the art would understand that with respect to any molecule described as containing one or more optional substituents, only sterically practical and/or synthetically feasible compounds are meant to be included.

As used herein, “optionally substituted” refers to all subsequent modifiers in a term or series of chemical groups. For example, in the term “optionally substituted arylalkyl, the “alkyl” portion and the “aryl” portion of the molecule may or may not be substituted, and for the series “optionally substituted alkyl, cycloalkyl, aryl and heteroaryl,” the alkyl, cycloalkyl, aryl, and heteroaryl groups, independently of the others, may or may not be substituted.

### Engineered Enzyme Polypeptides

Ketoreductase (KRED) or carbonyl reductase biocatalysts (EC 1.1.1.184) are useful for the synthesis of alcohols from aldehydes and ketones, and optically active secondary alcohols from the corresponding prostereoisomeric ketone substrates. KREDs may also catalyze the reverse reaction, (i.e., oxidation of an alcohol substrate to the corresponding aldehydes/ketone product). The reduction of aldehydes and ketones and the oxidation of alcohols by KREDs uses a co-factor, most commonly reduced nicotinamide adenine dinucleotide (NADH) or reduced nicotinamide adenine dinucleotide phosphate (NADPH), and nicotinamide adenine dinucleotide (NAD) or nicotinamide adenine dinucleotide phosphate (NADP+) for the oxidation reaction. NADH and NADPH serve as electron donors, while NAD+ and NADP+ serve as electron acceptors.

KREDs can be found in a wide range of bacteria and yeasts, as known in the art (See e.g., Hummel and Kula Eur. J. Biochem., 184:1-13 [1989]). Numerous KRED genes and enzyme sequences have been reported, including those of Candida magnoliae (Genbank Acc. No. JC7338; GI: 11360538); Candida parapsilosis (Genbank Acc. No. BAA24528.1; GI:2815409), Sporobolomyces salmonicolor (Genbank Acc. No. AF160799; GI:6539734), Lactobacillus kefr (Genbank Acc. No. AAP94029.1; GI: 33112056), Lactobacillus brevis (Genbank Acc. No. 1NXQ_A; GI: 30749782), and Thermoanaerobium brockii (Genbank Acc. No. P14941; GI: 1771790).

The stereoselectivity of ketoreductases have been applied to the preparation of important pharmaceutical building blocks (See e.g., Broussy et al., Org. Lett., 11:305-308 [2009]). Specific applications of naturally occurring or engineered KREDs in biocatalytic processes to generate useful chemical compounds have been demonstrated for reduction of 4-chloroacetoacetate esters (See e.g,. Zhou, J. Am. Chem. Soc.,105:5925-5926 [1983]; Santaniello, J. Chem. Res., (S)132-133 [1984]; U.S. Pat. Nos. 5,559,030, 5,700,670, 5,891,685), reduction of dioxocarboxylic acids (See e.g., U.S. Pat. No. 6,399,339), reduction oftert-butyl (S)-chloro-5-hydroxy-3-oxohexanoate (See e.g., U.S. Pat. No. 6,645,746; and WO 01/40450), reduction pyrrolotriazine-based compounds (See e.g., U.S. Appln. Publ. No. 2006/0286646); reduction of substituted acetophenones (See e.g., U.S. Pat. Nos. 6,800,477 and 8,748,143); and reduction of ketothiolanes (WO 2005/054491).

The present invention provides engineered ketoreductases capable of deracemizing the substrate compound (1), (6,7-dihydro-5H-pyrrolo[1,2-a]imidazol-7-ol), in one-pot, multi-enzyme system as shown in the following reaction and FIG. 1.

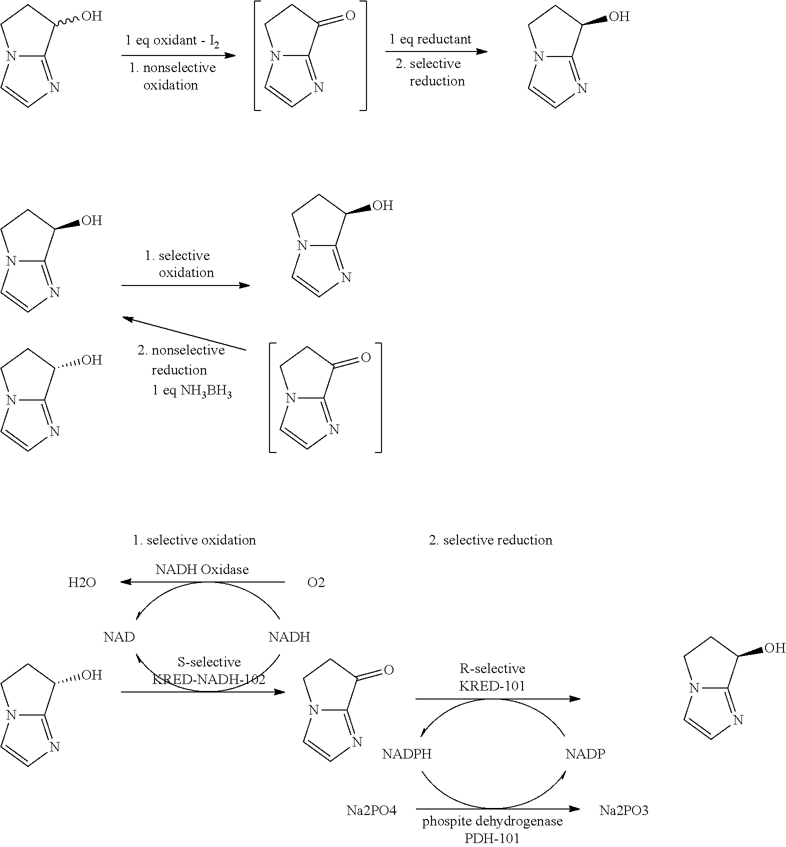

The present invention further provides improved ketoreductase enzymes and improved phosphite dehydrogenase enzymes, and methods for using the engineered ketoreductase and phosphite dehydrogenase enzymes to deracemize chiral compounds in one-pot, multi-enzyme system.

It is important to note that the desired product can be obtained in a one-pot, one-step, multi-enzyme system only if the oxidation and reduction reactions are orthogonal, compatible and non-interacting. These conditions are only satisfied if the oxidative ketoreductase and its corresponding recycling enzyme use one co-factor exclusively (e.g., NAD+), and reductive ketoreductase and its corresponding recycling enzyme use the opposite co-factor exclusively (i.e., NADPH).

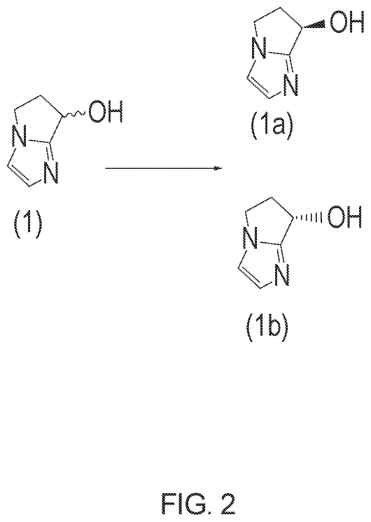

Compound (1) has one chiral center and can exist in two different diastereomeric forms (1a and 1b). The deracemization reaction by a tandem of ketoreductases can result in two different enantiomeric products (1a-1b), as shown in FIG. 2 and below.

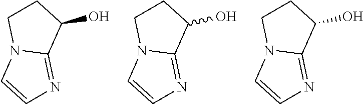

However, (1a) is the only desired product. The evolution program used in the development of the present invention was designed to improve activity of an S-selective ketoreductase that would oxidize the S-alcohol in the racemic mixture, generating a ketone substrate for an R-selective ketoreductase. Further, evolution program was designed to improve the selectivity, activity and cofactor preference of the R-selective ketoreductase. Evolution was also designed to improve activity, stability and cofactor preference of a phosphite dehydrogenase to enable deracemization of substrate (1) to product (1a) with minimal amount of ketone and (1b) in a one-pot, one-step, multi-enzyme process.

The ketoreductase polypeptide of SEQ ID NO:2 was selected as the initial backbone for development of the improved S-selective enzymes provided by the present invention. This enzyme was chosen as the starting backbone as ketone (2) was produced via oxidation of only (1b), leaving (1a). The ketoreductase polypeptide of SEQ ID NO:2 uses NAD⁺ as a co-factor with an efficiency greater than 200:1 over NADP⁺ and can be coupled with a commercially available NADH oxidase to recycle the co-factor.

The ketoreductase polypeptide of SEQ ID NO:2 was selected as the initial backbone for development of R-selective enzymes to reduce a ketone to product (1a) with initial selectivity of 92.7% e.e. Enantioselectivity values are calculated herein according to equation (1) provided below.

{[(1a amount)−(1b amount)]/[(1a amount)+(1b amount)]}×100 (1)

Indeed, the non-naturally occurring ketoreductase polypeptides of the present invention are ketoreductases engineered to have improved properties as compared to the naturally occurring ketoreductase of SEQ ID NO:2.

A phosphite dehydrogenase polypeptide was selected as the initial backbone for development of the improved PDH enzymes. This enzyme is equally efficient recycling both NADH and NADPH.

In some embodiments, the engineered ketoreductase polypeptides are capable of converting the substrate compound to product with an activity that is increased at least about 1.2 fold, 1.5 fold, 2 fold, 3 fold, 4 fold, 5 fold, 10 fold, 20 fold, 30 fold, 40 fold, 50 fold, or 100 fold relative to the activity of the reference polypeptide of SEQ ID NO:2 under suitable reaction conditions. In some embodiments, the engineered ketoreductase polypeptides are capable of converting the substrate compound to product with a percent conversion of at least about 40%, at least about 50%, at least about 60%, at least about 70%, at least about 80%, or at least about 90%, at least about 95%, at least about 98%, at least about 99%, in a reaction time of about 48 h, about 36 h, about 24 h, or even shorter length of time, under suitable reaction conditions.

In some embodiments, the engineered ketoreductases and phosphite dehydrogenases are capable of converting substrate compound (1) to product compound (1a) in enantiomeric excess over compound (1b) in a one-pot, one-step, multi-enzyme system. In some embodiments, the engineered ketoreductases and phosphite dehydrogenases are capable of converting compound (1) to compound (1a) in diastereomeric excess over compound (1b) under suitable reaction conditions.

As will be appreciated by those of skill in the art, some of the above-defined categories, unless otherwise specified, are not mutually exclusive. Thus, amino acids having side chains exhibiting two or more physico-chemical properties can be included in multiple categories. The appropriate classification of any amino acid or residue will be apparent to those of skill in the art, especially in light of the detailed invention provided herein.

In some embodiments, the improved engineered ketoreductase enzymes and engineered phosphite dehydrogenase enzymes comprise amino acid residue deletions in the naturally occurring ketoreductase or phosphite dehydrogenase polypeptides or deletions of amino acid residues in other engineered ketoreductase or phosphite dehydrogenase polypeptides. Thus, in some embodiments of the invention, the deletions comprise one or more amino acids, 2 or more amino acids, 3 or more amino acids, 4 or more amino acids, 5 or more amino acids, 6 or more amino acids, 8 or more amino acids, 10 or more amino acids, 15 or more amino acids, or 20 or more amino acids, up to 10% of the total number of amino acids, up to 10% of the total number of amino acids, up to 20% of the total number of amino acids, or up to 30% of the total number of amino acids of the ketoreductase polypeptides, as long as the functional activity of the ketoreductase or phosphite dehydrogenase activity is maintained. In some embodiments, the deletions can comprise, 1-2, 1-3, 1-4, 1-5, 1-6, 1-7, 1-8, 1-9, 1-10, 1-11, 1-12, 1-14, 1-15, 1-16, 1-18, 1-20, 1-22, 1-24, 1-25, 1-30, 1-35 or about 1-40 amino acid residues. In some embodiments, the number of deletions can be 1, 2, 3, 4, 5, 6, 7, 8, 9, 10, 11, 12, 14, 15, 16, 18, 20, 22, 24, 26, 30, 35 or about 40 amino acids. In some embodiments, the deletions can comprise deletions of 1, 2, 3, 4, 5, 6, 7, 8, 9, 10, 11, 12, 13, 14, 15, 16, 18, or 20 amino acid residues.

As described herein, the ketoreductase or phosphite dehydrogenase polypeptides of the invention can be in the form of fusion polypeptides in which the ketoreductases or phosphite dehydrogenase polypeptides are fused to other polypeptides, such as antibody tags (e.g., myc epitope) or purifications sequences (e.g., His tags). Thus, in some embodiments, the ketoreductase and/or phosphite dehydrogenase polypeptides find use with or without fusions to other polypeptides.

In some embodiments, the polypeptides described herein are not restricted to the genetically encoded amino acids. In addition to the genetically encoded amino acids, the polypeptides described herein may be comprised, either in whole or in part, of naturally-occurring and/or synthetic non-encoded amino acids. Certain commonly encountered non-encoded amino acids of which the polypeptides described herein may be comprised include, but are not limited to: the D-stereomers of the genetically-encoded amino acids; 2,3-diaminopropionic acid (Dpr); α-aminoisobutyric acid (Aib); ε-aminohexanoic acid (Aha); δ-aminovaleric acid (Ava); N-methylglycine or sarcosine (MeGly or Sar); ornithine (Orn); citrulline (Cit); t-butylalanine (Bua); t-butylglycine (Bug); N-methylisoleucine (MeIle); phenylglycine (Phg); cyclohexylalanine (Cha); norleucine (Nle); naphthylalanine (Nal); 2-chlorophenylalanine (Ocf); 3-chlorophenylalanine (Mcf); 4-chlorophenylalanine (Pcf); 2-fluorophenylalanine (Off); 3-fluorophenylalanine (Mff); 4-fluorophenylalanine (Pff); 2-bromophenylalanine (Obf); 3-bromophenylalanine (Mbf); 4-bromophenylalanine (Pbf); 2-methylphenylalanine (Omf); 3-methylphenylalanine (Mmf); 4-methylphenylalanine (Pmf); 2-nitrophenylalanine (Onf); 3-nitrophenylalanine (Mnf); 4-nitrophenylalanine (Pnf); 2-cyanophenylalanine (Ocf); 3-cyanophenylalanine (Mcf); 4-cyanophenylalanine (Pcf); 2-trifluoromethylphenylalanine (Otf); 3-trifluoromethylphenylalanine (Mtf); 4-trifluoromethylphenylalanine (Ptf); 4-aminophenylalanine (Paf); 4-iodophenylalanine (Pif); 4-aminomethylphenylalanine (Pamf); 2,4-dichlorophenylalanine (Opef); 3,4-dichlorophenylalanine (Mpcf); 2,4-difluorophenylalanine (Opff); 3,4-difluorophenylalanine (Mpff); pyrid-2-ylalanine (2pAla); pyrid-3-ylalanine (3pAla); pyrid-4-ylalanine (4pAla); naphth-1-ylalanine (1nAla); naphth-2-ylalanine (2nAla); thiazolylalanine (taAla); benzothienylalanine (bAla); thienylalanine (tAla); furylalanine (fAla); homophenylalanine (hPhe); homotyrosine (hTyr); homotryptophan (hTrp); pentafluorophenylalanine (5ff); styrylkalanine (sAla); authrylalanine (aAla); 3,3-diphenylalanine (Dfa); 3-amino-5-phenypentanoic acid (Afp); penicillamine (Pen); 1,2,3,4-tetrahydroisoquinoline-3-carboxylic acid (Tic); β-2-thienylalanine (Thi); methionine sulfoxide (Mso); N(w)-nitroarginine (nArg); homolysine (hLys); phosphonomethylphenylalanine (pmPhe); phosphoserine (pSer); phosphothreonine (pThr); homoaspartic acid (hAsp); homoglutanic acid (hGlu); 1-aminocyclopent-(2 or 3)-ene-4 carboxylic acid; pipecolic acid (PA), azetidine-3-carboxylic acid (ACA); 1-aminocyclopentane-3-carboxylic acid; allylglycine (aOly); propargylglycine (pgGly); homoalanine (hAla); norvaline (nVal); homoleucine (hLeu), homovaline (hVal); homoisolencine (hIle); homoarginine (hArg); N-acetyl lysine (AcLys); 2,4-diaminobutyric acid (Dbu); 2,3-diaminobutyric acid (Dab); N-methylvaline (MeVal); homocysteine (hCys); homoserine (hSer); hydroxyproline (Hyp) and homoproline (hPro). Additional non-encoded amino acids of which the polypeptides described herein may be comprised are apparent to those of skill in the art. These amino acids may be in either the L- or D-configuration.

Those of skill in the art will recognize that amino acids or residues bearing side chain protecting groups may also comprise the polypeptides described herein. Non-limiting examples of such protected amino acids, which in this case belong to the aromatic category, include (protecting groups listed in parentheses), but are not limited to: Arg(tos), Cys(methylbenzyl), Cys (nitropyridinesulfenyl), Glu(8-benzylester), Gln(xanthyl), Asn(N-δ-xanthyl), His(bom), His(benzyl), His(tos), Lys(finoc), Lys(tos), Ser(O-benzyl), Thr (O-benzyl) and Tyr(O-benzyl).

Non-encoding amino acids that are conformationally constrained of which the polypeptides described herein may be composed include, but are not limited to, N-methyl amino acids (L-configuration); 1-aminocyclopent-(2 or 3)-ene-4-carboxylic acid; pipecolic acid; azetidine-3-carboxylic acid; homoproline (hPro); and 1-aminocyclopentane-3-carboxylic acid.

As described above the various modifications introduced into the naturally occurring polypeptide to generate an engineered ketoreductase enzymes and engineered phosphite dehydrogenase enzymes can be targeted to a specific property of the enzyme.

### Polynucleotides Encoding Engineered Enzymes

In another aspect, the present invention provides polynucleotides encoding the engineered ketoreductase enzymes and engineered phosphite dehydrogenase enzymes. The polynucleotides may be operatively linked to one or more heterologous regulatory sequences that control gene expression to create a recombinant polynucleotide capable of expressing the polypeptide. Expression constructs containing a heterologous polynucleotide encoding the engineered ketoreductase and/or engineered phosphite dehydrogenase can be introduced into appropriate host cells to express the corresponding ketoreductase or phosphite dehydrogenase polypeptide.

Because of the knowledge of the codons corresponding to the various amino acids, availability of a protein sequence provides a description of all the polynucleotides capable of encoding the subject. The degeneracy of the genetic code, where the same amino acids are encoded by alternative or synonymous codons allows an extremely large number of nucleic acids to be made, all of which encode the improved ketoreductase enzymes and/or improved phosphite dehydrogenase enzymes disclosed herein. Thus, having identified a particular amino acid sequence, those skilled in the art could make any number of different nucleic acids by simply modifying the sequence of one or more codons in a way which does not change the amino acid sequence of the protein. In this regard, the present invention specifically contemplates each and every possible variation of polynucleotides that could be made by selecting combinations based on the possible codon choices, and all such variations are to be considered specifically disclosed for any polypeptide disclosed herein, including the amino acid sequences presented in the Tables in the Examples. In various embodiments, the codons are preferably selected to fit the host cell in which the protein is being produced. For example, preferred codons used in bacteria are used to express the gene in bacteria; preferred codons used in yeast are used for expression in yeast; and preferred codons used in mammals are used for expression in mammalian cells.

In some embodiments, the engineered ketoreductase or phosphite dehdyrogeanse sequences comprise sequences that comprise positions identified to be beneficial, as described in the Examples.

In some embodiments, isolated polynucleotides encoding an improved ketoreductase or phosphite dehydrogenase polypeptides are manipulated in a variety of ways to provide for improved expression and/or production of the polypeptides. Manipulation of the isolated polynucleotide prior to its insertion into a vector may be desirable or necessary, depending on the expression vector used. The techniques for modifying polynucleotides and nucleic acid sequences utilizing recombinant DNA methods are well known in the art.

For bacterial host cells, suitable promoters for directing transcription of the nucleic acid constructs of the present invention, include the promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus lichenformis penicillinase gene (penP), Bacillus subtilis xylA and xylB genes, and prokaryotic beta-lactamase gene (See e.g., Villa-Kamaroff et al., Proc. Natl. Acad. Sci. USA 75: 3727-3731 [1978]), as well as the tac promoter (See e.g., DeBoer et al., Proc. Natl Acad. Sci. USA 80: 21-25 [1983]). Additional suitable promoters are known to those in the art.

For filamentous fungal host cells, suitable promoters for directing the transcription of the nucleic acid constructs of the present invention include promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, and Fusarium oxysporum trypsin-like protease (WO 96/00787), as well as the NA2-tpi promoter (a hybrid of the promoters from the genes for Aspergillus niger neutral alpha-amylase and Aspergillus oryzae triose phosphate isomerase), and mutant, truncated, and hybrid promoters thereof.

In a yeast host, useful promoters include, but are not limited to those from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH2/GAP), and Saccharomyces cerevisiae 3-phosphoglycerate kinase, as well as other useful promoters for yeast host cells (See e.g., Romanos et al., Yeast 8:423-488 [1992]).

The control sequence may also be a suitable transcription terminator sequence, a sequence recognized by a host cell to terminate transcription. The terminator sequence is operably linked to the 3′ terminus of the nucleic acid sequence encoding the polypeptide. Any terminator that is functional in the host cell of choice may be used in the present invention.

For example, exemplary transcription terminators for filamentous fungal host cells can be obtained from the genes for Aspergillus oryzae TAKA amylase, Aspergillus niger glucoamylase, Aspergillus nidulans anthranilate synthase, Aspergillus niger alpha-glucosidase, and Fusarium oxysporum trypsin-like protease.

Exemplary terminators for yeast host cells can be obtained from the genes for Saccharomyces cerevisiae enolase, Saccharomyces cerevisiae cytochrome C (CYC1), and Saccharomyces cerevisiae glyceraldehyde-3-phosphate dehydrogenase, as well as other useful terminators for yeast host cells known in the art (See e.g,. Romanos et al., supra).

The control sequence may also be a suitable leader sequence, a nontranslated region of an mRNA that is important for translation by the host cell. The leader sequence is operably linked to the 5′ terminus of the nucleic acid sequence encoding the polypeptide. Any leader sequence that is functional in the host cell of choice may be used. Exemplary leaders for filamentous fungal host cells are obtained from the genes for Aspergillus oryzae TAKA amylase and Aspergillus nidulans triose phosphate isomerase. Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′ terminus of the nucleic acid sequence and which, when transcribed, is recognized by the host cell as a signal to add polyadenosine residues to transcribed mRNA. Any polyadenylation sequence which is functional in the host cell of choice may be used in the present invention. Exemplary polyadenylation sequences for filamentous fungal host cells can be from the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase, as well as additional useful polyadenylation sequences for yeast host cells known in the art (See e.g., Guo et al., Mol. Cell. Biol., 15:5983-5990 [1995]).

The control sequence may also be a signal peptide coding region that codes for an amino acid sequence linked to the amino terminus of a polypeptide and directs the encoded polypeptide into the cell's secretory pathway. The 5′ end of the coding sequence of the nucleic acid sequence may inherently contain a signal peptide coding region naturally linked in translation reading frame with the segment of the coding region that encodes the secreted polypeptide. Alternatively, the 5′ end of the coding sequence may contain a signal peptide coding region that is foreign to the coding sequence. The foreign signal peptide coding region may be required where the coding sequence does not naturally contain a signal peptide coding region.

Alternatively, the foreign signal peptide coding region may simply replace the natural signal peptide coding region in order to enhance secretion of the polypeptide. However, any signal peptide coding region which directs the expressed polypeptide into the secretory pathway of a host cell of choice may be used in the present invention.

Effective signal peptide coding regions for bacterial host cells are the signal peptide coding regions obtained from the genes for Bacillus NC1B 11837 maltogenic amylase, Bacillus stearothermophilus alpha-amylase, Bacillus licheniformis subtilisin, Bacillus licheniformis beta-lactamase, Bacillus stearothermophilus neutral proteases (nprT, nprS, nprM), and Bacillus subtilis prsA, as well as additional signal peptides known in the art (See e.g., Simonen et al., Microbiol. Rev., 57: 109-137 [1993]).

Effective signal peptide coding regions for filamentous fungal host cells include, but are not limited to the signal peptide coding regions obtained from the genes for Aspergillus oryzae TAKA amylase, Aspergillus niger neutral amylase, Aspergillus niger glucoamylase, Rhizomucor miehei aspartic proteinase, Humicola insolens cellulase, and Humicola lanuginosa lipase. Useful signal peptides for yeast host cells can be from the genes for Saccharomyces cerevisiae alpha-factor and Saccharomyces cerevisiae invertase, as well as additional useful signal peptide coding regions (See e.g., Romanos et al., 1992, supra).

The control sequence may also be a propeptide coding region that codes for an amino acid sequence positioned at the amino terminus of a polypeptide. The resultant polypeptide is known as a proenzyme or propolypeptide (or a zymogen in some cases). A propolypeptide is generally inactive and can be converted to a mature active polypeptide by catalytic or autocatalytic cleavage of the propeptide from the propolypeptide. The propeptide coding region may be obtained from the genes for Bacillus subtilis alkaline protease (aprE), Bacillus subtilis neutral protease (nprT), Saccharomyces cerevisiae alpha-factor, Rhizomucor miehei aspartic proteinase, and Myceliophthora thermophila lactase (WO 95/33836).

Where both signal peptide and propeptide regions are present at the amino terminus of a polypeptide, the propeptide region is positioned next to the amino terminus of a polypeptide and the signal peptide region is positioned next to the amino terminus of the propeptide region.

It may also be desirable to add regulatory sequences, which allow the regulation of the expression of the polypeptide relative to the growth of the host cell. Examples of regulatory systems are those which cause the expression of the gene to be turned on or off in response to a chemical or physical stimulus, including the presence of a regulatory compound. In prokaryotic host cells, suitable regulatory sequences include the lac, tac, and trp operator systems. In yeast host cells, suitable regulatory systems include, as examples, the ADH2 system or GAL1 system. In filamentous fungi, suitable regulatory sequences include the TAKA alpha-amylase promoter, Aspergillus niger glucoamylase promoter, and Aspergillus oryzae glucoamylase promoter.

Other examples of regulatory sequences are those which allow for gene amplification. In eukaryotic systems, these include the dihydrofolate reductase gene, which is amplified in the presence of methotrexate, and the metallothionein genes, which are amplified with heavy metals. In these cases, the nucleic acid sequence encoding the KRED polypeptide of the present invention or the PDH polypeptide of the present invention would be operably linked with the regulatory sequence.

Thus, in some embodiments, the present invention is also directed to a recombinant expression vector comprising a polynucleotide encoding an engineered ketoreductase polypeptide or a variant thereof, or an engineered phosphite dehydrogenase polypeptide or a variant thereof, and one or more expression regulating regions such as a promoter and a terminator, a replication origin, etc., depending on the type of hosts into which they are to be introduced. The various nucleic acid and control sequences described above may be joined together to produce a recombinant expression vector which may include one or more convenient restriction sites to allow for insertion or substitution of the nucleic acid sequence encoding the polypeptide at such sites. Alternatively, the nucleic acid sequence of the present invention may be expressed by inserting the nucleic acid sequence or a nucleic acid construct comprising the sequence into an appropriate vector for expression. In creating the expression vector, the coding sequence is located in the vector so that the coding sequence is operably linked with the appropriate control sequences for expression.

The recombinant expression vector may be any vector (e.g., a plasmid or virus), which can be conveniently subjected to recombinant DNA procedures and can bring about the expression of the polynucleotide sequence. The choice of the vector will typically depend on the compatibility of the vector with the host cell into which the vector is to be introduced. The vectors may be linear or closed circular plasmids.

The expression vector may be an autonomously replicating vector (i.e., a vector that exists as an extrachromosomal entity), the replication of which is independent of chromosomal replication, (e.g., a plasmid, an extrachromosomal element, a minichromosome, or an artificial chromosome). The vector may contain any means for assuring self-replication. Alternatively, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used.

The expression vector of the present invention preferably contains one or more selectable markers, which permit easy selection of transformed cells. A selectable marker can be a gene the product of which provides for biocide or viral resistance, resistance to heavy metals, prototrophy to auxotrophs, and the like. Examples of bacterial selectable markers are the dal genes from Bacillus subtilis or Bacillus licheniformis, or markers, which confer antibiotic resistance such as ampicillin, kanamycin, chloramphenicol, or tetracycline resistance. Suitable markers for yeast host cells are ADE2, HIS3, LEU2, LYS2, MET3, TRP1, and URA3.

Selectable markers for use in a filamentous fungal host cell include, but are not limited to, amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Embodiments for use in an Aspergillus cell include the amdS and pyrG genes ofAspergillus nidulans or Aspergillus oryzae and the bar gene of Streptomyces hygroscopicus.

The expression vectors of the present invention can contain an element(s) that permits integration of the vector into the host cell's genome or autonomous replication of the vector in the cell independent of the genome. For integration into the host cell genome, the vector may rely on the nucleic acid sequence encoding the polypeptide or any other element of the vector for integration of the vector into the genome by homologous or nonhomologous recombination.

Alternatively, the expression vector may contain additional nucleic acid sequences for directing integration by homologous recombination into the genome of the host cell. The additional nucleic acid sequences enable the vector to be integrated into the host cell genome at a precise location(s) in the chromosome(s). To increase the likelihood of integration at a precise location, the integrational elements should preferably contain a sufficient number of nucleic acids, such as 100 to 10,000 base pairs, preferably 400 to 10,000 base pairs, and most preferably 800 to 10,000 base pairs, which are highly homologous with the corresponding target sequence to enhance the probability of homologous recombination. The integrational elements may be any sequence that is homologous with the target sequence in the genome of the host cell. Furthermore, the integrational elements may be non-encoding or encoding nucleic acid sequences. On the other hand, the vector may be integrated into the genome of the host cell by non-homologous recombination.

For autonomous replication, the vector may further comprise an origin of replication enabling the vector to replicate autonomously in the host cell in question. Examples of bacterial origins of replication are P15A on or the origins of replication of plasmids pBR322, pUC19, pACYC177 (which plasmid has the P15A on), or pACYC184 permitting replication in E. coli, and pUB110, pE194, pTA1060, or pAMβ1 permitting replication in Bacillus. Examples of origins of replication for use in a yeast host cell are the 2 micron origin of replication, ARS1, ARS4, the combination of ARS1 and CEN3, and the combination of ARS4 and CEN6. The origin of replication may be one having a mutation which makes its functioning temperature-sensitive in the host cell (See e.g., Ehrlich, Proc. Natl. Acad. Sci. USA 75:1433 [1978]).

More than one copy of a nucleic acid sequence of the present invention may be inserted into the host cell to increase production of the gene product. An increase in the copy number of the nucleic acid sequence can be obtained by integrating at least one additional copy of the sequence into the host cell genome or by including an amplifiable selectable marker gene with the nucleic acid sequence where cells containing amplified copies of the selectable marker gene, and thereby additional copies of the nucleic acid sequence, can be selected for by cultivating the cells in the presence of the appropriate selectable agent.

Many of the expression vectors for use in the present invention are commercially available. Suitable commercial expression vectors include, but are not limited to p3×FLAGTM™ expression vectors (Sigma-Aldrich), which include a CMV promoter and hGH polyadenylation site for expression in mammalian host cells and a pBR322 origin of replication and ampicillin resistance markers for amplification in E. coli. Other commercially available suitable expression vectors include but are not limited to the pBluescriptII SK(−) and pBK-CMV vectors (Stratagene), and plasmids derived from pBR322 (Gibco BRL), pUC (Gibco BRL), pREP4, pCEP4 (Invitrogen) or pPoly (See, Lathe et al., Gene 57:193-201 [1987]).

### Host Cells for Expression of Engineered Polypeptides

The present invention also provides a host cell comprising a polynucleotide encoding an improved ketoreductase polypeptide or an improved phosphite dehydrogenase polypeptide of the present invention, the polynucleotide being operatively linked to one or more control sequences for expression of the ketoreductase enzyme or the phosphite dehydrogenase enzyme in the host cell. Host cells for use in expressing the KRED polypeptides encoded by the expression vectors of the present invention or the PDH polypeptides encoded by the expression vectors of the present invention are well known in the art and include but are not limited to, bacterial cells, such as E. coli, Lactobacillus kefir, Lactobacillus brevis, Lactobacillus minor, Streptomyces and Salmonella typhimurium cells; fungal cells, such as yeast cells (e.g., Saccharomyces cerevisiae or Pichia pastoris (ATCC Accession No. 201178)); insect cells such as Drosophila S2 and Spodoptera Sf9 cells; animal cells such as CHO, COS, BHK, 293, and Bowes melanoma cells; and plant cells. Appropriate culture media and growth conditions for the above-described host cells are well known in the art.

Polynucleotides for expression of the ketoreductase or the phosphite dehydrogenase may be introduced into cells by various methods known in the art. Techniques include among others, electroporation, biolistic particle bombardment, liposome mediated transfection, calcium chloride transfection, and protoplast fusion. Various methods for introducing polynucleotides into cells will be apparent to the skilled artisan.

Escherichia coli W3110 is a host strain that finds use in the present invention, although it is not intended that the present invention be limited to this specific host strain. The expression vector was created by operatively linking a polynucleotide encoding an improved enzyme into the plasmid pCK110900 operatively linked to the lac promoter under control of the lacI repressor. The expression vector also contained the P15a origin of replication and the chloramphenicol resistance gene. Cells containing the subject polynucleotide in Escherichia coli W3110 can be isolated by subjecting the cells to chloramphenicol selection.

### Methods of Generating Engineered Ketoreductase Polypeptides and Engineered Phosphite Dehydrogenase Polypeptides.

In some embodiments, to make the improved KRED polynucleotides and polypeptides of the present invention, the naturally-occurring ketoreductase enzyme that catalyzes the reduction reaction is obtained (or derived) from Candida parasilosis or Sporodiobolus salmonicolor. In some embodiments, the parent polynucleotide sequence is codon optimized to enhance expression of the ketoreductase in a specified host cell. As an illustration, the parental polynucleotide sequence encoding the wild-type KRED polypeptide of Sporodiobolus salmonicolor was constructed from oligonucleotides prepared based upon the known polypeptide sequence of Sporodiobolus salmonicolor KRED sequence available from the Genbank database. The parental polynucleotide sequence was codon optimized for expression in E. coli and the codon-optimized polynucleotide cloned into an expression vector, placing the expression of the ketoreductase gene under the control of the lac promoter and lacI repressor gene. Clones expressing the active ketoreductase in E. coli were identified and the genes sequenced to confirm their identity.

In some embodiments, the engineered ketoreductases are obtained by subjecting the polynucleotide encoding the naturally occurring ketoreductase to mutagenesis and/or directed evolution methods, as discussed above. Mutagenesis may be performed in accordance with any of the techniques known in the art, including random and site-specific mutagenesis. Directed evolution can be performed with any of the techniques known in the art to screen for improved promoter variants including shuffling. Mutagenesis and directed evolution methods are well known in the art (See e.g., U.S. Pat. Nos. 5,605,793, 5,811,238, 5,830,721, 5,834,252, 5,837,458, 5,928,905, 6,096,548, 6,117,679, 6,132,970, 6,165,793, 6,180,406, 6,251,674, 6,265,201, 6,277,638, 6,287,861, 6,287,862, 6,291,242, 6,297,053, 6,303,344, 6,309,883, 6,319,713, 6,319,714, 6,323,030, 6,326,204, 6,335,160, 6,335,198, 6,344,356, 6,352,859, 6,355,484, 6,358,740, 6,358,742, 6,365,377, 6,365,408, 6,368,861, 6,372,497, 6,337,186, 6,376,246, 6,379,964, 6,387,702, 6,391,552, 6,391,640, 6,395,547, 6,406,855, 6,406,910, 6,413,745, 6,413,774, 6,420,175, 6,423,542, 6,426,224, 6,436,675, 6,444,468, 6,455,253, 6,479,652, 6,482,647, 6,483,011, 6,484,105, 6,489,146, 6,500,617, 6,500,639, 6,506,602, 6,506,603, 6,518,065, 6,519,065, 6,521,453, 6,528,311, 6,537,746, 6,573,098, 6,576,467, 6,579,678, 6,586,182, 6,602,986, 6,605,430, 6,613,514, 6,653,072, 6,686,515, 6,703,240, 6,716,631, 6,825,001, 6,902,922, 6,917,882, 6,946,296, 6,961,664, 6,995,017, 7,024,312, 7,058,515, 7,105,297, 7,148,054, 7,220,566, 7,288,375, 7,384,387, 7,421,347, 7,430,477, 7,462,469, 7,534,564, 7,620,500, 7,620,502, 7,629,170, 7,702,464, 7,747,391, 7,747,393, 7,751,986, 7,776,598, 7,783,428, 7,795,030, 7,853,410, 7,868,138, 7,783,428, 7,873,477, 7,873,499, 7,904,249, 7,957,912, 7,981,614, 8,014,961, 8,029,988, 8,048,674, 8,058,001, 8,076,138, 8,108,150, 8,170,806, 8,224,580, 8,377,681, 8,383,346, 8,457,903, 8,504,498, 8,589,085, 8,762,066, 8,768,871, 9,593,326, and all related non-US counterparts; Ling et al., Anal. Biochem., 254(2):157-78 [1997]; Dale et al., Meth. Mol. Biol., 57:369-74 [1996]; Smith, Ann. Rev. Genet., 19:423-462 [1985]; Botstein et al., Science, 229:1193-1201 [1985]; Carter, Biochem. J., 237:1-7 [1986]; Kramer et al., Cell, 38:879-887 [1984]; Wells et al., Gene, 34:315-323 [1985]; Minshull et al., Curr. Op. Chem. Biol., 3:284-290 [1999]; Christians et al., Nat. Biotechnol., 17:259-264 [1999]; Crameri et al., Nature, 391:288-291 [1998]; Crameri, et al., Nat. Biotechnol., 15:436-438 [1997]; Zhang et al., Proc. Nat. Acad. Sci. U.S.A., 94:4504-4509 [1997]; Crameri et al., Nat. Biotechnol., 14:315-319 [1996]; Stemmer, Nature, 370:389-391 [1994]; Stemmer, Proc. Nat. Acad. Sci. USA, 91:10747-10751 [1994]; WO 95/22625; WO 97/0078; WO 97/35966; WO 98/27230; WO 00/42651; WO 01/75767; and WO 2009/152336, all of which are incorporated herein by reference).

The clones obtained following mutagenesis treatment are screened for engineered ketoreductases having a desired improved enzyme property. Measuring enzyme activity from the expression libraries can be performed using the standard biochemistry technique of monitoring the rate of decrease (via a decrease in absorbance or fluorescence) of NADH or NADPH concentration, as it is converted into NAD⁺ or NADP⁺. In this reaction, the NADH or NADPH is consumed (oxidized) by the ketoreductase as the ketoreductase reduces a ketone substrate to the corresponding hydroxyl group. The rate of decrease of NADH or NADPH concentration, as measured by the decrease in absorbance or fluorescence, per unit time indicates the relative (enzymatic) activity of the KRED polypeptide in a fixed amount of the lysate (or a lyophilized powder made therefrom). The stereochemistry of the products can be ascertained by various known techniques, and as provided in the Examples. Where the improved enzyme property desired is thermal stability, enzyme activity may be measured after subjecting the enzyme preparations to a defined temperature and measuring the amount of enzyme activity remaining after heat treatments. Clones containing a polynucleotide encoding a ketoreductase are then isolated, sequenced to identify the nucleotide sequence changes (if any), and used to express the enzyme in a host cell.

Where the sequence of the engineered polypeptide is known, the polynucleotides encoding the enzyme can be prepared by standard solid-phase methods, according to known synthetic methods. In some embodiments, fragments of up to about 100 bases can be individually synthesized, then joined (e.g., by enzymatic or chemical ligation methods, or polymerase mediated methods) to form any desired continuous sequence. For example, polynucleotides and oligonucleotides of the invention can be prepared by chemical synthesis (e.g., using the classical phosphoramidite method described by Beaucage et al., Tet. Lett., 22:1859-69 [1981], or the method described by Matthes et al., EMBO J., 3:801-05 [1984], as it is typically practiced in automated synthetic methods). According to the phosphoramidite method, oligonucleotides are synthesized (e.g., in an automatic DNA synthesizer), purified, annealed, ligated and cloned in appropriate vectors. In addition, essentially any nucleic acid can be obtained from any of a variety of commercial sources (e.g., The Midland Certified Reagent Company, Midland, TX, The Great American Gene Company, Ramona, CA, ExpressGen Inc. Chicago, IL, Operon Technologies Inc., Alameda, CA, and many others).

Engineered ketoreductase enzymes and engineered phosphite dehydrogenase enzymes expressed in a host cell can be recovered from the cells and or the culture medium using any one or more of the well known techniques for protein purification, including, among others, lysozyme treatment, sonication, filtration, salting-out, ultra-centrifugation, and chromatography. Suitable solutions for lysing and the high efficiency extraction of proteins from bacteria, such as E. coli, are commercially available under the trade name CelLytic B™ (Sigma-Aldrich).

Chromatographic techniques for isolation of the ketoreductase and/or phosphite dehydrogenase polypeptides include, among others, reverse phase chromatography high performance liquid chromatography, ion exchange chromatography, gel electrophoresis, and affinity chromatography. Conditions for purifying a particular enzyme will depend, in part, on factors such as net charge, hydrophobicity, hydrophilicity, molecular weight, molecular shape, etc., and will be apparent to those having skill in the art.

In some embodiments, affinity techniques are used to isolate the improved ketoreductase enzymes and/or improved phosphite dehydrogenase enzymes. For affinity chromatography purification, any antibody which specifically binds the ketoreductase polypeptide or the phosphite dehydrogenase polypeptide may be used. For the production of antibodies, various host animals, including but not limited to rabbits, mice, rats, etc., may be immunized by injection with the ketoreductase or the phosphite dehydrogenase. The ketoreductase polypeptide may be attached to a suitable carrier, such as BSA, by means of a side chain functional group or linkers attached to a side chain functional group. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (Bacillus Calmette Guerin) and Corynebacterium parvum.

The ketoreductases and/or the phosphite dehydrogenases may be prepared and used in the form of cells expressing the enzymes, as crude extracts, or as isolated or purified preparations. The ketoreductases and/or the phosphite dehydrogenases may be prepared as lyophilizates, in powder form (e.g., acetone powders), or prepared as enzyme solutions. In some embodiments, the ketoreductases or the phosphite dehydrogenases can be in the form of substantially pure preparations.

In some embodiments, the ketoreductase polypeptides and/or the phosphite dehydrogenase polypeptides can be attached to a solid substrate. The substrate can be a solid phase, surface, and/or membrane. A solid support can be composed of organic polymers such as polystyrene, polyethylene, polypropylene, polyfluoroethylene, polyethyleneoxy, and polyacrylamide, as well as co-polymers and grafts thereof. A solid support can also be inorganic, such as glass, silica, controlled pore glass (CPG), reverse phase silica or metal, such as gold or platinum. The configuration of the substrate can be in the form of beads, spheres, particles, granules, a gel, a membrane or a surface. Surfaces can be planar, substantially planar, or non-planar. Solid supports can be porous or non-porous, and can have swelling or non-swelling characteristics. A solid support can be configured in the form of a well, depression, or other container, vessel, feature, or location. A plurality of supports can be configured on an array at various locations, addressable for robotic delivery of reagents, or by detection methods and/or instruments.

As is known by those of skill in the art, ketoreductase-catalyzed reduction reactions typically require a cofactor. Reduction reactions catalyzed by the engineered ketoreductase enzymes described herein also typically require a cofactor, although many embodiments of the engineered ketoreductases require far less cofactor than reactions catalyzed with wild-type ketoreductase enzymes. As used herein, the term “cofactor” refers to a non-protein compound that operates in combination with a ketoreductase enzyme. Cofactors suitable for use with the engineered ketoreductase enzymes described herein include, but are not limited to, NADP⁺ (nicotinamide adenine dinucleotide phosphate), NADPH (the reduced form of NADP⁺), NAD⁺ (nicotinamide adenine dinucleotide) and NADH (the reduced form of NAD⁺). Generally, the reduced form of the cofactor is added to the reaction mixture. The reduced NAD(P)H form can be optionally regenerated from the oxidized NAD(P)⁺ form using a cofactor regeneration system. The term “cofactor regeneration system” refers to a set of reactants that participate in a reaction that reduces the oxidized form of the cofactor (e.g., NADP⁺ to NADPH). Cofactors oxidized by the ketoreductase-catalyzed reduction of the keto substrate are regenerated in reduced form by the cofactor regeneration system. Cofactor regeneration systems comprise a stoichiometric reductant that is a source of reducing hydrogen equivalents and is capable of reducing the oxidized form of the cofactor. The cofactor regeneration system may further comprise a catalyst, for example an enzyme catalyst, that catalyzes the reduction of the oxidized form of the cofactor by the reductant. Cofactor regeneration systems to regenerate NADH or NADPH from NAD⁺ or NADP⁺, respectively, are known in the art and may be used in the methods described herein.

### Experimental

Various features and embodiments of the invention are illustrated in the following representative examples, which are intended to be illustrative, and not limiting.

In the experimental disclosure below, the following abbreviations apply: ppm (parts per million); M (molar); mM (millimolar), uM and μM (micromolar); nM (nanomolar); mol (moles); gm and g (gram); mg (milligrams); ug and μg (micrograms); L and 1 (liter); ml and mL (milliliter); cm (centimeters); mm (millimeters); um and μm (micrometers); sec. (seconds); min(s) (minute(s)); h(s) and hr(s) (hour(s)); U (units); MW (molecular weight); rpm (rotations per minute); ° C. (degrees Centigrade); RT (room temperature); CDS (coding sequence); DNA (deoxyribonucleic acid); RNA (ribonucleic acid); HPLC (high performance liquid chromatography); FIOPC (fold improvement over positive control); HTP (high throughput); LB (Luria broth); Sigma-Aldrich (Sigma-Aldrich, St. Louis, MO); Millipore (Millipore, Corp., Billerica MA); Difco (Difco Laboratories, BD Diagnostic Systems, Detroit, MI); Daicel (Daicel, West Chester, PA); Genetix (Genetix USA, Inc., Beaverton, OR); Molecular Devices (Molecular Devices, LLC, Sunnyvale, CA); Applied Biosystems (Applied Biosystems, part of Life Technologies, Corp., Grand Island, NY), Agilent (Agilent Technologies, Inc., Santa Clara, CA); Thermo Scientific (part of Thermo Fisher Scientific, Waltham, MA); Corning (Corning, Inc., Palo Alto, CA); and Bio-Rad (Bio-Rad Laboratories, Hercules, CA).

### Example 1

### Ketoreductase and Phosphite Dehydrogenase Gene Construction and Expression Vectors

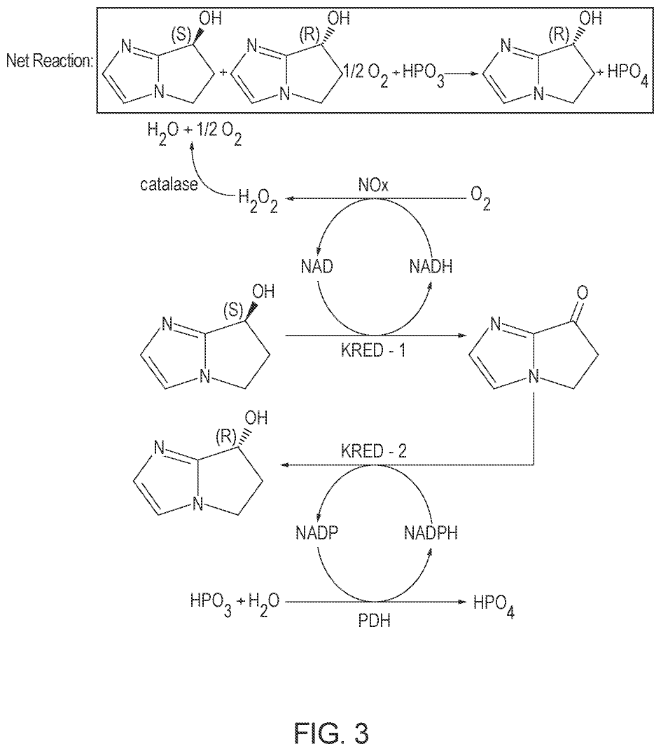

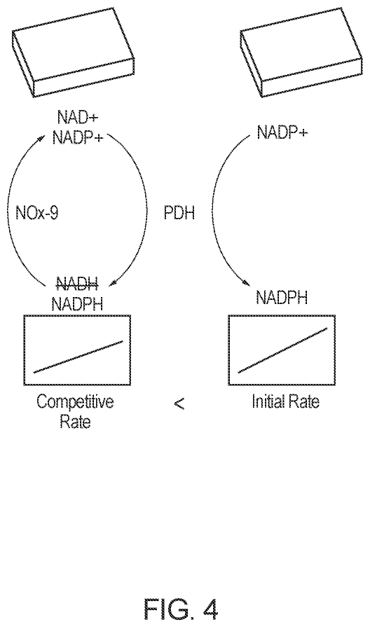

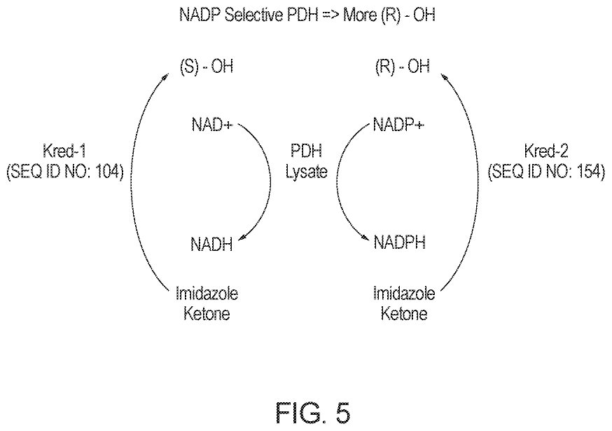

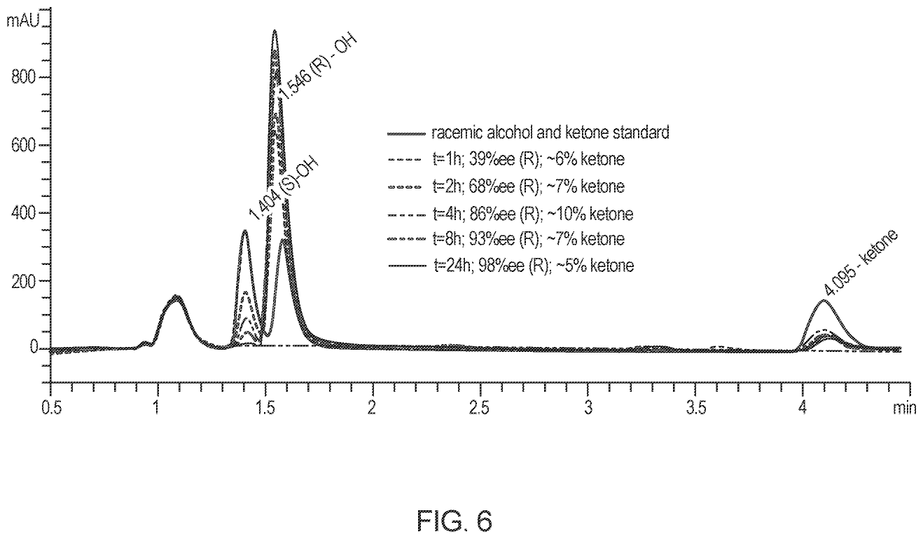

The wild-type Candida parapsilois ketoreductase (KRED) encoding gene was amplified from genomic DNA and cloned into expression vector pCK11 0900 (See, FIG. 3 of US Pat. Appln. Publn. No. 2006/0195947, herein incorporated by reference) under the control of a lac promoter. The expression vector also contained the P15a origin of replication and the chloramphenicol resistance gene. The activity of the wild-type ketoreductase was confirmed as described in WO2008/042876. Polynucleotides encoding engineered ketoreductases of the present invention were likewise cloned into vector pCK11 0900 for expression in E. coli W311 0. Directed evolution of the KRED gene was carried out by first selecting the parent gene (i.e., SEQ ID NOS: 2, 6, 104) followed by library construction of variant genes in which positions associated with certain structural features were subjected to mutagenesis. These libraries were then plated, grown-up, and screened using HTP assays as described in Examples 2, 5 and 12.

The wild-type Sporidiobolus salmonicolor ketoreductase (KRED) encoding gene was synthesized for expression in E. coli based on the reported amino acid sequence of the ketoreductase and a codon optimization algorithm as described in Example 1 of WO2008/042876, incorporated herein by reference. The gene was synthesized using oligonucleotides composed of 42 nucleotides and cloned into expression vector pCK11 0900 (See, FIG. 3 of US Pat. Appln. Pubin. No. 2006/0195947, herein incorporated by reference) under the control of a lac promoter. The expression vector also contained the P15a origin of replication and the chloramphenicol resistance gene. The activity of the wild-type ketoreductase was confirmed as described in WO2008/042876. Polynucleotides encoding engineered ketoreductases of the present invention were likewise cloned into vector pCK11 0900 for expression in E. coli W311 0. Directed evolution of the KRED gene was caried out by first selecting the parent gene (i.e., SEQ ID NOS: 112, 124, 138) followed by library construction of variant genes in which positions associated with certain structural features were subjected to mutagenesis. These libraries were then plated, grown, and screened using HTP assays as described in Examples 3, 6, 7, 8 and 12.

A variant of the wild-type Pseudomonas stutzeri phosphite dehydrogenase (PDH) encoding gene was cloned into expression vector pCK11 0900 (See, FIG. 3 of US Pat. Appln. Publn. No. 2006/0195947, herein incorporated by reference) under the control of a lac promoter. The expression vector also contained the P15a origin of replication and the chloramphenicol resistance gene. The activity of the phosphite dehydrogenase was confirmed as described in WO2008/042876.

Polynucleotides encoding engineered phosphite dehydrogenases of the present invention were likewise cloned into vector pCK11 0900 for expression in E. coli W311 0. Directed evolution of the PDH gene was carried out by first selecting the parent gene (i.e., SEQ ID NOS: 172, 182, 200, 208, 260) followed by library construction of variant genes in which positions associated with certain structural features were subjected to mutagenesis. These libraries were then plated, grown, and screened using HTP assays as described in Examples 4, and 9 through 12.

### Example 2

### Production and Analysis of Engineered KRED Polypeptides for Oxidation

Plasmid libraries obtained through directed evolution and containing evolved ketoreductase genes were transformed into E. coli W3110 and placed on Luria-Bertani (LB) agar medium containing 1% glucose and 30 μg/ml chloramphenicol (CAM). After incubation for at least 16 h at 30° C., colonies were picked using a Q-bot® robotic colony picker (Genetix) into a 96-well shallow well microtiter plate containing 200 μL of LB, 1% glucose, and 30 μg/ml CAM. Cells were grown 18-20 h at 30° C., with shaking at 200 rpm. Twenty L of this culture was then transferred to 360 μL of Terrific Broth (TB), 1 mM MgCl₂, 2 mM ZnSO₄ and 30 μg/ml CAM. After incubation of deep well plates at 30° C. with shaking at 250 rpm for 2.5 h (OD₆₀₀ 0.6-0.8), recombinant gene expression was induced by isopropyl thioglycoside (IPTG) to a final concentration of 1 mM. The plates were then incubated at 30° C. with shaking at 250 rpm for 18-21 h.

Cell cultures were pelleted at 3500×g for 20 min, and their supernatants were discarded. Cell pellets were lysed in 300 μL of 20 mM Tris, 2 mM ZnSO₄, 1 mM MgCl₂ pH 7.5 with 1 g/L lysozyme and 0.5 g/L polymixin B sulfate by shaking at RT for 2 h. Samples were centrifuged at 3500×g for 20 min to clarify cellular debris, and the supernatant was used to carry out the transformations described in Examples 5 and 12.

### Example 3

### Production and Analysis of Engineered KRED Polypeptides for Reduction

Plasmid libraries obtained through directed evolution and containing evolved ketoreductase genes were transformed into E. coli W3110 and placed on Luria-Bertani (LB) agar medium containing 1% glucose and 30 μg/ml chloramphenicol (CAM). After incubation for at least 16 h at 30° C., colonies were picked using a Q-bot® robotic colony picker (Genetix) into a 96-well shallow well microtiter plate containing 200 μL of LB, 1% glucose, and 30 μg/ml CAM. Cells were grown 18-20 h at 30° C., with shaking at 200 rpm. Twenty L of this culture was then transferred to 360 μL of Terrific Broth (TB), 1 mM MgSO₄, and 30 μg/ml CAM. After incubation of deep well plates at 30° C. with shaking at 250 rpm for 2.5 h (OD₆₀₀ 0.6-0.8), recombinant gene expression was induced by isopropyl thioglycoside (IPTG) to a final concentration of 1 mM. The plates were then incubated at 30° C. with shaking at 250 rpm for 18-21 h.

Cell cultures were pelleted at 3500×g for 20 min, and their supernatants were discarded. Cell pellets were lysed in 300 μL of 20 mM Tris, 1 mM MgSO₄, pH 7.5 with 1 g/L lysozyme and 0.5 g/L polymixin B sulfate by shaking at RT for 2 h. Samples were centrifuged at 3500×g for 20 min to clarify cellular debris, and the supernatant was used to carry out the transformations described in Examples 6 through 8, and Example 12.

### Example 4

### Production and Analysis of Engineered Phosphite Dehydrogenase Polypeptides

Plasmid libraries obtained through directed evolution and containing evolved phosphite dehydrogenase genes were transformed into E. coli W3110 and placed on Luria-Bertani (LB) agar medium containing 1% glucose and 30 μg/ml chloramphenicol (CAM). After incubation for at least 16 h at 30° C., colonies were picked using a Q-bot® robotic colony picker (Genetix) into a 96-well shallow well microtiter plate containing 200 μL of LB, 1% glucose, and 30 μg/ml CAM. Cells were grown 18-20 h at 30° C., with shaking at 200 rpm. Twenty L of this culture was then transferred to 360 μL of Terrific Broth (TB) and 30 μg/ml CAM. After incubation of deep well plates at 30° C. with shaking at 250 rpm for 2.5 h (OD₆₀₀ 0.6-0.8), recombinant gene expression was induced by isopropyl thioglycoside (IPTG) to a final concentration of 1 mM. The plates were then incubated at 30° C. with shaking at 250 rpm for 18-21 h.

Cell cultures were pelleted at 3500×g for 20 min, and their supernatants were discarded. Cell pellets were lysed in 300 μL of 20 mM Tris, pH 7.5 with 1 g/L lysozyme and 0.5 g/L polymixin B sulfate by shaking at RT for 2 h. Samples were centrifuged at 3500×g for 20 min to clarify cellular debris, and the supernatant was used to carry out the transformations described in Examples 9 through 12.

### Example 5

### KRED Variants of SEQ ID NO:2

E. coli KRED variants were generated as described in Example 1. To analyze the activity of the variants, 20 μL of supernatant produced as described in Example 2 were added to a mixture of 180 μL racemic alcohol substrate (50 g/L), with 4 g/L NAD⁺, 10 g/L commercially available NADH oxidase (NOx-9) and 100 mM FAD in 100 mM sodium phosphite pH 8.0. Reactions were incubated at 30° C. for 16-18h, and quenched via addition of 200 μL of 1M HCl. The quenched mixture was added to the sample and briefly mixed. Reaction samples were analyzed by UPLC to quantify residual substrate and products as described above. Significantly improved variants are provided in Table 5.1, below.

TABLE 5.1
Variants With Improved Activity Compared to SEQ ID NO: 2

| SEQ ID | Amino Acid Substitutions | |
|---|---|---|
| NO: | (Relative to SEQ ID NO: 2) | Improvement |
| 4 | R309F | +++ |
| 6 | C57L | +++ |
| 8 | G114K | +++ |
| 10 | G272V | +++ |
| 12 | G263Y | +++ |
| 14 | L276F | +++ |
| 16 | C57I | +++ |
| 18 | G272P | +++ |
| 20 | G272L | +++ |
| 22 | G114M | ++ |
| 24 | G272S | +++ |
| 26 | G272Q | +++ |
| 28 | G272H | +++ |
| 30 | G272T | +++ |
| 32 | G114H | +++ |
| 34 | G272I | +++ |
| 36 | C57X/W286X | +++ |
| 38 | G272W | ++ |
| 40 | I279H | ++ |
| 42 | G263H | +++ |
| 44 | H45R | +++ |
| 46 | S268M | ++ |
| 48 | S268W | ++ |
| 50 | L274V | ++ |
| 52 | V83I | ++ |
| 54 | Y52D | ++ |
| 56 | I279R | +++ |
| 58 | Y52S | +++ |
| 60 | I279Q | ++ |
| 62 | L274I | ++ |
| 64 | D56L | +++ |
| 66 | K110T | + |
| 68 | P228S | ++ |
| 70 | S138V/A146S/M258V/T289S | + |
| 72 | K211R | + |
| 74 | K37R | + |
| 76 | K37R/K211R/G229R | ++ |
| 78 | K211R/G229R | + |
| 80 | G229R | ++ |
| 82 | K37R/G229R | + |
| 84 | K37R/K211R | ++ |
| 86 | L276M | +++ |
| 88 | I79T/V83S/A275N/L276M | +++ |
| 90 | V83S/A275N/L276M | ++ |
| 92 | V83S/L276M | ++ |
| 94 | A275N/L276M | +++ |
| 96 | L55F/C57A/L276M | + |
| 98 | A104G | ++ |
| 100 | C57I/A104G/G114H | ++++ |
| 102 | C57L/A104G/G114H/G229R | ++++ |
| 104 | Y52S/C57L/G272H\/I279H/L296F | ++++ |
| 106 | Y52D/C57L/G272H | ++++ |
| 108 | Y52S/C57L/G272H/L274V/I279H/L296F | ++++ |
| 110 | Y52S/C57L/K110T/G272H/L296F | ++++ |

Key for Table 5.1

| | |
|---|---|
| ++++ | >6 |
| +++ | >4 and <6 |
| ++ | >2.5 and <4 |
| + | >1.2 and <2.5 |

### Example 6

### KRED Variants of SEQ ID NO:112

E. coli KRED variants were generated as described in Example 1. To analyze the activity of the variants, 5 μL supernatant produced as described in Example 3 were added to 95 μL of 0.3 M phosphite buffer pH 7.9 containing 0.25 mM NADPH; 19 g/L ketone substrate and 5 g/L PDH. Reactions were incubated at room temperature for 16-18 hours with gentle shaking. Reactions were quenched via addition of 100 μL of 1M HCl. The quenched mixture (10 μL) was diluted into 190 μL of water. Reaction samples (10 μL) were analyzed by HPLC to quantify residual substrates and products as described above. Significantly improved variants are provided in Table 6.1, below.

TABLE 6.1
Variants With Improved Activity and Selectivity
Compared to SEQ ID NO: 112

| SEQ ID | Amino Acid Substitutions | Activity | Selectivity |
|---|---|---|---|
| NO: | (Relative to SEQ ID NO: 112) | Improvement | Improvement |
| 114 | V24I/S220G/P314R/S315A | + | n.d. |
| 116 | V24I/T106P/S136A/S220G/ | + | n.d. |
| | L258V/C260A/P314R/S315A | | |
| 118 | V24I/T106P/F214L/A250V/ | + | n.d. |
| | L258V/C260A/P314R/S315A | | |
| 120 | T122E/I159V/L316E/I318L | ++ | n.d. |
| 122 | I159V/V251Q/Y272F/T277P/ | + | + |
| | L316E/I318L/I330L | | |
| 124 | N207G | +++ | +++ |
| 126 | N207G | +++ | +++ |
| 128 | V135F | ++ | ++ |
| 130 | V135F | ++ | ++ |
| 132 | I139V/N207S | +++ | +++ |

Key for Table 6.1

| | | |
|---|---|---|
| | Activity | Selectivity |
| +++ | >4 | >5 |
| ++ | >2.5 and <4 | >2 and <5 |
| + | >1.5 and <2.5 | >1 and <2 |

### Example 7

### KRED Variants of SEQ ID NO:124

E. coli KRED variants were generated as described in Example 1. To analyze the activity of the variants, 7.5 μL supernatant produced as described in Example 3 were added to 192.5 μL of 0.3 M phosphite buffer pH 7.9 containing 0.25 mM NADPH; 50 g/L ketone substrate and 5 g/L PDH. Reactions were incubated at room temperature for 16-18 hours with gentle shaking. Reactions were quenched via addition of 100 μL of 1M HCl. The quenched mixture (10 μL) was diluted into 190 μL of water. Reaction samples (10 μL) were analyzed by HPLC to quantify residual substrates and products as described above. Significantly improved variants are provided in Table 7.1, below.

TABLE 7.1
Variants With Improved Activity Compared to SEQ ID NO: 124

| SEQ ID | Amino Acid Substitutions | Activity |
|---|---|---|
| NO: | (Relative to SEQ ID NO: 124) | Improvement |
| 134 | V95T | +++ |
| 136 | V24I/V95T/M228T | +++ |
| 138 | V95T/V135F/I139V/G207N | ++++ |
| 140 | K3Y/V95T | +++ |
| 142 | K3Y/V95T/M228T/P314R | +++ |
| 144 | A2T/Y101P/A179L/T182M/ | ++ |
| | M228R/A238L/T282E | |
| 146 | I159V/M228L/K309Q/I330L | + |

Key for Table 7.1

| | |
|---|---|
| ++++ | >4 |
| +++ | >3 and <4 |
| ++ | >2 and <3 |
| + | >1.5 and <2 |

### Example 8

### KRED Variants of SEQ ID NO:138

E. coli KRED variants were generated as described in Example 1. To analyze the co-factor preference of the variants, four separate assays were utilized. First, 10 μL supernatant produced as described in Example 3 were added to 90 μL of 0.2 M phosphite buffer pH 7.9 containing 1 g/L ketone and 1 g/L of NADPH. The initial rate of NADPH consumption of the samples was analyzed via fluorescence with Ex λ=330 nm Em λ=445 nm, acquired for 180 seconds every 21 seconds.

Second, 20 μL supernatant produced as described in Example 3 were added to 190 μL of 0.2 M phosphite buffer pH 7.9 containing 1 g/L racemic alcohol and 2 g/L of NAD⁺. The initial rate of NAD⁺ consumption was analyzed via kinetic readings at UV 340 nm, data were acquired every 9 seconds for 5 minutes.

Third, 20 μL supernatant produced as described in Example 3 were added to 180 μL of 500 mM sodium phosphite containing 2 g/L imidazole ketone and 16.4 mM of NADPH; the samples were incubated at room temperature for 2 hr, shaking at 300 rpm. Reactions were quenched via addition of 200 μL of MeCN. After shaking for 5 minutes, 100 μL of the quenched reaction was transferred to a Millipore filter plate (45 micron pore size) with a co-star round bottom plate containing 100 μL of water to collect the filtrate and the mixture was spun at 4000 rpm for 2 minutes. Reaction samples (10 μL) were analyzed by HPLC to quantify residual substrate and product as described above.

Fourth, 20 μL superatant produced as described in Example 3 were added to 180 IL of 500 mM sodium phosphite containing 2 g/L imidazole ketone and 16.4 mM of NADPH; the samples were incubated at room temperature for 2 hr, shaking at 300 rpm. Reactions were quenched via addition of 200 μL of MeCN. After shaking for 5 minutes, 100 μL of the quenched reaction was transferred to a Millipore filter plate (45 micron pore size) with a co-star round bottom plate containing 100 μL of water to collect the filtrate and the mixture was spun at 4000 rpm for 2 minutes. Reaction samples (10 μL) were analyzed by HPLC to quantify residual substrate and product as described above. Co-factor specificity was calculated as

(amount of product generated with NADPH)/(amount of product generated with NADH)

Significantly improved variants are provided in Table 8.1, below.

TABLE 8.1
Variants With Improved Activity and Co-factor
Specificity Compared to SEQ ID NO: 138

| | | | Co-factor |
|---|---|---|---|
| SEQ ID | Amino Acid Substitutions | Activity | Specificity |
| NO: | (Relative to SEQ ID NO: 138) | Improvement | Improvement |
| 148 | V24I/A43V/S47E/L49N/A67V/ | + | + |
| | V68E/E70P/I91V/S220G | | |
| 150 | V24I/V68E/I91V/T218N/S220G | + | ++ |
| 152 | Y78F/P107G | + | ++ |
| 154 | K74A/Q75E/Y78F/A108V | ++ | +++ |
| 156 | Q75E/Y78F/N99P/ | ++ | +++ |
| | A108V/D215S/S224A | | |
| 158 | G19S | +++ | ++ |
| 160 | T95C | +++ | + |
| 162 | S96G | +++ | + |
| 164 | G19S | +++ | + |
| 166 | M72Q | +++ | ++ |
| 168 | A67W | + | ++ |
| 170 | N114V | + | ++ |

Key for Table 8.1

| | | |
|---|---|---|
| | Activity Improvement | Co-factor Specificity Improvement |
| +++ | >4 | >3 |
| ++ | >2 and <4 | >2 and <3 |
| + | >1 and <2 | >1 and <2 |

### Example 9

### PDH Variants of SEQ ID NO: 172

E. coli PDH variants were generated as described in Example 1. To analyze the activity of the variants, 5 μL supermatant produced as described in Example 3 were added to 95 μL of 0.5 M sodium phosphite buffer pH 7.9 containing 0.25 mM NADPH; 50 g/L ketone substrate and 2 g/L KRED of SEQ ID NO: 138. Reactions were incubated at 25° C. for 16-18 hours with gentle shaking. Reactions were quenched via addition of 100 μL of 1M HCL. The quenched mixture (10 μL) was diluted into 190 μL of water. Reaction samples (10 μL) were analyzed by HPLC to quantify residual substrates and products as described above. Significantly improved variants are provided in Table 9.1, below.

TABLE 9.1
Variants With Improved Activity Compared to SEQ ID NO: 172

| SEQ ID | Amino Acid Substitutions | Activity |
|---|---|---|
| NO: | (Relative to SEQ ID NO: 172) | Improvement |
| 174 | R10K/C73A/R137Q | + |
| 176 | R10K/C73A/F78Y/V233I/N323D | + |
| 178 | R137Q/V233I/E303A/N323D | + |
| 180 | R10K/C73A/F78Y/R137Q/N323D/V325A | + |
| 182 | R44A/R132Q/N145G | + |
| 184 | E13D/R41A/Q63A/R132Q/A193S/S195E | + |
| 186 | R41A/R44A/A88R/A193S/S195E | + |
| 188 | E266V | + |
| 190 | E266W | + |
| 192 | E266S | + |
| 194 | R44A/R132Q/P136D/R137Q/N145G/I293L | ++ |
| 196 | R44A/R132Q/R137I/N145G/V233I/A308V/N323D | ++ |
| 198 | R44A/R132Q/Q135A/P136D/R137I/N145G/I293L | ++ |
| 200 | R44A/R132Q/R137I/N145G/I293L/N323D | ++ |
| 202 | R44A/R132Q/N145G/S195E/I293L/N323D | ++ |
| 204 | R44A/V113S/R132Q/N145G | ++ |
| 206 | L18M/R44A/L119F/A124E/R132Q/R137I/N145G/I293L/N323D/A334K/C336R | +++ |
| 208 | R44A/L119F/R132Q/R137I/N145G/L158K/A175S/K177T/I293L/A317R/N323D | +++ |
| 210 | L18M/R44A/L119F/A124E/R132Q/R137I/N145G/L158K/K177T/I293L/N323D | +++ |
| 212 | L18M/R44A/L119F/A124E/R132Q/R137I/N145G/L158K/A175S/K177T/I293L/ | +++ |
| | A317R/N323D | |
| 214 | R44A/R69K/R120V/R132Q/R137I/N145G/A175T/S195E/I293L/N323D | +++ |
| 216 | S32V/R44A/R132Q/R137I/N145G/R186T/V233I/I293L/N323D/C336S | +++ |

Key for Table 9.1

| | |
|---|---|
| +++ | >4 |
| ++ | >2 and <4 |
| + | >1 and <2 |

### Example 10

### PDH Variants of SEQ ID NO:208

E. coli PDH variants were generated as described in Example 1. To analyze the co-factor preference of the variants, supernatant produced as described in Example 3 was diluted 4-fold with 50 mM Tris-HCl buffer, pH 7.5. Twenty μL of the diluted lysate was added to 180 μL of 0.1 M sodium phosphite buffer pH 7.9 and incubated overnight to consume residual NAD⁺ and NADP⁺ present in the lysate. The variants were then screened in three separate assays to analyze their co-factor specificity. First, for the initial rate NADP⁺ assay, 0.2 mM NADP⁺ in 0.1 M sodium phosphite buffer pH 7.9 was added and initial rate measured via fluorescence assay over 2 minutes. Second, for initial rate NAD⁺ assay, 0.2 mM NAD⁺ in 0.1M sodium phosphite buffer pH 7.9 was added and initial rate measured via fluorescence assay over 2 minutes. Third, a co-factor competition assay was performed. For this assay, 100 mM phosphite pH 7.9 containing 100 uM NADP, 1 mM NAD and 1 g/L NADH oxidase NOx-9 was added to the reaction. NOx-9 consumes all NADH immediately, leaving only NADPH signal, reduced by competition between NADP⁺ and NAD⁺. Reactions were quenched via addition of 100 μL of 1M HCl. The quenched mixture (10 μL) was diluted into 190 μL of water. Diluted reaction samples (10 μL) were analyzed by HPLC to quantify residual substrates and products as described above. Significantly improved variants are provided in Table 10.1, below.

TABLE 10.1
Variants With Improved Co-factor Specificity Compared to SEQ ID NO: 208

| SEQ ID | Amino Acid Substitutions | NADP+ Initial | Cofactor Specificity |
|---|---|---|---|
| NO: | (Relative to SEQ ID NO: 208) | Rate Improvement | Improvement |
| 218 | F78Y/F150I/F198L/R327S/L328P | + | + |
| 220 | N211A/D213Q/I322Q | + | + |
| 222 | A178P/C194L/N211A/D213Q/I322Q | + | + |
| 224 | F95I/N211A/D213Q/I322M | + | + |
| 226 | S32V/A59M/A124E/T177S/Q191H/R327D | + | + |
| 228 | L215P | + | + |
| 230 | L206N | + | + |
| 232 | T104F | ++ | + |
| 234 | T104L | ++ | + |
| 236 | E266S | + | + |
| 238 | V262P | + | + |
| 240 | V262D | + | + |
| 242 | V83A/E266A | + | + |
| 244 | D323N | + | + |

Key for Table 10.1

| | |
|---|---|
| ++ | >2 |
| + | >1 and <2 |

### Example 11

### Additional PDH Variants of SEQ ID NO:208

E. coli PDH variants were generated as described in Example 1. To analyze the co-factor preference of the variants, supernatant produced as described in Example 3 was diluted 4-fold with 50 mM Tris-HCl buffer, pH 7.5. Twenty μL of the diluted lysate was added to 180 μL of 0.1 M sodium phosphite buffer pH 7.9 and incubated overnight to consume residual NAD⁺ and NADP⁺ present in the lysate. The variants were then screened in three separate assays to analyze their co-factor specificity. First, for the initial rate NADP⁺ assay 0.2 mM NADP⁺ in 0.1 M sodium phosphite buffer pH 7.9 was added and initial rate measured via fluorescence assay over 2 minutes. Second, for initial rate NAD⁺ assay, 0.2 mM NAD⁺ in 0.1M sodium phosphite buffer pH 7.9 was added and initial rate measured via fluorescence assay over 2 minutes. Third, a co-factor competition assay was performed. For this assay, three μL of pre-incubated lysate was added to 97 μL of 200 mM phosphite pH 7.9 containing 2 mM NAD, 0.2 mM NADP, 2 g/L KRED of SEQ ID NO:138, 4 g/L KRED of SEQ ID NO: 104 and 10 g/L ketone (2). Reactions were quenched via addition of 100 μL of 1M HCl. The quenched mixture (10 μL) was diluted into 190 μL of water. Diluted reaction samples (10 μL) were analyzed by reverse phase HPLC to quantify residual substrate and both enantiomers of the product as described above. Significantly improved variants are provided in Table 11.1, below.

| SEQ ID | Amino Acid Substitutions | Cofactor Specificity |
|---|---|---|
| NO: | (Relative to SEQ ID NO: 208) | Improvement |
| 246 | V83A/T104L/L206N | +++ |
| 248 | A74T/V83A/L206N | ++ |
| 250 | T104L/V262L | +++ |
| 252 | T104L/L206N | +++ |
| 254 | S295R | +++ |
| 256 | V96G | +++ |
| 258 | T104M | + |

Key for Table 11.1

| | |
|---|---|
| +++ | >8 |
| ++ | >4 and <8 |
| + | >2 and <4 |

### Example 12

### Production of Engineered Polypeptides and Performance Validation

Plasmids comprising variants obtained through directed evolution of the KRED of SEQ ID NO:2 and containing evolved ketoreductase genes were transformed into E. coli W3110 and placed on Luria-Bertani (LB) agar medium containing 1% glucose and 30 μg/ml chloramphenicol (CAM). After incubation for at least 16 h at 30° C., a single colony was picked into 5 mL of LB, 1% glucose, and 30 μg/ml CAM. Cells were grown 18-20 h at 30° C., with shaking at 250 rpm. This culture was then transferred into Terrific Broth (TB), 2 mM ZnSO₄, 1 mM MgSO₄, and 30 μg/ml CAM at a final OD₆₀₀ of ˜0.02 and a final volume of 250 mL. After incubation of the flasks at 30° C. with shaking at 250 rpm for 3.5 h (OD₆₀₀ 0.6-0.8), recombinant gene expression was induced by isopropyl thioglycoside (IPTG) to a final concentration of 1 mM. The flask was then incubated at 30° C. with shaking at 250 rpm for 18-21 h. Cells were pelleted at 3500×g for 20 min, and the supernatant was discarded. The cell pellet was washed in 50 mL ice cold 50 mM sodium phosphate pH 7.5 containing 2 mM ZnSO₄ and 1 mM MgSO₄, resuspended in 30 ml of the same buffer, and lysed using a cell disruptor at 18-20 kpsi. Lysates were clarified at 10000×g for 60 min, and clarified supernatants were lyophilized to an off white powder.

Plasmids of comprising variants obtained through directed evolution of the KRED of SEQ ID NOS: 112 and 138, and containing evolved ketoreductase genes were transformed into E. coli W3110 and placed on Luria-Bertani (LB) agar medium containing 1% glucose and 30 μg/ml chloramphenicol (CAM). After incubation for at least 16 h at 30° C., a single colony was picked into 5 mL of LB, 1% glucose, and 30 μg/ml CAM. Cells were grown 18-20 h at 30° C., with shaking at 250 rpm. This culture was then transferred into Terrific Broth (TB), and 30 μg/ml CAM at a final OD₆₀₀ of ˜0.02 and a final volume of 250 mL. After incubation of the flasks at 30° C. with shaking at 250 rpm for 3.5 h (OD₆₀₀ 0.6-0.8), recombinant gene expression was induced by isopropyl thioglycoside (IPTG) to a final concentration of 1 mM. The flask was then incubated at 30° C. with shaking at 250 rpm for 18-21 h. Cells were pelleted at 3500×g for 20 min, and the supernatant was discarded. The cell pellet was washed in 50 mL ice cold 50 mM sodium phosphate pH 7.5, resuspended in 30 ml of the same buffer, and lysed using a cell disruptor at 18-20 kpsi. Lysates were clarified at 10000×g for 60 min, and clarified supernatants were lyophilized to an off white powder.

Plasmids comprising variants obtained through directed evolution of the PDH of SEQ ID NOS: 172 and 208, and containing evolved phosphite dehydrogenase genes were transformed into E. coli W3110 and placed on Luria-Bertani (LB) agar medium containing 1% glucose and 30 μg/ml chloramphenicol (CAM). After incubation for at least 16 h at 30° C., a single colony was picked into 5 mL of LB, 1% glucose, and 30 μg/ml CAM. Cells were grown 18-20 h at 30° C., with shaking at 250 rpm. This culture was then transferred into Terrific Broth (TB), and 30 μg/ml CAM at a final OD₆₀₀ of ˜0.02 and a final volume of 250 mL. After incubation of the flasks at 30° C. with shaking at 250 rpm for 3.5 h (OD₆₀₀ 0.6-0.8), recombinant gene expression was induced by isopropyl thioglycoside (IPTG) to a final concentration of 1 mM. The flask was then incubated at 30° C. with shaking at 250 rpm for 18-21 h. Cells were pelleted at 3500×g for 20 min, and the supernatant was discarded. The cell pellet was washed in 50 mL ice cold 50 mM sodium phosphate pH 7.5, resuspended in 30 ml of the same buffer, and lysed using a cell disruptor at 18-20 kpsi. Lysates were clarified at 10000×g for 60 min, and clarified supernatants were lyophilized to an off white powder.

To evaluate the final compound under process like conditions, 50 g/L of racemic alcohol substrate in 500 mM sodium phosphite buffer pH 7.9, 0.1 g/L NAD, 0.1 g/L NADP, 2.5 g/L KRED of SEQ ID NO: 104, 10 g/L commercially available NADH oxidase NOx-9, 2.5 g/L KRED of SEQ ID: 154, 10 g/L PDH of SEQ ID NO:250 was stirred under stream of oxygen with 1% v/v antifoam at room temperature for 24 hours resulting in 93% conversion of substrate and 99.5% enantiomeric excess of (R)-alcohol 1a. Reaction samples were analyzed by reverse phase HPLC to quantify residual substrate and products as described above.

While various specific embodiments have been illustrated and described, it will be appreciated that various changes can be made without departing from the spirit and scope of the invention(s).

All publications, patents, patent applications and other documents cited in this application are hereby incorporated by reference in their entireties for all purposes to the same extent as if each individual publication, patent, patent application or other document were individually indicated to be incorporated by reference for all purposes.

1. An engineered phosphite dehydrogenase variant having at least 85%, 90%, 91%, 92%, 93%, 94%, 95%, 96%, 97%, 98%, 99% or more sequence identity to the polypeptide sequence SEQ ID NO: 172 and/or 208.

2. The engineered phosphite dehydrogenase variant of claim 1, comprising a polypeptide sequence having at least 85%, 90%, 91%, 92%, 93%, 94%, 95%, 96%, 97%, 98%, 99% or more sequence identity to SEQ ID NO: 172, and at least one substitution or substitution set at one or more positions selected from positions 10/73/78/137/323/325, 10/73/78/233/323, 10/73/137, 13/41/63/132/193/195, 18/44/119/124/132/137/145/158/175/177/293/317/323, 18/44/119/124/132/137/145/158/177/293/323, 18/44/119/124/132/137/145/293/323/334/336, 32/44/132/137/145/186/233/293/323/336, 41/44/88/193/195, 44/69/120/132/137/145/175/195/293/323, 44/113/132/145, 44/119/132/137/145/158/175/177/293/317/323, 44/132/135/136/137/145/293, 44/132/136/137/145/293, 44/132/137/145/233/308/323, 44/132/137/145/293/323, 44/132/145, 44/132/145/195/293/323, 137/233/303/323, and 266, wherein said positions are numbered with reference to SEQ ID NO: 172.

3. The engineered phosphite dehydrogenase variant of claim 1, comprising a polypeptide sequence having at least 85%, 90%, 91%, 92%, 93%, 94%, 95%, 96%, 97%, 98%, 99% or more sequence identity to SEQ ID NO:208, and at least one substitution or substitution set at one or more positions selected from positions 32/59/124/177/191/327, 78/150/198/327/328, 83/266, 95/211/213/322, 104, 178/194/211/213/322, 206, 211/213/322, 215, 262, 266, and 323, wherein said positions are numbered with reference to SEQ ID NO:208.

4. The engineered phosphite dehydrogenase variant of claim 1, comprising a polypeptide sequence comprising a sequence having at least 90% sequence identity to SEQ ID NO:172 and/or 208.

5. The engineered phosphite dehydrogenase variant of claim 1, comprising a polypeptide sequence comprising a sequence having at least 95% sequence identity to SEQ ID NO:172 and/or 208.

6. The engineered phosphite dehydrogenase variant of claim 1, comprising a polypeptide sequence set forth in SEQ ID NO: 172 or 208.

7. The engineered phosphite dehydrogenase variant of claim 1, wherein said engineered phosphite dehydrogenase variant comprises a polypeptide sequence encoding a variant provided in Table 9.1, 10.1, and/or 11.1.

8. The engineered phosphite dehydrogenase variant of claim 1, wherein said engineered phosphite dehydrogenase variant comprises a polypeptide sequence selected from the even-numbered sequences set forth in SEQ ID NOS: 172 to 260.

9. A composition comprising at least one phosphite dehydrogenase variant set forth in claim 1.

10. An engineered polynucleotide sequence encoding the engineered phosphite dehydrogenase variant of claim 1.

11. The engineered polynucleotide sequence of claim 10, wherein said sequence comprises a polynucleotide sequence that is at least 85%, 90%, 91%, 92%, 93%, 94%, 95%, 96%, 97%, 98%, 99% or more identical to a sequence selected from the odd-numbered sequences set forth in SEQ ID NOS: 171 to 259.

12. A vector comprising the engineered polynucleotide sequence of claim 10.

13. The vector of claim 12, further comprising at least one control sequence.

14. A host cell comprising the vector of claim 12.

15. A method for producing the engineered phosphite dehydrogenase variant of claim 1, comprising culturing a host cell, under conditions that said engineered phosphite dehydrogenase variant is produced by said host cell.

16. The method of claim 15, further comprising the step of recovering said engineered phosphite dehydrogenase variant produced by said host cell.

17. A method for deracemization of a chiral alcohol comprising at least one engineered ketoreductase variant, comprising at least one engineered phosphite dehydrogenase variant of claim 1, at least one chiral alcohol, and at least one co-factor, under conditions such that said chiral alcohol is deracemized.

18. The method of claim 17, wherein said method is conducted in one pot reaction.
